SEQUENCE LISTING (70 sequences)

SEQ ID NO: 1 - DNA, 4526 bp, Homo sapiens
acggctgcgcagatgccgactttagaggaggcggagtttcggccttcgcctgctggaaaagcagtaggatcggccagtggcgacagcaggagctgagcctaagccctggcggggctttgggctgtagattcctgtctgactaaagggacctcaaaaaggagggaaaatggcttctgagtctgaaactctgaatcccagtgctaggataatgaccttttatccaactatggaagagttccgaaacttcagtagatacattgcctacattgaatcccaaggagctcatcgggcagggctagccaaggttgttcctccaaaagagtggaagccacgagcatcctatgatgacattgatgatttggtcattcctgcccccattcaacagctggtgacggggcagtctggcctctttactcagtacaacatacagaagaaagccatgactgttcgagagttccgcaagatagccaatagcgataagtactgtaccccacgctatagtgagtttgaagagctcgagcggaaatactggaaaaatcttacattcaatcctccaatctatggtgcagatgtgaatggtaccctctatgaaaagcatgttgatgagtggaatattggccggctgagaacaatcctggacttggtggaaaaggagagtgggatcaccattgagggtgtgaacaccccatacctgtactttggcatgtggaagacatcctttgcttggcacactgaagacatggacctctacagcatcaactacctgcactttggagaaccaaagtcctggtactctgttccacctgagcatggaaagcggttggaacgcctcgccaaaggctttttcccaggaagtgctcaaagctgtgaggcatttctccgccacaagatgaccctgatttccccgttaatgctgaagaaatatggaattccctttgacaaggtgactcaagaggctggagagtttatgatcactttcccttatggttaccatgccggctttaaccatggttttaactgtgcggagtctaccaattttgctacccgtcggtggattgagtacggcaagcaagctgtgctgtgctcctgtagaaaggacatggtgaagatctccatggatgtgtttgtgagaaagttccagccagaaaggtacaaactttggaaagctgggaaggacaacacagttattgaccatactctgcccacgccagaagcagctgagtttcttaaggagagtgaactgcctccaagagctggcaacgaggaggagtgcccagaggaggacatggaaggggtggaggatggagaggaaggagacctgaagacaagcctggccaagcaccgaatagggacaaagaggcaccgagtttgtcttgaaataccacaggaggtgagtcagagtgagctcttccccaaggaggatctgagttctgagcagtatgagatgacggagtgcccggcagccctcgcccctgtgaggcccacccatagctctgtgcggcaagttgaggatggtcttaccttcccagattattctgactccactgaagtcaaatttgaagagcttaaaaatgtcaaactagaagaggaggatgaggaggaagaacaagcagcagctgccttggatctttctgtgaatcctgcgtctgtagggggacgccttgtcttctcaggctccaaaaagaaatcatcttctagcctgggctctggctcttcacgggattctatctcttctgattcagaaactagtgagcctctctcctgccgagcccaagggcaaacgggagttctcactgtgcacagttatgccaaaggggatggcagggtcactgtgggagagccatgcacgaggaagaaaggaagcgccgctagaagtttcagtgagcgggagctggcagaggttgcagatgaatacatgttttccctagaagagaataagaagtccaagggacgccgtcagcctttaagcaagctcccccgccatcacccacttgtgctgcaggagtgtgtcagtgatgatgagacatctgaacagctgacccctgaggaagaggctgaggagacagaggcctgggccaagcctctgagccaactgtggcagaaccgacctccaaactttgaggctgagaaggaattcaatgagaccatggcccaacaggcccctcactgcgctgtctgtatgatcttccagacttatcatcaggttgaatttggaggctttaatcagaactgtggaaatgcttcagatttagccccccagaagcagaggaccaagccattgattccagaaatgtgcttcacttcgactggctgcagcacggacatcaacctttctactccttatcttgaggaggatggcaccagcatactcgtttcctgcaagaagtgcagcgtccgggtccatgccagttgctatggggtcccccctgcaaaggcttctgaagactggatgtgttctcggtgttcagccaatgccctagaggaggactgctgtttatgctcattacgaggaggggccctgcagagagcaaatgatgacaggtgggtccacgtttcatgtgctgtggcaattctggaagcaaggtttgtcaacattgcagaaagaagtccggtggatgtgagcaaaatccccctgccccgcttcaaactgaaatgtatcttctgtaagaagcggaggaaaagaactgctggctgctgtgtgcagtgttctcacggccgctgcccaactgccttccatgtgagctgcgcccaggctgccggtgtgatgatgcagcctgacgactggccttttgtggtcttcattacctgctttcggcacaagattcctaatttggagcgtgccaagggggccttgcaaagcatcactgcaggccagaaagtcattagcaagcataagaacgggcgcttctaccagtgtgaagtggtcaggctcaccaccgagaccttctatgaagtcaactttgatgatggctccttcagcgacaatctttatcctgaggacatagtgagccaggactgtctccagtttggtcctcctgctgaaggggaagtggtccaagtgagatggacagacggccaagtctatggagccaagtttgtggcctcccaccctatccaaatgtaccaggtggagtttgaggatggctcacaacttgtggttaagagagatgatgtatacacactggatgaagagcttcccaagagagtcaaatctagactgtcagtagcctcagacatgcgcttcaatgagattttcacagagaaagaggttaagcaagaaaagaaacggcaacgagttatcaactcaagataccgggaagattatattgagcctgcactataccgggccatcatggagtaggtgcttccagggtccaagggattctcagccatccaggcaagagcactctgggttccacagcacagcagacatggaacgctgaagtctctgaaagtgaagttgtaaaaagaaaaggaatgaaataaccgacccatcatcttctcacccaccctcattgcattccgctgtagtgaaaggacgagccatttctgggcacgtggcagcagtcgctgatctcccagctgaggggctgagcactggaatgctgtggctgcactggccccagtccatagaggggtcaactatgctggctggactggctgccttgttcctggcctaggacttagcttcataactatcacctgcaccgactaggctgaggtgctggtacttgccccaacccctacttttgtatttatatgtgtgtgtgtgtgtgcgtgcgtgcgtgcgtgcgtgtatgtttggtctggaccagcttctgccagcccctggcctttactttcttccttgcctatgcagggcaaacaaaatgtgaaattctgccctcagctgagctgagtaagggctcctgggggttggctggagatgggtgtggcatctgtccaggcctggaaccgtctcaagacagtgctggcaaagctgcagtattgagatgctaaggagctgatgccacctctttgtcttcccctaaaggagaacatggggataacatgggtgtgtgcccacaacactctaggtgcagagcccctgtggcaaagtattacagggtgtgggtggggattaccctgaatcggggattttaatgatggaagcaggcagagcctggtgggtgattctgtcaacagaaaattgcaatcatgcaggggctgggagggttaggatgaaaaaactggggccattggaggcccactgtaggtgggagggagctgattttggggtggggggtgggactagagggcaatactgaaggggttaaacaggtttttgctcctcaagaatttgtttgcctgggcccaggattggagggcttcacaccaataccctgtgtatacaagaatcagatttataatacttccccttttttgttacgtatgaacactataaaccaaattattttgaaaactggtgcatcaccttgtccttagcaataaaatgtgttgagcagaggaaaaaaaaaaaaaaaaaa

SEQ ID NO: 2 - DNA, 5675 bp, Homo sapiens
agggctcggtcgccagcaaccgagcggggcccggcccgagcggggcctgggggtgcgacgccgagggcgggggagagcgcgccgctgctcccggaccgggccgcgcacgccgcctcaggaaccatcactgttgctggaggcacctgacaaatcctagcgaatttttggagcatctccacccaggaacctcgccatccagaagtgtgcttcccgcacagctgcagccatggggtctgaggaccacggcgcccagaaccccagctgtaaaatcatgacgtttcgcccaaccatggaagaatttaaagacttcaacaaatacgtggcctacatagagtcgcagggagcccaccgggcgggcctggccaagatcatccccccgaaggagtggaagccgcggcagacgtatgatgacatcgacgacgtggtgatcccggcgcccatccagcaggtggtgacgggccagtcgggcctcttcacgcagtacaatatccagaagaaggccatgacagtgggcgagtaccgccgcctggccaacagcgagaagtactgtaccccgcggcaccaggactttgatgaccttgaacgcaaatactggaagaacctcacctttgtctccccgatctacggggctgacatcagcggctctttgtatgatgacgacgtggcccagtggaacatcgggagcctccggaccatcctggacatggtggagcgcgagtgcggcaccatcatcgagggcgtgaacacgccctacctgtacttcggcatgtggaagaccaccttcgcctggcacaccgaggacatggacctgtacagcatcaactacctgcactttggggagcctaagtcctggtacgccatcccaccagagcacggcaagcgcctggagcggctggccatcggcttcttccccgggagctcgcagggctgcgacgccttcctgcggcataagatgaccctcatctcgcccatcatcctgaagaagtacgggatccccttcagccggatcacgcaggaggccggggaattcatgatcacatttccctacggctaccacgccggcttcaatcacgggttcaactgcgcagaatctaccaacttcgccaccctgcggtggattgactacggcaaagtggccactcagtgcacgtgccggaaggacatggtcaagatctccatggacgtgttcgtgcgcatcctgcagcccgagcgctacgagctgtggaagcagggcaaggacctcacggtgctggaccacacgcggcccacggcgctcaccagccccgagctgagctcctggagtgcatcccgggcctcgctgaaggccaagctcctccgcaggtctcaccggaaacggagccagcccaagaagccgaagcccgaagaccccaagttccctggggagggtacggctggggcagcgctcctagaggaggctgggggcagcgtgaaggaggaggctgggccggaggttgaccccgaggaggaggaggaggagccgcagccactgccacacggccgggaggccgagggcgcagaagaggacgggaggggcaagctgcggccaaccaaggccaagagcgagcggaagaagaagagcttcggcctgctgcccccacagctgccgcccccgcctgctcacttcccctcagaggaggcgctgtggctgccatccccactggagcccccggtgctgggcccaggccctgcagccatggaggagagccccctgccggcaccccttaatgtcgtgccccctgaggtgcccagtgaggagctagaggccaagcctcggcccatcatccccatgctgtacgtggtgccgcggccgggcaaggcagccttcaaccaggagcacgtgtcctgccagcaggcctttgagcactttgcccagaagggtccgacctggaaggaaccagtttcccccatggagctgacggggccagaggacggtgcagccagcagtggggcaggtcgcatggagaccaaagcccgggccggagaggggcaggcaccgtccacattttccaaattgaagatggagatcaagaagagccggcgccatcccctgggccggccgcccacccggtccccactgtcggtggtgaagcaggaggcctcaagtgacgaggaggcatcccctttctccggggaggaagatgtgagtgacccggacgccttgaggccgctgctgtctctgcagtggaagaacagggcggccagcttccaggccgagaggaagttcaacgcagcggctgcgcgcacggagccctactgcgccatctgcacgctcttctacccctactgccaggccctacagactgagaaggaggcacccatagcctccctcggagagggctgcccggccacattaccctccaaaagccgtcagaagacccgaccgctcatccctgagatgtgcttcacctctggcggtgagaacacggagccgctgcctgccaactcctacatcggcgacgacgggaccagccccctgatcgcctgcggcaagtgctgcctgcaggtccatgccagttgctatggcatccgtcccgagctggtcaatgaaggctggacgtgttcccggtgcgcggcccacgcctggactgcggagtgctgcctgtgcaacctgcgaggaggtgcgctgcagatgaccaccgataggaggtggatccacgtgatctgtgccatcgcagtccccgaggcgcgcttcctgaacgtgattgagcgccaccctgtggacatcagcgccatccccgagcagcggtggaagctgaaatgcgtgtactgccggaagcggatgaagaaggtgtcaggtgcctgtatccagtgctcctacgagcactgctccacgtccttccacgtgacctgcgcccacgccgcaggcgtgctcatggagccggacgactggccctatgtggtctccatcacctgcctcaagcacaagtcggggggtcacgctgtccaactcctgagggccgtgtccctaggccaggtggtcatcaccaagaaccgcaacgggctgtactaccgctgtcgcgtcatcggtgccgcctcgcagacctgctacgaagtgaacttcgacgatggctcctacagcgacaacctgtaccctgagagcatcacgagtagggactgtgtccagctgggacccccttccgagggggagctggtggagctccggtggactgacggcaacctctacaaggccaagttcatctcctccgtcaccagccacatctaccaggtggagtttgaggacgggtcccagctgacggtgaagcgtggggacatcttcaccctggaggaggagctgcccaagagggtccgctctcggctgtcactgagcacgggggcaccgcaggagcccgccttctcgggggaggaggccaaggccgccaagcgcccgcgtgtgggcaccccgcttgccacggaggactccgggcggagccaggactacgtggccttcgtggagagcctcctgcaggtgcagggccggcccggagcccccttctaggacagctggccgctcaggcgaccctcagcccggcggggaggccatggcatgccccgggcgttcgcttgctgtgaattcctgtcctcgtgtccccgacccccgagaggccacctccaagccgcgggtgccccctagggcgacaggagccagcgggacgccgcacgcggccccagactcagggagcagggccaggcgggctcgggggccggccaggggagcaccccactcaactactcagaattttaaaccatgtaagctctcttcttctcgaaaaggtgctactgcaatgccctactgagcaacctttgagattgtcacttctgtacataaaccacctttgtgaggctctttctataaatacatattgtttaaaaaaaagcaagaaaaaaaggaaaacaaaggaaaatatccccaaagttgttttctagatttgtggctttaagaaaaacaaaacaaaacaaacacattgtttttctcagaaccaggattctctgagaggtcagagcatctcgctgtttttttgttgttgttttaaaatattatgatttggctacagaccaggcagggaaagagacccggtaattggagggtgagcctcggggggggggcaggacgccccggtttcggcacagcccggtcactcacggcctcgctctcgcctcaccccggctcctgggctttgatggtctggtgccagtgcctgtgcccactctgtgcctgctgggaggaggcccaggctctctggtggccgcccctgtgcacctggccaggggaagcccgggggtctggggcctccctccgtctgcgcccacctttgcagaataaactctctcctggggtttgtctatctttgtttctctcacctgagagaaacgcaggtgttccagaggcttccttgcagacaaagcacccctgcacctcctatggctcaggatgagggaggcccccaggcccttctggttggtagtgagtgtggacagcttcccagctcttcgggtacaaccctgagcaggtcgggggacacagggccgaggcaggccttcggggcccctttcgcctgcttccgggcagggacgaggcctggtgtcctcgctccacccacccacgctgctgtcacctgaggggaatctgcttcttaggagtgggttgagctgatagagaaaaaacggccttcagcccaggctgggaagcgccttctccaggtgcctctccctcaccagctctgcacccctctggggagccttccccaccttagctgtctcctgccccagggagggatggaggagataatttgcttatattaaaaacaaaaaatggctgaggcaggagtttgggaccagcctgggctatatagcaagaccccatcactacaaattttttacaaattagctaggtgtggtggtgcgcacctgtggtcccagctactcgggaggctgtggtgggaggattgcttgagtccaggaggttgaggctgcagtcagctcagattgcaccactgcactccagcctgggcaacagagcgagaccctgtctccaaaaaaaaaaaaaagcaatgtttatattataaaagagtgtcctaacagtccccgggctagagaggactaaggaaaacagagagagtgttacgcaggagcaagcctttcatttccttggtgggggaggggggcggttgccctggagagggccggggtcggggaggttggggggtgtcagccaaaacgtggaggtgtccctctgcacgcagccctcgcccggcgtggcgctgacactgtattcttatgttgtttgaaaatgctatttatattgtaaagaagcgggcgggtgcccctgctgcccttgtcccttgggggtcacacccatcccctggtgggctcctgggcggcctgcgcagatgggccacagaagggcaggccggagctgcacactctccccacgaaggtatctctgtgtcttactctgtgcaaagacgcggcaaaacccagtgccctggtttttccccacccgagatgaaggatacgctgtattttttgcctaatgtccctgcctctaggttcataatgaattaaaggttcatgaacgctgcgaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 4687 bp, Homo sapiens
gccataggtgcgcgtcggcgcccaggaggacgtgtggcgcgtggactacatcaggtccagccctgcgggaccccagccagcgcttccgggcaaggttctgtgcacctgttttctccttctacgcgagtatctttcccctccggaaagaatgggatatgcctgtgtccaaaggacaagaagatgcgcgccagcaagcctaagttaaccacagcgcggaagttgagcccaaagcaagagcgtgccgggcacctttaagctgtttgtaagcccacgtgactcaccaagtgcgggccccagcggtcacgtgacggcgcgcgcgccctcgcgcagggagagccggcggtgcgcgcgccttcgccgctgcctcccacccaccccctcgacgggagggtgaggcgcggcgcagtgatcgggcggccggggtcctgtgcgcgtgcgcagcgaacagctgtcacctagtgcggaacaagtctcccaaatttcccaaatctccctgggccggaggccactgtcttctcttcctcctccaccgagtcgtgctctcgccccaacccgcgcgccagacactgccctaaccatcatggaggtggccgaggtggaaagtcctctgaaccccagctgtaagataatgaccttcagaccctccatggaggagttccgggagttcaacaaataccttgcatacatggagtctaaaggagcccatcgtgcgggtcttgcaaaggtgattcctcctaaggagtggaagccaagacagtgctatgatgacattgataatttgctcattccagcaccaattcagcagatggtcacagggcagtcaggactgttcactcagtacaacatccagaaaaaagcgatgactgtgaaggagttcaggcagctggccaacagtggcaaatattgtactccaagatacttggattacgaagatttggagcgcaagtactggaagaacttaacttttgtggcacctatctatggtgcagatattaatgggagcatatatgatgagggtgtggatgaatggaacatagctcgcctcaatacagtcttggatgtggttgaagaagagtgtggcatttctattgagggtgtaaataccccatatctctattttggcatgtggaagaccacgtttgcatggcacaccgaagacatggacctctatagcattaattatctccactttggagagcccaagtcttggtatgctatacctccggagcatggaaaacgacttgaaagactagctcaaggttttttcccaagcagctcccaagggtgtgatgcatttcttcgccacaagatgacattgatttctccatcagtattgaagaaatatggtattccctttgacaagataacccaggaggctggagaattcatgatcactttcccatatggctaccatgctggttttaatcatggtttcaactgtgcagaatctacaaattttgctactgtcagatggattgactatggaaaagttgccaaattgtgcacttgcaggaaagacatggtgaagatttcaatggatatctttgtgaggaaatttcagccagacagatatcagctttggaaacaaggaaaggatatatacaccattgatcacacgaagcctactccagcatccacccctgaagtaaaagcatggctgcagaggaggaggaaagtaagaaaagcatcccgaagcttccagtgtgctaggtctacctctaaaaggcctaaggctgatgaggaagaggaagtgtcagatgaagtcgatggggcagaggtccctaaccccgactcagtcacagatgacctcaaggtcagtgaaaagtcagaagcagcagtgaagctgaggaacacagaagcatcttcagaagaagagtcatctgctagcaggatgcaggtggagcagaatttatcagatcatatcaaactctcaggaaacagctgcttaagtacatctgtaacagaagacataaaaactgaggatgacaaagcttatgcatatagaagtgtaccttctatatccagtgaggctgatgattccattccattgtctagtggctatgagaagcccgagaaatcagacccatccgagctttcatggccaaagtcacctgagtcatgctcatcagtggcagagagtaatggtgtgttaacagagggagaagagagtgatgtggagagccatgggaatggccttgaacctggggaaatcccagcggtccccagtggagagagaaatagcttcaaagtccccagtatagcagagggagagaacaaaacctctaagagttggcgccatccacttagcaggcctccagcaagatctccgatgactcttgtgaagcagcaggcgccaagtgatgaagaattgcctgaggttctgtccattgaggaggaagtggaagaaacagagtcttgggcgaaacctctcatccacctttggcagacgaagtcccctaacttcgcagctgagcaagagtataatgcaacagtggccaggatgaagccacactgtgccatctgcactctgctcatgccgtaccacaagccagatagcagcaatgaagaaaatgatgctagatgggagacaaaattagatgaagtcgttacatcggagggaaagactaagcccctcataccagagatgtgttttatttatagtgaagaaaatatagaatattctccacccaatgccttccttgaagaggatggaacaagtctccttatttcctgtgcaaagtgctgcgtacgggttcatgcaagttgttatggtattccttctcatgagatctgtgatggatggctgtgtgcccggtgcaaaagaaatgcgtggacagcagaatgctgtctctgcaatttgagaggaggtgctcttaagcaaacgaagaacaataagtgggcccatgtcatgtgcgccgttgcggtcccagaagttcgattcactaatgtcccagaaaggacacaaatagatgtaggcagaatacctttacagaggttaaaattgaaatgcatcttctgcagacaccgggttaagagggtctctggagcctgcatccagtgttcctacggtcgctgcccggcctccttccatgtcacttgtgcccatgctgctggggtactgatggagcctgatgactggccttatgtggtgaacattacatgctttcgacataaggtcaaccccaacgtgaagtccaaggcttgcgagaaggtcatttccgtgggtcaaacggtcatcacgaagcatcggaacacccggtattacagttgcagagtgatggctgtgacatcgcagaccttctatgaggtcatgtttgatgatggctcctttagcagagacacatttcctgaggatatcgtgagccgagactgtctgaagctgggcccacctgctgagggagaagtcgtccaagtcaagtggcccgatggcaaactctatggagcaaaatattttggatcaaatattgcccacatgtaccaggttgagtttgaagatggatcccagatagcaatgaagagagaggacatctacactttagatgaagagttacccaagagagtgaaagctcgattttccacagcctctgacatgcgatttgaagacacgttttatggagcagacattatccaaggggagagaaagagacaaagagtgctgagctccaggtttaagaatgaatatgtggccgaccctgtataccgcacttttttgaagagctctttccagaagaagtgccagaagagacagtagtctgcatacatcgctgcaggccacagagcagcttgggttggaagagagaagatgaagggacatccttggggctgtgccgtgagttttgctggcataggtgacagggtgtgtctctgacagtggtaaatcgggtttccagagtttggtcaccaaaaatacaaaatacacccaatgaattggacgcagcaatctgaaatcatctctagtcttgctttcacttgtgagcagttgtcttctatgatcccaaagaagttttctaagtgaaaggaaatactagtgaatcacccacaaggaaaagccactgccacagaggaggcgggtccccttgtgcggcttagggccctgtcaggaaacacacggggacctctctctctagctccagcaggtggcacctcggtacccagcgggtagggcgataatttatatattttccacagtcagggaaggactctcacttatttgtttcaaattgcagtttttataaaacatttttaaaacacaaatggcatgtatgctaatgagatttacccgtgtgctatctgtatttcccttgtacagaacttttacatttttgaatattcctattacttttgattgtgtctgatgggaactgagttgttggcctttgtgaaatgaaatttttggctcttgagaaagaattcttatgaattgttatgcgaattttatatatttaaagagggagatctggggctgttatttttaaacactttttttcataatacatattccgagtagatatttataaaatatatgtttctttcattatgtgtttgtaaaattagagtttaaataaatatgctttgatgcatagttttgaactaatgtaacatgatttttcttttttaaaacagcctgaaaatgtactagtgtttaaaaataaagatttccattttctccaaaaaaaaaaaaaaaaaa

SEQ ID NO: 4 - DNA, 1572 bp, Homo sapiens
atggaaactatgaagtctaaggccaactgtgcccagaatccaaattgtaacataatgatatttcatccaaccaaagaagagtttaatgattttgataaatatattgcttacatggaatcccaaggtgcacacagagctggcttggctaagataattccacccaaagaatggaaagccagagagacctatgataatatcagtgaaatcttaatagccactcccctccagcaggtggcctctgggcgggcaggggtgtttactcaataccataaaaaaaagaaagccatgactgtgggggagtatcgccatttggcaaacagtaaaaaatatcagactccaccacaccagaatttcgaagatttggagcgaaaatactggaagaaccgcatctataattcaccgatttatggtgctgacatcagtggctccttgtttgatgaaaacactaaacaatggaatcttgggcacctgggaacaattcaggacctgctggaaaaggaatgtggggttgtcatagaaggcgtcaatacaccctacttgtactttggcatgtggaaaaccacgtttgcttggcatacagaggacatggacctttacagcatcaactacctgcaccttggggagcccaaaacttggtatgtggtgcccccagaacatggccagcgcctggaacgcctggccagggagctcttcccaggcagttcccggggttgtggggccttcctgcggcacaaggtggccctcatctcgcctacagttctcaaggaaaatgggattcccttcaatcgcataactcaggaggctggagagttcatggtgacctttccctatggctaccatgctggcttcaaccatggtttcaactgcgcagaggccatcaattttgccactccgcgatggattgattatggcaaaatggcctcccagtgtagctgtggggaggcaagggtgaccttttccatggatgccttcgtgcgcatcctgcaacctgaacgctatgacctgtggaaacgtgggcaagaccgggcagttgtggaccacatggagcccagggtaccagccagccaagagctgagcacccagaaggaagtccagttacccaggagagcagcgctgggcctgagacaactcccttcccactgggcccggcattccccttggcctatggctgcccgcagtgggacacggtgccacacccttgtgtgctcttcactcccacgccgatctgcagttagtggcactgctacgcagccccgggctgctgctgtccacagctctaagaagcccagctcaactccatcatccacccctggtccatctgcacagattatccacccgtcaaatggcagacgtggtcgtggtcgccctcctcagaaactgagagctcaggagctgaccctccagactccagccaagaggcccctcttggcgggcacaacatgcacagcttcgggcccagaacctgagcccctacctgaggatggggctttgatggacaagcctgtaccactgagcccagggctccagcatcctgtcaaggcttctgggtgcagctgggcccctgtgccctaa

SEQ ID NO: 5 - protein, 412 aa, Homo sapiens
MAENLKGCSVCCKSSWNQLQDLCRLAKLSCPALGISKRNLYDFEVEYLCDYKKIREQEYYLVKWRGYPDSESTWEPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 6 - protein, 350 aa, Homo sapiens
MEYYLVKWKGWPDSTNTWEPLQNLKCPLLLQQFSNDKHNYLSQVKKGKAITPKDNNKTLKPAIAEYIVKKAKQRIALQRWQDELNRRKNHKGMIFVENTVDLEGPPSDFYYINEYKPAPGISLVNEATFGCSCTDCFFQKCCPAEAGVLLAYNKNQQIKIPPGTPIYECNSRCQCGPDCPNRIVQKGTQYSLCIFRTSNGRGWGVKTLVKIKRMSFVMEYVGEVITSEEAERRGQFYDNKGITYLFDLDYESDEFTVDAARYGNVSHFVNHSCDPNLQVFNVFIDNLDTRLPRIALFSTRTINAGEELTFDYQMKGSGDISSDSIDHSPAKKRVRTVCKCGAVTCRGYLN

SEQ ID NO: 7 - DNA, 21 bp, Artificial Sequence
  Description of Artificial Sequence Synthetic oligonucleotide
  Description of Combined DNA/RNA Molecule Synthetic oligonucleotide
gaaacgaguccguauugaatt

SEQ ID NO: 8 - DNA, 21 bp, Artificial Sequence
  Description of Artificial Sequence Synthetic oligonucleotide
  Description of Combined DNA/RNA Molecule Synthetic oligonucleotide
uucaauacggacucguuuctt

SEQ ID NO: 9 - protein, 1064 aa, Homo sapiens
MASESETLNPSARIMTFYPTMEEFRNFSRYIAYIESQGAHRAGLAKVVPPKEWKPRASYDDIDDLVIPAPIQQLVTGQSGLFTQYNIQKKAMTVREFRKIANSDKYCTPRYSEFEELERKYWKNLTFNPPIYGADVNGTLYEKHVDEWNIGRLRTILDLVEKESGITIEGVNTPYLYFGMWKTSFAWHTEDMDLYSINYLHFGEPKSWYSVPPEHGKRLERLAKGFFPGSAQSCEAFLRHKMTLISPLMLKKYGIPFDKVTQEAGEFMITFPYGYHAGFNHGFNCAESTNFATRRWIEYGKQAVLCSCRKDMVKISMDVFVRKFQPERYKLWKAGKDNTVIDHTLPTPEAAEFLKESELPPRAGNEEECPEEDMEGVEDGEEGDLKTSLAKHRIGTKRHRVCLEIPQEVSQSELFPKEDLSSEQYEMTECPAALAPVRPTHSSVRQVEDGLTFPDYSDSTEVKFEELKNVKLEEEDEEEEQAAAALDLSVNPASVGGRLVFSGSKKKSSSSLGSGSSRDSISSDSETSEPLSCRAQGQTGVLTVHSYAKGDGRVTVGEPCTRKKGSAARSFSERELAEVADEYMFSLEENKKSKGRRQPLSKLPRHHPLVLQECVSDDETSEQLTPEEEAEETEAWAKPLSQLWQNRPPNFEAEKEFNETMAQQAPHCAVCMIFQTYHQVEFGGFNQNCGNASDLAPQKQRTKPLIPEMCFTSTGCSTDINLSTPYLEEDGTSILVSCKKCSVRVHASCYGVPPAKASEDWMCSRCSANALEEDCCLCSLRGGALQRANDDRWVHVSCAVAILEARFVNIAERSPVDVSKIPLPRFKLKCIFCKKRRKRTAGCCVQCSHGRCPTAFHVSCAQAAGVMMQPDDWPFVVFITCFRHKIPNLERAKGALQSITAGQKVISKHKNGRFYQCEVVRLTTETFYEVNFDDGSFSDNLYPEDIVSQDCLQFGPPAEGEVVQVRWTDGQVYGAKFVASHPIQMYQVEFEDGSQLVVKRDDVYTLDEELPKRVKSRLSVASDMRFNEIFTEKEVKQEKKRQRVINSRYREDYIEPALYRAIME

SEQ ID NO: 10 - protein, 1096 aa, Homo sapiens
MGSEDHGAQNPSCKIMTFRPTMEEFKDFNKYVAYIESQGAHRAGLAKIIPPKEWKPRQTYDDIDDVVIPAPIQQVVTGQSGLFTQYNIQKKAMTVGEYRRLANSEKYCTPRHQDFDDLERKYWKNLTFVSPIYGADISGSLYDDDVAQWNIGSLRTILDMVERECGTIIEGVNTPYLYFGMWKTTFAWHTEDMDLYSINYLHFGEPKSWYAIPPEHGKRLERLAIGFFPGSSQGCDAFLRHKMTLISPIILKKYGIPFSRITQEAGEFMITFPYGYHAGFNHGFNCAESTNFATLRWIDYGKVATQCTCRKDMVKISMDVFVRILQPERYELWKQGKDLTVLDHTRPTALTSPELSSWSASRASLKAKLLRRSHRKRSQPKKPKPEDPKFPGEGTAGAALLEEAGGSVKEEAGPEVDPEEEEEEPQPLPHGREAEGAEEDGRGKLRPTKAKSERKKKSFGLLPPQLPPPPAHFPSEEALWLPSPLEPPVLGPGPAAMEESPLPAPLNVVPPEVPSEELEAKPRPIIPMLYVVPRPGKAAFNQEHVSCQQAFEHFAQKGPTWKEPVSPMELTGPEDGAASSGAGRMETKARAGEGQAPSTFSKLKMEIKKSRRHPLGRPPTRSPLSVVKQEASSDEEASPFSGEEDVSDPDALRPLLSLQWKNRAASFQAERKFNAAAARTEPYCAICTLFYPYCQALQTEKEAPIASLGEGCPATLPSKSRQKTRPLIPEMCFTSGGENTEPLPANSYIGDDGTSPLIACGKCCLQVHASCYGIRPELVNEGWTCSRCAAHAWTAECCLCNLRGGALQMTTDRRWIHVICAIAVPEARFLNVIERHPVDISAIPEQRWKLKCVYCRKRMKKVSGACIQCSYEHCSTSFHVTCAHAAGVLMEPDDWPYVVSITCLKHKSGGHAVQLLRAVSLGQVVITKNRNGLYYRCRVIGAASQTCYEVNFDDGSYSDNLYPESITSRDCVQLGPPSEGELVELRWTDGNLYKAKFISSVTSHIYQVEFEDGSQLTVKRGDIFTLEEELPKRVRSRLSLSTGAPQEPAFSGEEAKAAKRPRVGTPLATEDSGRSQDYVAFVESLLQVQGRPGAPF

SEQ ID NO: 11 - protein, 1056 aa, Homo sapiens
MEVAEVESPLNPSCKIMTFRPSMEEFREFNKYLAYMESKGAHRAGLAKVIPPKEWKPRQCYDDIDNLLIPAPIQQMVTGQSGLFTQYNIQKKAMTVKEFRQLANSGKYCTPRYLDYEDLERKYWKNLTFVAPIYGADINGSIYDEGVDEWNIARLNTVLDVVEEECGISIEGVNTPYLYFGMWKTTFAWHTEDMDLYSINYLHFGEPKSWYAIPPEHGKRLERLAQGFFPSSSQGCDAFLRHKMTLISPSVLKKYGIPFDKITQEAGEFMITFPYGYHAGFNHGFNCAESTNFATVRWIDYGKVAKLCTCRKDMVKISMDIFVRKFQPDRYQLWKQGKDIYTIDHTKPTPASTPEVKAWLQRRRKVRKASRSFQCARSTSKRPKADEEEEVSDEVDGAEVPNPDSVTDDLKVSEKSEAAVKLRNTEASSEEESSASRMQVEQNLSDHIKLSGNSCLSTSVTEDIKTEDDKAYAYRSVPSISSEADDSIPLSSGYEKPEKSDPSELSWPKSPESCSSVAESNGVLTEGEESDVESHGNGLEPGEIPAVPSGERNSFKVPSIAEGENKTSKSWRHPLSRPPARSPMTLVKQQAPSDEELPEVLSIEEEVEETESWAKPLIHLWQTKSPNFAAEQEYNATVARMKPHCAICTLLMPYHKPDSSNEENDARWETKLDEVVTSEGKTKPLIPEMCFIYSEENIEYSPPNAFLEEDGTSLLISCAKCCVRVHASCYGIPSHEICDGWLCARCKRNAWTAECCLCNLRGGALKQTKNNKWAHVMCAVAVPEVRFTNVPERTQIDVGRIPLQRLKLKCIFCRHRVKRVSGACIQCSYGRCPASFHVTCAHAAGVLMEPDDWPYVVNITCFRHKVNPNVKSKACEKVISVGQTVITKHRNTRYYSCRVMAVTSQTFYEVMFDDGSFSRDTFPEDIVSRDCLKLGPPAEGEVVQVKWPDGKLYGAKYFGSNIAHMYQVEFEDGSQIAMKREDIYTLDEELPKRVKARFSTASDMRFEDTFYGADIIQGERKRQRVLSSRFKNEYVADPVYRTFLKSSFQKKCQKRQ

SEQ ID NO: 12 - protein, 523 aa, Homo sapiens
METMKSKANCAQNPNCNIMIFHPTKEEFNDFDKYIAYMESQGAHRAGLAKIIPPKEWKARETYDNISEILIATPLQQVASGRAGVFTQYHKKKKAMTVGEYRHLANSKKYQTPPHQNFEDLERKYWKNRIYNSPIYGADISGSLFDENTKQWNLGHLGTIQDLLEKECGVVIEGVNTPYLYFGMWKTTFAWHTEDMDLYSINYLHLGEPKTWYVVPPEHGQRLERLARELFPGSSRGCGAFLRHKVALISPTVLKENGIPFNRITQEAGEFMVTFPYGYHAGFNHGFNCAEAINFATPRWIDYGKMASQCSCGEARVTFSMDAFVRILQPERYDLWKRGQDRAVVDHMEPRVPASQELSTQKEVQLPRRAALGLRQLPSHWARHSPWPMAARSGTRCHTLVCSSLPRRSAVSGTATQPRAAAVHSSKKPSSTPSSTPGPSAQIIHPSNGRRGRGRPPQKLRAQELTLQTPAKRPLLAGTTCTASGPEPEPLPEDGALMDKPVPLSPGLQHPVKASGCSWAPVP

SEQ ID NO: 13 - protein, 423 aa, Homo sapiens
METMKSKANCAQNPNCNIMIFHPTKEEFNDFDKYIAYMESQGAHRAGLAKIIPPKEWKARETYDNISEILIATPLQQVASGRAGVFTQYHKKKKAMTVGEYRHLANSKKYQTPPHQNFEDLERKYWKNRIYNSPIYGADISGSLFDENTKQWNLGHLGTIQDLLEKECGVVIEGVNTPYLYFGMWKTTFAWHTEDMDLYSINYLHLGEPKTWYVVPPEHGQRLERLARELFPGSSRGCGAFLRHKVALISPTVLKENGIPFNRITQEAGEFMVTFPYGYHAGFNHGFNCAEAINFATPRWIDYGKMASQCSCGEARVTFSMDAFVRILQPERYDLWKRGQDRAVVDHMEPRVPASQELSTQKEVQLPRRAALGLRQLPSHWARHSPWPMAARSGTRCHTLVCSSLPRRSAVSGTATQPRAAAV

SEQ ID NO: 14 - DNA, 2752 bp, Homo sapiens
cgctcttctcgcgaggccggctaggcccgaatgtcgttagccgtggggaaagatggcggaaaatttaaaaggctgcagcgtgtgttgcaagtcttcttggaatcagctgcaggacctgtgccgcctggccaagctctcctgccctgccctcggtatctctaagaggaacctctatgactttgaagtcgagtacctgtgcgattacaagaagatccgcgaacaggaatattacctggtgaaatggcgtggatatccagactcagagagcacctgggagccacggcagaatctcaagtgtgtgcgtatcctcaagcagttccacaaggacttagaaagggagctgctccggcggcaccaccggtcaaagaccccccggcacctggacccaagcttggccaactacctggtgcagaaggccaagcagaggcgggcgctccgtcgctgggagcaggagctcaatgccaagcgcagccatctgggacgcatcactgtagagaatgaggtggacctggacggccctccgcgggccttcgtgtacatcaatgagtaccgtgttggtgagggcatcaccctcaaccaggtggctgtgggctgcgagtgccaggactgtctgtgggcacccactggaggctgctgcccgggggcgtcactgcacaagtttgcctacaatgaccagggccaggtgcggcttcgagccgggctgcccatctacgagtgcaactcccgctgccgctgcggctatgactgcccaaatcgtgtggtacagaagggtatccgatatgacctctgcatcttccgcacggatgatgggcgtggctggggcgtccgcaccctggagaagattcgcaagaacagcttcgtcatggagtacgtgggagagatcattacctcagaggaggcagagcggcggggccagatctacgaccgtcagggcgccacctacctctttgacctggactacgtggaggacgtgtacaccgtggatgccgcctactatggcaacatctcccactttgtcaaccacagttgtgaccccaacctgcaggtgtacaacgtcttcatagacaaccttgacgagcggctgccccgcatcgctttctttgccacaagaaccatccgggcaggcgaggagctcacctttgattacaacatgcaagtggaccccgtggacatggagagcacccgcatggactccaactttggcctggctgggctccctggctcccctaagaagcgggtccgtattgaatgcaagtgtgggactgagtcctgccgcaaatacctcttctagcccttagaagtctgaggccagactgactgagggggcctgaagctacatgcacctcccccactgctgccctcctgtcgagaatgactgccagggcctcgcctgcctccacctgcccccacctgctcctacctgctctacgttcagggctgtggccgtggtgaggaccgactccaggagtcccctttccctgtcccagccccatctgtgggttgcacttacaaacccccacccaccttcagaaatagtttttcaacatcaagactctctgtcgttgggattcatggcctattaaggaggtccaaggggtgagtcccaacccagccccagaatatatttgtttttgcacctgcttctgcctggagattgaggggtctgctgcaggcctcctccctgctgccccaaaggtatggggaagcaaccccagagcaggcagacatcagaggccagagtgcctagcccgacatgaagctggttccccaaccacagaaactttgtactagtgaaagaaagggggtccctgggctacgggctgaggctggtttctgctcgtgcttacagtgctgggtagtgttggccctaagagctgtagggtctcttcttcagggctgcatatctgagaagtggatgcccacatgccactggaagggaagtgggtgtccatgggccactgagcagtgagaggaaggcagtgcagagctggccagccctggaggtaggctgggaccaagctctgccttcacagtgcagtgaaggtacctagggctcttgggagctctgcggttgctaggggccctgacctggggtgtcatgaccgctgacaccactcagagctggaaccaagatctagatagtccgtagatagcacttaggacaagaatgtgcattgatggggtggtgatgaggtgccaggcactgggtagagcacctggtccacgtggattgtctcagggaagccttgaaaaccacggaggtggatgccaggaaagggcccatgtggcagaaggcaaagtacaggccaagaattgggggtgggggagatggcttccccactatgggatgacgaggcgagagggaagcccttgctgcctgccattcccagaccccagccctttgtgctcaccctggttccactggtctcaaaagtcacctgcctacaaatgtacaaaaggcgaaggttctgatggctgccttgctccttgctcccccaccccctgtgaggacttctctaggaagtccttcctgactacctgtgcccagagtgcccctacatgagactgtatgccctgctatcagatgccagatctatgtgtctgtctgtgtgtccatcccgccggccccccagactaacctccaggcatggactgaatctggttctcctcttgtacacccctcaaccctatgcagcctggagtgggcatcaataaaatgaactgtcgactgaacaaaaaaaaaaaaaaaaaa

SEQ ID NO: 15 - DNA, 3093 bp, Homo sapiens
cggggccgaggcgcgaggaggtgaggctggagcgcggccccctcgccttccctgttcccaggcaagctcccaaggcccgggcggcggggccgtcccgcgggccagccagatggcgacgtggcggttccccgcccgccgcgaccccaactccgggacgcacgctgcggacgcctatcctcccccaggccgctgacccgcctccctgcccggccggctcccgccgcggaggatatggaatattatcttgtaaaatggaaaggatggccagattctacaaatacttgggaacctttgcaaaatctgaagtgcccgttactgcttcagcaattctctaatgacaagcataattatttatctcaggtaaagaaaggcaaagcaataactccaaaagacaataacaaaactttgaaacctgccattgctgagtacattgtgaagaaggctaaacaaaggatagctctgcagagatggcaagatgaactcaacagaagaaagaatcataaaggaatgatatttgttgaaaatactgttgatttagagggcccaccttcagacttctattacattaacgaatacaaaccagctcctggaatcagcttagtcaatgaagctacctttggttgttcatgcacagattgcttctttcaaaaatgttgtcctgctgaagctggagttcttttggcttataataaaaaccaacaaattaaaatcccacctggtactcccatctatgaatgcaactcaaggtgtcagtgtggtcctgattgtcccaataggattgtacaaaaaggcacacagtattcgctttgcatctttcgaactagcaatggacgtggctggggtgtaaagacccttgtgaagattaaaagaatgagttttgtcatggaatatgttggagaggtaatcacaagtgaagaagctgaaagacgaggacagttctatgacaacaagggaatcacgtatctctttgatctggactatgagtctgatgaattcacagtggatgcggctcgatacggcaatgtgtctcattttgtgaatcacagctgtgacccaaatcttcaggtgttcaatgttttcattgataacctcgatactcgtcttccccgaatagcattgttttccacaagaaccataaatgctggagaagagctgacttttgattatcaaatgaaaggttctggagatatatcttcagattctattgaccacagcccagccaaaaagagggtcagaacagtatgtaaatgtggagctgtgacttgcagaggttacctcaactgaactttttcaggaaatagagctgatgattataatatttttttcctaatgttaacatttttaaaaatacatatttgggactcttattatcaaggttctacctatgttaatttacaattcatgtttcaagacatttgccaaatgtattaccgatgcctctgaaaagggggtcactgggtctcatagactgatatgaagtcgacatatttatagtgcttagagaccaaactaatggaaggcagactatttacagcttagtatatgtgtacttaagtctatgtgaacagagaaatgcctcccgtagtgtttgaaagcgttaagctgataatgtaattaacaactgctgagagatcaaagattcaacttgccatacacctcaaattcggagaaacagttaatttgggcaaatctacagttctgtttttgctactctattgtcattcctgtttaatactcactgtacttgtatttgagacaaataggtgatactgaattttatactgttttctacttttccattaaaacattggcacctcaatgataaagaaatttaaggtataaaattaaatgtaaaaattaatttcagcttcatttcgtatttcgaagcaatctagactgttgtgatgagtgtatgtctgaacctgtaattcttaaaagacttcttaatcttctagaagaaaaatctccgaagagctctctctagaagtccaaaatggctagccattatgcttctttgaaaggacatgataatgggaccaggatggttttttggagtaccaagcaaggggaatggagcactttaagggcgcctgttagtaacatgaattggaaatctgtgtcgagtacctctgatctaaacggtaaaacaagctgcctggagagcagctgtacctaacaatactgtaatgtacattaacattacagcctctcaatttcaggcaggtgtaacagttcctttccaccagatttaatatttttatacttcctgcaggttcttcttaaaaagtaatctatatttttgaactgatacttgttttatacataaattttttttagatgtgataaagctaaacttggccaaagtgtgtgcctgaattattagacctttttattagtcaacctacgaagactaaaatagaatatattagttttcaagggagtgggaggcttccaacatagtattgaatctcaggaaaaactattctttcatgtctgattctgagatttctaattgtgttgtgaaaatgataaatgcagcaaatctagctttcagtattcctaatttttacctaagctcattgctccaggctttgattacctaaaataagcttggataaaattgaaccaacttcaagaatgcagcacttcttaatctttagctctttcttgggagaagctagactttattcattatattgctatgacaacttcactctttcataatatataggataaattgtttacatgattggaccctcagattctgttaaccaaaattgcagaatggggggccaggcctgtgtggtggctcacacctgtgatcccagcactttgggaggctgaggtaggaggatcacgtgaggtcgggagttcaagaccagcctggccatcatggtgaaaccctgtctctactgaaaatacaaaaattagccgggcgtggtggcacacgcctgtagtcccagctactcaggaggctgaggcaggagaatcacttgaattcaggaggcggaggttgcagtgagccaagatcataccactgcactgcagcctgagtgacacagtaagactgtctccaaaaaaaaaaaaaaaaaa

SEQ ID NO: 16 - DNA, 4449 bp, Homo sapiens
ggcactaaaggtttgcttccgggcgtttcttttgcttccccttccctctttcacgcttcctcccctccccctcctcccttatcccttcgctttcgctcttttccgtcgaggccgacccctgagttgtgagtctggggtctggttggtgaaaaagagcccttgaagctggaagacgggagaggacaaaagcatgtcttcccttcctgggtgcattggtttggatgcagcaacagctacagtggagtctgaagagattgcagagctgcaacaggcagtggttgaggaactgggtatctctatggaggaacttcggcatttcatcgatgaggaactggagaagatggattgtgtacagcaacgcaagaagcagctagcagagttagagacatgggtaatacagaaagaatctgaggtggctcacgttgaccaactctttgatgatgcatccagggcagtgactaattgtgagtctttggtgaaggacttctactccaagctgggactacaataccgggacagtagctctgaggacgaatcttcccggcctacagaaataattgagattcctgatgaagatgatgatgtcctcagtattgattcaggtgatgctgggagcagaactccaaaagaccagaagctccgtgaagctatggctgccttaagaaagtcagctcaagatgttcagaagttcatggatgctgtcaacaagaagagcagttcccaggatctgcataaaggaaccttgagtcagatgtctggagaactaagcaaagatggtgacctgatagtcagcatgcgaattctgggcaagaagagaactaagacttggcacaaaggcacccttattgccatccagacagttgggccagggaagaaatacaaggtgaaatttgacaacaaaggaaagagtctactgtcggggaaccatattgcctatgattaccaccctcctgctgacaagctgtatgtgggcagtcgggtggtcgccaaatacaaagatgggaatcaggtctggctctatgctggcattgtagctgagacaccaaacgtcaaaaacaagctcaggtttctcattttctttgatgatggctatgcttcctatgtcacacagtcggaactgtatcccatttgccggccactgaaaaagacttgggaggacatagaagacatctcctgccgtgacttcatagaggagtatgtcactgcctaccccaaccgccccatggtactgctcaagagtggccagcttatcaagactgagtgggaaggcacgtggtggaagtcccgagttgaggaggtggatggcagcctagtcaggatcctcttcctggatgacaaaagatgtgagtggatctatcgaggctctacacggctggagcccatgttcagcatgaaaacatcctcagcctctgcactggagaagaagcaaggacagctcaggacacgtccaaatatgggtgctgtgaggagcaaaggccctgttgtccagtacacacaggatctgaccggtactggaacccagttcaagccagtggaacccccacagcctacagctccacctgccccacctttcccacctgctccacctctatccccccaagcaggtgacagtgacttggaaagccagcttgcccagtcacggaagcaggtagccaaaaagagcacgtcctttcgaccaggatctgtgggctctggtcattcctcccctacatctcctgcactcagtgaaaatgtctctggtgggaaacctgggatcaaccagacatatagatcacctttaggctccacagcctctgccccagcaccctcagcactcccggcccctccagcacccccagtcttccatggcatgctggagcgggccccagcagagccctcctaccgtgctcccatggagaagcttttctacttacctcatgtctgcagctatacctgtctgtctcgagtcagacctatgaggaatgagcagtaccggggcaagaaccctctgctggtcccgttactatatgacttccggcggatgacagcccggcgtcgagttaaccgcaagatgggctttcatgttatctataagacaccttgtggtctctgccttcggacaatgcaggagatagaacgctaccttttcgagactggctgtgacttcctcttcctggagatgttctgtttggatccatatgttcttgtggaccgaaagtttcagccctataagcctttttactatattttggacatcacttatgggaaggaagatgttcccctatcctgtgtcaatgagattgacacaacccctccaccccaggtggcctacagcaaggaacgtatcccgggcaagggtgttttcattaacacaggccctgaatttctggttggctgtgactgcaaggatgggtgtcgggacaagtccaagtgtgcctgccatcaactaactatccaggctacagcctgtaccccaggaggccaaatcaaccctaactctggctaccagtacaagagactagaagagtgtctacccacaggggtatatgagtgtaacaaacgctgcaaatgtgacccaaacatgtgcacaaaccggttggtgcaacatggactacaagttcggctacagctattcaagacacagaacaagggctggggtatccgctgcttggatgacattgccaaaggctcttttgtttgtatttatgcaggcaaaatcctgacagatgactttgcagacaaggagggtctggaaatgggtgatgagtactttgcaaatctggaccatatcgagagcgtggagaacttcaaagaaggatatgagagtgatgccccctgttcctctgacagcagtggtgtagacttgaaggaccaggaagatggcaacagcggtacagaggaccctgaagagtccaatgatgatagctcagatgataacttctgtaaggatgaggacttcagcaccagttcagtgtggcggagctatgctacccggaggcagacccggggccagaaagagaacggactctctgagacaacttccaaggactcccaccccccagatcttggacccccacatattcctgttcctccctcaatccctgtaggtggctgcaatccaccttcctccgaagagacacccaagaacaaggtggcctcatggttgagctgcaatagtgtcagtgaaggtggttttgctgactctgatagccattcatccttcaagactaatgaaggtggggagggccgggctgggggaagccgaatggaggctgagaaggcctccacctcaggactaggcatcaaggatgagggagacatcaaacaggccaagaaagaggacactgacgaccgaaacaagatgtcagtagttactgaaagctctcgaaattacggttacaatccttctcctgtgaagcctgaaggacttcgccgcccacctagtaagactagtatgcatcaaagccgaagactcatggcttctgctcagtccaaccctgatgatgtcctgacactgtccagcagcacagaaagtgagggggaaagtgggaccagccgaaagcccactgctggtcagacttcggctacagcggttgacagtgatgatatccagaccatatcctctggctctgaaggggatgactttgaggacaagaagaacatgactggtccaatgaagcgtcaagtggcagtaaaatcaacccgaggctttgctcttaaatcaacccatgggattgcaattaaatcaaccaacatggcctctgtggacaagggggagagcgcacctgttcgtaagaacacacgccaattctatgatggcgaggagtcttgctacatcattgatgccaagcttgaaggcaacctgggccgctacctcaaccacagttgcagccccaacctgtttgtccagaatgtcttcgtggatacccatgatcttcgcttcccctgggtggccttctttgccagcaaaagaatccgggctgggacagaacttacttgggactacaactacgaggtgggcagtgtggaaggcaaggagctactctgttgctgtggggccattgaatgcagaggacgtcttctttagaggacagccttcttcccaacccttcttgaactgtcgtttcctcaggaactgggtcttcctgattgttgaaccctgacccgaagtctctgggctagctactccccccagctcctagttgatagaaatgggggttctggaccagatgatcccttccaatgtggtgctagcaggcaggatcccttctccacctccaaaggccctaaagggtggggagagatcaccactctaacctcggcctgacatccctcccatcccatatttgtccaagtgttcctgcttctaacagactttgttcttagaatggagcctgtgtatctactatctccagtttgtattatttcttgaaagtcttttaacaatatgataaaactaagattgtgaaa

SEQ ID NO: 17 - protein, 1291 aa, Homo sapiens
MSSLPGCIGLDAATATVESEEIAELQQAVVEELGISMEELRHFIDEELEKMDCVQQRKKQLAELETWVIQKESEVAHVDQLFDDASRAVTNCESLVKDFYSKLGLQYRDSSSEDESSRPTEIIEIPDEDDDVLSIDSGDAGSRTPKDQKLREAMAALRKSAQDVQKFMDAVNKKSSSQDLHKGTLSQMSGELSKDGDLIVSMRILGKKRTKTWHKGTLIAIQTVGPGKKYKVKFDNKGKSLLSGNHIAYDYHPPADKLYVGSRVVAKYKDGNQVWLYAGIVAETPNVKNKLRFLIFFDDGYASYVTQSELYPICRPLKKTWEDIEDISCRDFIEEYVTAYPNRPMVLLKSGQLIKTEWEGTWWKSRVEEVDGSLVRILFLDDKRCEWIYRGSTRLEPMFSMKTSSASALEKKQGQLRTRPNMGAVRSKGPVVQYTQDLTGTGTQFKPVEPPQPTAPPAPPFPPAPPLSPQAGDSDLESQLAQSRKQVAKKSTSFRPGSVGSGHSSPTSPALSENVSGGKPGINQTYRSPLGSTASAPAPSALPAPPAPPVFHGMLERAPAEPSYRAPMEKLFYLPHVCSYTCLSRVRPMRNEQYRGKNPLLVPLLYDFRRMTARRRVNRKMGFHVIYKTPCGLCLRTMQEIERYLFETGCDFLFLEMFCLDPYVLVDRKFQPYKPFYYILDITYGKEDVPLSCVNEIDTTPPPQVAYSKERIPGKGVFINTGPEFLVGCDCKDGCRDKSKCACHQLTIQATACTPGGQINPNSGYQYKRLEECLPTGVYECNKRCKCDPNMCTNRLVQHGLQVRLQLFKTQNKGWGIRCLDDIAKGSFVCIYAGKILTDDFADKEGLEMGDEYFANLDHIESVENFKEGYESDAPCSSDSSGVDLKDQEDGNSGTEDPEESNDDSSDDNFCKDEDFSTSSVWRSYATRRQTRGQKENGLSETTSKDSHPPDLGPPHIPVPPSIPVGGCNPPSSEETPKNKVASWLSCNSVSEGGFADSDSHSSFKTNEGGEGRAGGSRMEAEKASTSGLGIKDEGDIKQAKKEDTDDRNKMSVVTESSRNYGYNPSPVKPEGLRRPPSKTSMHQSRRLMASAQSNPDDVLTLSSSTESEGESGTSRKPTAGQTSATAVDSDDIQTISSGSEGDDFEDKKNMTGPMKRQVAVKSTRGFALKSTHGIAIKSTNMASVDKGESAPVRKNTRQFYDGEESCYIIDAKLEGNLGRYLNHSCSPNLFVQNVFVDTHDLRFPWVAFFASKRIRAGTELTWDYNYEVGSVEGKELLCCCGAIECRGRLL

SEQ ID NO: 18 - DNA, 21 bp, Artificial Sequence
  Description of Artificial Sequence Synthetic oligonucleotide
  Description of Combined DNA/RNA Molecule Synthetic oligonucleotide
gcucacauguaaaucgauutt

SEQ ID NO: 19 - DNA, 21 bp, Artificial Sequence
  Description of Artificial Sequence Synthetic oligonucleotide
  Description of Combined DNA/RNA Molecule Synthetic oligonucleotide
aaucgauuuacaugugagctt

SEQ ID NO: 20 - DNA, 21 bp, Artificial Sequence
  Description of Artificial Sequence Synthetic oligonucleotide
  Description of Combined DNA/RNA Molecule Synthetic oligonucleotide
gguguacaacguauucauatt

SEQ ID NO: 21 - DNA, 21 bp, Artificial Sequence
  Description of Artificial Sequence Synthetic oligonucleotide
  Description of Combined DNA/RNA Molecule Synthetic oligonucleotide
uaugaauacguuguacacctg

SEQ ID NO: 22 - DNA, 21 bp, Artificial Sequence
  Description of Artificial Sequence Synthetic oligonucleotide
  Description of Combined DNA/RNA Molecule Synthetic oligonucleotide
gguccuuugucuauaucaatt

SEQ ID NO: 23 - DNA, 21 bp, Artificial Sequence
  Description of Artificial Sequence Synthetic oligonucleotide
  Description of Combined DNA/RNA Molecule Synthetic oligonucleotide
uugauauagacaaaggacctt

SEQ ID NO: 24 - DNA, 21 bp, Artificial Sequence
  Description of Artificial Sequence Synthetic oligonucleotide
  Description of Combined DNA/RNA Molecule Synthetic oligonucleotide
gcucacauguaaaucgauutt

SEQ ID NO: 25 - DNA, 21 bp, Artificial Sequence
  Description of Artificial Sequence Synthetic oligonucleotide
  Description of Combined DNA/RNA Molecule Synthetic oligonucleotide
aaucgauuuacaugugagctt

SEQ ID NO: 26 - DNA, 21 bp, Artificial Sequence
  Description of Artificial Sequence Synthetic oligonucleotide
  Description of Combined DNA/RNA Molecule Synthetic oligonucleotide
gugucgauguggaccugaatt

SEQ ID NO: 27 - DNA, 21 bp, Artificial Sequence
  Description of Artificial Sequence Synthetic oligonucleotide
  Description of Combined DNA/RNA Molecule Synthetic oligonucleotide
uucagguccacaucgacacct

SEQ ID NO: 28 - DNA, 21 bp, Artificial Sequence
  Description of Artificial Sequence Synthetic oligonucleotide
  Description of Combined DNA/RNA Molecule Synthetic oligonucleotide
ggacuacaguaucaugacatt

SEQ ID NO: 29 - RNA, 21 nt, Artificial Sequence
  Description of Artificial Sequence Synthetic oligonucleotide
ugucaugauacuguaguccca

SEQ ID NO: 30 - DNA, 21 bp, Artificial Sequence
  Description of Artificial Sequence Synthetic oligonucleotide
  Description of Combined DNA/RNA Molecule Synthetic oligonucleotide
ggacgaugcaggagauagatt

SEQ ID NO: 31 - RNA, 21 nt, Artificial Sequence
  Description of Artificial Sequence Synthetic oligonucleotide
ucuaucuccugcaucguccga

SEQ ID NO: 32 - DNA, 21 bp, Artificial Sequence
  Description of Artificial Sequence Synthetic oligonucleotide
  Description of Combined DNA/RNA Molecule Synthetic oligonucleotide
ggaugggugucgggauaaatt

SEQ ID NO: 33 - DNA, 21 bp, Artificial Sequence
  Description of Artificial Sequence Synthetic oligonucleotide
  Description of Combined DNA/RNA Molecule Synthetic oligonucleotide
uuuaucccgacacccaucctt

SEQ ID NO: 34 - DNA, 21 bp, Artificial Sequence
  Description of Artificial Sequence Synthetic oligonucleotide
  Description of Combined DNA/RNA Molecule Synthetic oligonucleotide
gcaccuuugucugcgaauatt

SEQ ID NO: 35 - RNA, 21 nt, Artificial Sequence
  Description of Artificial Sequence Synthetic oligonucleotide
uauucgcagacaaaggugccc

SEQ ID NO: 36 - DNA, 21 bp, Artificial Sequence
  Description of Artificial Sequence Synthetic oligonucleotide
  Description of Combined DNA/RNA Molecule Synthetic oligonucleotide
gaucaaaccugcucggaaatt

SEQ ID NO: 37 - RNA, 21 nt, Artificial Sequence
  Description of Artificial Sequence Synthetic oligonucleotide
uuuccgagcagguuugaucca

SEQ ID NO: 38 - DNA, 21 bp, Artificial Sequence
  Description of Artificial Sequence Synthetic oligonucleotide
  Description of Combined DNA/RNA Molecule Synthetic oligonucleotide
gaauuugccuucuuaugcatt

SEQ ID NO: 39 - DNA, 21 bp, Artificial Sequence
  Description of Artificial Sequence Synthetic oligonucleotide
  Description of Combined DNA/RNA Molecule Synthetic oligonucleotide
ugcauaagaaggcaaauuctt

SEQ ID NO: 40 - DNA, 21 bp, Artificial Sequence
  Description of Artificial Sequence Synthetic oligonucleotide
  Description of Combined DNA/RNA Molecule Synthetic oligonucleotide
gaggaauucuagucccguatt

SEQ ID NO: 41 - RNA, 21 nt, Artificial Sequence
  Description of Artificial Sequence Synthetic oligonucleotide
uacgggacuagaauuccucaa

SEQ ID NO: 42 - DNA, 5123 bp, Homo sapiens
gcgcgggaggggcggggccacgctgcgggcccgggccatggccgccgccgatgccgaggcagttccggcgaggggggagcctcagcaggattgctgtgtgaaaaccgagctgctgggagaagagacacctatggctgccgatgaaggctcagcagagaaacaggcaggagaggcccacatggctgcggacggtgagaccaatgggtcttgtgaaaacagcgatgccagcagtcatgcaaatgctgcaaagcacactcaggacagcgcaagggtcaacccccaggatggcaccaacacactaactcggatagcggaaaatggggtttcagaaagagactcagaagcggcgaagcaaaaccacgtcactgccgacgactttgtgcagacttctgtcatcggcagcaacggatacatcttaaataagccggccctacaggcacagcccttgaggactaccagcactctggcctcttcgctgcctggccatgctgcaaaaacccttcctggaggggctggcaaaggcaggactccaagcgcttttccccagacgccagccgccccaccagccacccttggggaggggagtgctgacacagaggacaggaagctcccggcccctggcgccgacgtcaaggtccacagggcacgcaagaccatgccgaagtccgtcgtgggcctgcatgcagccagtaaagatcccagagaagttcgagaagctagagatcataaggaaccaaaagaggagatcaacaaaaacatttctgactttggacgacagcagcttttaccccccttcccatcccttcatcagtcgctacctcagaaccagtgctacatggccaccacaaaatcacagacagcttgcttgccttttgttttagcagctgcagtatctcggaagaaaaaacgaagaatgggaacctatagcctggttcctaagaaaaagaccaaagtattaaaacagaggacggtgattgagatgtttaagagcataactcattccactgtgggttccaagggggagaaggacctgggcgccagcagcctgcacgtgaatggggagagcctggagatggactcggatgaggacgactcagaggagctcgaggaggacgacggccatggtgcagagcaggcggccgcgttccccacagaggacagcaggacttccaaggagagcatgtcggaggctgatcgcgcccagaagatggacggggagtccgaggaggagcaggagtccgtggacaccggggaggaggaggaaggcggtgacgagtctgacctgagttcggaatccagcattaagaagaaatttctcaagaggaaaggaaagaccgacagtccctggatcaagccagccaggaaaaggaggcggagaagtagaaagaagcccagcggtgccctcggttctgagtcgtataagtcatctgcaggaagcgctgagcagacggcaccaggagacagcacagggtacatggaagtttctctggactccctggatctccgagtcaaaggaattctgtcttcacaagcagaagggttggccaacggtccagatgtgctggagacagacggcctccaggaagtgcctctctgcagctgccggatggaaacaccgaagagtcgagagatcaccacactggccaacaaccagtgcatggctacagagagcgtggaccatgaattgggccggtgcacaaacagcgtggtcaagtatgagctgatgcgcccctccaacaaggccccgctcctcgtgctgtgtgaagaccaccggggccgcatggtgaagcaccagtgctgtcctggctgtggctacttctgcacagcgggtaattttatggagtgtcagcccgagagcagcatctctcaccgtttccacaaagactgtgcctctcgagtcaataacgccagctattgtccccactgtggggaggagagctccaaggccaaagaggtgacgatagctaaagcagacaccacctcgaccgtgacaccagtccccgggcaggagaagggctcggccctggagggcagggccgacaccacaacgggcagtgctgccgggccaccactctcggaggacgacaagctgcagggtgcagcctcccacgtgcccgagggctttgatccaacgggacctgctgggcttgggaggccaactcccggcctttcccagggaccagggaaggaaaccttggagagcgctctcatcgccctcgactcggaaaaacccaagaagcttcgcttccacccaaagcagctgtacttctccgccaggcaaggggagcttcagaaggtgctcctcatgctggtggacggaattgaccccaacttcaaaatggagcaccagaataagcgctctccactgcacgccgcggcagaggctggacacgtggacatctgccacatgctggttcaggcgggcgctaatattgacacctgctcagaagaccagaggaccccgttgatggaagcagccgaaaacaaccatctggaagcagtgaagtacctcatcaaggctggggccctggtggatcccaaggacgcagagggctctacgtgtttgcacctggctgccaagaaaggccactacgaagtggtccagtacctgctttcaaatggacagatggacgtcaactgtcaggatgacggaggctggacacccatgatctgggccacagagtacaagcacgtggacctcgtgaagctgctgctgtccaagggctctgacatcaacatccgagacaacgaggagaacatttgcctgcactgggcggcgttctccggctgcgtggacatagccgagatcctgctggctgccaagtgcgacctccacgccgtgaacatccacggagactcgccactgcacattgccgcccgggagaaccgctacgactgtgtcgtcctctttctttctcgggattcagatgtcaccttaaagaacaaggaaggagagacgcccctgcagtgtgcgagcctcaactctcaggtgtggagcgctctgcagatgagcaaggctctgcaggactcggcccccgacaggcccagccccgtggagaggatagtgagcagggacatcgctcgaggctacgagcgcatccccatcccctgtgtcaacgccgtggacagcgagccatgccccagcaactacaagtacgtctctcagaactgcgtgacgtcccccatgaacatcgacagaaatatcactcatctgcagtactgcgtgtgcatcgacgactgctcctccagcaactgcatgtgcggccagctcagcatgcgctgctggtacgacaaggatggccggctcctgccagagttcaacatggcggagcctcccttgatcttcgaatgcaaccacgcgtgctcctgctggaggaactgccgaaatcgcgtcgtacagaatggtctcagggcaaggctgcagctctaccggacgcgggacatgggctggggcgtgcggtccctgcaggacatcccaccaggcacctttgtctgcgagtatgttggggagctgatttcagactcagaagccgacgttcgagaggaagattcttacctctttgatctcgacaataaggacggggaggtttactgcatcgacgcgcggttctacgggaacgtcagccggttcatcaaccaccactgcgagcccaacctggtgcccgtgcgcgtgttcatggcccaccaggacctgcggttcccccggatcgccttcttcagcacccgcctgatcgaggccggcgagcagctcgggtttgactatggagagcgcttctgggacatcaaaggcaagctcttcagctgccgctgcggctcccccaagtgccggcactcgagcgcggccctggcccagcgtcaggccagcgcggcccaggaggcccaggaggacggcttgcccgacaccagctccgcggctgccgccgaccccctatgagacgccgccggccagcggggcgctcgggagccagggaccgccgcgtcgccgattagaggacgaggaggagagattccgcacgcaaccgaaagggtccttcggggctgcgccgccggcttcctggaggggtcggaggtgaggctgcagcccctgcgggcgggtgtggatgcctcccagccaccttcccagacctgcggcctcaccgcgggcccagtgcccaggctggagcgcacactttggtccgcgcgccagagacgctgggagtccgcactggcatcaccttctgagtttctgatgctgatttgtcgttgcgaagtttctcgtttcttcctctgacctccgaggtccccgctgcaccacggggttgctctgttctcctgtccggcccagactcttctgtgtggcgccgccgaagccaccgttagcgcgagctgctccgttcgccctgcccacggcctgcgtggctggggccgagtcccaggggccgcacggagggcacagtctcctgtcaggctcggagaggtcaggagaccgaccccaccactaactttggagaaaatgtgggtttgctttttaaaggaatcctatatctagtcctatatatcaaacctctaactgacgtttcttttcgaggaagtggcttggtgggtgcagcccccgccggttccgttgacgctggcaccttctgttgattttttaagccacatgctatgatgaataaactgatttattttctaccattactgaacattaggacaaacacaaaataaaaaacaaaacacagacaacggtgctgattctggtgtggtttctactcaccacgtgaaataaactatcaactgtataaagagaacaaagtgattttagaataaaatgcaggaaaaacttttttaaagatgttagtcttgtagcgtgaataaatttgccatcaccttttgtgtggtggcctggcaggtcatatacttttttttggcatatacctttttaaagactgtaattagtgcagtaacagtggggttttttttgtgcaactcttctaaaaacattcataatgcagtcatgtttatttttttctgttaaaatgtttttgacagttttaagagcagtcttttggctctgaccatttcttgttctgtttccaatgaaatcaataaaaaaaaagaagtactttaaaaaaaaaaaaaaaaaa

SEQ ID NO: 43 - DNA, 7970 bp, Homo sapiens
gtaaaagtgacattctaaatgttcctcactctgcgaggcttattttttagggactttgctataattctgaaagacttagttttacagtacatctgaaagtaggagttttcagaagtatggctcttgggataaatttagattcttaattgtgaagctctgttaccacttgttagaaggcaggtcagctcacctgcttggggaggtaaatatatgaatgcactctcgagtaatttaatggagccctacctcaatgtacagaatgacagtatcacagatcaagaatggagtacgagtgattttcggctatggtgggggtaggtaggtcacttgtcccctgttgtctcttactatttgtaaagtgaagactatgattagtctttttgatcgggatggtttgagatgaataaagaataggcaggcaatttggatactttaggcttttcaagaacattagtaacattttttcttagatatttctcctaatacaatgagtgttgtgaaataacatggcagttattgttgagagaaaagccttcccagttatgtattgagtccttaggcgttttgaccttccctccactcttacagaacttggtggaaggggccactatgttttctacctccttccgtgcctttcacaaagccacatcctgcaccgtctacccttctctgtggatatttttccgcttggcaatttcctttcctgaggcacccacttgggacatctgaatctccatctccatgttgatggcccgtttgtgcttggacgtgttcttccacttgagactgagggttcatgtaatcaaagaagtttctttgttgtgtgtatctttacagaacacaacaggaattgaaaatgaatcagaacactactgagcctgtggcggccaccgagaccctggctgaggtacccgaacatgtgctgcgaggacttccggaggaagtgaggcttttcccttctgctgttgacaagacccggattggtgtctgggccactaaaccaattttaaaaggcaaaaaatttgggccatttgttggtgataagaaaaaaagatctcaggttaagaataatgtatacatgtgggaggtgtattacccaaatttgggatggatgtgcattgatgccactgatccagagaagggaaactggctgcgatatgtgaattgggcttgctcaggagaagagcaaaatttattcccactggaaatcaacagagccatttactataaaactttaaagccaatcgcgccgggcgaggagctcctggtctggtacaatggggaagacaaccctgagatagcagctgcgattgaggaagagcgagccagcgcccggagcaagcggagctcccccaagagccggaaagggaagaaaaaatcccaggaaaataaaaacaaaggaaacaaaatccaagacatacaactgaagacaagtgagccagatttcacctctgcaaatatgagagattctgcagaaggtcctaaagaagacgaagagaagccttcagcctcagcacttgagcagccggccaccctccaggaggtggccagtcaggaggtgcctccagaactagcaacccctgcccctgcctgggagccacagccagaaccagacgagcgattagaagcggcagcttgtgaggtgaatgatttgggggaagaggaggaggaggaagaggaggaggatgaagaagaagaagaagatgatgatgatgatgagttggaagacgagggggaagaagaagccagcatgccaaatgaaaattctgtgaaagagccagaaatacggtgtgatgagaagccagaagatttattagaggaaccaaaaacaacttcagaagaaactcttgaagactgctcagaggtaacacctgccatgcaaatccccagaactaaagaagaggccaatggtgatgtatttgaaacgtttatgtttccgtgtcaacattgtgaaaggaagtttacaaccaaacaggggcttgagcgtcacatgcatatccatatatccaccgtcaatcatgctttcaaatgcaagtactgtgggaaagcctttggcacacagattaaccggcggcgacatgagcggcgccatgaagcagggttaaagcggaaacccagccaaacactacagccgtcagaggatctggctgatggcaaagcatctggagaaaacgttgcttcaaaagatgattcgagtcctcccagtcttgggccagactgtctgatcatgaattcagagaaggcttcccaagacacaataaattcttctgtcgtagaagagaatggggaagttaaagaacttcatccgtgcaaatattgtaaaaaggtttttggaactcatactaatatgagacggcatcagcgtagagttcacgaacgtcatctgattcccaaaggtgtacggcgaaaaggaggccttgaagagccccagcctccagcagaacaggcccaggccacccagaacgtgtatgtaccaagcacagagccggaggaggaaggggaagcagatgatgtgtacatcatggacatttctagcaatatctctgaaaacttaaattactatattgatggtaaaattcaaactaataacaacactagtaactgtgatgtgattgagatggagtctgcttcggcagatttgtatggtataaattgtctgctcactccagttacagtggaaattactcaaaatataaagaccacacaggtccctgtaacagaagatcttcctaaagagcctttgggcagcacaaatagtgaggccaagaagcggagaactgcgagcccacctgcactgcccaaaattaaggccgaaacagactctgaccccatggtcccctcttgctctttaagtcttcctcttagcatatcaacaacagaggcagtgtctttccacaaagagaaaagtgtttatttgtcatcaaagctcaaacaacttcttcaaacccaagataaactaactcctgcagggatttcagcaactgaaatagctaaattaggtcctgtttgtgtgtctgctcctgcatcaatgttgcctgtgacctcaagtaggtttaagaggcggaccagctctcctcccagttctccacagcacagtcctgcccttcgagactttggaaagccaagtgatgggaaagcagcatggaccgatgccgggctgacttccaaaaaatccaaattagaaagtcacagcgactcaccagcatggagtttgtctgggagagatgagagagaaactgtgagccctccatgctttgatgaatataaaatgtctaaagagtggacagctagttctgcttttagcagtgtgtgcaaccagcagccactggatttatccagcggtgtcaaacagaaggctgagggtacaggcaagactccagtccagtgggaatctgtcttagatctcagtgtgcataaaaagcattgtagtgactctgaaggcaaggaattcaaagaaagtcattcagtgcagcctacgtgtagtgctgtaaagaaaaggaaaccaaccacctgcatgctgcagaaggttcttctcaatgaatataatggcatcgatttacctgtagaaaaccctgcagatgggaccaggagcccaagtccttgtaaatccctagaagctcagccagatcctgacctcggtccgggctctggtttccctgcccctactgttgagtccacacctgatgtttgtccttcatcacctgccctgcagacaccctccctttcatccggtcagctgcctcctctcttgatccccacagatccctcttcccctccaccctgtcccccggtattaactgttgccactccgccccctcccctccttcctaccgtacctcttccagccccctcttccagtgcatctccacacccatgcccctctccactctcaaatgccaccgcacagtccccacttccaattctgtccccaacagtgtccccctctccctctcccattcctcccgtggagcccctgatgtctgccgcctcacccgggcctccaacactttcttcttcctcctcttcatcttcctcctcctcttcgttttcttcttcatcttcctcctcttctccttctccacctcctctctccgcaatatcatctgttgtttcctctggtgataatctggaggcttctctccccatgatatctttcaaacaggaggaattagagaatgaaggtctgaaacccagggaagagccccagtctgctgctgaacaggatgttgttgttcaggaaacattcaacaaaaactttgtttgcaacgtctgtgaatcaccttttctttccattaaagatctaaccaaacatttatctattcatgctgaagaatggcccttcaaatgtgaattttgtgtgcagctttttaaggataaaacggacttgtcagaacatcgctttttgcttcatggagttgggaatatctttgtgtgttctgtttgtaaaaaagaatttgcttttttgtgcaatttgcagcagcaccagcgagatctccacccagataaggtgtgcacacatcacgagtttgaaagcgggactctgaggccccagaactttacagatcccagcaaggcccatgtagagcatatgcagagcttgccagaagatcctttagaaacttctaaagaagaagaggagttaaatgattcctctgaagagctttacacgactataaaaataatggcttctggaataaagacaaaagatccagatgttcgattgggcctcaatcagcattacccaagctttaaaccacctccatttcagtaccatcaccgtaaccccatggggattggtgtgacagccacaaatttcactacacacaatattccacagactttcactaccgccattcgctgcacaaagtgtggaaaaggtgtcgacaatatgccggagttgcacaaacatatcctggcttgtgcttctgcaagtgacaagaagaggtacacgcctaagaaaaacccagtaccattaaaacaaactgtgcaacccaaaaatggcgtggtggttttagataactctgggaaaaatgccttccgacgaatgggacagcccaaaaggcttaactttagtgttgagctcagcaaaatgtcgtcgaataagctcaaattaaatgcattgaagaaaaaaaatcagctagtacagaaagcaattcttcagaaaaacaaatctgcaaagcagaaggccgacttgaaaaatgcttgtgagtcatcctctcacatctgcccttactgtaatcgagagttcacttacattggaagcctgaataaacacgccgccttcagctgtcccaaaaaacccctttctcctcccaaaaaaaaagtttctcattcatctaagaaaggtggacactcatcacctgcaagtagtgacaaaaacagtaacagcaaccaccgcagacggacagcggatgcggagattaaaatgcaaagcatgcagactccgttgggcaagaccagagcccgcagctcaggccccacccaagtcccacttccctcctcatccttcaggtccaagcagaacgtcaagtttgcagcttcggtgaaatccaaaaaaccaagctcctcctctttaaggaactccagcccgataagaatggccaaaataactcatgttgaggggaaaaaacctaaagctgtggccaagaatcattctgctcagctttccagcaaaacatcacggagcctgcacgtgagggtacagaaaagcaaagctgttttacaaagcaaatccaccttggcgagtaagaaaagaacagaccggttcaatataaaatctagagagcggagtggggggccagtcacccggagccttcagctggcagctgctgctgacttgagtgagaacaagagagaggacggcagcgccaagcaggagctgaaggacttcagctacagcctccgcttggcgtcccgatgctctccaccagcggccccgtacatcaccaggcagtataggaaggtcaaagctccagctgcagcccagttccagggaccattcttcaaagagtagacactctggctgctccctgacagcacctgaagtgacctggaatcagtgaagccaaagggactggcagtctgccctgcagggagtaccgacctatcccagttgtgtgaggctgcgagagaaagggagtgcatgtgcgcgcgtgcatgtgtgcgtgcgtgtgtgttcacgtgttctcgtgcgggcgcgtgagtggtcttcaaacgagggtcccgatccccggggcggcaggaagggggccgactccacgctgtcctttgggatgatacttggatgcagctcttgggaccgtgttctgcagcccagccttcctgttggggtggggcctctcctactatgcaatttttcaagagctccttgaccctgctttttgcttcttgagttgtcttttgccattatggggactttggtttgacccaggggtcagccttaggaaggccttcaggaggaggccgagttccccttcagtaccacccctctctccccaccttccctctcccggcaacatctctgggaatcaacagcatattgacacgttggagccgagcctgaacatgcccctcggccccagcacatggaaaacccccttccttgcctaaggtgtctgagtttctggctcttgaggcatttccagacttgaaattctcatcagtccattgctcttgagtctttgcagagaacctcagatcaggtgcacctgggagaaagactttgtccccacttacagatctatctcctcccttgggaagggcagggaatggggacggtgtatggaggggagggatctcctgcgcccttcattgccacacttggtgggaccatgaacatctttagtgtctgagcttctcaaattagctgcaataggaaaaaaacaaattgggaaatgaaaaaaaaatgggaagattaaaaagcacagggggaagaagaagagatttcggaggccatcctgccaggggcggacggggctgactcctgctctctggaggacggtcagtccatgtctcggagaaacgggtgagctgagcttggcgtttggacccagttcagtgaggttcttgggttttgtgcctttggggcagaccccaggcaaggatgtctgagaccacttgggcgctgttttctcagctccaatttcaagagtgagctatcaaacccagagcggaaggagggagctctgatgagcacggtttgtcacacgataaagggattttttttttcagggctactacggttgatcttgcaactctgtaaatatgtatgtagacacttttaaaagcacgtatttatgtccctgactgtaaatgctccatttttaaagttttataacttgtgttatttaatgagtcagtcaatcggctgcagtatgggatctgataaggatctaggagaagggtctcatgcggaccctcacatgggcagaaaaatggtggtcattggccgacatcacagttttcctgtttcccacccagctaaaaaccgttgtttgctttaaattttcataaactggaatcctttcacccgctcctacagctaaccctcacaagcatgaagtgctgtggctgttccttatcctaatgatgcgcttttgtcccgtaaatgttaacactcatgaagcataccccggcctctcagttcttgagggcctccccaccgcagcagcaaggaaagctcacgaaccccaaacctggcaagtcacctgcagcccatggtgagctctgggaagtgtggttgaggccttggggtcactccttttttgcatgtgcaaatgtgctggtcacccttcaacgctcccagacggtcaggaaaactgttccaatcatgaaaaggggggatgattttgtaaaagtggcatttcctggtcagtggtggtcttcaagacgacagctctgtatctgccatgtgaagagaattaacaataaaagtgtgaagagcgattgtgaggaacaaaaaaaaaaaaaaa

SEQ ID NO: 44 - protein, 5 aa, Artificial Sequence
  Description of Artificial Sequence Synthetic peptide
GGGGC

SEQ ID NO: 45 - DNA, 2339 bp, Homo sapiens
ccaagcctgagaatcaggagaagcctcacagtgacacccccaactgaggaaactcacagagctgggacatactctacttcttcagaaaaaagtatactgactagagtggagtccccctggggagtcagaaagcctgtgaaagatctcacttgttcaaaagtccaagtgtgaattactgtctcacagataaaccaaagtattttgaaaaacaaaggggagaaaagaaattactccccagaactctcaggcatctagaggacacccaagaacgtgggagtcagctgcttcttgtgtgcagccatgaagtctgtgcactccagtccccagaacacgagtcataccatcatgacgttttacccaaccatggaagaatttgcagatttcaacacatatgttgcttacatggagtcccaaggcgcacatcaagctggccttgccaaggtaattccacccaaggaatggaaagccagacagatgtatgatgatatcgaagacatcttaatagccactcccctccagcaggtgacctctgggcagggaggtgtgtttactcaataccataaaaagaagaaagccatgagggtggggcagtatcgccgcttggcaaacagtaaaaaatatcagactccgccacaccagaattttgcagatttggagcaacgatactggaagagccaccccggtaatccaccaatttatggtgctgatatcagcggctccttatttgaagaaagcactaaacaatggaacctaggacacctgggaacaattctggacctgttggagcaggaatgtggggttgtcatcgagggtgtcaacacaccctacctgtactttggcatgtggaagaccacgtttgcctggcacacagaggacatggacctttacagcatcaactacctgcactttggggagcccaaaacttggtacgtggtgcccccagaacatggtcagcacctggaacgcctggccagggagctcttcccagacatttctcggggctgtgaggccttcctgcggcacaaagtggccctcatctcgcctacagttctcaaggaaaatgggattcccttcaattgcatgactcaggaggctggggagttcatggtgacctttccctatggctaccatgctggcttcaatcacggcttcaactgcgcagaagccattaattttgccactccacgatggattgattatggcaaaatggcctctcagtgtagctgtggggagtcgacagtgaccttttccatggacccctttgtgcgcattgtgcaacccgagagttatgagctctggaaacacaggcaagacttggccattgtggaacacacagagcccagggttgcagaaagccaagagctgagcaactggagagatgatatagtacttagaagagctgctctgggcctgaggcttctcccaaacctcacagcccagtgtcccacacagcctgtgtcctcagggcactgttacaacccaaaaggctgtggcactgatgctgtgcctggatccgcattccaaagctctgcatatcatacccagacccagtcacttaccctggggatgtcagccagggttcttctcccttccactggaagctggggttctggtcgtggtcgtggtcgtggtcaaggtcaaggtcgaggttgcagtcgtggtcgtggtcatggttgttgtactcgagaactggggactgaggagccaactgttcagcctgcatccaagaggcgccttttaatgggtacaaggagtagagctcaaggccacaggcctcagctcccgcttgccaatgatttgatgacaaatctgtccctttgagtggtggccttcagcatcttgccaaggcttctggctgctgctgtgtccctgatcttcaactcctggggcccccactggatcgtgatgaaaccatgcaccctggcctgtgcctgctatccctcaacagcactactagtaatctccctgatgttgtctgcatgactcctcccaatgtcattgtgcctttgattaagttttccagggacactggtggggactggaactgattaagttcaccagggacacttgcctggtgaacatgggcaaggctgtagcaatggaccacttttacggctctagggttctgactccaactaagttttccagaatctcctgggctcctgactcatctgctgggtctaaagacactgagtttagggatattttcctccaatacatgatcaatcctctggatccacggctatggaatatggtgacaaatgtcagtgtctctcttattccaaccccaggatcagagaagattctttacctgcagtaactgacacatttccaaggcc

SEQ ID NO: 46 - protein, 506 aa, Homo sapiens
MKSVHSSPQNTSHTIMTFYPTMEEFADFNTYVAYMESQGAHQAGLAKVIPPKEWKARQMYDDIEDILIATPLQQVTSGQGGVFTQYHKKKKAMRVGQYRRLANSKKYQTPPHQNFADLEQRYWKSHPGNPPIYGADISGSLFEESTKQWNLGHLGTILDLLEQECGVVIEGVNTPYLYFGMWKTTFAWHTEDMDLYSINYLHFGEPKTWYVVPPEHGQHLERLARELFPDISRGCEAFLRHKVALISPTVLKENGIPFNCMTQEAGEFMVTFPYGYHAGFNHGFNCAEAINFATPRWIDYGKMASQCSCGESTVTFSMDPFVRIVQPESYELWKHRQDLAIVEHTEPRVAESQELSNWRDDIVLRRAALGLRLLPNLTAQCPTQPVSSGHCYNPKGCGTDAVPGSAFQSSAYHTQTQSLTLGMSARVLLPSTGSWGSGRGRGRGQGQGRGCSRGRGHGCCTRELGTEEPTVQPASKRRLLMGTRSRAQGHRPQLPLANDLMTNLSL

SEQ ID NO: 47 - DNA, 2999 bp, Homo sapiens
gatcaactatccacgctgctcgaatcacagcatgctggagggcctggctgggtgctctgactgactgatcacctgacagacggtgcggtcagtcggatgctgagaatgactgacgatgtgatgaggggcggattgaacgagtcacaggccagctggccaggagcaaaatcggcatagctgtctgactcgatggctgtacgtggttacggactgtctgccctgatagaatctcagcttcaacgcatcagaggagactgacttgaccaatggtggggatgagtcgcctgagaaatgacagactggctgacccactgacaggctgcagcgtgtgttgcaagtcttcttggaatcagctgcaggacctgtgccgcctggccaagctctcctgccctgccctcggtatctctaagaggaacctctatgactttgaagtcgagtacctgtgcgattacaagaagatccgcgaacaggaatattacctggtgaaatggcgtggatatccagactcagagagcacctgggagccacggcagaatctcaagtgtgtgcgtatcctcaagcagttccacaaggacttagaaagggagctgctccggcggcaccaccggtcaaagaccccccggcacctggacccaagcttggccaactacctggtgcagaaggccaagcagaggcgggcgctccgtcgctgggagcaggagctcaatgccaagcgcagccatctgggacgcatcactgtagagaatgaggtggacctggacggccctccgcgggccttcgtgtacatcaatgagtaccgtgttggtgagggcatcaccctcaaccaggtggctgtgggctgcgagtgccaggactgtctgtgggcacccactggaggctgctgcccgggggcgtcactgcacaagtttgcctacaatgaccagggccaggtgcggcttcgagccgggctgcccatctacgagtgcaactcccgctgccgctgcggctatgactgcccaaatcgtgtggtacagaagggtatccgatatgacctctgcatcttccgcacggatgatgggcgtggctggggcgtccgcaccctggagaagattcgcaagaacagcttcgtcatggagtacgtgggagagatcattacctcagaggaggcagagcggcggggccagatctacgaccgtcagggcgccacctacctctttgacctggactacgtggaggacgtgtacaccgtggatgccgcctactatggcaacatctcccactttgtcaaccacagttgtgaccccaacctgcaggtgtacaacgtcttcatagacaaccttgacgagcggctgccccgcatcgctttctttgccacaagaaccatccgggcaggcgaggagctcacctttgattacaacatgcaagtggaccccgtggacatggagagcacccgcatggactccaactttggcctggctgggctccctggctcccctaagaagcgggtccgtattgaatgcaagtgtgggactgagtcctgccgcaaatacctcttctagcccttagaagtctgaggccagactgactgagggggcctgaagctacatgcacctcccccactgctgccctcctgtcgagaatgactgccagggcctcgcctgcctccacctgcccccacctgctcctacctgctctacgttcagggctgtggccgtggtgaggaccgactccaggagtcccctttccctgtcccagccccatctgtgggttgcacttacaaacccccacccaccttcagaaatagtttttcaacatcaagactctctgtcgttgggattcatggcctattaaggaggtccaaggggtgagtcccaacccagccccagaatatatttgtttttgcacctgcttctgcctggagattgaggggtctgctgcaggcctcctccctgctgccccaaaggtatggggaagcaaccccagagcaggcagacatcagaggccagagtgcctagcccgacatgaagctggttccccaaccacagaaactttgtactagtgaaagaaagggggtccctgggctacgggctgaggctggtttctgctcgtgcttacagtgctgggtagtgttggccctaagagctgtagggtctcttcttcagggctgcatatctgagaagtggatgcccacatgccactggaagggaagtgggtgtccatgggccactgagcagtgagaggaaggcagtgcagagctggccagccctggaggtaggctgggaccaagctctgccttcacagtgcagtgaaggtacctagggctcttgggagctctgcggttgctaggggccctgacctggggtgtcatgaccgctgacaccactcagagctggaaccaagatctagatagtccgtagatagcacttaggacaagaatgtgcattgatggggtggtgatgaggtgccaggcactgggtagagcacctggtccacgtggattgtctcagggaagccttgaaaaccacggaggtggatgccaggaaagggcccatgtggcagaaggcaaagtacaggccaagaattgggggtgggggagatggcttccccactatgggatgacgaggcgagagggaagcccttgctgcctgccattcccagaccccagccctttgtgctcaccctggttccactggtctcaaaagtcacctgcctacaaatgtacaaaaggcgaaggttctgatggctgccttgctccttgctcccccaccccctgtgaggacttctctaggaagtccttcctgactacctgtgcccagagtgcccctacatgagactgtatgccctgctatcagatgccagatctatgtgtctgtctgtgtgtccatcccgccggccccccagactaacctccaggcatggactgaatctggttctcctcttgtacacccctcaaccctatgcagcctggagtgggcatcaataaaatgaactgtcgactgaacaaaaaaaaaaaaaaaaaa

SEQ ID NO: 48 - protein, 423 aa, Homo sapiens
MVGMSRLRNDRLADPLTGCSVCCKSSWNQLQDLCRLAKLSCPALGISKRNLYDFEVEYLCDYKKIREQEYYLVKWRGYPDSESTWEPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 49 - DNA, 3148 bp, Homo sapiens
aacaagccccggcccccaagtcccgcgcgggccggccaggggcggggcgtcgggccagctgagctatcccgtcagaccgcgccagtttgaatgaaagctctacaagatggcggcggtcggggccgaggcgcgaggagcttggtgtgtgccttgcctagtttcacttgatactcttcaggaattatgtagaaaagaaaagctcacatgtaaatcgattggaatcaccaaaaggaatctaaacaattatgaggtggaatacttgtgtgactacaaggtagtaaaggatatggaatattatcttgtaaaatggaaaggatggccagattctacaaatacttgggaacctttgcaaaatctgaagtgcccgttactgcttcagcaattctctaatgacaagcataattatttatctcaggtaaagaaaggcaaagcaataactccaaaagacaataacaaaactttgaaacctgccattgctgagtacattgtgaagaaggctaaacaaaggatagctctgcagagatggcaagatgaactcaacagaagaaagaatcataaaggaatgatatttgttgaaaatactgttgatttagagggcccaccttcagacttctattacattaacgaatacaaaccagctcctggaatcagcttagtcaatgaagctacctttggttgttcatgcacagattgcttctttcaaaaatgttgtcctgctgaagctggagttcttttggcttataataaaaaccaacaaattaaaatcccacctggtactcccatctatgaatgcaactcaaggtgtcagtgtggtcctgattgtcccaataggattgtacaaaaaggcacacagtattcgctttgcatctttcgaactagcaatggacgtggctggggtgtaaagacccttgtgaagattaaaagaatgagttttgtcatggaatatgttggagaggtaatcacaagtgaagaagctgaaagacgaggacagttctatgacaacaagggaatcacgtatctctttgatctggactatgagtctgatgaattcacagtggatgcggctcgatacggcaatgtgtctcattttgtgaatcacagctgtgacccaaatcttcaggtgttcaatgttttcattgataacctcgatactcgtcttccccgaatagcattgttttccacaagaaccataaatgctggagaagagctgacttttgattatcaaatgaaaggttctggagatatatcttcagattctattgaccacagcccagccaaaaagagggtcagaacagtatgtaaatgtggagctgtgacttgcagaggttacctcaactgaactttttcaggaaatagagctgatgattataatatttttttcctaatgttaacatttttaaaaatacatatttgggactcttattatcaaggttctacctatgttaatttacaattcatgtttcaagacatttgccaaatgtattaccgatgcctctgaaaagggggtcactgggtctcatagactgatatgaagtcgacatatttatagtgcttagagaccaaactaatggaaggcagactatttacagcttagtatatgtgtacttaagtctatgtgaacagagaaatgcctcccgtagtgtttgaaagcgttaagctgataatgtaattaacaactgctgagagatcaaagattcaacttgccatacacctcaaattcggagaaacagttaatttgggcaaatctacagttctgtttttgctactctattgtcattcctgtttaatactcactgtacttgtatttgagacaaataggtgatactgaattttatactgttttctacttttccattaaaacattggcacctcaatgataaagaaatttaaggtataaaattaaatgtaaaaattaatttcagcttcatttcgtatttcgaagcaatctagactgttgtgatgagtgtatgtctgaacctgtaattcttaaaagacttcttaatcttctagaagaaaaatctccgaagagctctctctagaagtccaaaatggctagccattatgcttctttgaaaggacatgataatgggaccaggatggttttttggagtaccaagcaaggggaatggagcactttaagggcgcctgttagtaacatgaattggaaatctgtgtcgagtacctctgatctaaacggtaaaacaagctgcctggagagcagctgtacctaacaatactgtaatgtacattaacattacagcctctcaatttcaggcaggtgtaacagttcctttccaccagatttaatatttttatacttcctgcaggttcttcttaaaaagtaatctatatttttgaactgatacttgttttatacataaattttttttagatgtgataaagctaaacttggccaaagtgtgtgcctgaattattagacctttttattagtcaacctacgaagactaaaatagaatatattagttttcaagggagtgggaggcttccaacatagtattgaatctcaggaaaaactattctttcatgtctgattctgagatttctaattgtgttgtgaaaatgataaatgcagcaaatctagctttcagtattcctaatttttacctaagctcattgctccaggctttgattacctaaaataagcttggataaaattgaaccaacttcaagaatgcagcacttcttaatctttagctctttcttgggagaagctagactttattcattatattgctatgacaacttcactctttcataatatataggataaattgtttacatgattggaccctcagattctgttaaccaaaattgcagaatggggggccaggcctgtgtggtggctcacacctgtgatcccagcactttgggaggctgaggtaggaggatcacgtgaggtcgggagttcaagaccagcctggccatcatggtgaaaccctgtctctactgaaaatacaaaaattagccgggcgtggtggcacacgcctgtagtcccagctactcaggaggctgaggcaggagaatcacttgaattcaggaggcggaggttgcagtgagccaagatcataccactgcactgcagcctgagtgacacagtaagactgtctccaaaaaaaaaaaaaaaaaa

SEQ ID NO: 50 - DNA, 33 bp, Artificial Sequence
  Description of Artificial Sequence Synthetic oligonucleotide
  modified_base (3)..(31): a, c, t, g, unknown or other
aannnnnnnnnnnnnnnnnnnnnnnnnnnnntt

SEQ ID NO: 51 - DNA, 3106 bp, Homo sapiens
agtttgaatgaaagctctacaagatggcggcggtcggggccgaggcgcgaggaggtgaggctggagcgcggccccctcgccttccctgttcccagcttggtgtgtgccttgcctagtttcacttgatactcttcaggaattatgtagaaaagaaaagctcacatgtaaatcgattggaatcaccaaaaggaatctaaacaattatgaggtggaatacttgtgtgactacaaggtagtaaaggatatggaatattatcttgtaaaatggaaaggatggccagattctacaaatacttgggaacctttgcaaaatctgaagtgcccgttactgcttcagcaattctctaatgacaagcataattatttatctcaggtaaagaaaggcaaagcaataactccaaaagacaataacaaaactttgaaacctgccattgctgagtacattgtgaagaaggctaaacaaaggatagctctgcagagatggcaagatgaactcaacagaagaaagaatcataaaggaatgatatttgttgaaaatactgttgatttagagggcccaccttcagacttctattacattaacgaatacaaaccagctcctggaatcagcttagtcaatgaagctacctttggttgttcatgcacagattgcttctttcaaaaatgttgtcctgctgaagctggagttcttttggcttataataaaaaccaacaaattaaaatcccacctggtactcccatctatgaatgcaactcaaggtgtcagtgtggtcctgattgtcccaataggattgtacaaaaaggcacacagtattcgctttgcatctttcgaactagcaatggacgtggctggggtgtaaagacccttgtgaagattaaaagaatgagttttgtcatggaatatgttggagaggtaatcacaagtgaagaagctgaaagacgaggacagttctatgacaacaagggaatcacgtatctctttgatctggactatgagtctgatgaattcacagtggatgcggctcgatacggcaatgtgtctcattttgtgaatcacagctgtgacccaaatcttcaggtgttcaatgttttcattgataacctcgatactcgtcttccccgaatagcattgttttccacaagaaccataaatgctggagaagagctgacttttgattatcaaatgaaaggttctggagatatatcttcagattctattgaccacagcccagccaaaaagagggtcagaacagtatgtaaatgtggagctgtgacttgcagaggttacctcaactgaactttttcaggaaatagagctgatgattataatatttttttcctaatgttaacatttttaaaaatacatatttgggactcttattatcaaggttctacctatgttaatttacaattcatgtttcaagacatttgccaaatgtattaccgatgcctctgaaaagggggtcactgggtctcatagactgatatgaagtcgacatatttatagtgcttagagaccaaactaatggaaggcagactatttacagcttagtatatgtgtacttaagtctatgtgaacagagaaatgcctcccgtagtgtttgaaagcgttaagctgataatgtaattaacaactgctgagagatcaaagattcaacttgccatacacctcaaattcggagaaacagttaatttgggcaaatctacagttctgtttttgctactctattgtcattcctgtttaatactcactgtacttgtatttgagacaaataggtgatactgaattttatactgttttctacttttccattaaaacattggcacctcaatgataaagaaatttaaggtataaaattaaatgtaaaaattaatttcagcttcatttcgtatttcgaagcaatctagactgttgtgatgagtgtatgtctgaacctgtaattcttaaaagacttcttaatcttctagaagaaaaatctccgaagagctctctctagaagtccaaaatggctagccattatgcttctttgaaaggacatgataatgggaccaggatggttttttggagtaccaagcaaggggaatggagcactttaagggcgcctgttagtaacatgaattggaaatctgtgtcgagtacctctgatctaaacggtaaaacaagctgcctggagagcagctgtacctaacaatactgtaatgtacattaacattacagcctctcaatttcaggcaggtgtaacagttcctttccaccagatttaatatttttatacttcctgcaggttcttcttaaaaagtaatctatatttttgaactgatacttgttttatacataaattttttttagatgtgataaagctaaacttggccaaagtgtgtgcctgaattattagacctttttattagtcaacctacgaagactaaaatagaatatattagttttcaagggagtgggaggcttccaacatagtattgaatctcaggaaaaactattctttcatgtctgattctgagatttctaattgtgttgtgaaaatgataaatgcagcaaatctagctttcagtattcctaatttttacctaagctcattgctccaggctttgattacctaaaataagcttggataaaattgaaccaacttcaagaatgcagcacttcttaatctttagctctttcttgggagaagctagactttattcattatattgctatgacaacttcactctttcataatatataggataaattgtttacatgattggaccctcagattctgttaaccaaaattgcagaatggggggccaggcctgtgtggtggctcacacctgtgatcccagcactttgggaggctgaggtaggaggatcacgtgaggtcgggagttcaagaccagcctggccatcatggtgaaaccctgtctctactgaaaatacaaaaattagccgggcgtggtggcacacgcctgtagtcccagctactcaggaggctgaggcaggagaatcacttgaattcaggaggcggaggttgcagtgagccaagatcataccactgcactgcagcctgagtgacacagtaagactgtctccaaaaaaaaaaaaaaaaaa

SEQ ID NO: 52 - DNA, 2608 bp, Homo sapiens
aacaagccccggcccccaagtcccgcgcgggccggccaggggcggggcgtcgggccagctgagctatcccgtcagaccgcgccagtttgaatgaaagctctacaagatggcggcggtcggggccgaggcgcgaggagcttggtgtgtgccttgcctagtttcacttgatactcttcaggaattatgtagaaaagaaaagctcacatgtaaatcgattggaatcaccaaaaggaatctaaacaattatgaggtggaatacttgtgtgactacaaggtagtaaaggatatggaatattatcttgtaaaatggaaaggatggccagattctacaaatacttgggaacctttgcaaaatctgaagtgcccgttactgcttcagcaattctctaatgacaagcataattatttatctcaggtaatcacaagtgaagaagctgaaagacgaggacagttctatgacaacaagggaatcacgtatctctttgatctggactatgagtctgatgaattcacagtggatgcggctcgatacggcaatgtgtctcattttgtgaatcacagctgtgacccaaatcttcaggtgttcaatgttttcattgataacctcgatactcgtcttccccgaatagcattgttttccacaagaaccataaatgctggagaagagctgacttttgattatcaaatgaaaggttctggagatatatcttcagattctattgaccacagcccagccaaaaagagggtcagaacagtatgtaaatgtggagctgtgacttgcagaggttacctcaactgaactttttcaggaaatagagctgatgattataatatttttttcctaatgttaacatttttaaaaatacatatttgggactcttattatcaaggttctacctatgttaatttacaattcatgtttcaagacatttgccaaatgtattaccgatgcctctgaaaagggggtcactgggtctcatagactgatatgaagtcgacatatttatagtgcttagagaccaaactaatggaaggcagactatttacagcttagtatatgtgtacttaagtctatgtgaacagagaaatgcctcccgtagtgtttgaaagcgttaagctgataatgtaattaacaactgctgagagatcaaagattcaacttgccatacacctcaaattcggagaaacagttaatttgggcaaatctacagttctgtttttgctactctattgtcattcctgtttaatactcactgtacttgtatttgagacaaataggtgatactgaattttatactgttttctacttttccattaaaacattggcacctcaatgataaagaaatttaaggtataaaattaaatgtaaaaattaatttcagcttcatttcgtatttcgaagcaatctagactgttgtgatgagtgtatgtctgaacctgtaattcttaaaagacttcttaatcttctagaagaaaaatctccgaagagctctctctagaagtccaaaatggctagccattatgcttctttgaaaggacatgataatgggaccaggatggttttttggagtaccaagcaaggggaatggagcactttaagggcgcctgttagtaacatgaattggaaatctgtgtcgagtacctctgatctaaacggtaaaacaagctgcctggagagcagctgtacctaacaatactgtaatgtacattaacattacagcctctcaatttcaggcaggtgtaacagttcctttccaccagatttaatatttttatacttcctgcaggttcttcttaaaaagtaatctatatttttgaactgatacttgttttatacataaattttttttagatgtgataaagctaaacttggccaaagtgtgtgcctgaattattagacctttttattagtcaacctacgaagactaaaatagaatatattagttttcaagggagtgggaggcttccaacatagtattgaatctcaggaaaaactattctttcatgtctgattctgagatttctaattgtgttgtgaaaatgataaatgcagcaaatctagctttcagtattcctaatttttacctaagctcattgctccaggctttgattacctaaaataagcttggataaaattgaaccaacttcaagaatgcagcacttcttaatctttagctctttcttgggagaagctagactttattcattatattgctatgacaacttcactctttcataatatataggataaattgtttacatgattggaccctcagattctgttaaccaaaattgcagaatggggggccaggcctgtgtggtggctcacacctgtgatcccagcactttgggaggctgaggtaggaggatcacgtgaggtcgggagttcaagaccagcctggccatcatggtgaaaccctgtctctactgaaaatacaaaaattagccgggcgtggtggcacacgcctgtagtcccagctactcaggaggctgaggcaggagaatcacttgaattcaggaggcggaggttgcagtgagccaagatcataccactgcactgcagcctgagtgacacagtaagactgtctccaaaaaaaaaaaaaaaaaa

SEQ ID NO: 53 - DNA, 2566 bp, Homo sapiens
agtttgaatgaaagctctacaagatggcggcggtcggggccgaggcgcgaggaggtgaggctggagcgcggccccctcgccttccctgttcccagcttggtgtgtgccttgcctagtttcacttgatactcttcaggaattatgtagaaaagaaaagctcacatgtaaatcgattggaatcaccaaaaggaatctaaacaattatgaggtggaatacttgtgtgactacaaggtagtaaaggatatggaatattatcttgtaaaatggaaaggatggccagattctacaaatacttgggaacctttgcaaaatctgaagtgcccgttactgcttcagcaattctctaatgacaagcataattatttatctcaggtaatcacaagtgaagaagctgaaagacgaggacagttctatgacaacaagggaatcacgtatctctttgatctggactatgagtctgatgaattcacagtggatgcggctcgatacggcaatgtgtctcattttgtgaatcacagctgtgacccaaatcttcaggtgttcaatgttttcattgataacctcgatactcgtcttccccgaatagcattgttttccacaagaaccataaatgctggagaagagctgacttttgattatcaaatgaaaggttctggagatatatcttcagattctattgaccacagcccagccaaaaagagggtcagaacagtatgtaaatgtggagctgtgacttgcagaggttacctcaactgaactttttcaggaaatagagctgatgattataatatttttttcctaatgttaacatttttaaaaatacatatttgggactcttattatcaaggttctacctatgttaatttacaattcatgtttcaagacatttgccaaatgtattaccgatgcctctgaaaagggggtcactgggtctcatagactgatatgaagtcgacatatttatagtgcttagagaccaaactaatggaaggcagactatttacagcttagtatatgtgtacttaagtctatgtgaacagagaaatgcctcccgtagtgtttgaaagcgttaagctgataatgtaattaacaactgctgagagatcaaagattcaacttgccatacacctcaaattcggagaaacagttaatttgggcaaatctacagttctgtttttgctactctattgtcattcctgtttaatactcactgtacttgtatttgagacaaataggtgatactgaattttatactgttttctacttttccattaaaacattggcacctcaatgataaagaaatttaaggtataaaattaaatgtaaaaattaatttcagcttcatttcgtatttcgaagcaatctagactgttgtgatgagtgtatgtctgaacctgtaattcttaaaagacttcttaatcttctagaagaaaaatctccgaagagctctctctagaagtccaaaatggctagccattatgcttctttgaaaggacatgataatgggaccaggatggttttttggagtaccaagcaaggggaatggagcactttaagggcgcctgttagtaacatgaattggaaatctgtgtcgagtacctctgatctaaacggtaaaacaagctgcctggagagcagctgtacctaacaatactgtaatgtacattaacattacagcctctcaatttcaggcaggtgtaacagttcctttccaccagatttaatatttttatacttcctgcaggttcttcttaaaaagtaatctatatttttgaactgatacttgttttatacataaattttttttagatgtgataaagctaaacttggccaaagtgtgtgcctgaattattagacctttttattagtcaacctacgaagactaaaatagaatatattagttttcaagggagtgggaggcttccaacatagtattgaatctcaggaaaaactattctttcatgtctgattctgagatttctaattgtgttgtgaaaatgataaatgcagcaaatctagctttcagtattcctaatttttacctaagctcattgctccaggctttgattacctaaaataagcttggataaaattgaaccaacttcaagaatgcagcacttcttaatctttagctctttcttgggagaagctagactttattcattatattgctatgacaacttcactctttcataatatataggataaattgtttacatgattggaccctcagattctgttaaccaaaattgcagaatggggggccaggcctgtgtggtggctcacacctgtgatcccagcactttgggaggctgaggtaggaggatcacgtgaggtcgggagttcaagaccagcctggccatcatggtgaaaccctgtctctactgaaaatacaaaaattagccgggcgtggtggcacacgcctgtagtcccagctactcaggaggctgaggcaggagaatcacttgaattcaggaggcggaggttgcagtgagccaagatcataccactgcactgcagcctgagtgacacagtaagactgtctccaaaaaaaaaaaaaaaaaa

SEQ ID NO: 54 - protein, 410 aa, Homo sapiens
MAAVGAEARGAWCVPCLVSLDTLQELCRKEKLTCKSIGITKRNLNNYEVEYLCDYKVVKDMEYYLVKWKGWPDSTNTWEPLQNLKCPLLLQQFSNDKHNYLSQVKKGKAITPKDNNKTLKPAIAEYIVKKAKQRIALQRWQDELNRRKNHKGMIFVENTVDLEGPPSDFYYINEYKPAPGISLVNEATFGCSCTDCFFQKCCPAEAGVLLAYNKNQQIKIPPGTPIYECNSRCQCGPDCPNRIVQKGTQYSLCIFRTSNGRGWGVKTLVKIKRMSFVMEYVGEVITSEEAERRGQFYDNKGITYLFDLDYESDEFTVDAARYGNVSHFVNHSCDPNLQVFNVFIDNLDTRLPRIALFSTRTINAGEELTFDYQMKGSGDISSDSIDHSPAKKRVRTVCKCGAVTCRGYLN

SEQ ID NO: 55 - protein, 350 aa, Homo sapiens; identical to SEQ ID NO: 6

SEQ ID NO: 56 - protein, 230 aa, Homo sapiens
MAAVGAEARGAWCVPCLVSLDTLQELCRKEKLTCKSIGITKRNLNNYEVEYLCDYKVVKDMEYYLVKWKGWPDSTNTWEPLQNLKCPLLLQQFSNDKHNYLSQVITSEEAERRGQFYDNKGITYLFDLDYESDEFTVDAARYGNVSHFVNHSCDPNLQVFNVFIDNLDTRLPRIALFSTRTINAGEELTFDYQMKGSGDISSDSIDHSPAKKRVRTVCKCGAVTCRGYLN

SEQ ID NO: 57 - protein, 170 aa, Homo sapiens
MEYYLVKWKGWPDSTNTWEPLQNLKCPLLLQQFSNDKHNYLSQVITSEEAERRGQFYDNKGITYLFDLDYESDEFTVDAARYGNVSHFVNHSCDPNLQVFNVFIDNLDTRLPRIALFSTRTINAGEELTFDYQMKGSGDISSDSIDHSPAKKRVRTVCKCGAVTCRGYLN

SEQ ID NO: 58 - DNA, 33 bp, Homo sapiens
aaggattagactgaaccgaattggtatatagtt

SEQ ID NO: 59 - DNA, 32 bp, Homo sapiens
aaggattagactgagctgaattggtatatagt

SEQ ID NO: 60 - DNA, 33 bp, Homo sapiens
caaactaccacttacctccctcaccaaagccca

SEQ ID NO: 61 - DNA, 34 bp, Homo sapiens
caaactaccacttacctccctcaccaaagcccat

SEQ ID NO: 62 - DNA, 33 bp, Homo sapiens
caaactaccacctacctccctcaccaaagccca

SEQ ID NO: 63 - DNA, 32 bp, Homo sapiens
attaatgcaaacaatacctaacagacccacag

SEQ ID NO: 64 - DNA, 31 bp, Homo sapiens
attaatgcaaacaatacctaacagacccaca

SEQ ID NO: 65 - DNA, 32 bp, Homo sapiens
attaatgcaaacagtacctaacaaacctacag

SEQ ID NO: 66 - DNA, 33 bp, Homo sapiens
gtactcccgattgaaacccccattcgtataata

SEQ ID NO: 67 - DNA, 34 bp, Homo sapiens
gtactcccgattgaaacccccattcgtataataa

SEQ ID NO: 68 - DNA, 33 bp, Homo sapiens
gtactcccgattgaagcccccattcgtataata

SEQ ID NO: 69 - DNA, 33 bp, Homo sapiens
ctccctaggaggcctgcccccgctaaccggctt

SEQ ID NO: 70 - DNA, 33 bp, Homo sapiens
ctccctaggaggcctacccccgctaaccggctt

(19) United States
(12) Patent Application Publication
(10) Pub. No.: US 2018/0291400 A1
(43) Pub. Date: Oct. 11, 2018

(54) METHODS AND COMPOSITIONS TO INCREASE HUMAN SOMATIC CELL NUCLEAR TRANSFER (SCNT) EFFICIENCY BY REMOVING HISTONE H3-LYSINE TRIMETHYLATION, AND DERIVATION OF HUMAN NT-ESC

(71) Applicants: CHILDREN'S MEDICAL CENTER CORPORATION, Boston, MA (US); SUNG KWANG MEDICAL FOUNDATION, Seoul (KR)

(72) Inventors: Yi ZHANG, Newton, MA (US); Dong Ryul LEE, Seoul (KR); Shogo MATOBA, Brookline, MA (US); Young CHUNG, Shrewbury, MA (US)

(21) Appl. No.: 15/765,860

(22) PCT Filed: Oct. 7, 2016
(86) PCT No.: PCT/US16/55890

§ 371 Date: Apr. 4, 2018

Related U.S. Application Data
(60) Provisional application No. 62/239,318, filed on Oct. 9, 2015.
(60) Provisional application No. 62/242,050, filed on Oct. 15, 2015.

(51) Int. Cl. C12N 15/873; C12N 9/02; C12N 5/10; A61K 35/545

(52) U.S. Cl. CPC C12N 15/873; C12N 9/0071; C12Y 114/11027; A61K 35/545; C12Y 201/01043; C12N 5/10

(57) ABSTRACT

The present invention provides methods and compositions to improve the efficiency of somatic cell nuclear transfer (SCNT) of human cells and the consequent production of human nuclear transfer ESC (hNT-ESCs). More specifically, the present invention relates to the discovery that trimethylation of Histone H3-Lysine 9 (H3K9me3) in reprogramming resistant regions (RRRs) in the nuclear genetic material of human donor somatic cells prevents efficient human somatic cell nuclear reprogramming or SCNT. The present invention provide methods and compositions to decrease H3K9me3 in methods to improve efficacy of hSCNT by exogenous or overexpression of the demethylase KDM4 family and/or inhibiting methylation of H3K9me3 by inhibiting the histone methyltransferases SUV39h1 and/or SUV39h2.

### CROSS REFERENCED TO RELATED APPLICATIONS

This Application claims the benefit under 35 U.S.C. § 119(e) of U.S. Provisional Application Ser. No. 62/239,318 filed on Oct. 9, 2015, and U.S. Provisional Application 62/242,050 filed on Oct. 15, 2015, the contents of each are incorporated herein in their entirety by reference.

### SEQUENCE LISTING

The instant application contains a Sequence Listing which has been submitted in ASCII format via EFS-Web and is hereby incorporated by reference in its entirety. Said ASCII copy, created on Oct. 7, 2016, is named 701039-085852-PCT_SL.txt and is 157,721 bytes in size.

### FIELD OF THE INVENTION

The present invention relates generally to the field of somatic cell nuclear transfer (SCNT), more specifically to increasing efficiency of human SCNT and producing human nuclear transfer ESCs (hNT-ESCs) by overexpression of the demethylase KDM4 family and/or inhibiting methylation of H3K9me3 by inhibiting SUV39h1 and/or SUV39h2 histone methyltransferases.

### BACKGROUND OF THE INVENTION

The differentiated somatic cell genome can be reprogrammed back into an embryonic state when the nucleus is exposed to the molecular milieu of the oocyte cytoplasm via somatic cell nuclear transfer (SCNT) (Gurdon, 1962), thereby enabling the generation of pluripotent embryonic stem cells (ESCs) from terminally-differentiated somatic cells (Wakayama et al., 2001). Because SCNT derived ESCs (NT-ESCs) are genetically autologous to the nuclear donor somatic cells, hSCNT has great potential in therapeutic and regenerative medicine, including disease modeling and cell/tissue replacement therapy (Hochedlinger and Jaenisch, 2003; Yang et al., 2007). Thus, hSCNT can be used to fix mitochondria gene-related defects, which cannot be done through transcription factor-based reprogramming (Ma et al., 2015). Despite the great potential of human NT-ESCs, technical difficulties makes its application to human therapeutics extremely difficult (French et al., 2008; Noggle et al., 2011; Simerly et al., 2003).

The first NT-ESCs were generated by the Mitalipov group using differentiated fetal and infant fibroblasts as nuclear donor (Tachibana et al., 2013). Using their optimized conditions, the inventors and others succeeded in deriving human NT-ESCs from adult and aged patient somatic cells (Chung et al., 2014; Yamada et al., 2014). However, derivation of NT-ESCs still remains a very difficult task due to the extremely low rate of SCNT embryos to develop to the blastocyst stage. Currently only oocytes with the highest quality from certain females can support the development of SCNT embryos to the blastocyst stage (Chung et al., 2014; Tachibana et al., 2013), limiting the useful oocyte donor pools.

Terminally differentiated somatic cells can be reprogrammed to the totipotent state when transplanted into enucleated oocytes by the means of somatic cell nuclear transfer (SCNT) (Gurdon, 1962). Because SCNT allows the generation of an entire animal from a single nucleus of differentiated somatic cell, it has great potential in agriculture, biomedical industry, and endangered species conservation (Yang et al., 2007). Indeed, more than 20 mammalian species have been cloned through SCNT (Rodriguez-Osorio et al., 2012) since the first successful mammalian cloning in sheep in 1997 (Wilmut et al., 1997). Moreover, because pluripotent embryonic stem cells can be established from SCNT-generated blastocysts (Wakayama et al., 2001), SCNT holds great promise in human therapies (Hochedlinger and Jaenisch, 2003). This promise is closer to reality after the recent success in derivation of the first human nuclear transfer embryonic stem cells (hNT-ESCs) (Tachibana et al., 2013), as well as the generation of human hNT-ESCs from aged adult or human patient cells (Chung et al., 2014; Yamada et al., 2014). These hNT-ESCs can serve as valuable cell sources for in vitro disease modeling as well as a source of cells for regenerative therapy and cell/tissue-replacement therapies.

Despite its tremendous potential, several technical problems have prevented the practical use of SCNT, in particular, it has an extremely low efficiency in producing cloned animals. For example, approximately half of mouse SCNT embryos display developmental arrest prior to implantation, and only 1-2% of embryos transferred to surrogate mothers develop to term (Ogura et al., 2013). With the exception of bovine species, which have a higher rate of reproductive cloning efficiency (5 to 20%), the overall reproductive cloning efficiency in all other species is very low (1 to 5%) (Rodriguez-Osorio et al., 2012). Furthermore, the success rate of hNT-ESCs establishment is also low owing to their poor preimplantation development (10 to 25% to the blastocyst stage; Tachibana et al., 2013; Yamada et al., 2014).

To realize the application potential of SCNT, efforts have been taken to improve SCNT cloning efficiency. First, transient treatment of 1-cell SCNT embryos with histone deacetylase (HDAC) inhibitors, such as Tricostatin A (TSA) or scriptaid, has been reported to improve reprogramming efficiency of various mammalian species including mouse (Kishigami et al., 2006; Van Thuan et al., 2009), pig (Zhao et al., 2009), bovine (Akagi et al., 2011) and humans (Tachibana et al., 2013; Yamada et al., 2014). Secondly, knockout or knockdown of Xist has been reported to improve postimplantation development of mouse SCNT embryos (Inoue et al., 2010; Matoba et al., 2011). However, neither of these methods improve the cloning efficiency of human SCNT enough for human SCNT to be useful for the generation of human totipotent and pluripotent stem cells (e.g. human NT-ESCs) for therapeutic cloning or regenerative therapies.

The developmental defects of SCNT embryos start to appear at the time of zygotic gene activation (ZGA), which occurs at the 2-cell stage in mouse and at the 4- to 8-cell stage in pig, bovine and human (Schultz, 2002). SCNT embryos have difficulties in ZGA due to undefined epigenetic barriers pre-existing in the genome of donor cells. Although a number of dysregulated genes in mouse 2-cell SCNT embryos (Inoue et al., 2006; Suzuki et al., 2006; Vassena et al., 2007), and in the late cleavage stage human SCNT embryos (Noggle et al., 2011) have been identified, the nature of the “pre-existing epigenetic barriers” and their relationship with impaired ZGA in SCNT embryos are unknown.

Accordingly, there is a need to improve human SCNT cloning efficiency by removing such epigenetic barriers in the genome of the donor cell nuclei so that the human SCNT embryo can proceed efficiently through zygotic gene activation (ZGA) without developmental arrest and successfully develop through the 2-, 4- and 8-cell stage to blastocyst without developmental defects or loss of viability.

### SUMMARY OF THE INVENTION

The present invention is based, in part, upon the discovery that in human somatic cells, H3K9me3 also serves as a barrier in human SCNT reprogramming. The inventors have demonstrated that KDM4A overexpression (e.g., by injection of exogenous KDM4A mRNA) stops developmental arrest at the time of zygotic gene activation (ZGA) and significantly improves human SCNT embryo development, allowing efficient production of patient-specific human NT-ESCs using human oocytes obtained from donors whose oocytes, in controlled experiments, failed to develop to blastocyst without the help of KDM4A overexpression. Thus, the inventors have discovered a method to expanded the usability of human oocyte donors for human SCNT (hSCNT) and establishes the histone demethylase-assisted SCNT, e.g., by overexpressing a member of the KDM4 family can be used in a method for improving human SCNT for therapeutic cloning and production of human nuclear-transfer ESC (NT-ESC), in particular, patient-derived human NT-ESCS for both therapeutic use and in research and disease modeling. The present invention is not intended for reproductive cloning of a human.

Mammalian (non-human) oocytes can reprogram somatic cells into a totipotent state, which allows animal reproductive cloning through somatic cell nuclear transfer (SCNT), or the production of ES cell lines (NT-ESC) from blastocyst developed from SCNT embryos. However, the majority of SCNT embryos fail to develop into blastocyst or to term due to undefined reprogramming defects. The inefficiency of mammalian SCNT is a critical limitation to the development of patient-specific hESC lines for regenerative medicine applications.

Although the production of human SCNT-derived human blastocysts using human donor somatic cells has been reported, the blastocyst quality and developmental efficiency was insufficient to allow the production of a human embryonic stem cell line (human ntESC, also called or hNT-ESC) (French A J et al., Stem Cells 26, 485-493 (2008)). Human nuclear transfer embryonic stem cells (hNT-ESCs) have been reported (Tachibana et al., 2013), as well as the generation of human hNT-ESCs from aged adult or human patient cells (Chung et al., 2014; Yamada et al., 2014). However, the success rate for human hNT-ESCs establishment is very low due to poor pre-implantation development (only 10 to 25% develop to the blastocyst stage; Tachibana et al., 2013; Yamada et al., 2014). The refinement of human SCNT techniques is therefore critical to improve the development to human SCNT embryos to blastocyst stage, to reduce the number of donor oocytes required for SCNT, and successfully produce human and patient-specific isogenic embryonic stem cell lines for research and cell based therapies.

The extremely low efficiency of human embryonic stem cell (hNT-ESCs) derivation using somatic cell nuclear transfer (SCNT) significantly limits its potential application. Blastocyst formation from human SCNT embryos occurs at a low rate and with only some oocyte donors. The poor developmental potential of SCNT embryos is not limited to human, but is also commonly observed in all examined mammalian species (Rodriguez-Osorio et al., 2012).

Through comparative transcriptomic and epigenomic analyses of mouse in vitro fertilization (IVF) and SCNT embryos, the inventors have previously identified that histone H3 lysine 9 trimethylation (H3K9me3) in the donor somatic cell genome functions as a barrier preventing transcriptional reprogramming of mouse cells by SCNT, leading to failure of zygotic genome activation (ZGA) and preimplantation development (Matoba et al., 2014). The inventors also previously demonstrated that this epigenetic barrier in mouse donor somatic cells could be removed by ectopically overexpressing mouse KDM4d, a H3K9me3 demethylase. Removal of H3K9me3 facilitated ZGA and consequently improved the development of mouse SCNT embryos to reach the blastocyst stage, leading to an increased rate and efficiency of mouse NT-ESC production (mNT-ESC) (Matoba et al., 2014).

More specifically, the inventors previously demonstrated in mice, that reduction of histone H3 lysine 9 trimethylation (H3K9me3) through ectopic expression of the H3K9me3 demethylase KDM4d greatly improves SCNT mouse embryo development, which is disclosed in International Application WO2016/044271, which is incorporated herein in its entirety by reference.

In contrast to the previous study, herein the inventors demonstrate that overexpression of the H3K9me3 demethylase KDM4A in human cells surprisingly improves human SCNT, and that H3K9me3 in the human somatic cell genome there is a SCNT reprogramming barrier that prevents human SCNT embryos from proceeding efficiently through zygotic gene activation (ZGA). This was unexpected as human and mouse ES cells are very different, and it could not be predicted that what worked in mice cells would work in human cells.

More specifically, as zygotic gene activation (ZGA) occurs at different times in mice and human cells, it cannot be predicted that a reprogramming method that removes the ZGA barrier in mouse cells would also work in removing the ZGA barrier at a completely different timeframe in human cells. As shown in FIG. 2A and FIG. 2E herein, the procedure and/or methods for increasing the efficiency of SCNT in mouse cells (see, FIG. 2A) is different to that for increasing SCNT efficiency in human cells (see, e.g., FIG. 2E). Herein, the inventors surprisingly demonstrate that overexpression of KDM4A significantly improves the blastocyst formation rate in human SCNT embryos by facilitating transcriptional reprogramming, allowing efficient derivation of human NT-ESCs from different human patient populations, e.g., the inventors have demonstrated the generation of hNT-ESC from adult Age-related Macular Degeneration (AMD) patient somatic nuclei donors. Thus the discovery herein of a method to increase the efficiency of human SCNT has many potential applications in a variety of contexts, including regenerative medicine and therapeutic cloning.

In particular, the inventors have discovered that histone H3 lysine 9 trimethylation (H3K9me3) in the genome of donor nuclei of a differentiated human somatic cell is a major pre-existing epigenetic barrier for efficient reprogramming of human cells by SCNT, and have discovered that decreasing H3K9me3 methylation in human donor nuclei, or in the activated SCNT embryo can increase the efficiency of human SCNT, in particular, increase the efficiency of pre-implantation development of human SCNT embryos to 8-cell or blastocyst stage.

More specifically, through comparative analysis the inventors have discovered genomic domains of human donor nuclei that are resistant to zygotic gene activation (ZGA) in human SCNT embryos. As opposed to in other mammals, such as mice, where ZGA which occurs at the 2-cell stage, and at the 4- to 8-cell stage in pig and bovine (Schultz, 2002), ZGA in humans occurs at the 8-cell stage (Schultz, 2002). The inventors herein have discovered that reprogramming resistant regions (RRRs) in human donor genetic material is enriched for the repressive histone modification, H3K9me3, and removal of this epigenetic marker in human donor somatic cells can increase the efficiency of human SCNT. Herein, two ways to improve efficacy of human SCNT are encompassed in the methods and compositions as disclosed herein, and include (i) increased expression of, or activation of an H3K9me3-specific demethylase, such as, overexpressing at least one member of the human KDM4 family (e.g., expressing exogenous human KMD4A, KDM2B, KDM4C, KDM4D or KDM4E mRNA) in oocytes or in an activated SCNT embryo (e.g., after a hybrid oocyte has been fused or activated) and/or (ii) knocking-down or inhibiting the expression or function of a human H3K9 methyltransferase, such as, e.g., human SUV39h1 or human SUV39h2 or both (i.e., SUV39h1/2), in human somatic donor nuclei. Such methods not only attenuate the ZGA defects in the human donor nuclei and reactivates the RRRs, and also greatly improves the efficiency of human SCNT, e.g., increases the % of SCNT embryos developing to 2-cell, 4-cell and 8-cell or blastocyst stage.

Thus, SUV39h1/2-mediated H3K9me3 is an “epigenetic barrier” of human SCNT and inhibition and/or removal of the trimethylation of H3K9me3 (via overexpression of KDM4A/JHDM3A, or any other member of the human KDM4 family (e.g., overexpression of any one or more of human KDM4A, human KDM4B, human KDM4C, human KDM4D, human KDM4E genes), and/or using an inhibitor of human SUV39h1/2 protein or gene, in either the nuclei of the human somatic donor cell, the recipient human oocyte, a hybrid oocyte or the human SCNT embryo, are useful in the methods, compositions and kits as disclosed herein for removing epigenetic barriers that occur in the ZGA in human cell reprogramming, in particular in reprogramming human somatic cells via human SCNT, and are encompassed for methods to improve human SCNT cloning efficiency.

Accordingly, the present invention is based on the inventor's discovery that in human cells, H3K9me3 is enriched in the RRRs in human somatic cells used in the production of SCNT embryos, and that the H3K9me3 barrier in human somatic cells can be removed by overexpression of a member of the KDM4D family.

Importantly, the inventors have demonstrated that removal of H3K9me3 by overexpression of at least one member of the human KDM4 family of proteins, e.g., human KDM4A, human KDM4B, human KDM4C, human KDM4D, human KDM4E (e.g., by introduction of exogenous mRNA encoding the KDM4 family member, e.g., KDM4A mRNA or cDNA) in the hSCNT embryo (e.g., at between 5-10 hpa, or between the 2 to 8-cell stage), the recipient oocyte, results in a surprisingly significant increase in the efficiency of human SCNT cloning. In particular, the inventors surprisingly demonstrate a greater than 20% increase in KDM4A injected hSCNT embryos developing into blastocysts (i.e., an increase from 4.2% to 26.8% with KDM4A injection), and 14% of KDM4A injected hSCNT embryos developing into the expanded blastocyst stage (as compared to none of the control hSCNT embryos).

Accordingly, aspects of the present invention are based on the discovery that the trimethylation of Histone H3-Lysine 9 (H3K9me3) in human donor somatic cells prevents efficient human somatic cell nuclear reprogramming (hSCNT). Herein, two ways to improve efficacy of human SCNT are encompassed in the methods and compositions as disclosed herein, and include (i) promoting demethylation of H3K9me3 by using overexpression (i.e., exogenous expression, or ectopic expression) of a member of the demethylase KDM4 family, e.g., KDM4A (also known as JMJD2A or JHDM3A), and/or (ii) inhibiting methylation of H3K9me3 by inhibiting the human histone methyltransferases SUV39H1 and/or SUV39H2, as the inventors previously demonstrated that inhibition of SUV39h1/2 in nuclei of the mouse donor somatic cells surprisingly increased the efficiency of mammalian SCNT efficiency (as disclosed in International application PCT/US2015/050178, filed on Sep. 15, 2015 and published as WO2016/044271, which is incorporated herein in its entirety by reference). Thus, overexpression of KDM4A/JHDM3A, or other members of the human KDM4 family (e.g., overexpression of any one or more of human KDM4A, human KDM4B, human KDM4C, human KDM4D, human KDM4E genes), and/or inhibition of human SUV39h1/2 proteins or genes are useful in the methods, compositions and kits as disclosed herein for removing epigenetic barriers that occur in the ZGA in human cell reprogramming, in particular in reprogramming human somatic cells via human SCNT.

Accordingly, aspects of the invention relate to methods, compositions and kits directed to increasing human SCNT efficiency by reducing H3K9me3 methylation in the human SCNT embryo by either (i) expressing histone demethylases which are capable of demethylating H3K9me3, e.g., for example, a member of the KDM4 family of histone demethylases, such as, for example but not limited to, JMJD2A/KDM4A and/or JMJD2D/KDM4D and/or JMJD2B/KDM4B and/or JMJD2C/KDM4C and/or JMJD2E/KDM4E and/or (ii) by inhibiting human histone methytransferases that are involved in the methylation of H3K9me3, for example, inhibition of any one or a combination of human SUV39h1, human SUV39h2 or human SETDB1. In some embodiment, an agent which increases the expression or activity of at least of the members of the KDM4 family of histone demethylases, e.g., JMJD2A/KDM4A and/or JMJD2D/KDM4D and/or JMJD2B/KDM4B and/or JMJD2C/KDM4C and/or JMJD2E/KDM4E is injected into, or contacted with the human SCNT embryo according to the methods as disclosed herein.

Although demethylation of H3K9me3 (by KDM4c/Jmjd2c) has been reported to be used to increase the efficiency of somatic cell reprogramming (e.g., the generation of induced pluripotent stem (iPS) cells (Sridharan et al., 2013)), the demethylation of H3K9me3 for increasing the efficiency of SCNT from terminally differentiated somatic cells has not yet been reported. Antony et al. report using KDM4B/JMJD2B in SCNT derived from donor nuclei from pluripotent ES cells (Antony et al., “Transient JMJD2B-Mediated Reduction of H3K9me3 Levels Improve Reprogramming of Embryonic Stem Cells in Cloned Embryos.” Mol. Cell Biol., 2013; 33(5); 974). A pluripotent ES cell is a developmentally immature cell that is not the same as a terminally differentiated somatic cell. Importantly, there are significant differences in the global epigenetic status of an embryonic stem (ES) cell or an induced pluripotent stem (iPS) cell as compared to a differentiated somatic cell. Pluripotent ES cells have less epigenetic barriers, (e.g., less methylation, in particular in the reprogramming resistant regions (RRRs)) and therefore the efficiency of SCNT embryos produced when a ES cell nuclei is used as the donor nuclei is very different from the efficiency of SCNT embryos produced when the nuclei from a terminally differentiated somatic cell is used (Rideout et al., 2000, Nature Genetics, 24(2), 109-10).

In contrast to the report by Antony et al., the inventors herein demonstrate that decreasing H3K9me3 levels (e.g., by overexpressing human KDM4A mRNA) in a hybrid oocytes, e.g., enucleated oocytes comprising donor somatic genetic material, either before activation or after activation results in a surprising increase in post-8-cell SCNT development, e.g., with 32% of treated human SCNT embryos developing to morula, 26.8% developing to blastocyst and 14.3% developing to, and beyond expanded blastocyst stage (as compared to 0% of non-treated human SCNT embryos reaching expended blastocyst stage). This is a 14% increase. This result is highly unexpected given that Antony et al, report only about a 9% improvement in pre-implantation development, even with ES-cell derived donor nuclei are used, which as discussed are developmentally immature cells not having the same epigenetic markers as terminally differentiated somatic cells.

Furthermore, while there have been numerous reports of demethylation of H3K9me3 to increase the efficiency of reprogramming somatic cells to an earlier developmental stage (e.g., the generation of induced pluripotent stem (iPS) cells) (e.g., US applications 2011/0136145 and 2012/0034192 which are incorporated herein in their entirety by reference), the mechanism of reprogramming somatic cells for the generation of iPS cells is significantly different from the mechanism of reprogramming somatic cells for the generation of SCNT embryos (as discussed in Pasque et al., 2011, Mechanisms of nuclear reprogramming by eggs and oocytes: a deterministic process? Nat. Rev. Mol. Cell Biol. 12, 453-459; and Apostolou, E., and Hochedlinger, K., 2013; Chromatin dynamics during cellular reprogramming. Nature 502, 462-471). Therefore what is learned from the demethylation of H3K9me3 in the generation of iPS cells is not relevant or applicable, and cannot be transferred to methods for the successful generation of SCNT human embryos, or for increasing both pre- and post-implantation efficiency of human SCNT embryos.

In particular, there are notable differences between the barriers that exist in human SCNT and human iPS reprogramming, as well notable differences in human SCNT reprogramming and mouse SCNT reprogramming. Firstly, the H3K9me3-barrier in mouse iPSC reprogramming is established primarily by SETDB1 (Chen et al., 2013; Sridharan et al., 2013). Secondly, the downstream gene networks necessary for successful iPSC and SCNT reprogramming are different. For instance, in iPSC reprogramming, key core pluripotency network genes, such as Nanog and Sox2, which are repressed by the H3K9me3 barrier are expressed during relatively late stages of reprogramming (Chen et al., 2013; Sridharan et al., 2013). In contrast, in SCNT reprogramming, key genes repressed by H3K9me3 are expressed and have a critical function at the 2-cell embryonic stage (discussed herein below). This distinction most likely stems from the differences in the set of transcription factors required for successful reprogramming in each context. Indeed, core transcription factors Oct4/Pou5fl which are required for iPSC reprogramming, have been demonstrated to be dispensable in SCNT reprogramming (Wu et al., 2013). Therefore, although H3K9m3 appears to be a common reprogramming barrier for both iPS cell generation and successful SCNT, its deposition and how it affects the reprogramming process are very different in the method of reprogramming to generate iPS cells and the method of reprogramming to generate SCNT embryos.

Therefore, even if removal of the H3K9me3 barrier in reprogrammed human somatic cells to human iPS cells has been demonstrated, because different reprogramming genes and reprogramming mechanisms are used in iPS cell generation, there is no indication that such a method would work for reprogramming human somatic cells in the generation of human SCNT embryos. In fact, both US applications 2011/0136145 and 2012/0034192 specifically state that their method only applies to reprogramming of somatic cells to iPSC and is not suitable for generation of totipotent cells or for the production of human SCNT embryos. Therefore both 2011/0136145 and 2012/0034192 US applications teach away from the present invention.

Furthermore, as well as the very different mechanisms used for somatic cell reprogramming in the generation of iPSC as compared to the generation of SCNT embryos, which are outlined below in Table 1 below, the stem cells produced from reprogramming somatic cells to produce iPSC are markedly different from stem cells obtained from a SCNT embryo (Ma et al., 2014, Abnormalities in human pluripotent cells due to reprogramming mechanisms. Nature, 511(7508), 177-183).

TABLE 1
A summary of key differences between SCNT- and iPS-mediated reprogramming.

| Reprogramming | | | |
|---|---|---|---|
| features | iPS | SCNT | Source |
| Speed | Slow | Fast | (Yamanaka & Blau, 2010) |
| | (days or weeks) | (hours) | |
| Efficiency | Low | High | (Pasque, Miyamoto, & |
| | | | Gurdon, 2010) |
| Factors | Oct4, Sox2, Klf4 | Not yet identified | (Apostolou & Hochedlinger, |
| | | (Not Oct4) | 2013; Jullien, Pasque, Halley- |
| | | | Stott, Miyamoto, & Gurdon, |
| | | | 2011) |
| Mode | Stochastic | Deterministic | (Jullien et al., 2011) |
| Potency | Pluripotency | Totipotency | (Mitalipov & Don Wolf, 2009) |

Accordingly, as discussed above, as the reprogramming genes and mechanisms of reprogramming human somatic cells to human iPS cells are significantly different from the reprogramming genes and mechanisms of reprogramming human somatic cells to human SCNT, and as the resulting cells are significantly different, there is no indication or reason to believe that methods which work for reprogramming to produce iPSC would work reprogramming for generation of human SCNT. In particular, normal iPSC retain residual DNA methylation patterns typical of parental somatic cells, whereas DNA methylation and transcriptome profiles of NT ES cells corresponded closely to IVF-derived ES cells (see Ma et al., Nature. 2014 Jul. 10; 511(7508): 177-183).

Accordingly, one aspect of the present invention relates to a method for increasing the efficiency of human somatic cell nuclear transfer (hSCNT) comprising contacting any one of a donor human somatic cell, a recipient human oocyte, a hybrid oocyte (e.g., human enucleated oocyte comprising donor genetic material prior to fusion or activation) or a human SCNT embryo (i.e., after fusion of the donor nuclei with the enucleated oocyte) with an agent which decreases H3K9me3 methylation in the donor human cell, recipient human oocyte or human SCNT embryo, thereby increasing the efficiency of human SCNT, e.g., increasing the efficiency of the resultant human SCNT to develop to blastocyst and beyond as compared to a non-treated human SCNT embryo.

In some embodiments, the present invention provides a method for increasing the efficiency of human somatic cell nuclear transfer (hSCNT) comprising at least one of: (i) contacting a donor human somatic cell or a recipient human oocyte with at least one agent (e.g., a KDM4A mRNA) which decreases H3K9me3 methylation in the donor human somatic cell or the recipient human oocyte; where the recipient human oocyte is a nucleated or enucleated oocyte; enucleating the recipient human oocyte if the human oocyte is nucleated; transferring the nuclei from the donor human somatic cell to the enucleated oocyte to form a hybrid oocyte; and activating the hybrid oocyte to form a human SCNT embryo; or (ii) contacting a hybrid oocyte with at least one agent which decreases H3K9me3 methylation in the hybrid oocyte, where the hybrid oocyte is an enucleated human oocyte comprising the genetic material of a human somatic cell, and activating the hybrid oocyte to form a human SCNT embryo; or (iii) contacting a human SCNT embryo after activation with at least one agent which decreases H3K9me3 methylation in the SCNT embryo, wherein the SCNT embryo is generated from the fusion of an enucleated human oocyte with the genetic material of a human somatic cell; and incubating the SCNT embryo for a sufficient amount of time to form a blastocyst. In some embodiments, at least one blastomere is collected from the blastocyst and cultured to form at least one human NT-ESC.

In some embodiments an agent which decreases H3K9me3 methylation is at least one of (i) an agent which increases the expression or activation or function of a member of the KDM4 family of histone demethylase and/or (ii) is a H3K9 methyltransferase-inhibiting agent, thereby removing the epigenetic barriers in the RRR and increasing the efficiency of the human SCNT.

In some embodiments, increasing the efficiency of human somatic cell nuclear transfer (SCNT) comprising contacting an SCNT embryo (e.g., after fusion of the human enucleated oocyte with the human genetic material of the donor cell), at least 5 hours post activation (5 hpa), or between 10-12 hpa (i.e. at 1-cell stage), or at about 20 hpa (i.e., early 2-cell stage) or between 20-28 hpa (i.e., 2-cell stage) with at least one of (i) a KDM4 family of histone demethylase (e.g., a KDM4A mRNA) and/or (ii) a H3K9 methyltransferase-inhibiting agent (e.g., inhibitor of human SUV39h1/2).

In some embodiments, the reducing the H3K9me3 methylation occurs by overexpressing or exogenous expression of a human KDM4 gene, e.g., hKDM4A, hKDM4B, hKDM4C, hKDM4D or hKDM4E, in any one of, or a combination of: the human donor oocyte (either pre-enucleation or after enucleation), or the hybrid oocyte (e.g., enucleated oocyte comprising donor genetic nuclear material, but prior to activation), or in the human SCNT embryo (e.g., after at least 5 hours post activation (5 hpa) or at 1-cell stage, or at 2-cell stage), or the donor human somatic cell before the genetic material is removed.

In some embodiments, exogenous expression of a human KDM4 gene, e.g., KDM4A, occurs in the human donor oocyte. In some embodiments, exogenous expression of a human KDM4 gene, e.g., KDM4A, occurs in an enucleated human donor oocyte, or in a hybrid oocyte (e.g., enucleated oocyte comprising donor genetic nuclear material, but prior to activation). In some embodiments, exogenous expression of a KDM4 gene, e.g., KDM4A, occurs in the SCNT embryo at any one of; 5 hpa, between 10-12 hpa (i.e. at 1-cell stage), at about 20 hpa (i.e., early 2-cell stage) or between 20-28 hpa (i.e., 2-cell stage). In some embodiments, where the human SCNT embryo is contacted with an agent which inhibits H3K9me3, such agent, e.g., agent that increases exogenous expression of a human KDM4 gene, e.g., KDM4A, (e.g., KDM4A mRNA or mod-RNA), each cell of the SCNT embryo (e.g., each cell of the 2-cell embryo, or each cell of a 4-cell embryo) is injected with the KDM4A activating or overexpressing agent (e.g., each cell of the SCNT embryo is injected with KDM4A mRNA).

In other embodiments, the methods as disclosed herein to reduce H3K9me3 methylation in the donor genetic material occurs by inhibiting the expression of SUV39h1 and/or SUV39h2, or both (SUV39h1/2), in any one of, or a combination of: the human donor oocyte (either pre-enucleation or after enucleation), or in the hybrid oocyte (i.e., enucleated oocyte comprising donor genetic material before activation), or in the SCNT embryo (e.g., after at least 5 hours post activation (5 hpa) or at 1-cell stage, or at 2-cell stage, or at 4-cell stage), or in the donor human somatic cell.

In some embodiments, inhibition of SUV39h1 and/or SUV39h2, or both (SUV39h1/2), occurs in the donor human somatic cell, e.g., at least about 24 hours, or at least about 48 hours, or at least about 3-days or at least about 4-days or more than 4-days before removal of the nuclei or genetic material for transfer to the enucleated human donor oocyte. In some embodiments, inhibiting the expression of SUV39h1 and/or SUV39h2, or both (SUV39h1/2) is by siRNA and occurs for at least 12 hours, or at least 24 hours or more, at the time periods prior to removal of the nuclei.

Another aspect of the present invention relates to a method for increasing the efficiency of human somatic cell nuclear transfer (SCNT) comprising contacting a human SCNT embryo, human oocyte or hybrid oocyte, or donor human somatic cell with an agent which decreases H3K9me3 methylation (e.g., KDM4A mRNA), thereby increasing the efficiency of the SCNT. In some embodiments, the recipient human oocyte is a human oocyte of poor quality that would not be of sufficient quality for successful fertilization using IVF procedures. In some embodiments, the human oocyte is contacted prior to the injection of a donor human nuclei or genetic material. In some embodiments, the recipient human oocyte is an enucleated human oocyte. In some embodiments, the SCNT embryo is a 1-cell stage, or 2-cell stage SCNT embryo. In some embodiments, the agent which decreases H3K9me3 methylation (e.g., KDM4A mRNA) contacts a recipient human oocyte or enucleated human oocyte prior to nuclear transfer with a nucleus or genetic material from a terminally differentiated human somatic cell.

In some embodiments, the agent which contacts a recipient human oocyte, hybrid oocyte, human somatic donor cell, or human SCNT embryo increases the expression or activity of at least one member of the KDM4 family of histone demethylases, for example, at least one member of the human KDM4 (JMJD2) family consisting of: human KDM4A (SEQ ID NO: 1), human KDM4B (SEQ ID NO: 2), human KDM4C (SEQ ID NO:3) or human KDM4D (SEQ ID NO: 4). In some embodiments, the agent which increases the expression or activity of the KDM4 family of histone demethylases increases the expression or activity of KDM4D (JMJD2D) or KDM4A (JMJD2A) or KDM4B or KDM4C. In some embodiment, the agent comprises a nucleic acid sequence of KDM4 from humans, e.g., KDM4A (SEQ ID NO: 1), human KDM4B (SEQ ID NO: 2), human KDM4C (SEQ ID NO:3) or human KDM4D (SEQ ID NO: 4) or human KDM4E (SEQ ID NO: 45), or a biologically active fragment or homologue of at least 80%, or at least about 85%, or at least about 90%, or at least about 95%, or at least about 98%, or at least about 99% sequence identity thereof which increases the efficiency of human SCNT to a similar or greater extent (e.g., at least about 110%, or at least about 120%, or at least about 130%, or at least about 140%, or at least about 150%, or more than 150% increased) as compared to the corresponding sequence of SEQ ID NO: 1-4 or SEQ ID NO: 45.

In some embodiments, the agent which contacts a recipient human oocyte or human SCNT embryo increases the expression of human KDM4A protein of SEQ ID NO: 9, and/or comprises a human KDM4A nucleic acid sequence corresponding of SEQ ID NO: 1, or a biologically active fragment thereof which increases the efficiency of human SCNT to a similar or greater extent (e.g., at least about 110%, or at least about 120%, or at least about 130%, or at least about 140%, or at least about 150%, or more than 150% increased) as compared to the nucleic acid sequence of SEQ ID NO: 1.

In some embodiments, an agent which contacts a recipient human oocyte or human SCNT embryo increases the expression of human KDM4D protein of SEQ ID NO: 12, and/or comprises a human KDM4D nucleic acid sequence corresponding of SEQ ID NO: 4, or a biologically active fragment thereof. In some embodiments, a biologically active fragment of KDM4D of SEQ ID NO: 12 comprises amino acids 1-424 of SEQ ID NO: 12, as disclosed in Antony et al., Nature, 2013. In some embodiments, a biologically active fragment of SEQ ID NO: 12 comprises amino acid 1-424 of SEQ ID NO: 12 that also lacks at least 1, or at least 2, or at least between 2-10, or at least between 10-20, or at least between 20-50, or at least between 50-100 amino acids at the C-terminal, or the N-terminal of amino acids 1-424 of SEQ ID NO: 12, or lacks at least 1, or at least 2, or at least between 2-10, or at least between 10-20, or at least between 20-50, or at least between 50-100 amino acids at the C-terminal and the N-terminal of amino acids 1-424 of SEQ ID NO: 12.

In alternative embodiments, an agent which contacts a donor human cell, e.g., a donor nuclei of a terminally differentiated cell, increases the expression or activity of the KDM4 family of histone demethylases, for example, but not limited to the KDM4 family consisting of: KDM4A, KDM4B, KDM4C, KDM4D or KDM4E as discussed above.

Another aspect of the present invention relates to a method for increasing the efficiency of human somatic cell nuclear transfer (SCNT) comprising contacting the nuclei of a donor human cell, e.g., a terminally differentiated somatic cell, with an agent which decreases H3K9me3 methylation in the nuclei of the donor human somatic cell, thereby increasing the efficiency of the SCNT.

In some embodiments of all aspects of the present invention, an agent which contacts a donor human somatic cell is an inhibitor of a H3K9 methyltransferase, for example, but not limited to, an inhibitor of the human SUV39h1, human SUV39h2 or human SETDB1 expression or protein function. In some embodiments, at least one or any combination of inhibitors of human SUV39h1, human SUV39h2 or human SETDB1 can be used in the methods to increase the efficiency of human SCNT. In some embodiments, an inhibitor of a H3K9 methyltransferase is not an inhibitor of human SETDB1.

In some embodiments, an inhibitor of H3K9 methyltransferase is selected from the group consisting of; a RNAi agent, an siRNA agent, shRNA, oligonucleotide, CRISPR/Cas9, CRISPR/cpfl, neutralizing antibody or antibody fragment, aptamer, small molecule, protein, peptide, small molecule, avidimir, and functional fragments or derivatives thereof etc. In some embodiments, the H3K9 methyltransferase inhibitor is a RNAi agent, e.g., siRNA or shRNA molecule. In some embodiments, the agent comprises a nucleic acid inhibitor to inhibit expression of human SUV39H1 protein (SEQ ID NO: 5 or SEQ ID NO: 48). In some embodiments, the agent comprises a nucleic acid inhibitor to inhibit expression of human SUV39H2 protein (SEQ ID NO: 6). In some embodiments, a siRNA inhibitor of human SUV39h1 comprises at least one of: SEQ ID NO: 7, SEQ ID NO; 8, SEQ ID NO: 20, SEQ ID NO: 21, SEQ ID NO: 22 or SEQ ID NO: 23 or a fragment of at least 10 consecutive nucleotides thereof, or nucleic acid sequence with at least 80% sequence identity (or at least about 85%, or at least about 90%, or at least about 95%, or at least about 98%, or at least about 99% sequence identity) to any of SEQ ID NO: 7, SEQ ID NO; 8, SEQ ID NO: 20, SEQ ID NO: 21, SEQ ID NO: 22 or SEQ ID NO: 23. In some embodiments, a siRNA inhibitor of human SUV39h1 comprises at least one of: SEQ ID NO; 8, SEQ ID NO: 21 or SEQ ID NO: 23 or a fragment of at least 10 consecutive nucleotides thereof, or nucleic acid sequence with at least 80% sequence identity (or at least about 85%, or at least about 90%, or at least about 95%, or at least about 98%, or at least about 99% sequence identity) to any of SEQ ID NO; 8, SEQ ID NO: 21 or SEQ ID NO: 23.

In some embodiments, a siRNA or other nucleic acid inhibitor hybridizes to in full or in part, a target sequence located within a region of nucleotides of any of SEQ ID NO: 14 or SEQ ID NO: 47 of human SUV39h1 (corresponding to SUV39h1 variants 2 and 1, respectively).

In some embodiments, a siRNA inhibitor of human SUV39h2 comprises at least one of: SEQ ID NO: 18 or SEQ ID NO: 19, or SEQ ID NO: 24, SEQ ID NO: 25, SEQ ID NO: 26, SEQ ID NO: 27, or a fragment of at least 10 consecutive nucleotides thereof, or nucleic acid sequence with at least 80% sequence identity (or at least about 85%, or at least about 90%, or at least about 95%, or at least about 98%, or at least about 99%) to SEQ ID NO: 18 or SEQ ID NO: 19, or SEQ ID NO: 24, SEQ ID NO: 25, SEQ ID NO: 26, SEQ ID NO: 27. In some embodiments, a siRNA inhibitor of human SUV39h2 comprises at least one of: SEQ ID NO: 19, SEQ ID NO: 25, SEQ ID NO: 27, or a fragment of at least 10 consecutive nucleotides thereof, or nucleic acid sequence with at least 80% sequence identity (or at least about 85%, or at least about 90%, or at least about 95%, or at least about 98%, or at least about 99%) to SEQ ID NO: 19, SEQ ID NO: 25, SEQ ID NO: 27.

In some embodiments, a siRNA or other nucleic acid inhibitor hybridizes in full or part, to a target sequence located within a region of nucleotides of any of SEQ ID NOS: 15, 49, 51, 52 and 53 of human SUV39h2 (hSUV39h2 variants 1-5).

In some embodiments, an agent can contact the SCNT embryo prior to, or at about 5 hours post activation, or when the human SCNT embryo is at the 1-cell stage, 2-cell or 4-cell stage. In alternative embodiments, an agent can contact the human SCNT embryo after 5 hours post activation or when the human SCNT embryo is at the 2-cell stage. In some embodiments, the recipient human oocyte, hybrid oocyte or human SCNT embryo is injected with the agent, for example, by injection of KDM4A mRNA into the nuclei and/or cytoplasm of the recipient human oocyte, hybrid oocyte or human SCNT embryo. In some embodiments, the agent increases the expression or activity of at least one member of the KDM4 family of histone demethylases.

In some embodiments, an agent which decreases H3K9me3 methylation (e.g., KDM4A mRNA) contacts or is injected into the donor human cell, e.g., the nuclei or cytoplasm of a terminally differentiated somatic cell, prior to injection of the nuclei of the donor human cell into an enucleated human oocyte. In some embodiments, such an agent contacts the donor human somatic cell for at least 1 hour, or at least 2 or more hours, where the contact occurs at least 1 day (24 hours), or at least 2 days, or at least 3 days, or more than 3 days, prior to the removal of the nuclei from the donor human somatic cell into an enucleated human oocyte.

In all aspects of the present invention, the human SCNT embryo is produced from the injection of a donor human somatic cell nuclei from a differentiated somatic cell (often a terminally differentiated cell, but not an ES cell or iPSC) into an enucleated human oocyte, where the donor nuclei is not from an embryonic stem (ES) cell or an induced pluripotent stem (iPS) cell, or a fetal cell. In all aspects of the present invention, the human SCNT embryo is generated by the injecting a donor nuclei from a terminally differentiated human somatic cell into an enucleated human oocyte. In some embodiments, the donor human somatic cell genetic material is injected into a non-human recipient oocyte. In some embodiments, the human SCNT embryo develops after activation (or fusion) of the hybrid oocyte. In some embodiments, the hybrid oocyte comprises an enucleated human oocyte comprising the genetic nuclear material from a somatic human donor cell, and also mitochondrial genetic material (e.g., mitochondrial DNA or mtDNA) from a third human donor (i.e., the mtDNA in not native to the enucleated oocyte).

In all aspects of the present invention, the donor somatic cell, recipient oocyte or SCNT embryo are human cells, e.g., are a human donor cell, a recipient human oocyte or human SCNT embryo.

Accordingly, in all aspects of the invention, the method results in an at least about a 5%, or at least about a 10%, or at least about a 13%, or at least about a 15%, or at least a 30% increase, or at least a 50% increase, or a 50%-80% increase, or a greater than 80% increase in efficiency of human SCNT as compared to human SCNT performed in the absence of an agent which decreases H3K9me3 methylation (i.e., in absence of an agent which increase the expression or activation of a member of the KDM4 family). Stated another way, the methods as disclosed herein increase the efficiency of pre-implantation development of SCNT embryos, or increases the development of hSCNT embryos to blastocyst stage, or increases the development of hSCNT embryos to expanded blastocyst stage, whereby at least about a 5%, or 7%, or 10%, or 12% or more than 12% develop to expanded blastocyst stage. In another embodiment, the methods increase the efficiency of development of human SCNT embryos, for example, at least a 3-fold, or at least a 4-fold, or at least a 5-fold, or at least about a 6-fold, or at least about a 7-fold, or at least about a 8-fold or more than 8-fold increase in the successful development to blastocyst stage, as compared to those hSCNT embryos prepared in the absence of an agent which decreases H3K9me3 methylation. In some embodiments, an increase in human SCNT efficiency provided by the methods and compositions as disclosed herein refers to an increase in the generation or yield of human SCNT embryo-derived embryonic stem cells (human NT-ESCs).

Another aspect of the present invention relates to a composition comprising at least one of: a human SCNT embryo, recipient human oocyte, or hybrid oocyte or a human blastocyst and at least one of: (i) an agent which increases the expression or activity of the KDM4 family (Jmjd2) of histone demethylases or (ii) an agent which inhibits a H3K9 methyltransferase.

In some embodiments, the composition comprises a recipient human oocyte which is an enucleated human oocyte or a human oocyte prior to the injection of a donor nucleus obtained from a terminally differentiated somatic cell. In some embodiments, the composition comprises a hybrid oocyte (e.g., human enucleated oocyte comprising donor nuclear genetic material prior to activation). In some embodiments, the human SCNT embryo is a 1-cell stage, or 2-cell, or 4-cell stage human SCNT embryo. In some embodiments, the composition comprises an agent which increases the expression of at least one gene encoding a member of the KDM4 family of histone demethylases, or increases the activity of at least one member of the KDM4 family of histone demethylases, for example, KDM4A, KDM4B, KDM4C, KDM4D or KDM4E. In some embodiment, the agent increases the expression or activity of KDM4D (JMJD2D) or KDM4A (JMJD2A), or is a biologically active fragment or homologue thereof which increases the efficiency of SCNT to a similar or greater extent as compared to the corresponding sequence of SEQ ID NO: 1-4 or SEQ ID NO: 45. In some embodiments, the composition comprises a human KDM4A nucleic acid sequence corresponding of SEQ ID NO: 1, or a biologically active fragment thereof which increases the efficiency of SCNT to a similar or greater extent as compared to the nucleic acid sequence of SEQ ID NO: 1.

In some embodiments, the composition comprises an agent which is an inhibitor of a H3K9 methyltransferase, for example, but not limited to an inhibitor of human SUV39h1, human SUV39h2 or human SETDB1. In some embodiments, at least one or any combination of inhibitors of human SUV39h1, human SUV39h2 or human SETDB1 can be used in the methods to increase the efficiency of human SCNT.

In some embodiments, the composition comprises an inhibitor of H3K9 methyltransferase selected from the group consisting of; an siRNA, shRNA, neutralizing antibody or antibody fragment, aptamer, small molecule, protein, peptide, small molecule etc. In some embodiments, the H3K9 methyltransferase inhibitor is a siRNA or shRNA molecule which inhibits human SUV39h1 or human SUV39h2 or human SETDB1. In some embodiments, the composition comprises a nucleic acid inhibitor hybridizes to, in full or in part, a target sequence located within a region of nucleotides of any of SEQ ID NO: 14 or SEQ ID NO: 47 of human SUV39h1 (corresponding to SUV39h1 variants 2 and 1, respectively), or SEQ ID NOS: 15, 49, 51, 52 and 53 of human SUV39h2 (hSUV39h2 variants 1-5).

In some embodiments, the composition comprises a siRNA inhibitor of human SUV39h1 that binds to, in full or in part, to the target sequence of SEQ ID NO: 7 or a fragment of at least 10 consecutive nucleotides thereof, or nucleic acid sequence with at least 80% sequence identity (or at least about 85%, or at least about 90%, or at least about 95%, or at least about 98%, or at least about 99% sequence identity) to SEQ ID NO: 7. In some embodiments, the composition comprises a siRNA inhibitor of human SUV39h1 that comprises SEQ ID NO: 8 or a fragment of at least 10 consecutive nucleotides thereof, or nucleic acid sequence with at least 80% sequence identity (or at least about 85%, or at least about 90%, or at least about 95%, or at least about 98%, or at least about 99% sequence identity) to SEQ ID NO: 8. In some embodiments, the composition comprises a siRNA or other nucleic acid inhibitor which hybridizes to, in full or in part, to a target sequence located within a region of nucleotides of any of SEQ ID NO: 14 or SEQ ID NO: 47 of human SUV39h1 (corresponding to SUV39h1 variants 2 and 1, respectively).

In some embodiments, the composition comprises a siRNA or other nucleic acid inhibitor which hybridizes in full or part, to a target sequence located within a region of nucleotides of any of SEQ ID NOS: 15, 49, 51, 52 and 53 of human SUV39h2 (hSUV39h2 variants 1-5).

In some embodiments, the composition comprises a human SCNT embryo that is at the 1-cell or 2-cell or 4-cell stage. In some embodiments, the composition comprises an enucleated human oocyte or hybrid oocyte. In some embodiments, the composition comprises a human SCNT embryo, recipient human oocyte, human hybrid oocyte or a human blastocyst.

Another embodiment related to a kit comprising (i) an agent which increases the expression or activity of the KDM4 family of histone demethylases, e.g., comprises a mRNA of a member of the human KDM4 family and/or (ii) an agent which inhibits a H3K9 methyltransferase.

The disclosure described herein, in a preferred embodiment, does not concern a process for cloning human beings, processes for modifying the germ line genetic identity of human beings, or use of human SCNT embryos for industrial or commercial purposes or processes for modifying the genetic identity of humans which are likely to cause them suffering without any substantial medical benefit to man, or humans resulting from such processes.

### BRIEF DESCRIPTION OF THE DRAWINGS

FIGS. 1A-1F show human reprogramming resistant regions (RRRs) are enriched for H3K9me3 in somatic cells. FIG. 1A is a schematic illustration of the experimental procedures. Samples used for RNA-seq are marked by dashed rectangles. FIG. 1B is a heatmap illustration of the transcriptome of IVF human preimplantation embryos. Each tile represents an average of peaks within the region obtained by sliding-window analysis. Shown are the 707 regions that are activated from the 4-cell to the 8-cell stage in IVF embryos. RNA-seq data sets were obtained from a previous publication (Xue et al., 2013). FIG. 1C is a heatmap illustration of the transcriptome comparing donor somatic cells, IVF and SCNT embryos at the 8-cell stage. Shown are the 707 regions identified in (FIG. 1A). These regions were classified into three groups based on the fold-change (FC) in transcription levels between SCNT- and IVF 8-cell embryos. FRRs, PRRs, and RRRs indicate fully reprogrammed regions (FC<=2), partially reprogrammed regions (2<FC<=5) and reprogramming resistant regions (FC>5), respectively. FIG. 1D shows the average ChIP-seq intensity of H3K9me3 and H3K4me3 in human fibroblast cells (Nhlf) are shown within FRR, PRR, and RRR compared with 200 kb flanking regions. Histone modification ChIP-seq data sets were obtained from the ENCODE project (Bernstein et al., 2012; The Encode Consortium Project, 2011). FIG. 1E and FIG. 1F are box plots comparing the average intensity of H3K9me3-ChIP-seq (FIG. 1E) and DNaseI-seq (FIG. 1F) within FRR, PRR and RRR in different somatic cell types. ChIP-seq and DNaseI-seq data sets were obtained from the ENCODE projects (ENCODE Project Consortium, 2011). Middle line in the colored space indicates the median, the edges indicate the 25th/75th percentiles, and the whiskers indicate the 2.5th/97.5th percentiles. *** p<0.001, ** p<0.01. See also FIG. 5, and Tables 5 and 6. (Abbreviations: RRR=reprogramming resistant regions, PRR=partially reprogrammed regions; FRR=fully-reprogrammed regions),

FIGS. 2A-2H show the injection of human KDM4A mRNA improves development of mouse and human SCNT embryos. FIG. 2A is a schematic illustration of the mouse SCNT procedures. FIG. 2B show representative nuclear images of 1-cell stage SCNT embryos stained with anti-H3K9me3 and DAPI 5 at hours after mRNA injection. FIG. 2C show that KDM4A mRNA injection greatly improves preimplantation development of mouse SCNT embryos. Shown is the percentage of embryos that reached the indicated stages. Error bars indicate s.d. FIG. 2D show representative images of SCNT embryos after 120 hours of culturing in vitro. Scale bar, 100 Jim. FIG. 2E is a schematic illustration of the human SCNT procedures. FIG. 2F is a bar graph showing the average developmental efficiency of human SCNT embryos obtained using oocytes from four different donors during 7 days of in vitro culture. The efficiency was calculated using the number of embryos that reached 2-cell stage. Blast: blastocyst, ExBlast: expanded blastocyst. Developmental rates were statistically analyzed by Fisher's exact test. FIG. 2G show representative images of SCNT embryos after 7 days of culturing in vitro. FIG. 2H show bar graphs of the developmental rate of human SCNT embryos derived from each oocyte-donor female. See also Tables 3 and 4.

FIGS. 3A-3J show the establishment and characterization of NTK-ESCs from AMD patients. FIG. 3A is a summary table of established NT-ESC lines using AMD patient fibroblasts as nuclear donor through KDM4A-assisted SCNT. FIG. 3B show representative phase contrast and immunostaining images of NTK-ESCs. Scale bar, 100 Jim. FIG. 3C are bar graphs showing expression levels of pluripotency-specific and fibroblast-specific genes based on RNA-seq data. FIG. 3D is a scatter plot comparing gene expression levels between a control ESC line (ESC15) and a representative NTK-ESC, NTK6. Differentially expressed genes (FC>3.0) are shown as black dots. FIG. 3E shows the hierarchical clustering of NTK-ESCs, control ESCs and donor dermal fibroblast cells based on RNA-seq data sets. FIG. 3F are representative images of immunostained embryoid bodies (EBs) spontaneously differentiated in vitro for 2 weeks. Scale bar, 100 Jim. FIG. 3G show representative histological images of teratoma derived from NTK6 at 12 weeks after transplantation. Scale bar, 100 μm. FIG. 3H shows representative images of cytogenetic G-banding analysis of NTK6. FIG. 3I shows the nuclear DNA genotyping using 16 STR markers. FIG. 3J shows the mitochondrial DNA genotyping of a representative single nucleotide polymorphism (SNP) site. See also FIGS. 6 and 7. FIG. 3J discloses rs2853826 (m. 10398 A>G) sequences as SEQ ID NOS 58, 58 and 59, respectively, in order of appearance, and rs2853826 (m. 10400 C>T) sequences as SEQ ID NOS 58, 58 and 59, respectively, in order of appearance.

FIGS. 4A-4C show partial restoration of transcription upon KDM4A mRNA injection in SCNT 8-cell embryos. FIG. 4A shows heatmap comparing transcription levels of the 318 RRRs at the late 8-cell stage. The expression levels of 158 out of the 318 RRRs are markedly (FC>2) increased in response to KDM4A mRNA injection. FIG. 4B shows gene ontology analysis of the 206 KDM4A-responsive genes (FC>2). FIG. 4C shows bar graphs and genome browser view of transcription levels of two representative KDM4A-responsive genes, UBTFL1 and THOC5, in IVF, or SCNT (with or without KDM4A mRNA injection) 8-cell embryos. See also Table 7.

FIGS. 5A-5E are related to FIG. 1 and shows RRRs (Reprogramming Resistant Regions) in human somatic cells possess heterochromatin features. FIG. 5A shows box plots comparing the average ChIP-seq signals of six histone modifications at FRR, PRR, and RRR in human fibroblast cells (Nhlf). FIGS. 5B and 5C show box plots comparing the average intensities of H3K9me3-ChIP-seq (FIG. 5B) and DNaseI-seq (FIG. 5C) within FRR, PRR and RRR in different somatic cell types. ChIP-seq and DNaseI-seq data sets were obtained from ENCODE projects (ENCODE Project Consortium, 2011). Note that H3K9me3 intensity is significantly enriched in RRRs compared to FRRs and PRRs, and DnaseI-seq intensity is significantly depleted in RRRs compared to FRRs and PRRs. *** p<0.001, ** p<0.01, * p<0.05. FIG. 5D shows box plots comparing the average percentage of exonic sequences, which represents the density of protein coding genes, in FRR, PRR and RRR in the human genome. *** p<0.001, * p<0.05. FIG. 5E shows box plots comparing the average percentage of repetitive sequence within FRR, PRR and RRR. *** p<0.001, * p<0.05, ns, not significant.

FIGS. 6A-6F are related to FIG. 1 and shows human NTK-ESCs exhibit normal pluripotency. FIG. 6A shows representative immunostaining images of NTK-ESCs and IVF-derived control ESCs. ESC colonies were co-stained with anti-SOX2, anti-SSEA4 antibodies and DAPI. Scale bar, 100μm. FIG. 6B is a Scatter plot evaluation of the reproducibility of RNA-seq of different biological replicates of the control ESCs and NTK-ESCs. FIG. 6C shows scatter plots comparing global gene expression patterns between control ESCs and NTK-ESCs. Differentially expressed genes (FC>3.0) are shown as black dots. Note that the correlation of each pair-wise comparison is extremely high (r=0.95-0.99). FIG. 6D show representative images of immunostained embryoid bodies (EBs) spontaneously differentiated in vitro for 2 weeks. EBs were stained with anti-TUJ1, anti-BRACHYURY or anti-AFP antibody together with DAPI. Scale bar, 100 μm. FIG. 6E shows representative histological images of teratoma derived from NTK-ESC#6 at 12 weeks after transplantation. Scale bar, 100 μm. FIG. 6F shows show representative histological images of teratoma derived from NTK7 and NTK8 cell lines at 12 weeks after transplantation.

FIGS. 7A-7C are related to FIG. 3 and shows human NTK-ESCs contain nuclear-donor derived genome and oocyte-donor derived mitochondria. FIG. 7A shows representative images of cytogenetic G-banding analysis showing normal karyotypes with expected sex chromosome compositions in the NTK-ESC lines NTK7 and NTK8. FIG. 7B shows nuclear DNA genotyping using 16 STR markers. Note that all STR markers of NTK-ESC NTK7 and NTK8 perfectly match those of the original nuclear donor fibroblast DFB-6 and DFB-8, respectively. FIG. 7C shows mitochondrial DNA genotyping of representative single nucleotide polymorphism (SNP) sites. Mitochondria of NTK-ESCs are exclusively derived from donor oocytes. FIG. 7C discloses rs1116907 (m. 8468 C>T) sequences as SEQ ID NOS 60-65, respectively, in order of appearance, and rs1116904 (m. 8027 G>A) sequences as SEQ ID NOS 66-69 and 69-70, respectively, in order of appearance.

### DETAILED DESCRIPTION OF THE INVENTION

Despite its enormous potential for both basic science and therapeutic use, the efficiency of cloning human somatic cells by somatic cell nuclear transfer (SCNT) remains extremely low, resulting in poor development to blastocyst and smaller cell number at expanded blastocyst. These deficits also contribute to the infrequent successful human ES cell line establishment from cloned human SCNT embryos. The incompetence of the cloned human embryos is largely due to incomplete nuclear programming and/or epigenetic barriers in the donor human nuclei.

The present invention is based on the discovery that trimethylation of Histone H3-Lysine 9 (H3K9me3) occurs in reprogramming resistant regions (RRR) in the nuclei of the human donor cell, and is an epigenetic barrier which prevents efficient human somatic cell nuclear reprogramming by SCNT. As disclosed herein, the inventors have demonstrated two ways to improve efficacy of human SCNT, firstly by promoting demethylation of H3K9me3 of the donor nuclear genetic material by using exogenous or increased expression (e.g., overexpression) of a member of the KDM4 demethylase family, e.g., KDM4A or KDM4D, and/or by inhibiting methylation of H3K9me3 by inhibiting a histone methyltransferase, e.g., SUV39h1 and/or SUV39h2. In some embodiments, a hybrid human oocyte (e.g., enucleated human oocyte comprising the nuclear genetic material from a human donor somatic cell prior to activation) and/or a human SCNT embryo is injected with an agent which increases the expression of KDM4A and/or KDM4D (e.g., mRNA encoding human KDM4A protein or a functional fragment of the KDM4A protein and/or mRNA encoding human KDM4D protein or a functional fragment of the KDM4D protein). In some embodiments, the agent is mRNA encoding the human KDM4A or KDM4D protein, or a homologue thereof, or another member of the human KDM4 family of histone demethyases.

In some embodiments, a donor human somatic cell, a recipient human oocyte, a hybrid oocyte (e.g., human enucleated oocyte comprising donor genetic material prior to fusion or activation) or a human SCNT embryo (i.e., after fusion of the donor nuclei with the enucleated oocyte) is injected with a mRNA encoding a member of the KDM4 family, or a mRNA or nucleic acid or nucleic acid analogue (including modified mRNA (also known as mod-RNA)). In some embodiment, a donor human somatic cell, a recipient human oocyte, a hybrid oocyte, or a human SCNT is injected with mRNA encoding human KDM4A protein or a functional fragment of the KDM4A protein and/or mRNA encoding human KDM4D protein or a functional fragment of the KDM4D protein. In some embodiments, where the hSCNT is injected, it can be done at any stage after activation, e.g., at 5 hpa, or 10-12 hpa, or 20-28 hpa, 1-cell stage, 2-cell stage or 4-cell stage of the hSCNT embryo.

Accordingly, the present invention relates to methods, compositions and kits comprising H3K9me3 histone demethylase activators, e.g., activators of the human KDM4/JMJD2 family and/or H3K9me3 methyltransferase inhibitors, e.g., inhibitors of human SUV39h1 or human SUV39h2 or human SETDB1 to remove the epigenetic barriers in human nuclear genomic material (e.g., in the human donor genome) thereby increasing the efficiency of successful human SCNT, including the development of the hSCNT embryos to blastocyst stage and beyond.

Accordingly, aspects of the invention relate to methods, compositions and kits directed to increasing efficiency of human SCNT by reducing H3K9me3 methylation by either (i) expressing histone demethylases which are capable of demethylating H3K9me3, e.g., for example, members of the KDM4 family of histone demethylases, such as, for example but not limited to, JMJD2A/KDM4A or JMJD2B/KDM4B, or JMJD2C/KDM4C or JMJD2D/KDM4D or JMJD2E/KDM4E and/or (ii) inhibiting histone methytransferases that are involved in the methylation of H3K9me3, for example, inhibition of any one or a combination of human SUV39h1, human SUV39h2 or human SETDB1. In some embodiment, the agent which increases the expression or activity of the human KDM4 family of histone demethylases increases the expression or activity of KDM4E(JMJD2E), KDM4D (JMJD2D), KDM4C (JMJD2C), KDM4B (JMJD2B) or KDM4A (JMJD2A).

Another aspect relates to uses of the human SCNT-embryos produced using the methods and compositions as disclosed herein to develop into one or more blastomeres, which can be removed or biopsied and/or used to generate human ES cells (i.e., human NT-ESCs). The NT-hESCs generated using the methods as disclosed herein can be used for a variety of purposes, e.g., for regenerative and/or cell-based therapy, for assays, and for use in disease modeling (e.g., where the hNT-ESCs are patient-specific hNT-ESC, where the hSCNT embryo was generated used genomic nuclear donor from a human donor subject that has a particular mutation or SNP and/or has a predisposition to have a particular disease). The hNT-ESC can also be used in assays, e.g., drug screening assays, including but not limited to personalized drug screening and/or disease specific drug screens. The hNT-ESCs generated using the methods and compositions as disclosed herein can be cryopreserved, as well as stored in a human NT-ESC bank.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The phrase “Somatic Cell Nuclear Transfer” or “SCNT” is also commonly referred to as therapeutic or reproductive cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

The term “nuclear transfer” as used herein refers to a gene manipulation technique allowing an identical characteristics and qualities acquired by artificially combining an enucleated oocytes with a cell nuclear genetic material or a nucleus of a somatic cell. In some embodiments, the nuclear transfer procedure is where a nucleus or nuclear genetic material from a donor somatic cell is transferred into an enucleated egg or oocyte (an egg or oocyte from which the nucleus/pronuclei have been removed). The donor nucleus can come from a somatic cell.

The term “nuclear genetic material” refers to structures and/or molecules found in the nucleus which comprise polynucleotides (e.g., DNA) which encode information about the individual. Nuclear genetic material includes the chromosomes and chromatin. The term also refers to nuclear genetic material (e.g., chromosomes) produced by cell division such as the division of a parental cell into daughter cells. Nuclear genetic material does not include mitochondrial DNA.

The term “SCNT embryo” refers to a cell, or the totipotent progeny thereof, of an enucleated oocyte which has been fused with the nucleus or nuclear genetic material of a somatic cell. The SCNT embryo can develop into a blastocyst and develop post-implantation into living offspring. The SCNT embryo can be a 1-cell embryo, 2-cell embryo, 4-cell embryo, or any stage embryo prior to becoming a blastocyst.

The term “parental embryo” is used to refer to a SCNT embryo from which a single blastomere is removed or biopsied. Following biopsy, the remaining parental embryo (the parental embryo minus the biopsied blastomere) can be cultured with the blastomere to help promote proliferation of the blastomere. The remaining, viable parental SCNT embryo may subsequently be frozen for long term or perpetual storage or for future use. Alternatively, the viable parental embryo may be used to create a pregnancy.

The term “donor human cell” or “donor human somatic cell” refers to a somatic cell or a nucleus of human cell which is transferred into a recipient oocyte as a nuclear acceptor or recipient.

The term “somatic cell” refers to a plant or animal cell which is not a reproductive cell or reproductive cell precursor. In some embodiments, a differentiated cell is not a germ cell. A somatic cell does not relate to pluripotent or totipotent cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

The term “differentiated cell” as used herein refers to any cell in the process of differentiating into a somatic cell lineage or having terminally differentiated. For example, embryonic cells can differentiate into an epithelial cell lining the intestine. Differentiated cells can be isolated from a fetus or a live born animal, for example.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The term “oocyte” as used herein refers to a mature oocyte which has reached metaphase II of meiosis. An oocyte is also used to describe a female gamete or germ cell involved in reproduction, and is commonly also called an egg. A mature egg has a single set of maternal chromosomes (23, X in a human primate) and is halted at metaphase II.

A “hybrid oocyte” refers to an enucleated oocyte that has the cytoplasm from a first human oocyte (termed a “recipient”) but does not have the nuclear genetic material of the recipient oocyte; it has the nuclear genetic material from another human cell, termed a “donor.” In some embodiments, the hybrid oocyte can also comprise mitochondrial DNA (mtDNA) that is not from the recipient oocyte, but is from a donor cell (which can be the same donor cell as the nuclear genetic material, or from a different donor, e.g., from a donor oocyte).

The term “enucleated oocyte” as used herein refers to an human oocyte which its nucleus has been removed.

The term “enucleation” as used herein refers to a process whereby the nuclear material of a cell is removed, leaving only the cytoplasm. When applied to an egg, enucleation refers to the removal of the maternal chromosomes, which are not surrounded by a nuclear membrane. The term “enucleated oocyte” refers to an oocyte where the nuclear material or nuclei is removed.

The “recipient human oocyte” as used herein refers to a human oocyte that receives a nucleus from a human nuclear donor cell after removing its original nucleus.

The term “fusion” as used herein refers to a combination of a nuclear donor cell and a lipid membrane of a recipient oocyte. For example, the lipid membrane may be the plasma membrane or nuclear membrane of a cell. Fusion may occur upon application of an electrical stimulus between a nuclear donor cell and a recipient oocyte when they are placed adjacent to each other or when a nuclear donor cell is placed in a perivitelline space of a recipient oocyte.

The term “activation” as used herein refers to stimulation of a cell to divide, before, during or after nuclear transfer. Preferably, in the present invention, it means stimulation of a cell to divide after nuclear transfer.

The term “living offspring” as used herein means an animal that can survive ex utero. Preferably, it is an animal that can survive for one second, one minute, one day, one week, one month, six months or more than one year. The animal may not require an in utero environment for survival.

The term “prenatal” refers to existing or occurring before birth. Similarly, the term “postnatal” is existing or occurring after birth.

The term “blastocyst” as used herein refers to a preimplantation embryo in placental mammals (about 3 days after fertilization in the mouse, about 5 days after fertilization in humans) of about 30-150 cells. The blastocyst stage follows the morula stage, and can be distinguished by its unique morphology. The blastocyst consists of a sphere made up of a layer of cells (the trophectoderm), a fluid-filled cavity (the blastocoel or blastocyst cavity), and a cluster of cells on the interior (the inner cell mass, or ICM). The ICM, consisting of undifferentiated cells, gives rise to what will become the fetus if the blastocyst is implanted in a uterus. These same ICM cells, if grown in culture, can give rise to embryonic stem cell lines. At the time of implantation the mouse blastocyst is made up of about 70 trophoblast cells and 30 ICM cells.

The term “blastula” as used herein refers to an early stage in the development of an embryo consisting of a hollow sphere of cells enclosing a fluid-filled cavity called the blastocoel. The term blastula sometimes is used interchangeably with blastocyst.

The term “blastomere” is used throughout to refer to at least one blastomere (e.g., 1, 2, 3, 4, etc.) obtained from a preimplantation embryo. The term “cluster of two or more blastomeres” is used interchangeably with “blastomere-derived outgrowths” to refer to the cells generated during the in vitro culture of a blastomere. For example, after a blastomere is obtained from a SCNT embryo and initially cultured, it generally divides at least once to produce a cluster of two or more blastomeres (also known as a blastomere-derived outgrowth). The cluster can be further cultured with embryonic or fetal cells. Ultimately, the blastomere-derived outgrowths will continue to divide. From these structures, ES cells, totipotent stem (TS) cells, and partially differentiated cell types will develop over the course of the culture method.

The term “karyoplast” as used herein refers to a cell nucleus, obtained from the cell by enucleation, surrounded by a narrow rim of cytoplasm and a plasma membrane.

The term “cell couplet” as used herein refers to an enucleated oocyte and a somatic or fetal karyoplast prior to fusion and/or activation.

The term “cleavage pattern” as used herein refers to the pattern in which cells in a very early embryo divide; each species of organism displays a characteristic cleavage pattern that can be observed under a microscope. Departure from the characteristic pattern usually indicates that an embryo is abnormal, so cleavage pattern is used as a criterion for preimplantation screening of embryos.

The term “clone” as used herein refers to an exact genetic replica of a DNA molecule, cell, tissue, organ, or entire plant or animal, or an organism that has the same nuclear genome as another organism.

The term “cloned (or cloning)” as used herein refers to a gene manipulation technique for preparing a new individual unit to have a gene set identical to another individual unit. In the present invention, the term “cloned” as used herein refers to a cell, embryonic cell, fetal cell, and/or animal cell has a nuclear DNA sequence that is substantially similar or identical to the nuclear DNA sequence of another cell, embryonic cell, fetal cell, differentiated cell, and/or animal cell. The terms “substantially similar” and “identical” are described herein. The cloned SCNT embryo can arise from one nuclear transfer, or alternatively, the cloned SCNT embryo can arise from a cloning process that includes at least one re-cloning step.

The term “transgenic organism” as used herein refers to an organism into which genetic material from another organism has been experimentally transferred, so that the host acquires the genetic traits of the transferred genes in its chromosomal composition.

The term “embryo splitting” as used herein refers to the separation of an early-stage embryo into two or more embryos with identical genetic makeup, essentially creating identical twins or higher multiples (triplets, quadruplets, etc.).

The term “morula” as used herein refers to the preimplantation embryo 3-4 days after fertilization, when it is a solid mass composed of 12-32 cells (blastomeres). After the eight-cell stage, the cells of the preimplantation embryo begin to adhere to each other more tightly, becoming “compacted”. The resulting embryo resembles a mulberry and is called a morula (Latin:morus=mulberry).

The term “embryonic stem cells” (ES cells) refers to pluripotent cells derived from the inner cell mass of blastocysts or morulae that have been serially passaged as cell lines. The ES cells may be derived from fertilization of an egg cell with sperm or DNA, nuclear transfer, e.g., SCNT, parthenogenesis etc. The term “human embryonic stem cells” (hES cells) refers to human ES cells. The term “ntESC” refers to embryonic stem cells obtained from the inner cell mass of blastocysts or morulae produced from SCNT embryos. “hNT-ESC” refers to embryonic stem cells obtained from the inner cell mass of blastocysts or morulae produced from human SCNT embryos. The generation of ESC is disclosed in U.S. Pat. Nos. 5,843,780; 6,200,806, and ESC obtained from the inner cell mass of blastocysts derived from somatic cell nuclear transfer are described in U.S. Pat. Nos. 5,945,577; 5,994,619; 6,235,970, which are incorporated herein in their entirety by reference. The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Such cells include hES cells, human embryo-derived cells (hEDCs), human SCNT-embryo derived stem cells and adult-derived stem cells. Pluripotent stem cells may be genetically modified or not genetically modified. Genetically modified cells may include markers such as fluorescent proteins to facilitate their identification. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

The term “totipotent” as used herein in reference to SCNT embryos refers to SCNT embryos that can develop into a live born animal.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “reprogramming” as used herein refers to the process that alters or reverses the differentiation state of a somatic cell, such that the developmental clock of a nucleus is reset; for example, resetting the developmental state of an adult differentiated cell nucleus so that it can carry out the genetic program of an early embryonic cell nucleus, making all the proteins required for embryonic development. In some embodiments, the donor human cell is terminally differentiated prior to the reprogramming by SCNT. Reprogramming as disclosed herein encompasses complete reversion of the differentiation state of a somatic cell to a pluripotent or totipotent cell. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult. In somatic cell nuclear transfer (SCNT), components of the recipient oocyte cytoplasm are thought to play an important role in reprogramming the somatic cell nucleus to carry out the functions of an embryonic nucleus.

The term “culturing” as used herein with respect to SCNT embryos refers to laboratory procedures that involve placing an embryo in a culture medium. The SCNT embryo can be placed in the culture medium for an appropriate amount of time to allow the SCNT embryo to remain static but functional in the medium, or to allow the SCNT embryo to grow in the medium. Culture media suitable for culturing embryos are well-known to those skilled in the art. See, e.g., U.S. Pat. No. 5,213,979, entitled “In vitro Culture of Bovine Embryos,” First et al., issued May 25, 1993, and U.S. Pat. No. 5,096,822, entitled “Bovine Embryo Medium,” Rosenkrans, Jr. et al., issued Mar. 17, 1992, incorporated herein by reference in their entireties including all figures, tables, and drawings.

The term “culture medium” is used interchangeably with “suitable medium” and refers to any medium that allows cell proliferation. The suitable medium need not promote maximum proliferation, only measurable cell proliferation. In some embodiments, the culture medium maintains the cells in a pluripotent or totipotent state.

The term “implanting” as used herein in reference to SCNT embryos as disclosed herein refers to impregnating a surrogate female animal with a SCNT embryo described herein. This technique is well known to a person of ordinary skill in the art. See, e.g., Seidel and Elsden, 1997, Embryo Transfer in Dairy Cattle, W. D. Hoard & Sons, Co., Hoards Dairyman. The embryo may be allowed to develop in utero, or alternatively, the fetus may be removed from the uterine environment before parturition.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting a human donor cell, a human recipient oocyte, hybrid oocyte, or a human SCNT embryo with an agent) is intended to include incubating the agent and the human cell, human oocyte, hybrid oocyte or hSCNT-embryo together in vitro (e.g., adding the agent to the donor human cell, human oocyte, hybrid oocyte or hSCNT-embryo in culture or in a container). In some embodiments, the term “contacting” is not intended to include the in vivo exposure of cells to the agent as disclosed herein that may occur naturally in a subject (i.e., exposure that may occur as a result of a natural physiological process). The step of contacting a human somatic cell, human oocyte, hybrid oocyte or hSCNT-embryo with an agent as disclosed herein can be conducted in any suitable manner. For example, a human somatic cell, human oocyte, hybrid oocyte or hSCNT-embryo may be treated in adherent culture, or in suspension culture. It is understood that a human somatic cell, human oocyte, hybrid oocyte or hSCNT-embryo can be contacted with an agent as disclosed herein can also be simultaneously or subsequently contacted with another agent, such as a growth factor or other differentiation agent or environments to stabilize the cells, or to differentiate the cells further. Similarly, a human somatic cell, human oocyte, hybrid oocyte or hSCNT-embryo can be contacted with an agent as disclosed herein (e.g., a KDM4 histone demethylase activator or mRNA) and then with a second agent as disclosed herein (e.g., a H3K9 methyltransferase inhibitor) or vice versa. In some embodiments, a human somatic cell, human oocyte, hybrid oocyte or hSCNT-embryo is contacted with an agent as disclosed herein and a second agent as disclosed herein and the contact is temporally separated. In some embodiments, a human donor cell, human somatic cell, human oocyte, hybrid oocyte or hSCNT-embryo is contacted with one or more agents as disclosed herein substantially simultaneously (e.g., contacted with a KDM4 histone demethylase activator (e.g., KDM4D mRNA) and a H3K9 methyltransferase inhibitor substantially simultaneously).

The term “exogenous” refers to a substance present in a cell or organism other than its native source or level. For example, the terms “exogenous nucleic acid” or “exogenous protein” refer to a nucleic acid or protein that has been introduced by a process involving the hand of man into a biological system such as a cell or organism in which it is not normally found in, or where the nucleic acid or protein which is introduced is normally found in lower amounts. A substance will be considered exogenous if it is introduced into a cell or an ancestor of the cell that inherits the substance. In contrast, the term “endogenous” refers to a substance that is native to the biological system or cell at that time. For instance, “exogenous KDM4A” refers to the introduction of KDM4A mRNA or cDNA which is not normally found or expressed at the level at which it is introduced in the cell or organism at that time.

The term “expression” refers to the cellular processes involved in producing RNA and proteins as applicable, for example, transcription, translation, folding, modification and processing. “Expression products” include RNA transcribed from a gene and polypeptides obtained by translation of mRNA transcribed from a gene.

The term “mitochondrial DNA” is used interchangeably with “mtDNA” refers the DNA of the mitochondrion, a structure situated in the cytoplasm of the cell rather than in the nucleus (where all the other chromosomes are located). In vivo, all mtDNA is inherited from the mother. There are 2 to 10 copies of the mtDNA genome in each mitochondrion. mtDNA is a double-stranded, circular molecule. It is very small relative to the chromosomes in the nucleus and includes only a limited number of genes, such as those encoding a number of the subunits in the mitochondrial respiratory-chain complex and the genes for some ribosomal RNAs and transfer RNAs. A cell includes mtDNA derived from the continued replication cytoplasmically based mitochondria, which in the case of spindle transfer are based in the recipient cytoplast.

The term “mitochondrial Disease” refers to diseases and disorders that affect the function of the mitochondria and/or are due to mitochondrial DNA. The mtDNA is exclusively maternally inherited. Generally these diseases are due to disorders of oxidative phosphorylation. Mitochondrial diseases are often cause by a pathogenic mutation in a mitochondrial gene. The mutations are usually heteroplasmic so there is a mixture of normal and mutant DNA, the level of which can differ among tissues. However, some of the mutations are homoplasmic, so they are present in 100% of the mtDNA. The percentage heteroplasmy of point mutations in the offspring is related to the mutation percentage in the mother. There is a genetic bottleneck, which occurs during oocyte development.

A “genetically modified” or “engineered” cell refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

The term “identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI. In some embodiments, a nucleic acid or amino acid sequence has at least 80%, or at least about 85%, or at least about 90%, or at least about 95%, or at least about 98% or at least about 99% sequence identity to the nucleic acid or amino acid sequence.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”. An “isolated cell” is a cell that has been removed from an organism in which it was originally found or is a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of definitive endoderm cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not definitive endoderm cells or their progeny as defined by the terms herein. In some embodiments, the present invention encompasses methods to expand a population of definitive endoderm cells, wherein the expanded population of definitive endoderm cells is a substantially pure population of definitive endoderm cells. Similarly, with regard to a “substantially pure” or “essentially purified” population of SCNT-derived stem cells or pluripotent stem cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not stem cell or their progeny as defined by the terms herein.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” as used herein describes a cell with a common ancestry or cells with a common developmental fate. In the context of a cell that is of endoderm origin or is “endodermal linage” this means the cell was derived from an endoderm cell and can differentiate along the endoderm lineage restricted pathways, such as one or more developmental lineage pathways which give rise to definitive endoderm cells, which in turn can differentiate into liver cells, thymus, pancreas, lung and intestine.

As used herein, the term “xenogeneic” refers to cells that are derived from different species.

The term “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “RNA interference” or “RNAi” is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. They can comprise a wide variety of modified nucleosides, nucleoside analogs and can comprise chemically or biologically modified bases, modified backbones, etc. Any modification recognized in the art as being useful for RNAi can be used. Some modifications result in increased stability, cell uptake, potency, etc. In certain embodiments the siRNA comprises a duplex about 19 nucleotides in length and one or two 3′ overhangs of 1-5 nucleotides in length, which may be composed of deoxyribonucleotides. shRNA comprise a single nucleic acid strand that contains two complementary portions separated by a predominantly non-self complementary region. The complementary portions hybridize to form a duplex structure and the non-self complementary region forms a loop connecting the 3′ end of one strand of the duplex and the 5′ end of the other strand. shRNAs undergo intracellular processing to generate siRNAs.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

The term “small molecule” refers to an organic compound having multiple carbon-carbon bonds and a molecular weight of less than 1500 daltons. Typically such compounds comprise one or more functional groups that mediate structural interactions with proteins, e.g., hydrogen bonding, and typically include at least an amine, carbonyl, hydroxyl or carboxyl group, and in some embodiments at least two of the functional chemical groups. The small molecule agents may comprise cyclic carbon or heterocyclic structures and/or aromatic or polyaromatic structures substituted with one or more chemical functional groups and/or heteroatoms.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “variant” in referring to a polypeptide or nucleic acid sequence could be, e.g., a polypeptide or nucleic acid sequence which has at least 80%, 85%, 90%, 95%, 98%, or 99% sequence identity to the full length polypeptide or nucleic acid sequence. In some embodiments, a variant can be a fragment of a full length polypeptide or nucleic acid sequence. In some embodiments, a variant could be a naturally occurring splice variant. For example, Suv39h1 (Gene ID: 6839) has two alternatively spliced variants, variant 1 produces Suv39h1 isoform 1 protein (long transcript and encodes a longer isoform) and corresponds to mRNA NM_001282166.1, and protein NP_001269095.1, whereas variant 2 produces Suv39h1 isoform 2, which differs in the 5′ UTR, lacks a portion of the 5′ coding region, and initiates translation at an alternate start codon as compared to variant 1. The encoded Suv39h1 isoform (2) protein is shorter and has a distinct N-terminus, compared to isoform 1. The mRNA for Suv39h1 isoform 2 is NM_003173.3, which encodes the isoform 2 protein corresponding to NP_003164.1.A variant could be a polypeptide or nucleic acid sequence which has at least 80%, 85%, 90%, 95%, 98%, or 99% sequence identity to a fragment of at least 50% the length of the full-length polypeptide or full-length nucleic acid sequence, wherein the fragment is at least 50%, 60%, 70%, 80%, 85%, 90%, 95%, 98%, or 99% as long as the full length wild type polypeptide or nucleic acid sequence having an activity of interest. For example, a variant of KDM4d that has the ability to increase the efficiency of SCNT to the same, or similar extent, as compared to the KDM4d polypeptide or KDM4d nucleic acid sequence.

The term “functional fragment” or “biologically active fragment” are used interchangeably herein refers to a polypeptide having amino acid sequence which is smaller in size than the polypeptide from which it is a fragment of, where the functional fragment polypeptide has about at least 50%, or 60% or 70% or at 80% or 90% or 100% or greater than 100%, for example 1.5-fold, 2-fold, 3-fold, 4-fold or greater than 4-fold the same biological action as the polypeptide from which it is a fragment of. Functional fragment polypeptides may have additional functions that can include decreased antigenicity, increased DNA binding (as in transcription factors), or altered RNA binding (as in regulating RNA stability or degradation). In some embodiments, the biologically active fragment is substantially homologous to the polypeptide it is a fragment of. Without being limited to theory, an exemplary example of a functional fragment of the KDM4 histone demethylase activator of KDM4A comprises a fragment of SEQ ID NO:9, (e.g., wherein the fragment is at least 50%, 60%, 70%, 80%, 85%, 90%, 95%, 98%, or 99% as long as SEQ ID NO: 9) which has about at least 50%, or 60% or 70% or at 80% or 90% or 100% or greater than 100%, for example 1.5-fold, 2-fold, 3-fold, 4-fold or greater than 4-fold the ability to increase the efficiency of SCNT as compared to a KDM4A polypeptide comprising the amino acids of SEQ ID NO: 9, using the same method and under the same conditions. In some embodiments, a biologically active fragment of SEQ ID NO: 9 lacks at least 1, or at least 2, or at least between 2-10, or at least between 10-20, or at least between 20-50, or at least between 50-100 amino acids at the C-terminal, or the N-terminal of SEQ ID NO: 9. In some embodiments, a biologically active fragment of SEQ ID NO: 9 lacks at least 1, or at least 2, or at least between 2-10, or at least between 10-20, or at least between 20-50, or at least between 50-100 amino acids at both the C-terminal and the N-terminal of SEQ ID NO: 9.In some embodiments, a biologically active fragment of KDM4D of SEQ ID NO: 12 can be used, such as, for example a biologically fragment of SEQ ID NO: 12 that comprises amino acids 1-424 of SEQ ID NO: 12, as disclosed in Antony et al., Nature, 2013. In some embodiments, a biologically active fragment of SEQ ID NO: 12 comprises amino acid 1-424 of SEQ ID NO: 12 (e.g., a fragment corresponding to SEQ ID NO: 13). In some embodiments, a biologically active fragment of SEQ ID NO: 12 also lacks at least 1, or at least 2, or at least between 2-10, or at least between 10-20, or at least between 20-50, or at least between 50-100 amino acids at the C-terminal, or the N-terminal of amino acids 1-424 of SEQ ID NO: 12. In some embodiments, a biologically active fragment of SEQ ID NO: 12 comprises amino acid 1-424 of SEQ ID NO: 12 that also lacks at least 1, or at least 2, or at least between 2-10, or at least between 10-20, or at least between 20-50, or at least between 50-100 amino acids at both the C-terminal and the N-terminal of amino acids 1-424 of SEQ ID NO: 12.

The term “functional fragment” or “biologically active fragment” as used herein with respect to a nucleic acid sequence refers to a nucleic acid sequence which is smaller in size than the nucleic acid sequence which it is a fragment of, where the nucleic acid sequence has about at least 50%, or 60% or 70% or at 80% or 90% or 100% or greater than 100%, for example 1.5-fold, 2-fold, 3-fold, 4-fold or greater than 4-fold the same biological action as the biologically active fragment from which it is a fragment of. Without being limited to theory, an exemplary example of a functional fragment of the nucleic acid sequence of the KDM4 histone demethylase activator of KDM4A comprises a fragment of SEQ ID NO: 1 (e.g., wherein the fragment is at least 50%, 60%, 70%, 80%, 85%, 90%, 95%, 98%, or 99% as long as SEQ ID NO: 1) which has about at least 50%, or 60% or 70% or at 80% or 90% or 100% or greater than 100%, for example 1.5-fold, 2-fold, 3-fold, 4-fold or greater than 4-fold the ability to increase the efficiency of human SCNT as compared to a KDM4A nucleic acid sequence of SEQ ID NO: 1, using the same method and under the same conditions.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill (suffers from a disease or other condition warranting medical/surgical attention) or injured, or at increased risk of becoming ill relative to an average member of the population and in need of such attention, care, or management. “Individual” is used interchangeably with “subject” herein. In any of the embodiments of the invention, the “individual” may be a human, e.g., one who suffers or is at risk of a disease for which cell therapy is of use (“indicated”).

The term “synchronized” or “synchronous” as used herein in reference to estrus cycle, refers to assisted reproductive techniques well known to a person of ordinary skill in the art. These techniques are fully described in the reference cited in the previous paragraph. Typically, estrogen and progesterone hormones are utilized to synchronize the estrus cycle of the female animal with the developmental cycle of the embryo. The term “developmental cycle” as used herein refers to embryos of the invention and the time period that exists between each cell division within the embryo. This time period is predictable for embryos, and can be synchronized with the estrus cycle of a recipient animal.

The term “substantially similar” as used herein in reference to nuclear DNA sequences refers to two nuclear DNA sequences that are nearly identical. The two sequences may differ by copy error differences that normally occur during the replication of a nuclear DNA. Substantially similar DNA sequences are preferably greater than 97% identical, more-preferably greater than 98% identical, and most preferably greater than 99% identical. Identity is measured by dividing the number of identical residues in the two sequences by the total number of residues and multiplying the product by 100. Thus, two copies of exactly the same sequence have 100% identity, while sequences that are less highly conserved and have deletions, additions, or replacements have a lower degree of identity. Those of ordinary skill in the art will recognize that several computer programs are available for performing sequence comparisons and determining sequence identity.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### KDM4 Histone Demethylase Activators

In one aspect, the invention provides a method of increasing the efficiency of human SCNT comprising: contacting the nuclei or cytoplasm of donor human somatic cell, a recipient human oocyte, a hybrid oocyte (e.g., human enucleated oocyte comprising donor genetic material prior to fusion or activation) or a human SCNT embryo (i.e., after fusion of the donor nuclei with the enucleated oocyte) with an agent that inhibits histone methylation, in particular, inhibits H3K9 methylation, in particular, inhibits H3H9me3 trimethylation. In some embodiments, the agent is a KDM4 histone demethylase activator.

In some embodiments, a KDM4 histone demethylase activator useful in the methods, compositions and kits as disclosed herein is an agent which increases the expression of genes encoding the KDM4 family of histone demethylases, or increases the activity of human KDM4 family of histone demethylases, for example, human KDM4A, human KDM4B, human KDM4C or human KDM4D. In some embodiment, the agent increases the expression or activity of KDM4D (JMJD2D) or KDM4A (JMJD2A).

In some embodiments, the KDM4 histone demethylase activator useful in the methods, compositions and kits as disclosed herein is a nucleic acid agent which encodes the KDM4A polypeptide, or a KDM4A polypeptide, or a variant or biological active fragment thereof. As used herein, the human KDM4A nucleotide sequence corresponds to Genbank Accession No. NM_014663.2, and refers to SEQ ID NO: 1. KDM4A is also known as lysine (K)-specific demethylase 4A, JMJD2, JMJD2A, “jumonji domain containing 2”, or “jumonji domain containing 2A”. The human KDM4A protein corresponds to Genebank Accession no. NP_055478.2 (SEQ ID NO: 9). Accordingly, the protein sequence of KDM4A is as follows:

(SEQ ID NO: 9)
MASESETLNPSARIMTFYPTMEEFRNFSRYIAYIESQGAHRAGL
AKVVPPKEWKPRASYDDIDDLVIPAPIQQLVTGQSGLFTQYNIQKKAMTV
REFRKIANSDKYCTPRYSEFEELERKYWKNLTFNPPIYGADVNGTLYEKH
VDEWNIGRLRTILDLVEKESGITIEGVNTPYLYFGMWKTSFAWHTEDMDL
YSINYLHFGEPKSWYSVPPEHGKRLERLAKGFFPGSAQSCEAFLRHKMTL
ISPLMLKKYGIPFDKVTQEAGEFMITFPYGYHAGFNHGFNGAESTNFATR
RWIEYGKQAVLCSCRKDMVKISMDVFVRKFQPERYKLWKAGKDNTVIDHT
LPTPEAAEFLKESELPPRAGNEEECPEEDMEGVEDGEEGDLKTSLAKHRI
GTKRHRVCLEIPQEVSQSELFPKEDLSSEQYEMTECPAALAPVRPTHSSV
RQVEDGLTFPDYSDSTEVKFEELKNVKLEEEDEEEEQAAAALDLSVNPAS
VGGRLVFSGSKKKSSSSLGSGSSRDSISSDSETSEPLSCRAQGQTGVLTV
HSYAKGDGRVTVGEPCTRKKGSAARSFSERELAEVADEYMFSLEENKKSK
GRRQPLSKLPRHHPLVLQECVSDDETSEQLTPEEEAEETEAWAKPLSQLW
QNRPPNFEAEKEFNETMAQQAPHCAVCMIFQTYHQVEFGGFNQNCGNASD
LAPQKQRTKPLIPEMCFTSTGCSTDINLSTPYLEEDGTSILVSCKKCSVR
VHASCYGVPPAKASEDWMCSRCSANALEEDCCLCSLRGGALQRANDDRWV
HVSCAVAILEARFVNIAERSPVDVSKIPLPRFKLKCIFCKKRRKRTAGCC
VQCSHGRCPTAFHVSCAQAAGVMMQPDDWPFVVFITCFRHKIPNLERAKG
ALQSITAGQKVISKHKNGRFYQCEVVRLTTETFYEVNFDDGSFSDNLYPE
DIVSQDCLQFGPPAEGEVVQVRWTDGQVYGAKFVASHPIQMYQVEFEDGS
QLVVKRDDVYTLDEELPKRVKSRLSVASDMRFNEIFTEKEVKQEKKRQRV
INSRYREDYIEPALYRAIME

In some embodiment, the agent comprises a nucleic acid sequence of human KDM4A (SEQ ID NO: 1, or is a biologically active fragment or homologue or variant thereof of at least 80% sequence identity (or at least about 85%, or at least about 90%, or at least about 95%, or at least about 98%, or at least about 99% sequence identity) thereto which increases the efficiency of human SCNT to a similar or greater extent as compared to the corresponding sequence of SEQ ID NO: 1. In some embodiments, the composition comprises a human KDM4A nucleic acid sequence corresponding of SEQ ID NO: 1, or a biologically active fragment thereof which increases the efficiency of human SCNT to a similar or greater extent as compared to the nucleic acid sequence of SEQ ID NO: 1.

In some embodiments, a histone demethylase activator for use in the methods as disclosed herein is selected from a nucleic acid agent which encodes any human KDM4A polypeptide, or encodes a variant or biological active fragment of a human KDM4A polypeptide. In some embodiments, a histone demethylase activator for use in the methods as disclosed herein is selected from a human KDM4A polypeptide, or a variant or biological active fragment of such a human KDM4A polypeptide. It is encompassed in the present invention that one of ordinary skill in the art can identify an appropriate human homologue of human KDM4A polypeptide, and the nucleic acid encoding such a human homologue for use in the methods and composition as disclosed herein.

In some embodiments, the KDM4 histone demethylase activator useful in the methods, compositions and kits as disclosed herein is a nucleic acid agent which encodes the KDM4B polypeptide, or a KDM4B polypeptide, or a variant or biological active fragment thereof. As used herein, the human KDM4B nucleic acid corresponds to Genbank Accession No. NM_015015.2, and refers to SEQ ID NO: 2 as disclosed herein. KDM4B is also known as lysine (K)-specific demethylase 4B, JMJD2B or “jumonji domain containing 2B”, KIAA0876, TDRD14B, or “tudor domain containing 14B. The human KDM4B protein corresponds to Genebank Accession no. NP_055830.1 (SEQ ID NO: 10). Accordingly, the protein sequence of KDM4B is as follows:

(SEQ ID NO: 10)
MGSEDHGAQNPSCKIMTFRPTMEEFKDFNKYVAYIESQGAHRAG
LAKIIPPKEWKPRQTYDDIDDVVIPAPIQQVVTGQSGLFTQYNIQKKAMT
VGEYRRLANSEKYCTPRHQDFDDLERKYWKNLTFVSPIYGADISGSLYDD
DVAQWNIGSLRTILDMVERECGTIIEGVNTPYLYFGMWKTTFAWHTEDMD
LYSINYLHFGEPKSWYAIPPEHGKRLERLAIGFFPGSSQGCDAFLRHKMT
LISPIILKKYGIPFSRITQEAGEFMITFPYGYHAGFNHGFNCAESTNFAT
LRWIDYGKVATQCTCRKDMVKISMDVFVRILQPERYELWKQGKDLTVLDH
TRPTALTSPELSSWSASRASLKAKLLRRSHRKRSQPKKPKPEDPKFPGEG
TAGAALLEEAGGSVKEEAGPEVDPEEEEEEPQPLPHGREAEGAEEDGRGK
LRPTKAKSERKKKSFGLLPPQLPPPPAHFPSEEALWLPSPLEPPVLGPGP
AAMEESPLPAPLNVVPPEVPSEELEAKPRPIIPMLYVVPRPGKAAFNQEH
VSCQQAFEHFAQKGPTWKEPVSPMELTGPEDGAASSGAGRMETKARAGEG
QAPSTFSKLKMEIKKSRRHPLGRPPTRSPLSVVKQEASSDEEASPFSGEE
DVSDPDALRPLLSLQWKNRAASFQAERKFNAAAARTEPYCAICTLFYPYC
QALQTEKEAPIASLGEGCPATLPSKSRQKTRPLIPEMCFTSGGENTEPLP
ANSYIGDDGTSPLIACGKCCLQVHASCYGIRPELVNEGWTCSRCAAHAWT
AECCLCNLRGGALQMTTDRRWIHVICAIAVPEARFLNVIERHPVDISAIP
EQRWKLKCVYCRKRMKKVSGACIQCSYEHCSTSFHVTCAHAAGVLMEPDD
WPYVVSITCLKHKSGGHAVQLLRAVSLGQVVITKNRNGLYYRCRVIGAAS
QTCYEVNFDDGSYSDNLYPESITSRDCVQLGPPSEGELVELRWTDGNLYK
AKFISSVTSHIYQVEFEDGSQLTVKRGDIFTLEEELPKRVRSRLSLSTGA
PQEPAFSGEEAKAAKRPRVGTPLATEDSGRSQDYVAFVESLLQVQGRPGA
PF

In some embodiment, the agent comprises a nucleic acid sequence of human KDM4B (SEQ ID NO: 2, or is a biologically active fragment or homologue or variant thereof of at least 80% sequence identity (or at least about 85%, or at least about 90%, or at least about 95%, or at least about 98%, or at least about 99% sequence identity) thereto which increases the efficiency of human SCNT to a similar or greater extent as compared to the corresponding sequence of SEQ ID NO: 2. In some embodiments, the composition comprises a human KDM4B nucleic acid sequence corresponding of SEQ ID NO: 2, or a biologically active fragment thereof which increases the efficiency of human SCNT to a similar or greater extent as compared to the nucleic acid sequence of SEQ ID NO: 2.

In some embodiments, a histone demethylase activator for use in the methods as disclosed herein is selected from a nucleic acid agent which encodes any human KDM4B polypeptide, or encodes a variant or biological active fragment of a human KDM4B polypeptide. In some embodiments, a histone demethylase activator for use in the methods as disclosed herein is selected from any human KDM4B polypeptide, or a variant or biological active fragment of such a human KDM4B polypeptide. It is encompassed in the present invention that one of ordinary skill in the art can identify an appropriate human homologue of human KDM4B polypeptide, and the nucleic acid encoding such a human homologue for use in the methods and composition as disclosed herein.

In some embodiments, the KDM4 histone demethylase activator useful in the methods, compositions and kits as disclosed herein is a nucleic acid agent which encodes the KDM4C polypeptide, or a KDM4C polypeptide, or a variant or biological active fragment thereof. As used herein, the human KDM4C nucleic acid sequence corresponds to Genbank Accession No. NM_015061.3 (SEQ ID NO: 3) as disclosed herein. KDM4C is also known as lysine (K)-specific demethylase C, JMJD2C or “jumonji domain containing 2C” GASC1, KIAA0780, TDRD14C or “tudor domain containing 14C. The human KDM4C protein corresponds to Genebank Accession no. NP_055876.2 (SEQ ID NO: 11). Accordingly, the protein sequence of KDM4C is as follows:

(SEQ ID NO: 11)
MEVAEVESPLNPSCKIMTFRPSMEEFREFNKYLAYMESKGAHRA
GLAKVIPPKEWKPRQCYDDIDNLLIPAPIQQMVTGQSGLFTQYNIQKKAM
TVKEFRQLANSGKYCTPRYLDYEDLERKYWKNLTFVAPIYGADINGSIYD
EGVDEWNIARLNTVLDVVEEECGISIEGVNTPYLYFGMWKTTFAWHTEDM
DLYSINYLHFGEPKSWYAIPPEHGKRLERLAQGFFPSSSQGCDAFLRHKM
TLISPSVLKKYGIPFDKITQEAGEFMITFPYGYHAGFNHGFNCAESTNFA
TVRWIDYGKVAKLCTCRKDMVKISMDIFVRKFQPDRYQLWKQGKDIYTID
HTKPTPASTPEVKAWLQRRRKVRKASRSFQCARSTSKRPKADEEEEVSDE
VDGAEVPNPDSVTDDLKVSEKSEAAVKLRNTEASSEEESSASRMQVEQNL
SDHIKLSGNSCLSTSVTEDIKTEDDKAYAYRSVPSISSEADDSIPLSSGY
EKPEKSDPSELSWPKSPESCSSVAESNGVLTEGEESDVESHGNGLEPGEI
PAVPSGERNSFKVPSIAEGENKTSKSWRHPLSRPPARSPMTLVKQQAPSD
EELPEVLSIEEEVEETESWAKPLIHLWQTKSPNFAAEQEYNATVARMKPH
CAICTLLMPYHKPDSSNEENDARWETKLDEVVTSEGKTKPLIPEMCFIYS
EENIEYSPPNAFLEEDGTSLLISCAKCCVRVHASCYGIPSHEICDGWLCA
RCKRNAWTAECCLCNLRGGALKQTKNNKWAHVMCAVAVPEVRFTNVPERT
QIDVGRIPLQRLKLKCIFCRHRVKRVSGACIQCSYGRCPASFHVTCAHAA
GVLMEPDDWPYVVNITCFRHKVNPNVKSKACEKVISVGQTVITKHRNTRY
YSCRVMAVTSQTFYEVMFDDGSFSRDTFPEDIVSRDCLKLGPPAEGEVVQ
VKWPDGKLYGAKYFGSNIAHMYQVEFEDGSQIAMKREDIYTLDEELPKRV
KARFSTASDMRFEDTFYGADIIQGERKRQRVLSSRFKNEYVADPVYRTFL
KSSFQKKCQKRQ

In some embodiment, the agent comprises a nucleic acid sequence of human KDM4C (SEQ ID NO: 3), or is a biologically active fragment or homologue or variant thereof of at least 80% sequence identity (or at least about 85%, or at least about 90%, or at least about 95%, or at least about 98%, or at least about 99% sequence identity) thereto which increases the efficiency of human SCNT to a similar or greater extent as compared to the corresponding sequence of SEQ ID NO: 3. In some embodiments, the composition comprises a human KDM4C nucleic acid sequence corresponding of SEQ ID NO: 3, or a biologically active fragment thereof which increases the efficiency of human SCNT to a similar or greater extent as compared to the nucleic acid sequence of SEQ ID NO: 3.

In some embodiments, a histone demethylase activator for use in the methods as disclosed herein is selected from a nucleic acid agent which encodes any human KDM4C polypeptide, or encodes a variant or biological active fragment of a human KDM4C polypeptide. In some embodiments, a histone demethylase activator for use in the methods as disclosed herein is selected from any human KDM4C polypeptide, or a variant or biological active fragment of such a human KDM4C polypeptide. It is encompassed in the present invention that one of ordinary skill in the art can identify an appropriate human homologue of human KDM4C polypeptide, and the nucleic acid encoding such a human homologue for use in the methods and composition as disclosed herein.

In some embodiments, the KDM4 histone demethylase activator useful in the methods, compositions and kits as disclosed herein is a nucleic acid agent which encodes the KDM4D polypeptide, or a KDM4D polypeptide, or a variant or biological active fragment thereof. As used herein, the human KDM4D nucleic acid sequence corresponds to Genbank Accession No. NM_018039.2, and refers to SEQ ID NO: 4 as disclosed herein. KDM4D is also known as lysine (K)-specific demethylase 4D, FLJ10251, JMJD2D or “jumonji domain containing 2D”. The human KDM4D protein corresponds to Genebank Accession no. NP_060509.2” (SEQ ID NO: 12). Accordingly, the protein sequence of KDM4D is as follows:

(SEQ ID NO: 12)
METMKSKANCAQNPNCNIMIFHPTKEEFNDFDKYIAYMESQGAH
RAGLAKIIPPKEWKARETYDNISEILIATPLQQVASGRAGVFTQYHKKKK
AMTVGEYRHLANSKKYQTPPHQNFEDLERKYWKNRIYNSPIYGADISGSL
FDENTKQWNLGHLGTIQDLLEKECGVVIEGVNTPYLYFGMWKTTFAWHTE
DMDLYSINYLHLGEPKTWYVVPPEHGQRLERLARELFPGSSRGCGAFLRH
KVALISPTVLKENGIPFNRITQEAGEFMVTFPYGYHAGFNHGFNCAEAIN
FATPRWIDYGKMASQCSCGEARVTFSMDAFVRILQPERYDLWKRGQDRAV
VDHMEPRVPASQELSTQKEVQLPRRAALGLRQLPSHWARHSPWPMAARSG
TRCHTLVCSSLPRRSAVSGTATQPRAAAVHSSKKPSSTPSSTPGPSAQII
HPSNGRRGRGRPPQKLRAQELTLQTPAKRPLLAGTTCTASGPEPEPLPED
GALMDKPVPLSPGLQHPVKASGCSWAPVP

In some embodiment, the agent comprises a nucleic acid sequence of human KDM4D (SEQ ID NO: 4, or is a biologically active fragment or homologue or variant thereof of at least 80% sequence identity (or at least about 85%, or at least about 90%, or at least about 95%, or at least about 98%, or at least about 99% sequence identity) thereto which increases the efficiency of SCNT to a similar or greater extent as compared to the corresponding sequence of SEQ ID NO: 4. In some embodiments, the composition comprises a human KDM4D nucleic acid sequence corresponding of SEQ ID NO: 4, or a biologically active fragment thereof which increases the efficiency of human SCNT to a similar or greater extent as compared to the nucleic acid sequence of SEQ ID NO: 4.

In some embodiments, the agent which contacts a donor human somatic cell, a recipient human oocyte, a hybrid oocyte (e.g., human enucleated oocyte comprising donor genetic material prior to fusion or activation) or a human SCNT embryo (i.e., after fusion of the donor nuclei with the enucleated oocyte) increases the expression of human KDM4A protein of SEQ ID NO: 9, or a human KDM4B protein of SEQ ID NO: 10, or a human KDM2C protein of SEQ ID NO: 11, or a human KDM4D protein of SEQ ID NO: 12, and/or comprises any one or a combination of: a human KDM4A nucleic acid sequence corresponding of SEQ ID NO: 1, a human KDM4B nucleic acid sequence corresponding of SEQ ID NO: 2, a human KDM4C nucleic acid sequence corresponding of SEQ ID NO: 3, a human KDM4D nucleic acid sequence corresponding of SEQ ID NO: 4, a human KDM4E nucleic acid sequence corresponding to SEQ ID NO: 45, or a biologically active fragment of SEQ ID NO: 1-4 or SEQ ID NO: 45 which increases the efficiency of human SCNT to a similar or greater extent (e.g., at least about 110%, or at least about 120%, or at least about 130%, or at least about 140%, or at least about 150%, or more than 150% increased) as compared to the nucleic acid sequence of SEQ ID NO: 1-4 or SEQ ID NO: 45.

In some embodiments, a biologically active fragment of SEQ ID NO: 12 comprises amino acids 1-424 of SEQ ID NO: 12, as disclosed in Antony et al., Nature, 2013. In some embodiments, a biologically active fragment of SEQ ID NO: 12 comprises amino acid 1-424 of SEQ ID NO: 12 that also lacks at least 1, or at least 2, or at least between 2-10, or at least between 10-20, or at least between 20-50, or at least between 50-100 amino acids at the C-terminal, or the N-terminal of amino acids 1-424 of SEQ ID NO: 12. In some embodiments, a biologically active fragment of SEQ ID NO: 12 comprises amino acid 1-424 of SEQ ID NO: 12 that also lacks at least 1, or at least 2, or at least between 2-10, or at least between 10-20, or at least between 20-50, or at least between 50-100 amino acids at both the C-terminal and the N-terminal of amino acids 1-424 of SEQ ID NO: 12. In some embodiments, a biologically active fragment of SEQ ID NO: 12 comprises SEQ ID NO: 64, wherein the protein sequence of SEQ ID NO: 13 comprises:

(SEQ ID NO: 13)
METMKSKANCAQNPNCNIMIFHPTKEEFNDFDKYIAYMESQGAH
RAGLAKIIPPKEWKARETYDNISEILIATPLQQVASGRAGVFTQYHKKKK
AMTVGEYRHLANSKKYQTPPHQNFEDLERKYWKNRIYNSPIYGADISGSL
FDENTKQWNLGHLGTIQDLLEKECGVVIEGVNTPYLYFGMWKTTFAWHTE
DMDLYSINYLHLGEPKTWYVVPPEHGQRLERLARELFPGSSRGCGAFLRH
KVALISPTVLKENGIPFNRITQEAGEFMVTFPYGYHAGFNHGFNCAEAIN
FATPRWIDYGKMASQCSCGEARVTFSMDAFVRILQPERYDLWKRGQDRAV
VDHMEPRVPASQELSTQKEVQLPRRAALGLRQLPSHWARHSPWPMAARSG
TRCHTLVCSSLPRRSAVSGTATQPRAAAV

In some embodiments, a biologically active fragment of SEQ ID NO: 12 comprises amino acids of SEQ ID NO: 13 that also lacks at least 1, or at least 2, or at least between 2-10, or at least between 10-20, or at least between 20-50 amino acids at the C-terminal of SEQ ID NO: 13. In some embodiments, a biologically active fragment of SEQ ID NO: 12 comprises amino acids of SEQ ID NO: 13 that also lacks at least 1, or at least 2, or at least between 2-10, or at least between 10-20, or at least between 20-50 amino acids at the N-terminal of SEQ ID NO: 13.

In some embodiments, a histone demethylase activator for use in the methods, compositions and kits as disclosed herein is selected from a nucleic acid agent which encodes any human KDM4D polypeptide, or encodes a variant or biological active fragment of a human KDM4D polypeptide. In some embodiments, a histone demethylase activator for use in the methods as disclosed herein is selected from any human KDM4D polypeptide, or a variant or biological active fragment of such a human KDM4D polypeptide. It is encompassed in the present invention that one of ordinary skill in the art can identify an appropriate human homologue of human KDM4D polypeptide, and the nucleic acid encoding such a human homologue for use in the methods and composition as disclosed herein.

In some embodiments, the KDM4 histone demethylase activator useful in the methods, compositions and kits as disclosed herein is a nucleic acid agent which encodes the KDM4E polypeptide, or a KDM4E polypeptide, or a variant or biological active fragment thereof. As used herein, the human KDM4E nucleic acid corresponds to Genbank Accession No. NM_001161630.1, and refers to SEQ ID NO: 45 as disclosed herein. KDM4E is also known as lysine (K)-specific demethylase 4E, JMJD2E or “jumonji domain containing 2E”, KDM4DL, or “lysine (K)-specific demethylase 4D-like”. The human KDM4B protein corresponds to Genebank Accession no. NP_001155102.1 (SEQ ID NO: 46). Accordingly, the protein sequence of human KDM4E is as follows:

(SEQ ID NO: 46)
MKSVHSSPQNTSHTIMTFYPTMEEFADFNTYVAYMESQGAHQAG
LAKVIPPKEWKARQMYDDIEDILIATPLQQVTSGQGGVFTQYHKKKKAMR
VGQYRRLANSKKYQTPPHQNFADLEQRYWKSHPGNPPIYGADISGSLFEE
STKQWNLGHLGTILDLLEQECGVVIEGVNTPYLYFGMWKTTFAWHTEDMD
LYSINYLHFGEPKTWYVVPPEHGQHLERLARELFPDISRGCEAFLRHKVA
LISPTVLKENGIPFNCMTQEAGEFMVTFPYGYHAGFNHGFNCAEAINFAT
PRWIDYGKMASQCSCGESTVTFSMDPFVRIVQPESYELWKHRQDLAIVEH
TEPRVAESQELSNWRDDIVLRRAALGLRLLPNLTAQCPTQPVSSGHCYNP
KGCGTDAVPGSAFQSSAYHTQTQSLTLGMSARVLLPSTGSWGSGRGRGRG
QGQGRGCSRGRGHGCCTRELGTEEPTVQPASKRRLLMGTRSRAQGHRPQL
PLANDLMTNLSL

In some embodiment, the agent comprises a nucleic acid sequence of human KDM4E (SEQ ID NO: 45, or is a biologically active fragment or homologue or variant thereof of at least 80% sequence identity (or at least about 85%, or at least about 90%, or at least about 95%, or at least about 98%, or at least about 99% sequence identity) thereto which increases the efficiency of human SCNT to a similar or greater extent as compared to the corresponding sequence of SEQ ID NO: 45. In some embodiments, the composition comprises a human KDM4E nucleic acid sequence corresponding of SEQ ID NO: 45, or a biologically active fragment thereof which increases the efficiency of human SCNT to a similar or greater extent as compared to the nucleic acid sequence of SEQ ID NO: 45.

In some embodiments, a histone demethylase activator for use in the methods as disclosed herein is selected from a nucleic acid agent which encodes any human KDM4E polypeptide, or encodes a variant or biological active fragment of a human KDM4E polypeptide. In some embodiments, a histone demethylase activator for use in the methods as disclosed herein is selected from any human KDM4E polypeptide, or a variant or biological active fragment of such a human KDM4E polypeptide. It is encompassed in the present invention that one of ordinary skill in the art can identify an appropriate human homologue of human KDM4E polypeptide, and the nucleic acid encoding such a human homologue for use in the methods and composition as disclosed herein.

As used in some embodiments, a histone demethylase activator for use in the methods as disclosed herein is selected from any of the group consisting of, AOF (LSD1), AOF1 (LSD2), FBXL11 (JHDM1A), Fbxl10 (JHDM1B), FBXL19 (JHDM1C), KIAA1718 (JHDM1D), PHF2 (JHDM1E), PHF8 (JHDM1F), JMJDIA (JHDM2A), JMJD1B (JHDM2B), JMJD1C (JHDM2C), KDM4A (JMJD2A; JHDM3A), KDM4B (JMJD2B; JHDM3B), KDM4C (JMJD2C; JHDM3C),KDM4D (JMJD2D; JHDM3D), KDM4E (JMJD2E), RBP2 (JARID1A), PLU1 (JARID1B), SMCX (JARID1C), SMCY (JARID1D), Jumonji (JARID2), UTX (UTX), UTY (UTY), JMJD3 (JMJD3), JMJD4 (JMJD4), JMJD5 (JMJD5), JMJD6 (JMJD6), JMJD7 (JMJD7), JMJD8 (JMJD8). Such histone demethylase activators are disclosed in US Application 2011/0139145, which is incorporated herein in its entirety by reference.

In some embodiments, a KDM4 histone demethylase activator is a polypeptide variant, or a nucleic acid sequence that encodes a polypeptide variant of at least 80%, 85%, 90%, 95%, 98%, or 99% identical to the full-length polypeptide, or a fragment of the polypeptide of any human KDM4 polypeptides of SEQ ID NOs: 9-12 or SEQ ID NO: 46 (human KDM4A-KDM4E) or encoded by any one of the nucleic acid sequences corresponding to SEQ ID NO: 1-4 or SEQ ID NO: 45.

In some embodiments, a KDM4 histone demethylase activator is a polypeptide variant, or a nucleic acid sequence that encodes a polypeptide variant, of at least 80%, 85%, 90%, 95%, 98%, or 99% identical to the full-length polypeptide, or a fragment of the polypeptide of KDM4 polypeptides of SEQ ID NOs: 9-12 or SEQ ID NO: 46 (human KDM4A-KDM4E). In some embodiments, a KDM4 histone demethylase is a fragment of at least 20 consecutive amino acids of SEQ ID NOs: 9-12 or SEQ ID NO: 46 (human KDM4A-KDM4E), or a fragment of human KDM4A, KDM4B, KDM4C, KDM4D or KDM4E which is at least 50%, 60%, 70%, 80%, 85%, 90%, 95%, 98%, or 99% as long as the full length wild type polypeptide or a domain thereof having an activity of interest, such as at least 80% or greater in ability to increase the efficiency of SCNT as compared to the efficiency of a protein of SEQ ID NOs: 9-12 or SEQ ID NO: 46 (human KDM4A-KDM4E) respectively.

In some embodiments, a biologically active fragment of human KDM4A comprises a fragment of SEQ ID NO:9, (e.g., wherein the fragment is at least 50%, 60%, 70%, 80%, 85%, 90%, 95%, 98%, or 99% as long as SEQ ID NO: 9) which has about at least 50%, or 60% or 70% or at 80% or 90% or 100% or greater than 100%, for example 1.5-fold, 2-fold, 3-fold, 4-fold or greater than 4-fold the ability to increase the efficiency of SCNT as compared to a KDM4A polypeptide comprising the amino acids of SEQ ID NO: 9, using the same method and under the same conditions.

In some embodiments, a biologically active fragment of human KDM4B comprises a fragment of SEQ ID NO: 10, (e.g., wherein the fragment is at least 50%, 60%, 70%, 80%, 85%, 90%, 95%, 98%, or 99% as long as SEQ ID NO: 10) which has about at least 50%, or 60% or 70% or at 80% or 90% or 100% or greater than 100%, for example 1.5-fold, 2-fold, 3-fold, 4-fold or greater than 4-fold the ability to increase the efficiency of SCNT as compared to a KDM4B polypeptide comprising the amino acids of SEQ ID NO: 10, using the same method and under the same conditions.

In some embodiments, a biologically active fragment of human KDM4C comprises a fragment of SEQ ID NO: 11 (e.g., wherein the fragment is at least 50%, 60%, 70%, 80%, 85%, 90%, 95%, 98%, or 99% as long as SEQ ID NO: 11) which has about at least 50%, or 60% or 70% or at 80% or 90% or 100% or greater than 100%, for example 1.5-fold, 2-fold, 3-fold, 4-fold or greater than 4-fold the ability to increase the efficiency of SCNT as compared to a KDM4C polypeptide comprising the amino acids of SEQ ID NO: 11, using the same method and under the same conditions.

In some embodiments, a biologically active fragment of human KDM4D comprises a fragment of SEQ ID NO: 12, (e.g., wherein the fragment is at least 50%, 60%, 70%, 80%, 85%, 90%, 95%, 98%, or 99% as long as SEQ ID NO: 12) which has about at least 50%, or 60% or 70% or at 80% or 90% or 100% or greater than 100%, for example 1.5-fold, 2-fold, 3-fold, 4-fold or greater than 4-fold the ability to increase the efficiency of SCNT as compared to a KDM4D polypeptide comprising the amino acids of SEQ ID NO: 12, using the same method and under the same conditions. In some embodiments, a biologically active fragment of SEQ ID NO: 12 comprises amino acids 1-424 of SEQ ID NO: 12, as disclosed in Antony et al., Nature, 2013. In some embodiments, a biologically active fragment of SEQ ID NO: 12 comprises amino acid 1-424 of SEQ ID NO: 12 that also lacks at least 1, or at least 2, or at least between 2-10, or at least between 10-20, or at least between 20-50, or at least between 50-100 amino acids at the C-terminal, or the N-terminal of amino acids 1-424 of SEQ ID NO: 12. In some embodiments, a biologically active fragment of SEQ ID NO: 12 comprises amino acid 1-424 of SEQ ID NO: 12 that also lacks at least 1, or at least 2, or at least between 2-10, or at least between 10-20, or at least between 20-50, or at least between 50-100 amino acids at both the C-terminal and the N-terminal of amino acids 1-424 of SEQ ID NO: 12. In some embodiments, a biologically active fragment of SEQ ID NO: 12 comprises SEQ ID NO: 13, wherein the protein sequence of SEQ ID NO: 13 comprises:

(SEQ ID NO: 13)
METMKSKANCAQNPNCNIMIFHPTKEEFNDFDKYIAYMESQGAH
RAGLAKIIPPKEWKARETYDNISEILIATPLQQVASGRAGVFTQYHKKKK
AMTVGEYRHLANSKKYQTPPHQNFEDLERKYWKNRIYNSPIYGADISGSL
FDENTKQWNLGHLGTIQDLLEKECGVVIEGVNTPYLYFGMWKTTFAWHTE
DMDLYSINYLHLGEPKTWYVVPPEHGQRLERLARELFPGSSRGCGAFLRH
KVALISPTVLKENGIPFNRITQEAGEFMVTFPYGYHAGFNHGFNCAEAIN
FATPRWIDYGKMASQCSCGEARVTFSMDAFVRILQPERYDLWKRGQDRAV
VDHMEPRVPASQELSTQKEVQLPRRAALGLRQLPSHWARHSPWPMAARSG
TRCHTLVCSSLPRRSAVSGTATQPRAAAV

In some embodiments, a biologically active fragment of SEQ ID NO: 12 comprises amino acids of SEQ ID NO: 13 that also lacks at least 1, or at least 2, or at least between 2-10, or at least between 10-20, or at least between 20-50 amino acids at the C-terminal of SEQ ID NO: 13. In some embodiments, a biologically active fragment of SEQ ID NO: 12 comprises amino acids of SEQ ID NO: 13 that also lacks at least 1, or at least 2, or at least between 2-10, or at least between 10-20, or at least between 20-50 amino acids at the N-terminal of SEQ ID NO: 13.

In some embodiments, a biologically active fragment of human KDM4E comprises a fragment of SEQ ID NO: 46 (e.g., wherein the fragment is at least 50%, 60%, 70%, 80%, 85%, 90%, 95%, 98%, or 99% as long as SEQ ID NO: 46) which has about at least 50%, or 60% or 70% or at 80% or 90% or 100% or greater than 100%, for example 1.5-fold, 2-fold, 3-fold, 4-fold or greater than 4-fold the ability to increase the efficiency of SCNT as compared to a KDM4E polypeptide comprising the amino acids of SEQ ID NO: 46, using the same method and under the same conditions.

In some embodiments, a biologically active variant of human KDM4A comprises a variant of SEQ ID NO: 9 which has at least 80% sequence identity (or at least about 85%, or at least about 90%, or at least about 95%, or at least about 98%, or at least about 99% sequence identity) to SEQ ID NO: 9, (e.g., wherein the variant is at least 85%, 90%, 95%, 98%, or 99% identical SEQ ID NO: 9) which has about at least 50%, or 60% or 70% or at 80% or 90% or 100% or greater than 100%, for example 1.5-fold, 2-fold, 3-fold, 4-fold or greater than 4-fold the ability to increase the efficiency of human SCNT as compared to a KDM4A polypeptide comprising the amino acids of SEQ ID NO: 9, using the same method and under the same conditions.

In some embodiments, a biologically active variant of human KDM4B comprises a variant of SEQ ID NO: 10 which has at least 80% sequence identity (or at least about 85%, or at least about 90%, or at least about 95%, or at least about 98%, or at least about 99% sequence identity) to SEQ ID NO: 10, (e.g., wherein the variant is at least 85%, 90%, 95%, 98%, or 99% identical SEQ ID NO: 10) which has about at least 50%, or 60% or 70% or at 80% or 90% or 100% or greater than 100%, for example 1.5-fold, 2-fold, 3-fold, 4-fold or greater than 4-fold the ability to increase the efficiency of human SCNT as compared to a KDM4B polypeptide comprising the amino acids of SEQ ID NO: 10, using the same method and under the same conditions.

In some embodiments, a biologically active variant of human KDM4C comprises a variant of SEQ ID NO: 11 which has at least 80% sequence identity (or at least about 85%, or at least about 90%, or at least about 95%, or at least about 98%, or at least about 99% sequence identity) to SEQ ID NO: 11, (e.g., wherein the variant is at least 85%, 90%, 95%, 98%, or 99% identical SEQ ID NO: 11) which has about at least 50%, or 60% or 70% or at 80% or 90% or 100% or greater than 100%, for example 1.5-fold, 2-fold, 3-fold, 4-fold or greater than 4-fold the ability to increase the efficiency of human SCNT as compared to a KDM4C polypeptide comprising the amino acids of SEQ ID NO: 11, using the same method and under the same conditions.

In some embodiments, a biologically active variant of human KDM4D comprises a variant of SEQ ID NO: 12 which has at least 80% sequence identity (or at least about 85%, or at least about 90%, or at least about 95%, or at least about 98%, or at least about 99% sequence identity) to SEQ ID NO: 12, (e.g., wherein the variant is at least 85%, 90%, 95%, 98%, or 99% identical SEQ ID NO: 12) which has about at least 50%, or 60% or 70% or at 80% or 90% or 100% or greater than 100%, for example 1.5-fold, 2-fold, 3-fold, 4-fold or greater than 4-fold the ability to increase the efficiency of human SCNT as compared to a KDM4D polypeptide comprising the amino acids of SEQ ID NO: 12, using the same method and under the same conditions.

In some embodiments, a biologically active variant of human KDM4E comprises a variant of SEQ ID NO: 46 which has at least 80% sequence identity (or at least about 85%, or at least about 90%, or at least about 95%, or at least about 98%, or at least about 99% sequence identity) to SEQ ID NO: 46, (e.g., wherein the variant is at least 85%, 90%, 95%, 98%, or 99% identical SEQ ID NO: 46) which has about at least 50%, or 60% or 70% or at 80% or 90% or 100% or greater than 100%, for example 1.5-fold, 2-fold, 3-fold, 4-fold or greater than 4-fold the ability to increase the efficiency of human SCNT as compared to a KDM4E polypeptide comprising the amino acids of SEQ ID NO: 46, using the same method and under the same conditions.

In some embodiments, the KDM4 histone demethylase activator useful in the methods and compositions and kits as disclosed herein is a nucleic acid agent, such as a RNA or modified RNA (modRNA) as disclosed in US Patent Application US2012/03228640, corresponding to sequences SEQ ID NO: 1-4 or SEQ ID NO: 45, or encoding a protein corresponding to SEQ ID NO: 9-12 or SEQ ID NO: 46 or a functional fragment, or a biologically active variant or fragment thereof. In some embodiments, a KDM4 histone demethylase activator comprises a nucleic acid agent selected from any of SEQ ID NO: 1-4 or SEQ ID NO: 45, or a nucleic acid variant which is has at least 80% sequence identity (or at least about 85%, or at least about 90%, or at least about 95%, or at least about 98%, or at least about 99% sequence identity) SEQ ID NO: 1-4 or SEQ ID NO: 45. In some embodiments, a KDM4 histone demethylase activator comprises a nucleic acid which is a fragment of at least 20 consecutive amino acids of any one of SEQ ID NO: 1-4 or SEQ ID NO: 45, e.g., a fragment of at least 20-, or at least 30- or at least 40- or at least 50 nucleic acids of SEQ ID NO: 1-4 or SEQ ID NO: 45. In some embodiments, a KDM4 histone demethylase activator which is a nucleic acid agent useful in the methods and compositions and kits is expressed from a vector, e.g., a viral vector.

In alternative embodiments, a KDM4 histone demethylase activator encompassed for use herein is a synthetic modified RNA (modRNA) corresponding to sequences SEQ ID NO: 1-4 or SEQ ID NO: 45, or encoding a protein corresponding to SEQ ID NO: 9-12 or SEQ ID NO: 46 or a functional fragment thereof. Synthetic modified RNA (modRNA) are described in U.S. applications US2012/03228640; US2009/0286852 and US2013/0111615 and U.S. Pat. Nos. 8,278,036; 8,691,966; 8,748,089; 8,835,108, which are incorporated herein in their entirety by reference. In some embodiments, the synthetic, modified RNA molecule is not expressed in a vector, and the synthetic, modified RNA molecule can be a naked synthetic, modified RNA molecule. In some embodiments, a composition can comprises at least one synthetic, modified RNA molecule present in a lipid complex.

In some embodiments, the synthetic, modified RNA molecule comprises at least two modified nucleosides, for example, at least two modified nucleosides are selected from the group consisting of 5-methylcytidine (5mC), N6-methyladenosine (m6A), 3,2′-O-dimethyluridine (m4U), 2-thiouridine (s2U), 2′ fluorouridine, pseudouridine, 2′-O-methyluridine (Um), 2′deoxy uridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m2,7G), N2, N2, 7-trimethylguanosine (m2,2,7G), and inosine (I). In some embodiments, the synthetic, modified RNA molecule further comprises a 5′ cap, such as a 5′ cap analog, e.g., a 5′ diguanosine cap. In some embodiments, a synthetic, modified RNA molecule for use in the methods and compositions as disclosed herein does not comprise a 5′ triphosphate. In some embodiments, a synthetic, modified RNA molecule for use in the methods and compositions as disclosed herein further comprises a poly(A) tail, a Kozak sequence, a 3′ untranslated region, a 5′ untranslated region, or any combination thereof, and in some embodiments, the a synthetic, modified RNA molecule can optionally treated with an alkaline phosphatase.

### H3K9 Methyltransferase Inhibitors.

In one aspect, the invention provides a method of increasing the efficiency of human SCNT comprising: contacting the nuclei or cytoplasm of a donor human somatic cell, a recipient human oocyte, a hybrid oocyte (e.g., human enucleated oocyte comprising donor genetic material prior to fusion or activation) or a human SCNT embryo (i.e., after fusion of the donor nuclei with the enucleated oocyte) with an agent that inhibits histone methylation, in particular, inhibits H3K9 methylation, in particular, inhibits H3H9me3 trimethylation in the human nuclear genetic material. In certain embodiments of the invention the agent inhibits histone methyltransferase activity. In certain embodiments of the invention the agent inhibits expression of a human histone methyltransferase. In certain embodiments of the invention the inhibitor is an inhibitor of a human H3K9 methyltransferase. As discussed herein, the inventors have discovered that inhibition of a H3K9 methyltransferase protein can be used to increase the efficiency of human SCNT. In some embodiments, an H3K9 methyltransferase inhibitor is a protein inhibitor, and in some embodiments, the inhibitor is any agent which inhibits the function of a H3K9 methyltransferase protein or the expression of a H3K9 methyltransferase from its gene.

In certain embodiments of the invention, the agent inhibits the expression or function of human histone methyltransferase SUV39h1 protein. SUV39h1 has two alternatively spliced variants (variant 1 and 2), which produce SUV39h1 isoform 1 and SUV39h1 isoform 2 proteins. In some embodiments, an agent for use in the methods, kits and compositions as disclosed herein inhibits the translation of the mRNA of variant 1 (SEQ ID NO: 47) or variant 2 (SEQ ID NO: 14) of SUV39h1. In some embodiments, an agent for use in the methods, kits and compositions as disclosed herein inhibits the function of isoform 1 (SEQ ID NO: 48) or isoform 2 (SEQ ID NO: 5) of SUV39h1 protein.

In certain embodiments of the invention, the agent inhibits the human histone methyltransferase SUV39h2 protein. In certain embodiments of the invention, the agent inhibits the expression or function of human histone methyltransferase SUV39h2 protein. SUV39h2 has five alternatively spliced variants (variants 1-5), which produce four isoforms of SUV39h2 (variants 2 and 3 both encode isoform 2). In some embodiments, an agent for use in the methods, kits and compositions as disclosed herein inhibits the translation of the mRNA of any one or more of SEQ ID NOS: 15, 49, 51, 52 and 53 (hSUV39h2 variants 1-5). In some embodiments, an agent for use in the methods, kits and compositions as disclosed herein inhibits the function of hSuv39h2 isoforms 1-4 corresponding to SEQ ID NOS: 6 and SEQ ID NOS: 54-57.

In certain embodiments of the invention, the agent is an inhibitor of the human histone methyltransferase EHMT1. In certain embodiments of the invention, the agent inhibits the human histone methyltransferase SETDB1. In certain embodiments at least two H3K9 methyltransferases (e.g., 2, 3, 4, etc.) are inhibited. In certain embodiments of the invention, both SUV39h1 and SUV39h2 are inhibited by the same agent (e.g., a SUV39h1/2 inhibitor) or by 2 or more separate agents. In certain embodiments of the invention the agent is a RNAi agent, e.g., a siRNA or shRNA that inhibits expression of any one or more of the H3K9 methyltransferase; human SUV39h1, human SUV39h2, or human SETDB1.

As used herein the term “SUV39h1” or “H3K9-histone methyltransferase SUV39h1” has its general meaning in the art and refers to the histone methyltransferase “suppressor of variegation 3-9 homolog 1 (Drosophila)” that methylates Lys-9 of histone H3 (Aagaard L, Laible G, Selenko P, Schmid M, Dorn R, Schotta G, Kuhfittig S, Wolf A, Lebersorger A, Singh P B, Reuter G, Jenuwein T (June 1999). “Functional mammalian homologues of the Drosophila PEV-modifier Su(var)3-9 encode centromere-associated proteins which complex with the heterochromatin component M31”. EMBO J 18 (7): 1923-38.). Said histone methyltransferase is also known as MG44, KMT1A, SUV39H, histone-lysine N-methyltransferase SUV39H1, H3-K9-HMTase 1, OTTHUMP00000024298, Su(var)3-9 homolog 1, lysine N-methyltransferase 1A, histone H3-K9 methyltransferase 1, position-effect variegation 3-9 homolog, histone-lysine N-methyltransferase, or H3 lysine-9 specific 1. The term encompasses all orthologs of SUV39h1 such as SU(VAR)3-9, and includes variant 1 and variant 2, which encode SUV39h1 isoform 1 and SUV39h1 isoform 2. As summarized in Table 8, and without wishing to be limited to theory, Suv39h1 (Gene ID: 6839) has two alternatively spliced variants, variant 1 produces Suv39h1 isoform 1 protein (long transcript and encodes a longer isoform) and corresponds to mRNA NM_001282166.1 (SEQ ID NO: 47), and protein NP_001269095.1 (SEQ ID NO: 48). Variant 2 of Suv39h1 encodes isoform 2 and differs in the 5′ UTR, lacks a portion of the 5′ coding region, and initiates translation at an alternate start codon as compared to variant 1. The encoded Suv39h1 isoform 2 protein is shorter and has a distinct N-terminus, compared to isoform 1 protein. The mRNA for Suv39h1 isoform 2 is NM_003173.3 (SEQ ID NO: 14), which encodes the isoform 2 protein corresponding to NP_003164.1 (SEQ ID NO: 5).

As used herein the term “SUV39h2” or “H3K9-histone methyltransferase SUV39h2” has its general meaning in the art and refers to the histone methyltransferase “suppressor of variegation 3-9 homolog 2 (Drosophila)” that methylates Lys-9 of histone H3. Said histone methyltransferase is also known as KMT1B, FLJ23414, H3-K9-HMTase 2, histone H3-K9 methyltransferase 2, lysine N-methyltransferase 1B, su(var)3-9 homolog 2. The term encompasses all homologues (Suv39h2 gene is conserved in chimpanzee, Rhesus monkeys, dog, cow, mouse, rat, chicken and frog), as well as alternatively spliced variants of SUV39h2 disclosed in Table 8. Without wishing to be limited to theory, Table 8 lists the five alternatively spliced human Suv39h2 (Gene ID: 79723) variants, which are as follows: variant 1 encode Suv39h2 isoform 1 protein (long transcript and encodes a longer isoform); variant 2 and variant 3 both encode Suv39h2 isoform 2; variant 4 encodes Suv39h2 isoform 3, and variant 5 encodes Sub39h2 isoform 4. The sequence identifiers of the mRNA for Suv39h2 variants and their corresponding proteins are shown in Table 8.

TABLE 8
Summary of sequence for hSUVh1 and hSUVh2 variants.

| | mRNA (Accession | Amino acid sequence | |
|---|---|---|---|
| | number) (common | (Accession number) | |
| hSUV39h1/2 gene | name) | (common name) | Description |
| hSUV39h1 variant | SEQ ID NO: 47 | SEQ ID NO: 48 | variant 1 produces Suv39h1 isoform 1 |
| 1/isoform 1 | (NM_001282166.1) | (NP_001269095.1) | protein (long transcript and encodes a |
| | (hSUV39h1 variant 1) | (hSUV39h1 isoform 1) | longer isoform) and corresponds to |
| | | | mRNA NM_001282166.1 |
| hSUV39h1 variant | SEQ ID NO: 14 | SEQ ID NO: 5 | variant 2 produces Suv39h1 isoform |
| 2/isoform 2 | (NM_003173.3) | (NP_003164.1) | 2, which differs in the 5′ UTR, lacks a |
| | (hSUV39h1 variant 2) | (hSUV39h1 isoform 2) | portion of the 5′ coding region, and |
| | | | initiates translation at an alternate start |
| | | | codon as compared to variant 1. The |
| | | | encoded Suv39h1 isoform (2) protein |
| | | | is shorter and has a distinct N- |
| | | | terminus, compared to isoform 1. |
| hSUV39h2 variant | SEQ ID NO: 49 | SEQ ID NO: 54 | Variant 1 encodes longest hSuv39h2 |
| 1/isoform 1 | (NM_001193424.1) | (NP_001180353.1) | isoform, isoform 1 |
| | (hSUV39h2 variant 1) | (hSUV39h2 isoform 1) | |
| hSUV39h2 variant | SEQ ID NO: 51 | SEQ ID NO: 55 | Variant 2 contains an alternate 5′ |
| 2/isoform 2 | NM_001193425.1) | (NP_001180354.1) | terminal exon compared to variant 1. |
| | (hSUV39h2 variant 2) | (hSUV39h2 isoform 2) | This results in translation initiation |
| | | | from an in-frame, downstream AUG, |
| | | | and encodes a shorter isoform 2 as |
| | | | compared to isoform 1. (Variants 2 |
| | | | and 3 encode the same isoform 2) |
| hSUV39h2 variant | SEQ ID NO: 15 | SEQ ID NO: 6 | Variant 3 contains an alternate 5′ |
| 3/isoform 2 | (NM_024670.3) | (NP_078946.1) | terminal exon, and is missing the |
| | (hSUV39h2 variant 3) | (hSUV39h2 isoform 2) | subsequent exon compared to variant |
| | | | 1. This results in translation initiation |
| | | | from an in-frame, downstream AUG, |
| | | | and a shorter isoform 2 as compared |
| | | | to isoform 1. (Variants 2 and 3 encode |
| | | | the same isoform). |
| hSUV39h2 variant | SEQ ID NO: 52 | SEQ ID NO: 56 | Variant 4 uses an alternate donor |
| 4/isoform 3 | (NM_001193426.1) | (NP_001180355.1) | splice site at an internal coding exon |
| | (hSUV39h2 variant 4) | (hSUV39h2 isoform 3) | compared to variant 1, maintaining the |
| | | | reading frame, and resulting in a |
| | | | shorter isoform 3 that misses an |
| | | | internal protein segment compared to |
| | | | isoform 1. |
| hSUV39h2 variant | SEQ ID NO: 53 | SEQ ID NO: 57 | Variant 5 contains an alternate 5′ |
| 5/isoform 4 | (NM_001193427.1) | (NP_001180356.1) | terminal exon, and uses an alternate |
| | (hSUV39h2 variant 5) | (hSUV39h2 isoform 4) | donor splice site at an internal coding |
| | | | exon compared to variant 1. This |
| | | | results in translation initiation from an |
| | | | in-frame, downstream AUG, and a |
| | | | shorter isoform 4 missing an internal |
| | | | protein segment as compared to |
| | | | isoform 1. |

According to the invention, the inhibitor of human SUV39h1 is selected from the group consisting of inhibitors of H3K9-histone methyltransferase SUV39h1 protein function or inhibitors of H3K9-histone methyltransferase SUV39h1 gene expression.

The term “inhibitor of H3K9-histone methyltransferase SUV39h1” refers to any compound (natural or not), having the ability to inhibit the methylation of Lys-9 of histone H3 by H3K9-histone methyltransferase SUV39h1. The term “inhibitor of H3K9-histone methyltransferase SUV39h2” refers to any compound (natural or otherwise), having the ability to inhibit the methylation of Lys-9 of histone H3 by H3K9-histone methyltransferase SUV39h2.

The inhibiting activity of a compound may be determined using various methods as described in Greiner D. Et al. Nat Chem Biol. 2005 August; 1(3):143-5 or Eskeland, R. et al. Biochemistry 43, 3740-3749 (2004), which is incorporated herein in its entirety by reference.

In some embodiments, inhibition of a H3K9 methyltransferase is by an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof.

In some embodiments, an inhibitor of H3K9 methyltransferase is selected from the group consisting of, a RNAi agent, an siRNA agent, shRNA, oligonucleotide, CRISPR/Cas9, CRISPR/Cpfl neutralizing antibody or antibody fragment, aptamer, small molecule, protein, peptide, small molecule, avidimir, avimir, and functional fragments or derivatives thereof etc. Commercially available sequences to knockout SUV39h1 and/or SUV39h2 via a CRISPR/Cas9 s or CRISPR/Cpfl system are available from Origene (product numbers KN202428 and KN317005) and Santa Cruz Biotechnology (product number: sc-401717) and are encompassed for use in the methods and compositions as disclosed herein.

Agents useful in the methods as disclosed herein can also inhibit gene expression (i.e. suppress and/or repress the expression of the gene). Such agents are referred to in the art as “gene silencers” and are commonly known to those of ordinary skill in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNAi, siRNA, micro RNAi (miRNA), antisense oligonucleotides, etc.

In some embodiments of all aspects of the present invention, an agent which contacts a donor human somatic cell, a recipient human oocyte, a hybrid oocyte (e.g., human enucleated oocyte comprising donor genetic material prior to fusion or activation) or a human SCNT embryo (i.e., after fusion of the donor nuclei with the enucleated oocyte) is an inhibitor of a H3K9 methyltransferase, for example, but not limited to, an inhibitor of any one of human SUV39h1, human SUV39h2 or human SETDB1. In some embodiments, at least one or any combination of inhibitors of human SUV39h1, human SUV39h2 or human SETDB1 can be used in the methods to increase the efficiency of human SCNT. In some embodiments, an inhibitor of SUV39h1, SUV39h2 or SETDB1 inhibits the expression of human SUV39h1, human SUV39h2 or human SETDB1 nucleic acid sequences (e.g., SEQ ID NO: 14-16, or SEQ ID NO: 47 or SEQ ID NO: 49, 51-53), or the activity of human SUV39h1 protein (SEQ ID NO: 5 or SEQ ID NO: 48), human SUV39h2 (SEQ ID NO:6 or SEQ ID NO: 54-57) or human SETDB1 proteins (SEQ ID NO: 17).

In the context of the present invention, inhibitors of H3K9-histone methyltransferase SUV39h1/2 are preferably selective for H3K9-histone methyltransferase SUV39h1/2 as compared to other molecules. By “selective” it is meant that the affinity of the inhibitor is at least 10-fold, preferably 25-fold, more preferably 100-fold, still preferably 500-fold higher than the affinity for other histone methyltransferases.

Typically, the inhibitor of H3K9-histone methyltransferase SUV39h1 and/or SUV39h2 is a small organic molecule. The term “small organic molecule” refers to a molecule of a size comparable to those organic molecules generally used in pharmaceuticals. The term excludes biological macromolecules (e. g., proteins, nucleic acids, etc.). Preferred small organic molecules range in size up to about 5000 Da, more preferably up to 2000 Da, and most preferably up to about 1000 Da.

In a particular embodiment, the inhibitor of H3K9-histone methyltransferase SUV39h1 is chaetocin (CAS 28097-03-2) as described by Greiner D, Bonaldi T, Eskeland R, Roemer E, Imhof A. Identification of a specific inhibitor of the histone methyltransferase SU(VAR)3-9. Nat Chem Biol. 2005 August; 1(3): 143-5. Epub 2005 Jul. 17; Weber, H. P., et al., The molecular structure and absolute configuration of chaetocin. Acta Cryst., B28, 2945-2951 (1972); Udagawa, S., et al., The production of chaetoglobosins, sterigmatocystin, O-methylsterigmatocystin, and chaetocin by Chaetomium spp. and related fungi. Can. J. microbiol., 25, 170-177 (1979); Gardiner, D. M., et al., The epipolythiodioxopiperazine (ETP) class of fungal toxins: distribution, mode of action, functions and biosynthesis. Microbiol., 151, 1021-1032 (2005). For example, chaetocin is commercially available from Sigma Aldrich.

In another embodiment, the inhibitor of H3K9-histone methyltransferase SUV39h1 is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Inhibitors of expression for use in the present invention may be based on anti-sense oligonucleotide constructs. Anti-sense oligonucleotides, including anti-sense RNA molecules and anti-sense DNA molecules, would act to directly block the translation of H3K9-histone methyltransferase SUV39h1 or HP1α mRNA by binding thereto and thus preventing protein translation or increasing mRNA degradation, thus decreasing the level of H3K9-histone methyltransferase SUV39h1 or HP1a, and thus activity, in a cell. For example, antisense oligonucleotides of at least about 15 bases and complementary to unique regions of the mRNA transcript sequence encoding H3K9-histone methyltransferase SUV39h1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Inhibitors of SUV39h1 are disclosed in US Patent Application 2015/0038496, which is incorporated herein in its entirety by reference. The small molecule, Veticillin is identified as a selective inhibitor for both human SUV39h1 and human SUV39h2 (i.e., inhibits SUV39h1/2), as disclosed in US application 2014/0161785, which is incorporated herein in its entirety by reference, and is encompassed for use in the methods, compositions and kits as disclosed herein.

Inhibitors of SUV39h2 and method of their identification are disclosed in US Patent Application US2014/0094387, which is incorporated herein in its entirety by reference.

RNAi Inhibitors of H3K9 Methyltransferases.

In some embodiments, the H3K9 methyltransferase inhibitor is a RNAi agent, e.g., siRNA or shRNA molecule. RNAi agents of human SUV39h1, human SUV39h2, human SETDB1, human EHMT1, and human PRDM2 are well known in the art. In some embodiments an inhibitor of a H3K9 methyltransferase is a RNAi agent. In some embodiments, a RNAi agent hybridizes to, in full or in part, a target sequence located within a region of nucleotides of any one of human SUV39h1 nucleic acid sequences (SEQ ID NO: 14 or SEQ ID NO: 47), human SUV39h2 protein (SEQ ID NOs: 15, 49, 51, 52, 53) or human SETDB1 protein (SEQ ID NO: 16) as disclosed herein.

In some embodiments, a RNAi agent inhibits the expression of any one of human SUV39h1 protein (SEQ ID NO: 5 or SEQ ID NO: 48), human SUV39h2 protein (SEQ ID NO: 6 or SEQ ID NOS: 54-57) or human SETDB1 protein (SEQ ID NO: 17) as disclosed herein

Inhibition of a H3K9 methyltransferase gene can be by gene silencing RNAi molecules according to methods commonly known by a skilled artisan. In some embodiments, the H3K9 methyltransferase inhibitor is a RNAi agent is any one or a combination of siRNA agents selected from Table 2.

For example, a gene silencing siRNA oligonucleotide duplexes target a region located within human SUV39h1 corresponding to NM_003173.3 (SEQ ID NO: 14) corresponding to variant 2, or NM_001282166.1 (SEQ ID NO: 47) corresponding to variant 1, can readily be used to knockdown human SUV39h1 expression. SUV39h1 mRNA can be successfully targeted using siRNAs; and other siRNA molecules may be readily prepared by those of skill in the art based on the known sequence of the target mRNA. To avoid doubt, the sequence of a human SUV39h1 is provided at, for example, GenBank Accession Nos. NM_003173.3 (SEQ ID NO: 14) (variant 2 encoding isoform 1) or NM_001282166.1 (SEQ ID NO: 47) (variant 1, encoding isoform 1). One of ordinary skill can select a RNAi agent to be used which inhibits the expression of mRNA which encodes human SUV39h1 protein (SEQ ID NO: 5 or SEQ ID NO: 48), or inhibits the expression of any other mammalian SUV39h1 protein.

To avoid doubt, the sequence of a human SUV39h1 cDNA is provided at, for example, GenBank Accession Nos.: NM_003173.3 (SEQ ID NO: 14) corresponding to variant 2, or NM_001282166.1 (SEQ ID NO: 47) corresponding to variant, and can be used to design a gene silencing RNAi modulator which inhibits human SUV39h1 mRNA expression for use as a H3K9 methyltransfer inhibitor in the methods and compositions as disclosed herein. In some embodiments, an inhibitor of human SUV39h1 is a siRNA agent, for example, a siRNA agent comprising at least one or both of GAAACGAGUCCGUAUUGAAtt (SEQ ID NO: 7) or UUCAAUACGGACUCGUUUCtt (SEQ ID NO: 8) and fragments or derivatives of at least 80% sequence identity thereof.

As used herein, the term “SUV39h1 protein” refers to the amino acid sequence of SEQ ID NO: 5 (isoform 2) or SEQ ID NO: 48 (isoform 1) as disclosed herein, and homologues thereof, including conservative substitutions, additions, deletions therein not adversely affecting the structure of function. In some embodiments, the SUV39h1 protein is encoded by the nucleic acid sequence for human SUV39h1 transcript (SEQ ID NO: 14) variant 2 (encoding Suv39h1 isoform 2 protein) is as follows:

| (SEQ ID NO: 14) | |
|---|---|
| 1 | cgctcttctc gcgaggccgg ctaggcccga atgtcgttag ccgtggggaa agatggcgga |
| 61 | aaatttaaaa ggctgcagcg tgtgttgcaa gtcttcttgg aatcagctgc aggacctgtg |
| 121 | ccgcctggcc aagctctcct gccctgccct cggtatctct aagaggaacc tctatgactt |
| 181 | tgaagtcgag tacctgtgcg attacaagaa gatccgcgaa caggaatatt acctggtgaa |
| 241 | atggcgtgga tatccagact cagagagcac ctgggagcca cggcagaatc tcaagtgtgt |
| 301 | gcgtatcctc aagcagttcc acaaggactt agaaagggag ctgctccggc ggcaccaccg |
| 361 | gtcaaagacc ccccggcacc tggacccaag cttggccaac tacctggtgc agaaggccaa |
| 421 | gcagaggcgg gcgctccgtc gctgggagca ggagctcaat gccaagcgca gccatctggg |
| 481 | acgcatcact gtagagaatg aggtggacct ggacggccct ccgcgggcct tcgtgtacat |
| 541 | caatgagtac cgtgttggtg agggcatcac cctcaaccag gtggctgtgg gctgcgagtg |
| 601 | ccaggactgt ctgtgggcac ccactggagg ctgctgcccg ggggcgtcac tgcacaagtt |
| 661 | tgcctacaat gaccagggcc aggtgcggct tcgagccggg ctgcccatct acgagtgcaa |
| 721 | ctcccgctgc cgctgcggct atgactgccc aaatcgtgtg gtacagaagg gtatccgata |
| 781 | tgacctctgc atcttccgca cggatgatgg gcgtggctgg ggcgtccgca ccctggagaa |
| 841 | gattcgcaag aacagcttcg tcatggagta cgtgggagag atcattacct cagaggaggc |
| 901 | agagcggcgg ggccagatct acgaccgtca gggcgccacc tacctctttg acctggacta |
| 961 | cgtggaggac gtgtacaccg tggatgccgc ctactatggc aacatctccc actttgtcaa |
| 1021 | ccacagttgt gaccccaacc tgcaggtgta caacgtcttc atagacaacc ttgacgagcg |
| 1081 | gctgccccgc atcgctttct ttgccacaag aaccatccgg gcaggcgagg agctcacctt |
| 1141 | tgattacaac atgcaagtgg accccgtgga catggagagc acccgcatgg actccaactt |
| 1201 | tggcctggct gggctccctg gctcccctaa gaagcgggtc cgtattgaat gcaagtgtgg |
| 1261 | gactgagtcc tgccgcaaat acctcttcta gcccttagaa gtctgaggcc agactgactg |
| 1321 | agggggcctg aagctacatg cacctccccc actgctgccc tcctgtcgag aatgactgcc |
| 1381 | agggcctcgc ctgcctccac ctgcccccac ctgctcctac ctgctctacg ttcagggctg |
| 1441 | tggccgtggt gaggaccgac tccaggagtc ccctttccct gtcccagccc catctgtggg |
| 1501 | ttgcacttac aaacccccac ccaccttcag aaatagtttt tcaacatcaa gactctctgt |
| 1561 | cgttgggatt catggcctat taaggaggtc caaggggtga gtcccaaccc agccccagaa |
| 1621 | tatatttgtt tttgcacctg cttctgcctg gagattgagg ggtctgctgc aggcctcctc |
| 1681 | cctgctgccc caaaggtatg gggaagcaac cccagagcag gcagacatca gaggccagag |
| 1741 | tgcctagccc gacatgaagc tggttcccca accacagaaa ctttgtacta gtgaaagaaa |
| 1801 | gggggtccct gggctacggg ctgaggctgg tttctgctcg tgcttacagt gctgggtagt |
| 1861 | gttggcccta agagctgtag ggtctcttct tcagggctgc atatctgaga agtggatgcc |
| 1921 | cacatgccac tggaagggaa gtgggtgtcc atgggccact gagcagtgag aggaaggcag |
| 1981 | tgcagagctg gccagccctg gaggtaggct gggaccaagc tctgccttca cagtgcagtg |
| 2041 | aaggtaccta gggctcttgg gagctctgcg gttgctaggg gccctgacct ggggtgtcat |
| 2101 | gaccgctgac accactcaga gctggaacca agatctagat agtccgtaga tagcacttag |
| 2161 | gacaagaatg tgcattgatg gggtggtgat gaggtgccag gcactgggta gagcacctgg |
| 2221 | tccacgtgga ttgtctcagg gaagccttga aaaccacgga ggtggatgcc aggaaagggc |
| 2281 | ccatgtggca gaaggcaaag tacaggccaa gaattggggg tgggggagat ggcttcccca |
| 2341 | ctatgggatg acgaggcgag agggaagccc ttgctgcctg ccattcccag accccagccc |
| 2401 | tttgtgctca ccctggttcc actggtctca aaagtcacct gcctacaaat gtacaaaagg |
| 2461 | cgaaggttct gatggctgcc ttgctccttg ctcccccacc ccctgtgagg acttctctag |
| 2521 | gaagtccttc ctgactacct gtgcccagag tgcccctaca tgagactgta tgccctgcta |
| 2641 | tcagatgcca gatctatgtg tctgtctgtg tgtccatccc gccggccccc cagactaacc |
| 2641 | tccaggcatg gactgaatct ggttctcctc ttgtacaccc ctcaacccta tgcagcctgg |
| 2701 | agtgggcatc aataaaatga actgtcgact gaacaaaaaa aaaaaaaaaa aa |

In some embodiments, the SUV39h1 protein is encoded by the nucleic acid sequence for human SUV39h1 transcript (SEQ ID NO: 47) variant 1 (encoding Suv39h1 isoform 1 protein) is as follows:

| (SEQ ID NO: 47) | |
|---|---|
| 1 | gatcaactat ccacgctgct cgaatcacag catgctggag ggcctggctg ggtgctctga |
| 61 | ctgactgatc acctgacaga cggtgcggtc agtcggatgc tgagaatgac tgacgatgtg |
| 121 | atgaggggcg gattgaacga gtcacaggcc agctggccag gagcaaaatc ggcatagctg |
| 181 | tctgactcga tggctgtacg tggttacgga ctgtctgccc tgatagaatc tcagcttcaa |
| 241 | cgcatcagag gagactgact tgaccaatgg tggggatgag tcgcctgaga aatgacagac |
| 301 | tggctgaccc actgacaggc tgcagcgtgt gttgcaagtc ttcttggaat cagctgcagg |
| 361 | acctgtgccg cctggccaag ctctcctgcc ctgccctcgg tatctctaag aggaacctct |
| 421 | atgactttga agtcgagtac ctgtgcgatt acaagaagat ccgcgaacag gaatattacc |
| 481 | tggtgaaatg gcgtggatat ccagactcag agagcacctg ggagccacgg cagaatctca |
| 541 | agtgtgtgcg tatcctcaag cagttccaca aggacttaga aagggagctg ctccggcggc |
| 601 | accaccggtc aaagaccccc cggcacctgg acccaagctt ggccaactac ctggtgcaga |
| 661 | aggccaagca gaggcgggcg ctccgtcgct gggagcagga gctcaatgcc aagcgcagcc |
| 721 | atctgggacg catcactgta gagaatgagg tggacctgga cggccctccg cgggccttcg |
| 781 | tgtacatcaa tgagtaccgt gttggtgagg gcatcaccct caaccaggtg gctgtgggct |
| 841 | gcgagtgcca ggactgtctg tgggcaccca ctggaggctg ctgcccgggg gcgtcactgc |
| 901 | acaagtttgc ctacaatgac cagggccagg tgcggcttcg agccgggctg cccatctacg |
| 961 | agtgcaactc ccgctgccgc tgcggctatg actgcccaaa tcgtgtggta cagaagggta |
| 1021 | tccgatatga cctctgcatc ttccgcacgg atgatgggcg tggctggggc gtccgcaccc |
| 1081 | tggagaagat tcgcaagaac agcttcgtca tggagtacgt gggagagatc attacctcag |
| 1141 | aggaggcaga gcggcggggc cagatctacg accgtcaggg cgccacctac ctctttgacc |
| 1201 | tggactacgt ggaggacgtg tacaccgtgg atgccgccta ctatggcaac atctcccact |
| 1261 | ttgtcaacca cagttgtgac cccaacctgc aggtgtacaa cgtcttcata gacaaccttg |
| 1321 | acgagcggct gccccgcatc gctttctttg ccacaagaac catccgggca ggcgaggagc |
| 1381 | tcacctttga ttacaacatg caagtggacc ccgtggacat ggagagcacc cgcatggact |
| 1441 | ccaactttgg cctggctggg ctccctggct cccctaagaa gcgggtccgt attgaatgca |
| 1501 | agtgtgggac tgagtcctgc cgcaaatacc tcttctagcc cttagaagtc tgaggccaga |
| 1561 | ctgactgagg gggcctgaag ctacatgcac ctcccccact gctgccctcc tgtcgagaat |
| 1621 | gactgccagg gcctcgcctg cctccacctg cccccacctg ctcctacctg ctctacgttc |
| 1681 | agggctgtgg ccgtggtgag gaccgactcc aggagtcccc tttccctgtc ccagccccat |
| 1741 | ctgtgggttg cacttacaaa cccccaccca ccttcagaaa tagtttttca acatcaagac |
| 1801 | tctctgtcgt tgggattcat ggcctattaa ggaggtccaa ggggtgagtc ccaacccagc |
| 1861 | cccagaatat atttgttttt gcacctgctt ctgcctggag attgaggggt ctgctgcagg |
| 1921 | cctcctccct gctgccccaa aggtatgggg aagcaacccc agagcaggca gacatcagag |
| 1981 | gccagagtgc ctagcccgac atgaagctgg ttccccaacc acagaaactt tgtactagtg |
| 2041 | aaagaaaggg ggtccctggg ctacgggctg aggctggttt ctgctcgtgc ttacagtgct |
| 2101 | gggtagtgtt ggccctaaga gctgtagggt ctcttcttca gggctgcata tctgagaagt |
| 2161 | ggatgcccac atgccactgg aagggaagtg ggtgtccatg ggccactgag cagtgagagg |
| 2221 | aaggcagtgc agagctggcc agccctggag gtaggctggg accaagctct gccttcacag |
| 2281 | tgcagtgaag gtacctaggg ctcttgggag ctctgcggtt gctaggggcc ctgacctggg |
| 2341 | gtgtcatgac cgctgacacc actcagagct ggaaccaaga tctagatagt ccgtagatag |
| 2401 | cacttaggac aagaatgtgc attgatgggg tggtgatgag gtgccaggca ctgggtagag |
| 2461 | cacctggtcc acgtggattg tctcagggaa gccttgaaaa ccacggaggt ggatgccagg |
| 2521 | aaagggccca tgtggcagaa ggcaaagtac aggccaagaa ttgggggtgg gggagatggc |
| 2581 | ttccccacta tgggatgacg aggcgagagg gaagcccttg ctgcctgcca ttcccagacc |
| 2641 | ccagcccttt gtgctcaccc tggttccact ggtctcaaaa gtcacctgcc tacaaatgta |
| 2701 | caaaaggcga aggttctgat ggctgccttg ctccttgctc ccccaccccc tgtgaggact |
| 2761 | tctctaggaa gtccttcctg actacctgtg cccagagtgc ccctacatga gactgtatgc |
| 2821 | cctgctatca gatgccagat ctatgtgtct gtctgtgtgt ccatcccgcc ggccccccag |
| 2881 | actaacctcc aggcatggac tgaatctggt tctcctcttg tacacccctc aaccctatgc |
| 2941 | agcctggagt gggcatcaat aaaatgaact gtcgactgaa caaaaaaaaa aaaaaaaaa |

In some embodiments, the agent comprises a nucleic acid inhibitor that inhibits or reduces the expression of human SUV39h1 mRNA (SEQ ID NO: 14 or SEQ ID NO: 47) by at least 50% (as compared to in the absence of the SUV39h1 inhibitor).

In some embodiments, the agent comprises a nucleic acid inhibitor that inhibits or decreases the level or function of the human SUV39h1 protein (SEQ ID NO: 5 (isoform 2) or SEQ ID NO: 48 (isoform 1). In some embodiments, the agent comprises a nucleic acid inhibitor that inhibits or decreases the level or function of a human SUV39h2 protein (i.e., any of SEQ ID NOS: 6, 54-57).

In some embodiments, a siRNA inhibitor of human SUV39h1 is SEQ ID NO: 8 or a fragment of at least 10 consecutive nucleotides thereof, or nucleic acid sequence with at least 80% sequence identity (or at least about 85%, or at least about 90%, or at least about 95%, or at least about 98%, or at least about 99% sequence identity) to SEQ ID NO: 8. In some embodiments, a siRNA or other nucleic acid inhibitor hybridizes, in full or in part, to a target sequence of SEQ ID NO: 7.

In some embodiments, a siRNA inhibitor of mouse SUV39h2 is SEQ ID NO: 19 or a fragment of at least 10 consecutive nucleotides thereof, or nucleic acid sequence with at least 80% sequence identity (or at least about 85%, or at least about 90%, or at least about 95%, or at least about 98%, or at least about 99%) to SEQ ID NO: 19. In some embodiments, a siRNA or other nucleic acid inhibitor hybridizes, in full or in part, to a target sequence of SEQ ID NO: 18.

In some embodiments, a siRNA inhibitor of human SUV39h1 is SEQ ID NO: 21 or a fragment of at least 10 consecutive nucleotides thereof, or nucleic acid sequence with at least 80% sequence identity (or at least about 85%, or at least about 90%, or at least about 95%, or at least about 98%, or at least about 99% sequence identity) to SEQ ID NO: 21. In some embodiments, a siRNA or other nucleic acid inhibitor hybridizes, in full or in part, to a target sequence of SEQ ID NO: 20.

In some embodiments, a siRNA or other nucleic acid inhibitor hybridizes in full or part, to a target sequence located within a region of nucleotides of any of SEQ ID NOS: 15, 49, 51, 52 and 53 of human SUV39h2 (hSUV39h2 variants 1-5).

Inhibition of a H3K9 methyltransferase gene can be by gene silencing RNAi molecules according to methods commonly known by a skilled artisan. Inhibition of human SUV39h1, human SUV39h2, human SETDB1, human EHMT1, and human PRDM2 are well known in the art. In some embodiments, the H3K9 methyltransferase inhibitor is a RNAi agent is any one or a combination of siRNA agents selected from Table 2.

In some embodiments, SUV39H1 can be targeted and inhibited by hsa-mir-98-5p (MIRT027407), hsa-mir-615-3p (MIRT040438), hsa-mir-331-3p (MIRT043442) or miR variants of at least 85% sequence identity thereto. Commercially available siRNA, RNAi and shRNA products that inhibit SUV39h1 and/or SUV39h2 in human cells are available from Origene, Qiagen and Santa Cruz Biotechnology, and can be used by one of ordinary skill in the art.

For example, a gene silencing siRNA oligonucleotide that binds to, and hybridize in part or full to a nucleic acid sequence located in any of human SUV39H2 variants 1-5 (SEQ ID NOS: 15, 49, 51, 52 and 53) can readily be used to knockdown SUV39h2 expression. SUV39h2 mRNA can be successfully targeted using siRNAs; and other siRNA molecules may be readily prepared by those of skill in the art based on the known sequence of the target mRNA. To avoid doubt, the sequences of human SUV39h2 variants are shown in Table 8. To avoid doubt, the sequences of human SUV39h2 variant cDNAs are provided at, for example, GenBank Accession Nos.: NM_024670.3 (SEQ ID NO: 15), NM_001193425.1 (SEQ ID NO: 51), NM_001193426.1 (SEQ ID NO: 52), NM_001193427.1 (SEQ ID NO: 53), and can be used to design a gene silencing RNAi modulator which inhibits human SUV39h2 mRNA expression for use as a H3K9 methyltransfer inhibitor in the methods and compositions as disclosed herein. In some embodiments, an inhibitor of SUV39h2 is a siRNA agent, for example, a siRNA can comprise at least one or both of the following sequences: GCUCACAUGUAAAUCGAUUtt (SEQ ID NO: 18) or AAUCGAUUUACAUGUGAGCtt (SEQ ID NO: 19) and a fragment or derivative of at least 80% sequence identity thereof. In some embodiments, an inhibitor of SUV39h2 is a siRNA agent that binds to at least the target sequence of GCUCACAUGUAAAUCGAUUtt (SEQ ID NO: 18). In some embodiments, an inhibitor of SUV39h2 is a siRNA agent comprises at least 5 consecutive nucleotides of part of AAUCGAUUUACAUGUGAGCtt (SEQ ID NO: 19) or fragments or derivatives of at least 80% sequence identity thereof.

As used herein, the term “SUV39H2 protein” refers to the amino acids of any of SEQ ID NO: 54 (isoform 1), SEQ ID NO: 6 or SEQ ID NO: 53 (isoform 2), SEQ ID NO: 56 (isoform 3) or SEQ ID NO: 57 (isoform 4) as disclosed herein, and homologues thereof, including conservative substitutions, additions, deletions therein not adversely affecting the structure of function. The Accession numbers for the hSUV39h2 variant nucleic acid sequence and their corresponding proteins are shown in Table 8. For example, the SUV39h2 isoform 2 protein is encoded by the nucleic acid sequence for human SUV39H2 variant 3 transcript (SEQ ID NO: 15), which is as follows:

| (SEQ ID NO: 15) | |
|---|---|
| 1 | cggggccgag gcgcgaggag gtgaggctgg agcgcggccc cctcgccttc cctgttccca |
| 61 | ggcaagctcc caaggcccgg gcggcggggc cgtcccgcgg gccagccaga tggcgacgtg |
| 121 | gcggttcccc gcccgccgcg accccaactc cgggacgcac gctgcggacg cctatcctcc |
| 181 | cccaggccgc tgacccgcct ccctgcccgg ccggctcccg ccgcggagga tatggaatat |
| 241 | tatcttgtaa aatggaaagg atggccagat tctacaaata cttgggaacc tttgcaaaat |
| 301 | ctgaagtgcc cgttactgct tcagcaattc tctaatgaca agcataatta tttatctcag |
| 361 | gtaaagaaag gcaaagcaat aactccaaaa gacaataaca aaactttgaa acctgccatt |
| 421 | gctgagtaca ttgtgaagaa ggctaaacaa aggatagctc tgcagagatg gcaagatgaa |
| 481 | ctcaacagaa gaaagaatca taaaggaatg atatttgttg aaaatactgt tgatttagag |
| 541 | ggcccacctt cagacttcta ttacattaac gaatacaaac cagctcctgg aatcagctta |
| 601 | gtcaatgaag ctacctttgg ttgttcatgc acagattgct tctttcaaaa atgttgtcct |
| 661 | gctgaagctg gagttctttt ggcttataat aaaaaccaac aaattaaaat cccacctggt |
| 721 | actcccatct atgaatgcaa ctcaaggtgt cagtgtggtc ctgattgtcc caataggatt |
| 781 | gtacaaaaag gcacacagta ttcgctttgc atctttcgaa ctagcaatgg acgtggctgg |
| 841 | ggtgtaaaga cccttgtgaa gattaaaaga atgagttttg tcatggaata tgttggagag |
| 901 | gtaatcacaa gtgaagaagc tgaaagacga ggacagttct atgacaacaa gggaatcacg |
| 961 | tatctctttg atctggacta tgagtctgat gaattcacag tggatgcggc tcgatacggc |
| 1021 | aatgtgtctc attttgtgaa tcacagctgt gacccaaatc ttcaggtgtt caatgttttc |
| 1081 | attgataacc tcgatactcg tcttccccga atagcattgt tttccacaag aaccataaat |
| 1141 | gctggagaag agctgacttt tgattatcaa atgaaaggtt ctggagatat atcttcagat |
| 1201 | tctattgacc acagcccagc caaaaagagg gtcagaacag tatgtaaatg tggagctgtg |
| 1261 | acttgcagag gttacctcaa ctgaactttt tcaggaaata gagctgatga ttataatatt |
| 1321 | tttttcctaa tgttaacatt tttaaaaata catatttggg actcttatta tcaaggttct |
| 1381 | acctatgtta atttacaatt catgtttcaa gacatttgcc aaatgtatta ccgatgcctc |
| 1441 | tgaaaagggg gtcactgggt ctcatagact gatatgaagt cgacatattt atagtgctta |
| 1501 | gagaccaaac taatggaagg cagactattt acagcttagt atatgtgtac ttaagtctat |
| 1561 | gtgaacagag aaatgcctcc cgtagtgttt gaaagcgtta agctgataat gtaattaaca |
| 1621 | actgctgaga gatcaaagat tcaacttgcc atacacctca aattcggaga aacagttaat |
| 1681 | ttgggcaaat ctacagttct gtttttgcta ctctattgtc attcctgttt aatactcact |
| 1741 | gtacttgtat ttgagacaaa taggtgatac tgaattttat actgttttct acttttccat |
| 1801 | taaaacattg gcacctcaat gataaagaaa tttaaggtat aaaattaaat gtaaaaatta |
| 1861 | atttcagctt catttcgtat ttcgaagcaa tctagactgt tgtgatgagt gtatgtctga |
| 1921 | acctgtaatt cttaaaagac ttcttaatct tctagaagaa aaatctccga agagctctct |
| 1981 | ctagaagtcc aaaatggcta gccattatgc ttctttgaaa ggacatgata atgggaccag |
| 2041 | gatggttttt tggagtacca agcaagggga atggagcact ttaagggcgc ctgttagtaa |
| 2101 | catgaattgg aaatctgtgt cgagtacctc tgatctaaac ggtaaaacaa gctgcctgga |
| 2161 | gagcagctgt acctaacaat actgtaatgt acattaacat tacagcctct caatttcagg |
| 2221 | caggtgtaac agttcctttc caccagattt aatattttta tacttcctgc aggttcttct |
| 2281 | taaaaagtaa tctatatttt tgaactgata cttgttttat acataaattt tttttagatg |
| 2341 | tgataaagct aaacttggcc aaagtgtgtg cctgaattat tagacctttt tattagtcaa |
| 2401 | cctacgaaga ctaaaataga atatattagt tttcaaggga gtgggaggct tccaacatag |
| 2461 | tattgaatct caggaaaaac tattctttca tgtctgattc tgagatttct aattgtgttg |
| 2521 | tgaaaatgat aaatgcagca aatctagctt tcagtattcc taatttttac ctaagctcat |
| 2581 | tgctccaggc tttgattacc taaaataagc ttggataaaa ttgaaccaac ttcaagaatg |
| 2641 | cagcacttct taatctttag ctctttcttg ggagaagcta gactttattc attatattgc |
| 2701 | tatgacaact tcactctttc ataatatata ggataaattg tttacatgat tggaccctca |
| 2761 | gattctgtta accaaaattg cagaatgggg ggccaggcct gtgtggtggc tcacacctgt |
| 2821 | gatcccagca ctttgggagg ctgaggtagg aggatcacgt gaggtcggga gttcaagacc |
| 2881 | agcctggcca tcatggtgaa accctgtctc tactgaaaat acaaaaatta gccgggcgtg |
| 2941 | gtggcacacg cctgtagtcc cagctactca ggaggctgag gcaggagaat cacttgaatt |
| 3001 | caggaggcgg aggttgcagt gagccaagat cataccactg cactgcagcc tgagtgacac |
| 3061 | agtaagactg tctccaaaaa aaaaaaaaaa aaa |

In some embodiments, an agent inhibits the mRNA expression of any of SEQ ID NOS: 15, 49, 51, 52 and 53 of human SUV39h2 (hSUV39h2 variants 1-5) as disclosed herein. In some embodiments, one of ordinary skill can select a RNAi agent to be used which inhibits the expression of mRNA which encodes the human SUV39h2 proteins of any one or more of SEQ ID NO: 6, 54-57.

Other exemplary siRNA sequences for inhibiting human SUV39H1 and SUV39H2 are disclosed in US application 2012/0034192 which is incorporated herein in its entirety by reference.

TABLE 2
exemplary siRNA sequences to inhibit
H3K9 methyltransfersases:

| | SEQ | | |
|---|---|---|---|
| | ID | | |
| Gene | NO: | name | siRNA sequence |
| Human | 7 | hSUV39h1 | GAAACGAGUCCGUAUUGAAtt |
| SUV39h1 | | siRNA | (sense) |
| | | (sense) | |
| Human | 8 | hSUV39h1 | UUCAAUACGGACUCGUUUCtt |
| SUV39h1 | | siRNA | (antisense) |
| | | (AS) | |
| Human | 18 | hSUV39h2 | GCUCACAUGUAAAUCGAUUtt |
| SUV39h2 | | siRNA | (sense) |
| | | (sense) | |
| Human | 19 | hSUV39h2 | AAUCGAUUUACAUGUGAGCtt |
| SUV39h2 | | siRNA | (antisense) |
| | | (AS) | |
| Human | 20 | hSUV39h1 | GGUGUACAACGUAUUCAUAtt |
| SUV39h1 | | siRNA | (sense) |
| | | (sense) | |
| Human | 21 | hSUV39h1 | UAUGAAUACGUUGUACACCtg |
| SUV39h1 | | siRNA | (antisense) |
| | | (AS) | |
| Human | 22 | hSUV39h1 | GGUCCUUUGUCUAUAUCAAtt |
| SUV39h1 | | siRNA | (sense) |
| | | (sense) | |
| Human | 23 | hSUV39h1 | UUGAUAUAGACAAAGGACCtt |
| SUV39h1 | | siRNA | (antisense) |
| | | (AS) | |
| Human | 24 | h5UV39h2 | GCUCACAUGUAAAUCGAUUtt |
| SUV39h2 | | siRNA | (sense) |
| | | (sense) | |
| Human | 25 | h5UV39h2 | AAUCGAUUUACAUGUGAGCtt |
| SUV39h2 | | siRNA | (antisense) |
| | | (AS) | |
| Human | 26 | h5UV39h2 | GUGUCGAUGUGGACCUGAAtt |
| SUV39h2 | | siRNA | (sense) |
| | | (sense) | |
| Human | 27 | hSUV39h2 | UUCAGGUCCACAUCGACACct |
| SUV39h2 | | siRNA | (antisense) |
| | | (AS) | |
| Human | 28 | hSETDB1 | GGACUACAGUAUCAUGACAtt |
| SETDB1 | | siRNA | (sense) |
| (ESET) | | (sense) | |
| Human | 29 | hSETDB1 | UGUCAUGAUACUGUAGUCCca |
| SETDB1 | | siRNA | (antisense) |
| (ESET) | | (AS) | |
| Human | 30 | hSETDB1 | GGACGAUGCAGGAGAUAGAtt |
| SETDB1 | | siRNA | (sense) |
| (ESET) | | (sense) | |
| Human | 31 | hSETDB1 | UCUAUCUCCUGCAUCGUCCga |
| SETDB1 | | siRNA | (antisense) |
| (ESET) | | (AS) | |
| Human | 32 | hSETDB1 | GGAUGGGUGUCGGGAUAAAtt |
| SETDB1 | | siRNA | (sense) |
| (ESET) | | (sense) | |
| Human | 33 | hSETDB1 | UUUAUCCCGACACCCAUCCtt |
| SETDB1 | | siRNA | (antisense) |
| (ESET) | | (AS) | |
| Human | 34 | hEHMT1 | GCACCUUUGUCUGCGAAUAtt |
| EHMT1 | | siRNA | (sense) |
| (GLP) | | (sense) | |
| Human | 35 | hEHMT1 | UAUUCGCAGACAAAGGUGCcc |
| EHMT1 | | siRNA | (antisense) |
| (GLP) | | (AS) | |
| Human | 36 | hEHMT1 | GAUCAAACCUGCUCGGAAAtt |
| EHMT1 | | siRNA | (sense) |
| (GLP) | | (sense) | |
| Human | 37 | hEHMT1 | UUUCCGAGCAGGUUUGAUCca |
| EHMT1 | | siRNA | (antisense) |
| (GLP) | | (AS) | |
| Human | 38 | hPRDM2 | GAAUUUGCCUUCUUAUGCAtt |
| PRDM2/ | | siRNA | (sense) |
| Riz1 | | (sense) | |
| Human | 39 | hPRDM2 | UGCAUAAGAAGGCAAAUUCtt |
| PRDM2/ | | siRNA | (antisense) |
| Riz1 | | (AS) | |
| Human | 40 | hPRDM2 | GAGGAAUUCUAGUCCCGUAtt |
| PRDM2/ | | siRNA | (sense) |
| Riz1 | | (sense) | |
| Human | 41 | hPRDM2 | UACGGGACUAGAAUUCCUCaa |
| PRDM2/ | | siRNA | (antisense) |
| Riz1 | | (AS) | |

To avoid doubt, the sequence of a human SETDB1 cDNA is provided at, for example, GenBank Accession Nos.: NM_001145415.1 (SEQ ID NO: 16) and can be used by one of ordinary skill in the art to design a gene silencing RNAi modulator which inhibits human SETDB1 mRNA expression for use as a H3K9 methyltransfer inhibitor in the methods and compositions as disclosed herein.

To avoid doubt, the sequence of a human EHMT1 cDNA is provided at, for example, GenBank Accession Nos.: NM_024757.4 (SEQ ID NO: 42) and can be used by one of ordinary skill in the art to design a gene silencing RNAi modulator which inhibits human EHMT1 mRNA expression for use as a H3K9 methyltransfer inhibitor in the methods and compositions as disclosed herein.

To avoid doubt, the sequence of a human PRDM2 cDNA is provided at, for example, GenBank Accession Nos.: NM_012231.4 (SEQ ID NO: 43) and can be used by one of ordinary skill in the art to design a gene silencing RNAi modulator which inhibits human PRDM2 mRNA expression for use as a H3K9 methyltransfer inhibitor in the methods and compositions as disclosed herein.

In some embodiments, an inhibitor of H3K9 methyltransferase is selected from the group consisting of, a RNAi agent, an siRNA agent, shRNA, oligonucleotide, CRISPR/Cas9, CRISPR/Cpfl neutralizing antibody or antibody fragment, aptamer, small molecule, protein, peptide, small molecule, avidimir, and functional fragments or derivatives thereof etc. In some embodiments, the H3K9 methyltransferase inhibitor is a RNAi agent, e.g., siRNA or shRNA molecule. In some embodiments, the agent comprises a nucleic acid inhibitor that reduces protein expression of human SUV39H1 protein (SEQ ID NO: 5 or SEQ ID NO: 48) or SUV29h1 mRNA (SEQ ID NO: 14 or SEQ ID NO: 47) or human SUV39H2 protein (SEQ ID NO: 6 or SEQ ID NOS: 54-57) or SUV39h2 mRNA (SEQ ID NO: 15 or SEQ ID NOS: 49, 51, 52, 53). In some embodiments, a siRNA inhibitor of human SUV39h1 is SEQ ID NO: 8 or a fragment of at least 10 consecutive nucleotides thereof, or nucleic acid sequence with at least 80% sequence identity (or at least about 85%, or at least about 90%, or at least about 95%, or at least about 98%, or at least about 99% sequence identity) to SEQ ID NO: 8. In some embodiments, a siRNA or other nucleic acid inhibitor hybridizes to in full or in part, a target sequence of SEQ ID NO: 7 of SUV39H1. In some embodiments, a siRNA inhibitor of human SUV39H2 comprises SEQ ID NO: 19 or a fragment of at least 10 consecutive nucleotides thereof, or nucleic acid sequence with at least 80% sequence identity (or at least about 85%, or at least about 90%, or at least about 95%, or at least about 98%, or at least about 99%) to SEQ ID NO: 19. In some embodiments, a siRNA or other nucleic acid inhibitor hybridizes in full or part, to a target sequence of SEQ ID NO: 18 or SEQ ID NO: 15 of SUV39h2.

In other embodiments of the above aspects, a H3K9 methyltransferase inhibitor inhibits any one of the following histone methyltransferases selected from the group consisting of: SUV39H1, SUV39H2, G9A (EHMT2), EHMT1, ESET (SETDB1), SETDB2, MLL, MLL2, MLL3, SETD2, NSD1, SMYD2, DOT1L, SETD8, SUV420H1, SUV420H2, EZH2, SETD7, PRDM2, PRMT1, PRMT2, PRMT3, PRMT4, PRMT5, PRMT6, PRMT7, PRMT8, PRMT9, PRMT10, PRMT11, CARM1.

In some embodiments, an agent that inhibits a H3K9 methyltransferase, e.g., inhibits human SUV39H1, human SUV39H2 or human SETDB1 is a nucleic acid. Nucleic acid inhibitors of H3K9 methyltransferases, e.g., SUV39H1, SUV39H2 OR SETDB1 include, for example, but not are limited to, RNA interference-inducing (RNAi) molecules, for example but are not limited to siRNA, dsRNA, stRNA, shRNA and modified versions thereof, where the RNA interference (RNAi) molecule silences the gene expression from any one of; human SUV39H1, human SUV39H2 and/or human SETDB1 genes.

Accordingly, in some embodiments, inhibitors of H3K9 methyltransferases, e.g., an inhibitor of human SUV39H1, human SUV39H2 or human SETDB1, can inhibit by any “gene silencing” methods commonly known by persons of ordinary skill in the art. In some embodiments, a nucleic acid inhibitor of H3K9 methyltransferases, e.g., e.g., an inhibitor of human SUV39H1, human SUV39H2 or human SETDB1, is an anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like. In alternative embodiments, the nucleic acid is DNA or RNA, and nucleic acid analogues, for example PNA, pcPNA and LNA. A nucleic acid can be single or double stranded, and can be selected from a group comprising nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

In some embodiments single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

RNA interference (RNAi) provides a powerful approach for inhibiting the expression of selected target polypeptides. RNAi uses small interfering RNA (siRNA) duplexes that target the messenger RNA encoding the target polypeptide for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent can be a cellular gene or genomic sequence, e.g. a H3K9 methyltransferase gene sequence of SUV39h1, SUV39h2 or SETDB1 gene sequence. A siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target sequence.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotides molecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting H3K9 methyltransferases, e.g., SUV39h1, SUV39h2 or SETDB1 gene can be readily designed and tested. Accordingly, siRNAs useful for the methods described herein include siRNA molecules of about 15 to about 40 or about 15 to about 28 nucleotides in length, which are homologous to the specific H3K9 methyltransferase gene, e.g., SUV39h1, SUV39h2 or SETDB1 gene. In some embodiments, a H3K9 methyltransferase targeting agent, e.g., SUV39h1, SUV39h2 or SETDB1 targeting siRNA molecules have a length of about 25 to about 29 nucleotides. In some embodiments, a H3K9 methyltransferase targeting siRNA, e.g., a SUV39h1, a SUV39h2 or a SETDB1 targeting siRNA molecules have a length of about 27, 28, 29, or 30 nucleotides. In some embodiments, a H3K9 methyltransferase targeting RNAi, e.g., SUV39h1, SUV39h2 or SETDB1 targeting siRNA molecules can also comprise a 3′ hydroxyl group. In some embodiments, a H3K9 methyltransferase targeting siRNA, e.g., a SUV39h1, a SUV39h2 or SETDB1 targeting siRNA molecules can be single-stranded or double stranded; such molecules can be blunt ended or comprise overhanging ends (e.g., 5′, 3′). In specific embodiments, the RNA molecule can be a double stranded and either blunt ended or comprises overhanging ends.

In one embodiment, at least one strand of the H3K9 methyltransferases, e.g., SUV39h1, SUV39h2 or SETDB1 targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment a human SUV39h1/2, SETDB1, EHMT1 or PRDM2 targeting RNA molecule is double stranded—one strand has a 3′ overhang and the other strand can be blunt-ended or have an overhang. In the embodiment in which a H3K9 methyltransferase, e.g., SUV39h1, SUV39h2 SETDB1, EHMT1 or PRDM2 RNAi agent is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

As disclosed herein, siRNAs to H3K9 methyltransferases SUV39h1, SUV39h2 and SETDB1 have been successfully used to increase the efficiency of mouse SCNT. In some embodiments, where gene silencing RNAi of H3K9 methyltransferases, e.g. RNAi agents to inhibit expression/gene silence human SUV39h1, human SUV39h2, human SETDB1, human EHMT1 or human PRDM2 are not commercially available, gene silencing RNAi agents targeting inhibition of human SUV39h1, human SUV39h2, human SETDB1, human EHMT1 or human PRDM2 or PRDM2 can be produced by one of ordinary skill in the art and according to the methods as disclosed herein. In some embodiments, the assessment of the expression and/or knock down of human SUV39h1, human SUV39h2, human SETDB1, human EHMT1 or human PRDM2 mRNA and/or protein can be determined using commercially available kits known by persons of ordinary skill in the art. Others can be readily prepared by those of skill in the art based on the known sequence of the target mRNA.

In some embodiments, an inhibitor of the H3K9 methyltransferases is a gene silencing RNAi agent which downregulates or decreases any one or more of human SUV39h1, human SUV39h2, human SETDB1, human EHMT1 or human PRDM2 mRNA levels and can be a 25-nt hairpin sequence. In some embodiments, a H3K9 methyltransferase inhibitor is a gene silencing RNAi, such as, for example, a shRNA sequence of any one or more of human SUV39h1, human SUV39h2, human SETDB1, human EHMT1 or human PRDM2.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

RNA interference molecules and nucleic acid inhibitors useful in the methods as disclosed herein can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.).

The terms “antimir” “microRNA inhibitor” or “miR inhibitor” are synonymous and refer to oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands.

In some embodiments, an agent is protein or polypeptide or RNAi agent which inhibits the expression of any one or a combination of human SUV39h1, human SUV39h2, human SETDB1, human EHMT1 or human PRDM2. In such embodiments cells can be modified (e.g., by homologous recombination) to provide increased expression of such an agent, for example by replacing, in whole or in part, the naturally occurring promoter with all or part of a heterologous promoter so that the cells express an inhibitor of human SUV39h1, human SUV39h2, human SETDB1, human EHMT1 or human PRDM2, for example a protein or RNAi agent (e.g. gene silencing-RNAi agent). Typically, a heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also can be engineered to express an endogenous gene comprising the inhibitor agent under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al. The agent can be prepared by culturing transformed host cells under culture conditions suitable to express the miRNA. The resulting expressed agent can then be purified from such culture (i.e., from culture medium or cell extracts) using known purification processes, such as gel filtration and ion exchange chromatography. The purification of the peptide or nucleic acid agent inhibitor of human SUV39h1, human SUV39h2, human SETDB1, human EHMT1 or human PRDM2 can also include an affinity column containing agents which will bind to the protein; one or more column steps over such affinity resins as concanavalin A-agarose, HEPARIN-TOYOPEARL™ or Cibacrom blue 3GA Sepharose; one or more steps involving hydrophobic interaction chromatography using such resins as phenyl ether, butyl ether, or propyl ether; immunoaffinity chromatography, or complementary cDNA affinity chromatography.

In one embodiment, a nucleic acid inhibitor of human SUV39h1, human SUV39h2, human SETDB1, human EHMT1 or human PRDM2, e.g. (gene silencing RNAi agent) can be obtained synthetically, for example, by chemically synthesizing a nucleic acid by any method of synthesis known to the skilled artisan. A synthesized nucleic acid inhibitor of a H3K9 methyltransferase such as human SUV39h1, human SUV39h2, human SETDB1, human EHMT1 or human PRDM2 can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability of a nucleic acid inhibitor is desired, nucleic acids having nucleic acid analogs and/or modified internucleoside linkages can be used. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), diinethylene-sulfoxide (—CH2-SO—CH2), dimethylene-sulfone (—CH2-SO2-CH2), 2′-O-alkyl, and 2′-deoxy-2‘-fluoro’ phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., U.S. Pat. No. 5,714,606 to Acevedo, et al, U.S. Pat. No. 5,378,825 to Cook, et al., U.S. Pat. Nos. 5,672,697 and 5,466, 786 to Buhr, et al., U.S. Pat. No. 5,777,092 to Cook, et al., U.S. Pat. No. 5,602,240 to De Mesmacker, et al., U.S. Pat. No. 5,610,289 to Cook, et al. and U.S. Pat. No. 5,858,988 to Wang, also describe nucleic acid analogs for enhanced nuclease stability and cellular uptake.

Synthetic siRNA molecules, including shRNA molecules, can also easily be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

In some embodiments, an inhibitor of a H3K9 methyltransferase is a gene silencing siRNA molecule which targets any one of human SUV39h1, human SUV39h2, human SETDB1, human EHMT1 or human PRDM2 genes and in specific embodiments, targets the coding mRNA sequence of human SUV39h1, human SUV39h2, human SETDB1, human EHMT1 or human PRDM2, beginning from about 25 to 50 nucleotides, from about 50 to 75 nucleotides, or from about 75 to 100 nucleotides downstream of the start codon. One method of designing a siRNA molecule of the present invention involves identifying the 29 nucleotide sequence motif AA(N29)TT (where N can be any nucleotide) (SEQ ID NO: 50), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis software such as OLIGOENGINE®, can also be used to select siRNA sequences against EST libraries to ensure that only one gene is targeted.

siRNAs useful for the methods described herein include siRNA molecules of about 15 to about 40 or about 15 to about 28 nucleotides in length, which are homologous to any one of the H3K9 methyltransferase such as human SUV39h1, human SUV39h2, human SETDB1, human EHMT1 or human PRDM2. Preferably, a targeting siRNA molecule to human SUV39h1, human SUV39h2, human SETDB1, human EHMT1 or human PRDM2 has a length of about 19 to about 25 nucleotides. More preferably, the targeting siRNA molecules have a length of about 19, 20, 21, or 22 nucleotides. The targeting siRNA molecules can also comprise a 3′ hydroxyl group. The targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of a H3K9 methyltransferase RNAi targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the targeting RNA molecule is double stranded—one strand has a 3′ overhang and the other strand can be blunt-ended or have an overhang. In the embodiment in which the targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

Oligonucleotide Modifications

Unmodified oligonucleotides can be less than optimal in some applications, e.g., unmodified oligonucleotides can be prone to degradation by e.g., cellular nucleases. Nucleases can hydrolyze nucleic acid phosphodiester bonds. However, chemical modifications to one or more of the subunits of oligonucleotide can confer improved properties, and, e.g., can render oligonucleotides more stable to nucleases.

Modified nucleic acids and nucleotide surrogates can include one or more of: (i) alteration, e.g., replacement, of one or both of the non-linking phosphate oxygens and/or of one or more of the linking phosphate oxygens in the phosphodiester backbone linkage. (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone; (vi) modification of the 3′ end or 5′ end of the oligonucleotide, e.g., removal, modification or replacement of a terminal phosphate group or conjugation of a moiety, e.g., a fluorescently labeled moiety, to either the 3′ or 5′ end of oligonucleotide; and (vii) modification of the sugar (e.g., six membered rings).

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule.

As oligonucleotides are polymers of subunits or monomers, many of the modifications described herein can occur at a position which is repeated within an oligonucleotide, e.g., a modification of a nucleobase, a sugar, a phosphate moiety, or the non-bridging oxygen of a phosphate moiety. It is not necessary for all positions in a given oligonucleotide to be uniformly modified, and in fact more than one of the aforementioned modifications can be incorporated in a single oligonucleotide or even at a single nucleoside within an oligonucleotide.

In some cases the modification will occur at all of the subject positions in the oligonucleotide but in many, and in fact in most cases it will not. By way of example, a modification can only occur at a 3′ or 5′ terminal position, can only occur in the internal region, can only occur in a terminal regions, e.g. at a position on a terminal nucleotide or in the last 2, 3, 4, 5, or 10 nucleotides of an oligonucleotide. A modification can occur in a double strand region, a single strand region, or in both. A modification can occur only in the double strand region of an oligonucleotide or can only occur in a single strand region of an oligonucleotide. E.g., a phosphorothioate modification at a non-bridging oxygen position can only occur at one or both termini, can only occur in a terminal regions, e.g., at a position on a terminal nucleotide or in the last 2, 3, 4, 5, or 10 nucleotides of a strand, or can occur in double strand and single strand regions, particularly at termini. The 5′ end or ends can be phosphorylated.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

In some embodiments it is particularly preferred, e.g., to enhance stability, to include particular nucleobases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Specific Modifications to Oligonucleotide

The Phosphate Group

The phosphate group is a negatively charged species. The charge is distributed equally over the two non-bridging oxygen atoms. However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In certain embodiments, one of the non-bridging phosphate oxygen atoms in the phosphate backbone moiety can be replaced by any of the following: S, Se, BR3 (R is hydrogen, alkyl, aryl), C (i.e. an alkyl group, an aryl group, etc. . . . ), H, NR2 (R is hydrogen, alkyl, aryl), or OR (R is alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. When the bridging oxygen is the 3′-oxygen of a nucleoside, replacement with carbon is preferred. When the bridging oxygen is the 5′-oxygen of a nucleoside, replacement with nitrogen is preferred.

Replacement of the Phosphate Group

The phosphate group can be replaced by non-phosphorus containing connectors. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Modified phosphate linkages where at least one of the oxygens linked to the phosphate has been replaced or the phosphate group has been replaced by a non-phosphorous group, are also referred to as “non-phosphodiester backbone linkage.”

Replacement of Ribophosphate Backbone

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine and peptide nucleic acid (PNA) nucleoside surrogates. A preferred surrogate is a PNA surrogate.

Sugar Modifications

An oligonucleotide can include modification of all or some of the sugar groups of the nucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH2CH2O)nCH2CH2OR; “locked” nucleic acids (LNA) in which the 2′ hydroxyl is connected, e.g., by a methylene bridge, to the 4′ carbon of the same ribose sugar; O-AMINE (AMINE=NH2; alkylamino, dialkylamino, heterocyclyl, arylamino, diaryl amino, heteroaryl amino, or diheteroaryl amino, ethylene diamine, polyamino) and aminoalkoxy, O(CH2)nAMINE, (e.g., AMINE=NH2; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH2CH2OCH3, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH2; alkylamino, dialkylamino, heterocyclyl, arylamino, diaryl amino, heteroaryl amino, diheteroaryl amino, or amino acid); NH(CH2CH2NH)nCH2CH2-AMINE (AMINE=NH2; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; thioalkyl; and alkyl, cycloalkyl, aryl, alkenyl and alkynyl, which can be optionally substituted with e.g., an amino functionality.

The sugar group can also contain one or more carbons that possess the opposite stereochemical configuration than that of the corresponding carbon in ribose. Thus, an oligonucleotide can include nucleotides containing e.g., arabinose, as the sugar. The monomer can have an alpha linkage at the 1′ position on the sugar, e.g., alpha-nucleosides. Oligonucleotides can also include “abasic” sugars, which lack a nucleobase at C-1′. These abasic sugars can also be further containing modifications at one or more of the constituent sugar atoms. Oligonucleotides can also contain one or more sugars that are in the L form, e.g. L-nucleosides.

Preferred substitutents are 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH2-(4′-C) (LNA), 2′-O—CH2CH2-(4′-C) (ENA), 2′-O-aminopropyl (2′-O-AP), 2′-O-dimethylaminoethyl (2′-O-DMAOE), 2′-O-dimethylaminopropyl (2′-O-DMAP) and 2′-O-dimethylaminoethyloxyethyl (2′-O-DMAEOE).

Terminal Modifications

The 3-prime (3′) and 5-prime (5′) ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a dsRNA, this array can substitute for a hairpin RNA loop in a hairpin-type RNA agent.

Terminal modifications useful for modulating activity include modification of the 5′ end with phosphate or phosphate analogs. E.g., in preferred embodiments antisense strands of dsRNAs, are 5′ phosphorylated or include a phosphoryl analog at the 5′ prime terminus. 5′-phosphate modifications include those which are compatible with RISC mediated gene silencing. Modifications at the 5′-terminal end can also be useful in stimulating or inhibiting the immune system of a subject. Suitable modifications include: 5′-monophosphate ((HO)2(O)P—O-5′); 5′-diphosphate ((HO)2(O)P—O—P(HO)(O)-0-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-beta-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-). Other embodiments include replacement of oxygen/sulfur with BH3, BH3- and/or Se.

Terminal modifications can also be useful for monitoring distribution, and in such cases the preferred groups to be added include fluorophores, e.g., fluorscein or an ALEXA® dye, e.g., ALEXA® 488. Terminal modifications can also be useful for enhancing uptake, useful modifications for this include cholesterol. Terminal modifications can also be useful for cross-linking an RNA agent to another moiety; modifications useful for this include mitomycin C.

Nucleobases

Adenine, guanine, cytosine and uracil are the most common bases found in RNA. These bases can be modified or replaced to provide RNA's having improved properties. For example, nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed. Examples include 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, 06-substituted purines, substituted 1,2,4-triazoles, or any O-alkylated or N-alkylated derivatives thereof;

Further purines and pyrimidines include those disclosed in U.S. Pat. No. 3,687,808, hereby incorporated by reference, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

Cationic Groups

Modifications to oligonucleotides can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH2; alkylamino, dialkylamino, heterocyclyl, arylamino, diaryl amino, heteroaryl amino, or diheteroaryl amino, ethylene diamine, polyamino); aminoalkoxy, e.g., O(CH2)nAMINE, (e.g., AMINE ═NH2; alkylamino, dialkylamino, heterocyclyl, arylamino, diaryl amino, heteroaryl amino, or diheteroaryl amino, ethylene diamine, polyamino); amino (e.g. NH2; alkylamino, dialkylamino, heterocyclyl, arylamino, diaryl amino, heteroaryl amino, diheteroaryl amino, or amino acid); or NH(CH2CH2NH)nCH2CH2-AMINE (AMINE=NH2; alkylamino, dialkylamino, heterocyclyl, arylamino, diaryl amino, heteroaryl amino, or diheteroaryl amino).

Placement within an Oligonucleotide

Some modifications can preferably be included on an oligonucleotide at a particular location, e.g., at an internal position of a strand, or on the 5′ or 3′ end of an oligonucleotide. A preferred location of a modification on an oligonucleotide, can confer preferred properties on the agent. For example, preferred locations of particular modifications can confer optimum gene silencing properties, or increased resistance to endonuclease or exonuclease activity.

One or more nucleotides of an oligonucleotide can have a 2′-5′ linkage. One or more nucleotides of an oligonucleotide can have inverted linkages, e.g. 3′-3′, 5′-5′, 2′-2′ or 2′-3′ linkages.

An oligonucleotide can comprise at least one 5′-pyrimidine-purine-3′ (5′-PyPu-3′) dinucleotide wherein the pyrimidine is modified with a modification chosen independently from a group consisting of 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH2-(4′-C) (LNA) and 2′-O—CH2CH2-(4′-C) (ENA).

In one embodiment, the 5′-most pyrimidines in all occurrences of sequence motif 5′-pyrimidine-purine-3′ (5′-PyPu-3′) dinucleotide in the oligonucleotide are modified with a modification chosen from a group consisting of 2″-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH2-(4′-C) (LNA) and 2′-O—CH2CH2-(4′-C) (ENA).

A double-stranded oligonucleotide can include at least one 5′-uridine-adenine-3′ (5′-UA-3′) dinucleotide wherein the uridine is a 2′-modified nucleotide, or a 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide, wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. Double-stranded oligonucleotides including these modifications are particularly stabilized against endonuclease activity.

### General References

The oligoribonucleotides and oligoribonucleosides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3, 2′-O-Methyloligoribonucleotide-s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

### Phosphate Group References

The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

### Sugar Group References

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

### Replacement of the Phosphate Group References

Methylenemethylimino linked oligoribonucleosides, also identified herein as MMI linked oligoribonucleosides, methylenedimethylhydrazo linked oligoribonucleosides, also identified herein as MDH linked oligoribonucleosides, and methylenecarbonylamino linked oligonucleosides, also identified herein as amide-3 linked oligoribonucleosides, and methyleneaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 1 1972, 1991. Carbamate replacements are described in Stirchak, E. P. Nucleic Acids Res. 1989, 17, 6129.

### Replacement of the Phosphate-Ribose Backbone References

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They can also be prepared in accordance with U.S. Pat. No. 5,539,083 which is incorporated herein in its entirety by reference.

### Terminal Modification References

Terminal modifications are described in Manoharan, M. et al. Antisense and Nucleic Acid Drug Development 12, 103-128 (2002) and references therein.

### Nuclebases References

N-2 substituted purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references are disclosed in the above section on base modifications

Oligonucleotide Production

The oligonucleotide compounds of the invention can be prepared using solution-phase or solid-phase organic synthesis. Organic synthesis offers the advantage that the oligonucleotide strands comprising non-natural or modified nucleotides can be easily prepared. Any other means for such synthesis known in the art can additionally or alternatively be employed. It is also known to use similar techniques to prepare other oligonucleotides, such as the phosphorothioates, phosphorodithioates and alkylated derivatives. The double-stranded oligonucleotide compounds of the invention can be prepared using a two-step procedure. First, the individual strands of the double-stranded molecule are prepared separately. Then, the component strands are annealed.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

Teachings regarding the synthesis of particular modified oligonucleotides can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

Delivery of RNA Interfering Agents:

Methods of delivering RNAi agents, e.g., an siRNA, or vectors containing an RNAi agent, to the target cells (e.g., basal cells or cells of the lung ad/or respiratory system or other desired target cells) are well known to persons of ordinary skill in the art. In some embodiments, a RNAi agent (e.g. gene silencing—RNAi agent) which is an inhibitor of H3K9 methyltransferase, such as an RNAi agent which inhibits any one of human SUV39h1, human SUV39h2, human SETDB1, human EHMT1 and/or human PRDM2 can be administered to a subject via aerosol means, for example using a nebulizer and the like. In alternative embodiments, administration of a RNAi agent (e.g. gene silencing—RNAi agent) which is aH3K9 methyltransferase inhibitor, e.g., an inhibitor of any one of SUV39h1, SUV39h2 SETDB1, EHMT1 and/or PRDM2 can include, for example (i) injection of a composition containing the RNA interfering agent, e.g., an siRNA, or (ii) directly contacting the cell, (e.g., the donor human cell, the recipient oocyte, or SCNT embryo) with a composition comprising an RNAi agent, e.g., an siRNA.

In some embodiments, administration the cell, oocyte or embryo can be by a single injection or by two or more injections. In some embodiments, a RNAi agent is delivered in a pharmaceutically acceptable carrier. One or more RNAi agents can be used simultaneously, e.g. one or more gene silencing RNAi agent inhibitors of a H3K9 methyltransferase such as SUV39h1, SUV39h2 SETDB1, EHMT1 and/or PRDM2 can be administered together. The RNA interfering agents, e.g., siRNA to inhibit any one of human SUV39h1, human SUV39h2, human SETDB1, human EHMT1 and/or human PRDM2, can be delivered singly, or in combination with other RNA interfering agents, e.g., siRNAs, such as, for example siRNAs directed to other cellular genes.

In some embodiments, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNAi effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen which is identified by the antibody.

In some embodiments, a siRNA or RNAi binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

In some embodiments, a viral-mediated delivery mechanism can also be employed to deliver siRNAs, e.g. siRNAs (e.g. gene silencing RNAi agents) inhibitors of human SUV39h1, human SUV39h2, human SETDB1, human EHMT1 and/or human PRDM2 to cells in vitro as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Alternatively, in other embodiments, a RNAi agent, e.g., a gene silencing—RNAi agent inhibitor of a H3K9 methyltransferase such as SUV39h1, SUV39h2 SETDB1, EHMT1 and/or PRDM2 can also be introduced into cells via the culturing the cells, oocyte or SCNT embryo with the RNAi agent inhibitor alone or a viral vector expressing the RNAi agent.

In general, any method of delivering a nucleic acid molecule can be adapted for use with an RNAi interference molecule (see e.g., Akhtar S. and Julian R L. (1992) Trends Cell. Biol. 2(5):139-144; WO94/02595, which are incorporated herein by reference in their entirety).

RNA interference molecules can be modified by chemical conjugation to lipophilic groups such as cholesterol to enhance cellular uptake and prevent degradation. In an alternative embodiment, the RNAi molecules can be delivered using drug delivery systems such as e.g., a nanoparticle, a dendrimer, a polymer, liposomes, or a cationic delivery system. Positively charged cationic delivery systems facilitate binding of an RNA interference molecule (negatively charged) and also enhance interactions at the negatively charged cell membrane to permit efficient uptake of an siRNA by the cell. Cationic lipids, dendrimers, or polymers can either be bound to an RNA interference molecule, or induced to form a vesicle or micelle (see e.g., Kim S H., et al (2008) Journal of Controlled Release 129(2): 107-116) that encases an RNAi molecule. The formation of vesicles or micelles further prevents degradation of the RNAi molecule when administered systemically. Methods for making and administering cationic-RNAi complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety).

The dose of the particular RNAi agent will be in an amount necessary to effect RNA interference, e.g., gene silencing of human SUV39h1, human SUV39h2, human SETDB1, human EHMT1 and/or human PRDM2, thereby leading to decrease in the gene expression level of human SUV39h1, human SUV39h2, human SETDB1, human EHMT1 and/or human PRDM2 and subsequent decrease in the respective protein expression level.

It is also known that RNAi molecules do not have to match perfectly to their target sequence. Preferably, however, the 5′ and middle part of the antisense (guide) strand of the siRNA is perfectly complementary to the target nucleic acid sequence of any one of human SUV39h1, human SUV39h2, human SETDB1, human EHMT1 and/or human PRDM2 genes.

Accordingly, the RNAi molecules functioning as gene silencing-RNAi agents inhibitors of human SUV39h1, human SUV39h2, human SETDB1, human EHMT1 and/or human PRDM2 as disclosed herein are for example, but are not limited to, unmodified and modified double stranded (ds) RNA molecules including short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also can contain 3′ overhangs, preferably 3′UU or 3′TT overhangs. In one embodiment, the siRNA molecules of the present invention do not include RNA molecules that comprise ssRNA greater than about 30-40 bases, about 40-50 bases, about 50 bases or more. In one embodiment, the siRNA molecules of the present invention are double stranded for more than about 25%, more than about 50%, more than about 60%, more than about 70%, more than about 80%, more than about 90% of their length.

In some embodiments, a gene silencing RNAi nucleic acid inhibitors of human SUV39h1, human SUV39h2, human SETDB1, human EHMT1 and/or human PRDM2 is any agent which binds to and inhibits the expression of human SUV39h1, human SUV39h2, human SETDB1, human EHMT1 and/or human PRDM2, where the expression of the respective methyltransferase gene is inhibited.

In another embodiment of the invention, an inhibitor of human SUV39h1, human SUV39h2, human SETDB1, human EHMT1 and/or human PRDM2 can be a catalytic nucleic acid construct, such as, for example ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding, the ribozyme cleaves the target in a site specific manner. The design and testing of ribozymes which specifically recognize and cleave sequences of the gene products described herein, for example for cleavage of a H3K9 methyltransferase such as human SUV39h1, human SUV39h2, human SETDB1, human EHMT1 and/or human PRDM2 by techniques well known to those skilled in the art (for example Lleber and Strauss, (1995) Mol Cell Biol 15:540.551, the disclosure of which is incorporated herein by reference).

Proteins and Peptide Inhibitors of H3K9 Methyltransferases

In some embodiments, a H3K9 methyltransferase inhibitor is a protein and/or peptide inhibitor of any one of H3K9 methyltransferases such as human SUV39h1, human SUV39h2, human SETDB1, human EHMT1 and/or human PRDM2 for example, but are not limited to mutated proteins; therapeutic proteins and recombinant proteins human SUV39h1, human SUV39h2, human SETDB1, human EHMT1 and/or human PRDM2 as well as dominant negative inhibitors (e.g., non-functional proteins of the H3K9 methyltransferase, or non-functional ligands of H3K9 methyltransferase which bind to, and competitively H3K9 methyltransferase). Proteins and peptides inhibitors can also include for example mutated proteins, genetically modified proteins, peptides, synthetic peptides, recombinant proteins, chimeric proteins, antibodies, humanized proteins, humanized antibodies, chimeric antibodies, modified proteins and fragments thereof.

As used herein, agents useful in the method as inhibitors of H3K9 methyltransferases, e.g., human SUV39h1, human SUV39h2, human SETDB1, human EHMT1 and/or human PRDM2 gene expression and/or inhibition of human SUV39h1, human SUV39h2, human SETDB1, human EHMT1 and/or human PRDM2 proteins function can be any type of entity, for example but are not limited to chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety.

In alternative embodiments, agents useful in the methods as disclosed herein are proteins and/or peptides or fragment thereof, which inhibit the gene expression or function of H3K9 methyltransferases, e.g., human SUV39h1, human SUV39h2, human SETDB1, human EHMT1 and/or human PRDM2. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof.

Alternatively, agents useful in the methods as disclosed herein as inhibitors human SUV39h1, human SUV39h2, human SETDB1, human EHMT1 and/or human PRDM2 can be a chemicals, small molecule, large molecule or entity or moiety, including without limitation synthetic and naturally-occurring non-proteinaceous entities. In certain embodiments the agent is a small molecule having the chemical moieties as disclosed herein.

In some embodiments, a H3K9 methyltransferase inhibitor for use in the methods and compositions as disclosed herein is a dominant negative variants of a H3K9 methyltransferase, for example a non-functional variant of human SUV39h1, human SUV39h2, human SETDB1, human EHMT1 and/or human PRDM2 can be a truncated or dominant negative protein comprising a fragment of consecutive amino acids of any of the amino acids of SEQ ID NOS: 5, 6, 48 and 54-57, such as, e.g., a fragment of at least about 50, or at least about 60, or at least about 70, or at least about 80 or at least about 90 or more than 90 amino acids of SEQ ID NOS: 5, 6, 48 and 54-57. In some embodiments, a dominant negative inhibitor of a H3K9 methyltransferase protein, such as human SUV39h1, human SUV39h2, human SETDB1, human EHMT1 and/or human PRDM2 protein is a soluble extracellular domain of the H3K9 methyltransferase protein.

Protein inhibitors, such as the gene product or protein of the DBC1 (Deleted Breast Cancer 1) gene binds to the SUV39H1 catalytic domain and inhibits its ability to methylate histone H3 in vitro and in vivo (Lu et al., Inhibition of SUV39H1 Methyltransferase Activity by DBC1, JBC, 2009, 284; 10361-10366), and is encompassed for use in the methods and compositions as disclosed herein.

Antibodies

In some embodiments, a H3K9 methyltransferase inhibitor useful in the methods of the present invention include, for example, antibodies, including monoclonal, chimeric humanized, and recombinant antibodies and antigen-binding fragments thereof. In some embodiments, neutralizing antibodies can be used as a H3K9 methyltransferase inhibitor. Antibodies are readily raised in animals such as rabbits or mice by immunization with the antigen. Immunized mice are particularly useful for providing sources of B cells for the manufacture of hybridomas, which in turn are cultured to produce large quantities of monoclonal antibodies. Commercially available antibody inhibitors of human SUV39h1 and/or SUV39h2 are encompassed for use in the present invention, for example, are available from Santa Cruz biotechnology and the like.

In one embodiment of this invention, the inhibitor to the gene products identified herein can be an antibody molecule or the epitope-binding moiety of an antibody molecule and the like. Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis.

Useful monoclonal antibodies and fragments can be derived from any species (including humans) or can be formed as chimeric proteins which employ sequences from more than one species. Human monoclonal antibodies or “humanized” murine antibody are also used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction the possibly of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

One limitation of scFv molecules is their monovalent interaction with target antigen. One of the easiest methods of improving the binding of a scFv to its target antigen is to increase its functional affinity through the creation of a multimer. Association of identical scFv molecules to form diabodies, triabodies and tetrabodies can comprise a number of identical Fv modules. These reagents are therefore multivalent, but monospecific. The association of two different scFv molecules, each comprising a VH and VL domain derived from different parent Ig will form a fully functional bispecific diabody. A unique application of bispecific scFvs is to bind two sites simultaneously on the same target molecule via two (adjacent) surface epitopes. These reagents gain a significant avidity advantage over a single scFv or Fab fragments. A number of multivalent scFv-based structures has been engineered, including for example, miniantibodies, dimeric miniantibodies, minibodies, (scFv)2, diabodies and triabodies. These molecules span a range of valence (two to four binding sites), size (50 to 120 kDa), flexibility and ease of production. Single chain Fv antibody fragments (scFvs) are predominantly monomeric when the VH and VL domains are joined by, polypeptide linkers of at least 12 residues. The monomer scFv is thermodynamically stable with linkers of 12 and 25 amino acids length under all conditions. The noncovalent diabody and triabody molecules are easy to engineer and are produced by shortening the peptide linker that connects the variable heavy and variable light chains of a single scFv molecule. The scFv dimers are joined by amphipathic helices that offer a high degree of flexibility and the miniantibody structure can be modified to create a dimeric bispecific (DiBi) miniantibody that contains two miniantibodies (four scFv molecules) connected via a double helix. Gene-fused or disulfide bonded scFv dimers provide an intermediate degree of flexibility and are generated by straightforward cloning techniques adding a C-terminal Gly4Cys (SEQ ID NO: 44) sequence. scFv-CH3 minibodies are comprised of two scFv molecules joined to an IgG CH3 domain either directly (LD minibody) or via a very flexible hinge region (Flex minibody). With a molecular weight of approximately 80 kDa, these divalent constructs are capable of significant binding to antigens. The Flex minibody exhibits impressive tumor localization in mice. Bi- and tri-specific multimers can be formed by association of different scFv molecules. Increase in functional affinity can be reached when Fab or single chain Fv antibody fragments (scFv) fragments are complexed into dimers, trimers or larger aggregates. The most important advantage of multivalent scFvs over monovalent scFv and Fab fragments is the gain in functional binding affinity (avidity) to target antigens. High avidity requires that scFv multimers are capable of binding simultaneously to separate target antigens. The gain in functional affinity for scFv diabodies compared to scFv monomers is significant and is seen primarily in reduced off-rates, which result from multiple binding to two or more target antigens and to rebinding when one Fv dissociates. When such scFv molecules associate into multimers, they can be designed with either high avidity to a single target antigen or with multiple specificities to different target antigens. Multiple binding to antigens is dependent on correct alignment and orientation in the Fv modules. For full avidity in multivalent scFvs target, the antigen binding sites must point towards the same direction. If multiple binding is not sterically possible then apparent gains in functional affinity are likely to be due the effect of increased rebinding, which is dependent on diffusion rates and antigen concentration. Antibodies conjugated with moieties that improve their properties are also contemplated for the instant invention. For example, antibody conjugates with PEG that increases their half-life in vivo can be used for the present invention. Immune libraries are prepared by subjecting the genes encoding variable antibody fragments from the B lymphocytes of naive or immunized animals or patients to PCR amplification. Combinations of oligonucleotides which are specific for immunoglobulin genes or for the immunoglobulin gene families are used. Immunoglobulin germ line genes can be used to prepare semisynthetic antibody repertoires, with the complementarity-determining region of the variable fragments being amplified by PCR using degenerate primers. These single-pot libraries have the advantage that antibody fragments against a large number of antigens can be isolated from one single library. The phage-display technique can be used to increase the affinity of antibody fragments, with new libraries being prepared from already existing antibody fragments by random, codon-based or site-directed mutagenesis, by shuffling the chains of individual domains with those of fragments from naive repertoires or by using bacterial mutator strains.

Alternatively, a SCID-hu mouse, for example the model developed by Genpharm, can be used to produce antibodies, or fragments thereof. In one embodiment, a new type of high avidity binding molecule, termed peptabody, created by harnessing the effect of multivalent interaction is contemplated. A short peptide ligand was fused via a semirigid hinge region with the coiled-coil assembly domain of the cartilage oligomeric matrix protein, resulting in a pentameric multivalent binding molecule. In preferred embodiment of this invention, ligands and/or chimeric inhibitors can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. Alternatively, two or more active agents and or inhibitors attached to targeting moieties can be administered, wherein each conjugate includes a targeting moiety, for example, a different antibody. Each antibody is reactive with a different target site epitope (associated with the same or a different target site antigen). The different antibodies with the agents attached accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

### Small Molecules

All of the applications set out in the above paragraphs are incorporated herein by reference. In some embodiments, one of ordinary skill in the art can use other agents as a H3K9 methyltransferase inhibitor, for example antibodies, decoy antibodies, or RNAi are effective in the methods, compounds and kits for increasing the efficiency of SCNT as disclosed herein.

In some embodiments, a H3K9 methyltransferase inhibitor useful in the methods, compositions and kits as disclosed herein is gliotoxin or a related epipolythiodioxopiperazines, or BIX-01294 (diazepin-quinazolin-amine derivative as disclosed in Takahashi et al., 2012, J. Antibiotics 65, 263-265 or Shaabam et al., Chemistry & Biology, Volume 14, Issue 3, March 2007, Pages 242-244, which are incorporated herein in their entirety by reference. BIX-01294 has the following chemical structure:

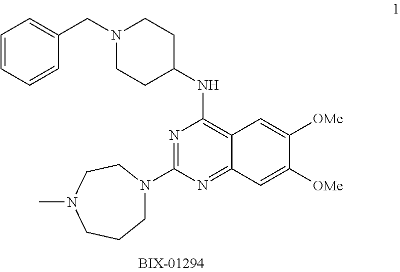 [SMILES: COc1cc2nc(N3CCCN(C)CC3)nc(NC3CCN(Cc4ccccc4)CC3)c2cc1OC]

Quinazoline, also known as UNC0638 also inhibits G9a, and is encompassed for use in the methods and compositions as disclosed herein. UNC0638 has the following structure:

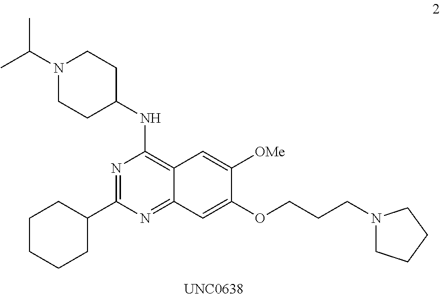 [SMILES: COc1cc2c(NC3CCN(C(C)C)CC3)nc(C3CCCCC3)nc2cc1OCCCN1CCCC1 ; "UNC0638"]

Small molecule inhibitors of SUV39h1 are disclosed in US Patent Application 2015/0038496, which is incorporated herein in its entirety by reference. The small molecule, verticillin A is identified as a selective inhibitor for both SUV39h1 and SUV39h2 (i.e., inhibits SUV39h1/2), as disclosed in US application 2014/0161785, which is incorporated herein in its entirety by reference, and is encompassed for use in the methods, compositions and kits as disclosed herein.

Other small molecule inhibitors of SUV39h1 include Chaetocin (chemical name: (3S,3'S,5aR,5aR,10bR, 10′bR,11aS,11′aS)-2,2′,3,3′,5a,5′a,6,6′-octahydro-3,3′-bis(hydroxymethyl)-2,2′-dimethyl-[10b, 10′b(11H,11′H)-bi3,11a-epidithio-11aH-pyrazino[1′,2′: 1,5]pyrrolo[2,3-b]indole]-1,1′,4,4′-tetrone) (see Bernhard et al., FEBS Letts, 2011, 585 (22); 3549-3554), which has the following chemical structure, and is encompassed for use in the methods and compositions as disclosed herein.

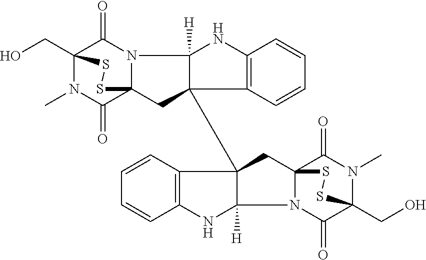

The compound A-366 (also referred to as CHEMBL3109630) (PubChem CID: 76285486), has also been found to be a potent inhibitor of EHMT2 (Euchromatic histone methyltransferase 2) also known as G9a, with a IC₅₀ of 3.3 nM, and having a greater than 1000-fold selectivity over 21 other methyltransferases (see: Sweis et al., Discovery and development of potent and selective inhibitors of histone methyltransferase G9a. ACS medical Chem Letts, 2014; 5(2); 205-209), and is encompassed for use in the methods and compositions as disclosed herein. The small molecule A-366 has the following structure;

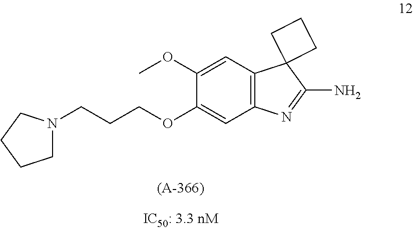

3-Deazaneplanocin A (DZNep) (CAS No: 102052-95-9) results in the decrease of SETDB1 H3K9me3 HMTase and results in the decrease in reduced levels of both H3K27me3 and H3K9me3 (Lee et al., Biochem Biophys Res Comm, 2013, 438(4); 647-652), and is encompassed for use in the methods and compositions as disclosed herein. DZNp has the formula as follows:

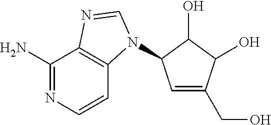 [SMILES: Nc1nccc2c1ncn2[C@@H]1C=C(CO)C(O)C1O]

The HMTase Inhibitor IV, UNC0638 (available from Calbiochem) minimally inhibits SUV39h2 (IC₅₀>10 μM) (see: Vedadi, M., et al. 2011. Nat. Chem. Biol. 7, 566; and Liu, F., et al. 2011. J. Med. Chem. 54, 6139), and is encompassed for use in the methods and compositions as disclosed herein. The HMTase Inhibitor IV is also known by synonyms: 2-Cyclohexyl-N-(1-isopropylpiperidin-4-yl)-6-methoxy-7-(3-(pyrrolidin-1-yl)propoxy)quinazolin-4-amine, DNA Methyltransferase Inhibitor III, DNA MTase Inhibitor III, EHMT1/GLP Inhibitor II, EHMT2/G9a Inhibitor IV and has a chemical formula as follows:

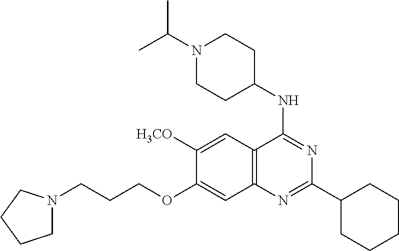 [SMILES: COc1cc2c(NC3CCN(C(C)C)CC3)nc(C3CCCCC3)nc2cc1OCCCN1CCCC1]

### SCNT

One of the objectives of the present invention is to provide a means of increasing the efficiency of human SCNT and production of human NT-ESCs from human SCNT embryos. The methods of the disclosure may be used for cloning a mammal, for obtaining totipotent or pluripotent cells, or for reprogramming a human cell.

Recipient Human Oocyte:

In certain embodiments, a recipient human oocyte for use in the methods, kits and compositions of the invention may be from a healthy human donor. In some embodiments, the cryopreserved oocytes are used as recipient oocyte cells. In certain embodiments, a recipient oocyte is human. Cryogenic preservation and thawing of oocytes are known to those skilled in the art (see Tucker et al., Curr Opin Obstet Gynecol. 1995 June; 7(3): 188-92). In some embodiments, the human recipient oocyte is obtained from a willing human female donor, for example an egg donor, e.g., an egg donor for an IVF clinic. In some embodiments, the oocyte is obtained from a female human subject who has undergone ovarian stimulation or overstimulation of the ovaries (i.e. ovulation induction or controlled ovarian hyperstimulation). Methods of controlled ovarian hyperstimulation are well known in the art, for example, as disclosed in U.S. Pat. No. 8,173,592, and international patent application WO2000/059542, and incorporated herein in their entirety by reference.

In some embodiments, a recipient human oocyte is an enucleated oocyte. Enucleation of the donor oocyte may be effected by known methods, such as described in U.S. Pat. No. 4,994,384 which is incorporated by reference herein. For example, metaphase II (MII) oocytes are either placed in HECM, optionally containing 7.5 micrograms per milliliter cytochalasin B, for immediate enucleation, or may be placed in a suitable medium, for example CR1aa, plus 10% estrus cow serum, and then enucleated later. Enucleation can also be accomplished microsurgically using a micropipette to remove the polar body and the adjacent cytoplasm. The cells may then be screened to identify those of which have been successfully enucleated. This screening may be effected by staining the cells with 1 microgram per milliliter 33342 Hoechst dye in HECM, and then viewing the cells under ultraviolet irradiation for less than 10 seconds. Cells that have been successfully enucleated can then be placed in a suitable culture medium.

In some embodiments, non-invasive approaches for oocyte enucleation can be used, for example, similar to a procedure for enucleation of oocytes from amphibians, where irradiation with ultraviolet light is used as a routine procedure (Gurdon Q. J. Microsc. Soc. 101 299-311 (1960)). In some embodiments, oocyte enucleation of human oocyte can be done using DNA-specific fluorochrome, with exposure of mouse oocytes to ultraviolet light for more than 30 seconds reduced the developmental potential of the cell (Tsunoda et al., J. Reprod. Fertil. 82 173 (1988)).

In some embodiments, an enucleated human oocyte has undergone “induced enucleation” which refers to enucleation of the oocyte by disrupting the meiotic spindle apparatus through the destabilization (e.g., depolymerization) of the microtubules of the meiotic spindle (see U.S. Patent Application No. 2006/0015950, which is incorporate herein in its entirety by reference). Destabilization of the microtubules prevents the chromatids from separating (e.g., prevents successful karyokinesis), and induces the oocyte genome (e.g., nuclear chromatin) to segregate unequally (e.g., skew) during meiotic maturation, whereby essentially all endogenous chromatin of the oocyte collects in the second polar body.

In some embodiments, oocyte donations are from a healthy woman, e.g., a healthy human female oocyte donor. In some embodiments, the human oocytes for use in the methods, compositions and kits as disclosed herein are excess oocytes obtained from fertility clinics, which are no longer needed in IVF procedures. In some embodiments, a human oocyte for use in the methods, compositions and kits as disclosed herein is of poor, or sub-optimal quality, in that, due to their poor quality, they are unlikely to be successfully fertilized by a sperm in vitro (e.g., a human oocyte can be of a poor quality that will likely fail in an IVF procedure). In some embodiments, a human oocyte selected for use in the methods, compositions and kits as disclosed herein is selected based on its quality, and in some embodiments, low quality oocytes that are predicted to be unlikely to be successfully fertilized by a sperm in vitro (e.g., in an IVF procedure) are selected. In some embodiments, high to medium quality oocytes are selected that are likely to be successfully fertilized by a sperm in vitro (e.g., in an IVF procedure). In some embodiments, the human oocytes are donated from post-menopause human females, which are predicted to be unlikely to be successfully fertilized in vitro are selected and encompassed for use in the methods, compositions and kits as disclosed herein.

In some embodiments, to bypass the need for human oocyte donors, cross-species SCNT has been explored where non-human oocytes have been reported for nuclear reprogramming of human donor somatic cell (Chung et al., Cloning and Stem Cells 11, 1-11 (2009)). Accordingly, in some embodiments, the donor oocyte is from a non-human primate, or a bovine oocyte, or any other non-human mammalian species, which can be a recipient oocyte for the nuclei or nuclear genetic material obtained from a human donor somatic cell.

In some embodiments, when humans are stimulated to produce oocytes (such as hormonally) and these oocytes are harvested, the oocytes that are collected can be in different phases. Some human oocytes are in metaphase I (MI) while other oocytes are in metaphase II (MII). In such cases, the human oocytes that are in metaphase I (MI) can be cultured until they reach metaphase II and then used for enucleation to serve as the recipient oocyte cell. Optionally, human oocytes that have been cultured to reach metaphase II are combined with the oocytes that were already at metaphase II when harvested for a pool of potential host cells. In other cases, only the human oocytes that are in metaphase II from the harvest are used for enucleation. Any of these human oocytes can be frozen for further use. Thus, the donor and/or the recipient oocyte can be cryopreserved prior to use.

Accordingly, in some embodiments, the recipient human oocyte is obtained from a different subject or individual from whom the donor human somatic cell is obtained. In some embodiments, the recipient human oocyte is obtained from the same subject that hNT-ESCs derived from the hSCNT embryo are implanted into. For example, patient-specific hNT-ESCs can be obtained from hSCNT embryos where the nuclear genetic material from the patient-donor human somatic cell is injected into a recipient human oocyte.

In some embodiments, the oocyte is obtained from a female subject who does not have a mitochondrial disease. In some embodiments, the oocyte is obtained from a female subject who has a mitochondrial disease. Mitochondrial diseases are inherited by a defect in the mitochondrial DNA (mtDNA) are well known by one of ordinary skill in the art.

In one embodiment, the recipient human oocyte is from a subject who does not have a mitochondrial DNA mutation, such as a homoplasmic or heteroplasmic mitochondrial disease. This can be determined, for example, by genetic assay, such as by assessing the mitochondrial DNA, or it can be determined by clinical evaluation. The nuclear genetic material such as the chromosomes can be isolated from a donor oocyte from a subject, such as a human subject, with a mitochondrial DNA disease, such as a homoplasmic or heteroplasmic mitochondrial disease.

In some embodiments, the mitochondrial disease can be associated with infertility. Examples of mitochondrial disease associated with infertility include Leber's hereditary optic neuropathy, myoclonic epilepsy, or Kearns-Sayre Syndrome. Thus in some examples, a recipient primate oocyte is from a subject that does not have Leber's hereditary optic neuropathy, myoclonic epilepsy, or Kearns-Sayre Syndrome.

In other example, the nuclear genetic material including the chromosomes is from a donor human oocyte from a primate subject that has Leber's hereditary optic neuropathy, myoclonic epilepsy, Neuropathy, ataxia and pigmentary retinopathy syndrome, Maternally inherited Leigh's syndrome (MILS), Myoclonic epilepsy syndrome with red-ripped fibers (MERRF), Mitochondrial encephalo-myopathy syndrome with lactic acidosis and cerebro-vascular accident episodes (MELAS), Maternally inherited diabetes with deafness, mitochondrial encephalomyopathy, chronic progressive external opthalmoplegia, Pearson's bone marrow-pancreas syndrome, diabetes insipidus, diabetes mellitus, optic atrophy and deafness (DIDMOAD), Chronic progressive external opthalmoplegia or Kearns-Sayre's Syndrome. Thus, the recipient human oocyte is isolated from a subject that does not have mitochondrial disease, such as Leber's hereditary optic neuropathy, myoclonic epilepsy, Neuropathy, ataxia and pigmentary retinopathy syndrome, Maternally inherited Leigh's syndrome (MILS), Myoclonic epilepsy syndrome with red-ripped fibers (MERRF), Mitochondrial encephalo-myopathy syndrome with lactic acidosis and cerebro-vascular accident episodes (MELAS), Maternally inherited diabetes with deafness, mitochondrial encephalomyopathy, chronic progressive external opthalmoplegia, Pearson's bone marrow-pancreas syndrome, diabetes insipidus, diabetes mellitus, optic atrophy and deafness (DIDMOAD), Chronic progressive external opthalmoplegia and Kearns-Sayre's Syndrome.

Leber's hereditary optic neuropathy (LHON) or Leber optic atrophy is a mitochondrially inherited (mother to all offspring) degeneration of retinal ganglion cells (RGCs) and their axons that leads to an acute or subacute loss of central vision; this affects predominantly young adult males. However, LHON is only transmitted through the mother as it is primarily due to mutations in the mitochondrial (not nuclear) genome and only the egg contributes mitochondria to the embryo. LHON is usually due to one of three pathogenic mitochondrial DNA (mtDNA) point mutations. These mutations are at nucleotide positions 11778 G to A, 3460 G to A and 14484 T to C, respectively in the ND4, ND1 and ND6 subunit genes of complex I of the oxidative phosphorylation chain in mitochondria. Clinically, there is an acute onset of visual loss, first in one eye, and then a few weeks to months later in the other. Onset is usually young adulthood, but age range at onset from 8-60 is reported. This typically evolves to very severe optic atrophy and permanent decrease of visual acuity.

Leigh's disease, also known as Subacute Necrotizing Encephalomyelopathy (SNEM), is a rare neurometabolic disorder that affects the central nervous system. It is an inherited disorder that usually affects infants between the age of three months and two years, but, in rare cases, teenagers and adults as well. In the case of the disease, mutations in mitochondrial DNA (mtDNA) or in nuclear DNA (gene SURF and some COX assembly factors) cause degradation of motor skills and eventually death. The disease is most noted for its degradation in one's ability to control one's movements. As it progresses rapidly, the earliest signs may be poor sucking ability and loss of head control and motor skills. Other symptoms include loss of appetite, vomiting, irritability, continuous crying (in infants), and seizures. A later sign can also be episodes of lactic acidosis, which can lead to impairment of respiratory and kidney function. Some children can present with loss of development skills or developmental regression and have often had investigations for failure to thrive. As the disease progresses in adults, it may also cause general weakness, kidney failure, and heart problems. Life expectancy is usually about a year within the onset of symptoms although both acute fulminating illness of a few days and prolonged survival have been reported.

Neuropathy, ataxia, and retinitis pigmentosa (NARP) is a condition that causes a variety of signs and symptoms chiefly affecting the nervous system. Beginning in childhood or early adulthood, most people with NARP experience numbness, tingling, or pain in the arms and legs (sensory neuropathy); muscle weakness; and problems with balance and coordination (ataxia). Many affected individuals also have vision loss caused by changes in the light-sensitive tissue that lines the back of the eye (the retina). In some cases, the vision loss results from a condition called retinitis pigmentosa. This eye disease causes the light-sensing cells of the retina gradually to deteriorate. Neuropathy, ataxia, and retinitis pigmentosa is a condition related to mutations in mitochondrial DNA, specifically in the MT-ATP6 gene.

Myoneurogenic gastrointestinal encephalopathy or MNGIE is another mitochondrial disease typically appearing between the second and fifth decades of life. MNGIE is a multisystem disorder causing ptosis, progressive external ophthalmoplegia, gastrointestinal dysmotility (often pseudoobstruction), diffuse leukoencephalopathy, thin body habitus, peripheral neuropathy, and myopathy.

In some embodiments, if the female subject has a mitochondrial DNA (mtDNA) defect, or mutation in the mtDNA, mitochondrial transfer can occur such that an ooplasm with healthy mitochondria and wildtype mtDNA can be introduced into a recipient oocyte via cytoplasmic transfer, also called ooplasmic transfer to result in a heteroplasmy oocyte (see: Sterneckert et al., Nat Reviews Genetics, Genetics 15, 625-639 (2014) and Ma et al., 2015; Metabolic rescue in pluripotent cells from patients with mtDNA disease, Nature 524, 234-238). Methods for cytoplasmic transfer are well known, e.g., are described in US patent application 2004/0268422, which is incorporated herein in its entirety by reference. Such a heteroplasmy oocyte can then be enucleated and used as the recipient oocyte for injection of the nuclear genetic material from the donor somatic cell. Accordingly, in some embodiments, the resultant SCNT embryo can be derived from 3 separate individuals; i.e., contain nuclear genetic material from the donor somatic cell, the cytoplasm from the recipient oocye and wild type or mutant mtDNA from a third individual or donor subject).

Donor Human Cells

The methods, kits and compositions as disclosed herein comprise a donor human cell, from which the nuclei is collected (harvested) and injected into an enucleated human oocyte to generate a human SCNT embryo. In some embodiments, the donor human cell is a terminally differentiated somatic cell. In some embodiments, the donor human cell is not an embryonic stem cell or an adult stem cell or an iPS cell. In some embodiments, the donor somatic cell is obtained from a male human subject, e.g., XY subject. In alternative embodiments, the donor of a somatic cell is obtained from a female human subject, e.g., XX subject. In some embodiments, the donor of the human somatic cell is obtained from a XXY human subject.

Human donor somatic cells useful in the present invention include, by way of example, epithelial, neural cells, epidermal cells, keratinocytes, hematopoietic cells, melanocytes, chondrocytes, lymphocytes (B and T lymphocytes), other immune cells, erythrocytes, macrophages, melanocytes, monocytes, mononuclear cells, fibroblasts, cardiac muscle cells, cumulus cells and other muscle cells, etc. In some embodiments, human somatic cells used for nuclear transfer may be obtained from different organs, e.g., skin, lung, pancreas, liver, stomach, intestine, heart, reproductive organs, bladder, kidney, urethra and other urinary organs, etc. These are just some examples of suitable human donor cells. Suitable donor cells, i.e., cells useful in the subject invention, may be obtained from any cell or organ of the body. This includes all somatic and in some embodiments, germ cells e.g., primordial germ cells, sperm cells. In some embodiments, the human donor cell or nucleus (i.e., nuclear genetic material) from the human donor cell is actively dividing, i.e., non-quiescent cells, as this has been reported to enhance cloning efficacy. Such donor somatic cells include those in the G1, G2 S or M cell phase. Alternatively, quiescent cells may be used. In some embodiments, such human donor cells will be in the G1 cell cycle. In certain embodiments, human donor and/or recipient cells of the application do not undergo a 2-cell block.

In some embodiments, the nuclear genetic material (i.e., the nucleus) of a human donor somatic cell is obtained from a cumulus cell, Sertoli cells or from an embryonic fibroblast or adult fibroblast cell.

In some embodiments, the nuclear genetic material is genetically modified, e.g., to correct for a genetic mutation or abnormality, or to introduce a genetic modification, for example, to study the effect of the genetic modification in a disease model, e.g., in NT-ESCs obtained from the human SCNT embryo. In such embodiments, the NT-ESCs are patient-specific NT-ESC, which can be used for therapeutic cloning, and/or to study a particular disease, where the patient has, or has a predisposition to develop a particular disease. In some embodiments, the nuclear genetic material of the human donor cell is genetically modified, e.g., to introduce a desired characteristic into the somatic donor cell. Methods to genetically modify a somatic cell are well known by persons of ordinary skill in the art and are encompassed for use in the methods and compositions as disclosed herein.

In some embodiments, a human donor somatic cell is selected according to the methods as disclosed in US patent Application US2004/0025193, which is incorporated herein in its entirety by reference, which discloses introducing a desired transgene into the human donor somatic cell and selecting the human somatic cells having the transgene prior to obtaining the nucleus for injection into the recipient oocyte.

In certain embodiments, human donor nuclei (e.g., the nuclear genetic material from the donor somatic cell) may be labeled. Cells may be genetically modified with a transgene encoding a easily visualized protein such as the Green Fluorescent protein (Yang, M., et al., 2000, Proc. Natl. Acad. Sci. USA, 97:1206-1211), or one of its derivatives, or modified with a transgene constructed from the Firefly (Photinus pyralis) luciferase gene (Fluc) (Sweeney, T. J., et al. 1999, Proc. Natl. Acad. Sci. USA, 96: 12044-12049), or with a transgene constructed from the Sea Pansey (Renilla reniformis) luciferase gene (Rluc) (Bhaumik, S., and Ghambhir, S. S., 2002, Proc. Natl. Acad. Sci. USA, 99:377-382).

One or more transgenes introduced into the nuclear genetic material of the donor somatic cell may be constitutively expressed using a “house-keeping gene” promoter such that the transgene(s) are expressed in many or all cells at a high level, or the transgene(s) may be expressed using a tissue specific and/or specific developmental stage specific gene promoter, such that only specific cell lineages or cells that have located into particular niches and developed into specific tissues or cell types express the transgene(s) and visualized (if the transgene is a reporter gene). Additional reporter transgenes or labeling reagents include, but are not limited to, luminescently labeled macromolecules including fluorescent protein analogs and biosensors, luminescent macromolecular chimeras including those formed with the green fluorescent protein and mutants thereof, luminescently labeled primary or secondary antibodies that react with cellular antigens involved in a physiological response, luminescent stains, dyes, and other small molecules. Labeled cells from a mosaic blastocyst can be sorted for example by flow cytometry to isolate the cloned population.

In some embodiments, human donor somatic cell can be from healthy human donors, e.g., healthy humans, or donors with pre-existing medical conditions (e.g., Parkinson's Disease (PD), ALS, Altzhiemer's disease, Huntington's disease, Rhumatoid arthritis (RA), Age Related Macular Degeneration (AMD), diabetes, obesity, cardiac disease, cystic fibrosis, an autoimmune disease (e.g., MS, Lupus), a neurodegenerative disease, any subject with a genetic or acquired disease) or any subject whom is in need to a regenerative therapy and/or a stem cell transplantation to treat an existing, or pre-existing or developing condition or disease. For example, in some embodiments, a donor human somatic cell is obtained from a subject who is to be in the future, a recipient of a stem cell transplant of SCNT-derived human ES cells (NT-ESCs), thereby allowing autologous transplantation of patient-specific hES cells. Accordingly, in some embodiments, the methods and compositions allow for the production of patient-specific isogenic embryonic stem cell lines (i.e., isogenic hNT-ESC lines).

Accordingly, the methods, compositions and kits as disclosed herein enable one to obtain a patient-specific human stem cell line, by functionally enucleating the human oocyte line and fusing with the nuclear genetic material obtained from a somatic cell collected from the human patient donor, thereby generating a hSCNT, which can be used to generate patient-specific NT-ESCs. In some embodiments, encompassed herein is a method of treatment by administering the patient-specific hNT-ESCs to the patient, where, in some embodiments, the patient was the donor of the human somatic cell where the nuclear genetic material was harvested for the SCNT procedure.

In some embodiments, the human donor somatic cell or nuclei (i.e., nuclear genetic material) are treated with a H3K9 methyltransferase inhibitor as disclosed herein, for example, any one of an inhibitor of human SUV39h1, human SUV39h2 or human SETDB1 according to the methods as disclosed herein. In certain embodiments, donor human cell or nuclei is not pretreated before nuclear transfer, and the hybrid oocyte, or hSCNT embryo is treated with a H3K9 methyltransferase inhibitor and/or KDM4 histone demethylase activator according to the methods as disclosed herein. In certain embodiments, a donor cell or nuclei are not pretreated with spermine, protamine, or putrescine before nuclear transfer or collection of the genetic material (or nucleus) for injection into the enucleated recipient oocyte.

Contacting the Donor Somatic Cell, Recipient Human Oocyte, Hybrid Oocyte or Human SCNT with an Agent which Decreases H3K9Me3 Methylation.

In some embodiments, a human donor somatic cell is treated with, or contacted with a H3K9 methyltransferase inhibitor and/or KDM4 histone demethylase activator. In some embodiments, the nuclei (or nuclear genetic material) of the donor human cell is treated with, or contacted with, a H3K9 methyltransferase inhibitor and/or KDM4 histone demethylase activator. In some embodiments, the cytoplasm and/or nuclei of the donor human cell is treated with, or contacted with, a H3K9 methyltransferase inhibitor as disclosed herein, for example, an inhibitor of any one or a combination of human SUV39h1, human SUV39h2 and/or human SETDB1. In some embodiments, the contact is microinjection of the H3K9 methyltransferase inhibitor and/or KDM4 histone demethylase activator into the cytoplasm and/or nucleus of the donor human somatic cell.

In some embodiments, the donor somatic cell is contacted with an inhibitor of human SUV39h1 and/or human SUV39h2, or both (SUV39h1/2) at least about 24 hours, or at least about 48 hours, or at least about 3-days or at least about 4-days or more than 4-days before removal of the nuclei for transfer to the enucleated human donor oocyte. In some embodiments, an inhibitor of SUV39h1 and/or SUV39h2, or both (SUV39h1/2) is by siRNA and inhibition of the expression of SUV39h1 and/or SUV39h2, or both (SUV39h1/2) occurs for a time period of at least 12 hours, or at least 24 hours or more prior to removal of the nuclei for injection into the recipient oocyte. In some embodiments, inhibition of SUV39h1 and/or SUV39h2, or both (SUV39h1/2), occurs in the donor somatic cell, e.g., at least about 24 hours, or at least about 48 hours, or at least about 3-days or at least about 4-days or more than 4-days before removal of the nuclei for transfer to the enucleated human donor oocyte. In some embodiments, inhibiting the expression of SUV39h1 and/or SUV39h2, or both (SUV39h1/2) is by siRNA and occurs for at least 12 hours, or at least 24 hours or more, at the time periods prior to removal of the nuclei.

In some embodiments, in some embodiments, a human oocyte is treated with or contacted with a H3K9 methyltransferase inhibitor and/or KDM4 histone demethylase activator. In some embodiments, a human oocyte is an enucleated oocyte which is treated with, or contacted with, a H3K9 methyltransferase inhibitor and/or KDM4 histone demethylase activator, e.g., by direct injection into the cytoplasm of the enucleated oocyte. In some embodiments, a human oocyte, or enucleated human oocyte is treated with or contacted with a KDM4 histone demethylase activator, for example, but not limited to, an agent which activates a member of the KDM4 family of histone demethylases, such as anyone or a combination of human KDM4A, human KDM4B, human KDM4C, human KDM4D or human KDM4E. In some embodiments, the enucleated oocyte has not been injected with, or received, the donor nuclear genetic material.

In alternative embodiments, a recipient human oocyte will be treated with a H3K9 methyltransferase inhibitor and/or KDM4 histone demethylase activator within the timeframe of about 40 hours prior to nuclear transfer (i.e., prior to being injected with the donor nuclear genetic material). Such contact can occur about 40 hours before nuclear transfer, or more preferably within the timeframe of about 12 or 24 hours before nuclear transfer, and most preferably from within the timeframe of about 4 to 9 hours before nuclear transfer. In some embodiments, a recipient human oocyte is contacted with a H3K9 methyltransferase inhibitor and/or KDM4 histone demethylase activator when the recipient oocyte is a hybrid oocyte (i.e., comprises the nuclear genetic material from the donor somatic cell, but is not yet activated). Such contact can occur about 40 hours after nuclear transfer, or more preferably within the timeframe of about 1-4, or 4-12 or any time within 24 hours after nuclear transfer, and most preferably from within the timeframe of about 1-4, or 4 to 9 hours after nuclear transfer, but before fusion or activation.

The recipient human oocyte can be treated with a H3K9 methyltransferase inhibitor and/or KDM4 histone demethylase activator either before, simultaneous, or after nuclear transfer of the nuclear genetic material obtained from the human donor somatic cell. In general, a recipient human oocyte will be treated within 5 hours of nuclei transfer or within 5 hours of activation or fusion (e.g., 5 hpa; 5 hours post activation). In some embodiments, activation (or fusion) occurs within 1-2 or 2-4 hours after injection of the genetic material from the donor somatic cell into an enucleated oocyte, and in that case, the SCNT embryo is contacted with a H3K9 methyltransferase inhibitor and/or KDM4 histone demethylase activator.

In some embodiments, the human SCNT embryo is treated with a H3K9 methyltransferase inhibitor and/or KDM4 histone demethylase activator. The human SCNT embryo is generated from the injection of a nuclei (e.g., nuclear genetic material) from a donor somatic cell into an enucleated recipient oocyte to form a “hybrid oocyte”, which is activated (or fused) to generate a SCNT embryo. In some embodiments, the hybrid oocyte (e.g., enucleated oocyte comprising donor nuclear genetic material prior to activation) is treated with a H3K9 methyltransferase inhibitor and/or KDM4 histone demethylase activator as disclosed herein.

The SCNT embryo is generated after activation (also known as fusion) of the donor nuclear genetic material with the cytoplasm of the recipient oocyte. In some embodiments, either, or both the cytoplasm or nuclei from a human donor cell and/or the enucleated oocyte have been treated or contacted with a H3K9 methyltransferase inhibitor and/or KDM4 histone demethylase activator as disclosed herein. In some embodiments, neither the donor cell and/or enucleated oocyte has been treated with a H3K9 methyltransferase inhibitor and/or KDM4 histone demethylase activator, as the hybrid oocyte is treated and/or the hSCNT embryo is treated.

In some embodiments, increasing the efficiency of human somatic cell nuclear transfer (hSCNT) comprising contacting a human SCNT embryo, e.g., at least 5 hpa, or between 10-12 hpa (i.e. at 1-cell stage), or at about 20 hpa (i.e., early 2-cell stage) or between 20-28 hpa (i.e., 2-cell stage) with at least one of (i) a KDM4 family of histone demethylase and/or (ii) a H3K9 methyltransferase-inhibiting agent. In some embodiments, exogenous expression of a KDM4 gene, e.g., KDM4A, occurs in the SCNT embryo at any one of 5 hpa, between 10-12 hpa (i.e. at 1-cell stage), at about 20 hpa (i.e., early 2-cell stage) or between 20-28 hpa (i.e., 2-cell stage). In some embodiments, where a hSCNT embryo is contacted with an agent which inhibits H3K9me3, such agent, e.g., agent that increases exogenous expression of a KDM4 gene, e.g., KDM4A, (e.g., KDM4A mRNA or mod-RNA), each cell of the SCNT embryo (e.g., each cell of the 2-cell embryo or 4-cell embryo) is injected with the KDM4A activating or overexpressing agent. In some embodiments, exogenous expression of a KDM4 gene, e.g., KDM4A, occurs in the human SCNT embryo at any one of 5 hpa, between 10-12 hpa (i.e. at 1-cell stage), at about 20 hpa (i.e., early 2-cell stage) or between 20-28 hpa (i.e., 2-cell stage) or later (e.g., at the 4-cell stage). In some embodiments, where the human SCNT embryo is contacted with an agent which inhibits H3K9me3, such agent, e.g., agent that increases exogenous expression of a KDM4 gene, e.g., KDM4A, (e.g., KDM4A mRNA or mod-RNA), each cell of the SCNT embryo (e.g., each cell of the 2-cell embryo, or 4-cell embryo) is injected with the KDM4d activating or overexpressing agent.

Method of Nuclear Transfer

One objective of the present invention is to provide a means of cloning human somatic cells more efficiently. The methods and compositions of the disclosure may be used for therapeutic cloning a human, e.g., for obtaining human pluripotent stem cells (PSCs) and human totipotent cells (TSCs), and for reprogramming a human somatic cell.

Nuclear transfer techniques or nuclear transplantation techniques are known in the literature. See, in particular, Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, and WO 90/03432, which are incorporated by reference in their entirety herein. Also, U.S. Pat. Nos. 4,944,384 and 5,057,420 describe procedures for bovine nuclear transplantation. See, also Cibelli et al, Science, Vol. 280:1256-1258 (1998).

Transferring the donor nucleus into a recipient fertilized embryo may be done with a microinjection device. In certain embodiments, minimal cytoplasm is transferred with the nucleus. Transfer of minimal cytoplasm is achievable when nuclei are transferred using microinjection, in contrast to transfer by cell fusion approaches. In one embodiment, the microinjection device includes a piezo unit. Typically, the piezo unit is operably attached to the needle to impart oscillations to the needle. However, any configuration of the piezo unit which can impart oscillations to the needle is included within the scope of the invention. In certain instances the piezo unit can assist the needle in passing into the object. In certain embodiments, the piezo unit may be used to transfer minimal cytoplasm with the nucleus. Any piezo unit suitable for the purpose may be used. In certain embodiments a piezo unit is a Piezo micromanipulator controller PMM150 (PrimeTech, Japan).

In some embodiments, the method includes a step of fusing the donor nuclei with enucleated oocyte. Fusion of the cytoplasts with the nuclei is performed using a number of techniques known in the art, including polyethylene glycol (see Pontecorvo “Polyethylene Glycol (PEG) in the Production of Mammalian Somatic Cell Hybrids” Cytogenet Cell Genet. 16(1-5):399-400 (1976), the direct injection of nuclei, Sendai viral-mediated fusion (see U.S. Pat. No. 4,664,097 and Graham Wistar Inst. Symp. Monogr. 9 19 (1969)), or other techniques known in the art such as electrofusion. Electrofusion of cells involves bringing cells together in close proximity and exposing them to an alternating electric field. Under appropriate conditions, the cells are pushed together and there is a fusion of cell membranes and then the formation of fusate cells or hybrid cells. Electrofusion of cells and apparatus for performing same are described in, for example, U.S. Pat. Nos. 4,441,972, 4,578,168 and 5,283,194, International Patent Application No. PCT/AU92/00473 [published as WO1993/05166], Pohl, “Dielectrophoresis”, Cambridge University Press, 1978 and Zimmerman et al., Biochimica et Bioplzysica Acta 641: 160-165, 1981.

Methods of SCNT, and activation (i.e. fusion) of the donor nuclear genetic material with the cytoplasm of the recipient oocyte are disclosed in US application 2004/0148648, which is incorporated herein in its entirety by reference.

Oocyte Collection.

Oocyte donors can be synchronized and superovulated as previously described (Gavin W.G., 1996), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml). Nuclear transfer can also utilize oocytes that could have been matured in vivo or in vitro. In vivo matured oocytes are derived as explained above, and in vitro matured oocytes are allowed to develop in vitro to a specific cell stage before they are harvested for use in the nuclear transfer.

Cytoplast Preparation and Enucleation.

Oocytes with attached cumulus cells are typically discarded. Cumulus-free oocytes were divided into two groups: arrested Metaphase-II (one polar body) and Telophase-II protocols (no clearly visible polar body or presence of a partially extruding second polar body). The oocytes in the arrested Metaphase-II protocol are enucleated first. The oocytes allocated to the activated Telophase-II protocols were prepared by culturing for 2 to 4 hours in M199/10% FBS. After this period, all activated oocytes (presence of a partially extruded second polar body) were grouped as culture-induced, calcium-activated Telophase-II oocytes (Telophase-II-Ca) and enucleated. Oocytes that had not activated during the culture period were subsequently incubated 5 minutes in M199, 10% FBS containing 7% ethanol to induce activation and then cultured in M199 with 10% FBS for an additional 3 hours to reach Telophase-II (Telophase-II-EtOH protocol). All oocytes are treated with cytochalasin-B 15 to 30 minutes prior to enucleation. Metaphase-II stage oocytes were enucleated with a glass pipette by aspirating the first polar body and adjacent cytoplasm surrounding the polar body (˜30% of the cytoplasm) to remove the metaphase plate. Telophase-II-Ca and Telophase-II-EtOH oocytes were enucleated by removing the first polar body and the surrounding cytoplasm (10 to 30% of cytoplasm) containing the partially extruding second polar body. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in M199 for 30 to 60 minutes before electrofusion and activation procedures. Reconstructed oocytes were equilibrated in fusion buffer (300 mM mannitol, 0.05 mM CaCl2, 0.1 mM MgSO4, 1 mM K2HPO4, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 gm gap; BTX-Genetronics, San Diego, Calif.) filled with fusion medium.

Fusion (e.g., activation) is performed using a fusion slide. The fusion slide is placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO2 in air. It should be noted that mannitol may be used in the place of cytocholasin-B throughout any of the protocols provided in the current disclosure (HEPES-buffered mannitol (0.3 mm) based medium with Ca+2 and BSA). Starting at between 10 to 90 minutes post-fusion, most preferably at 30 minutes post-fusion, the presence of an actual karyoplast/cytoplast fusion is determined for the development of a transgenic embryo for later implantation or use in additional rounds of nuclear transfer.

Following cycloheximide treatment, couplets are washed extensively with equilibrated SOF medium supplemented with at least 0.1% bovine serum albumin, preferably at least 0.7%, preferably 0.8%, plus 100 U/ml penicillin and 100 μg/ml streptomycin (SOF/BSA). Couplets were transferred to equilibrated SOF/BSA, and cultured undisturbed for 24-48 hours at 37-39° C. in a humidified modular incubation chamber containing approximately 6% 02, 5% CO2, balance Nitrogen. Nuclear transfer embryos with age appropriate development (1-cell up to 8-cell at 24 to 48 hours) were transferred to surrogate synchronized recipients.

Nuclear Transfer Embryo Culture and Transfer to Recipients.

Culture of SCNT Embryos

It has been suggested that embryos derived by hSCNT may benefit from, or even require culture conditions in vivo other than those in which embryos are usually cultured (at least in vivo). In routine multiplication of bovine embryos, reconstituted embryos (many of them at once) have been cultured in sheep oviducts for 5 to 6 days (as described by Willadsen, In Mammalian Egg Transfer (Adams, E. E., ed.) 185 CRC Press, Boca Raton, Fla. (1982)). In certain embodiments, the SCNT embryo may be embedded in a protective medium such as agar before transfer and then dissected from the agar after recovery from the temporary recipient. The function of the protective agar or other medium is twofold: first, it acts as a structural aid for the SCNT embryo by holding the zona pellucida together; and secondly it acts as barrier to cells of the recipient animal's immune system. Although this approach increases the proportion of embryos that form blastocysts, there is the disadvantage that a number of embryos may be lost. In some embodiments, hSCNT embryos can be co-cultured on monolayers of feeder cells, e.g., primary goat oviduct epithelial cells, in 50 μl droplets. Embryo cultures can be maintained in a humidified 39° C. incubator with 5% CO₂ for 48 hours before transfer of the hSCNT embryos are used for collection of blastomeres for generation of hNT-ESCs.

### Applications

Obtaining Totipotent Cells (TPCs).

SCNT experiments showed that nuclei from adult differentiated somatic cells can be reprogrammed to a totipotent state. Accordingly, a hSCNT embryo generated using the methods as disclosed herein can be cultured in a suitable in vitro culture medium for the generation of totipotent or embryonic stem cell or stem-like cells and cell colonies. Culture media suitable for culturing and maturation of embryos are well known in the art. Examples of known media, which may be used for bovine embryo culture and maintenance, include Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% fetal calf serum, 0.2 Ma pyruvate and 50 ug/ml gentamicin sulphate. Any of the above may also involve co-culture with a variety of cell types such as granulosa cells, oviduct cells, BRL cells and uterine cells and STO cells.

In particular, human epithelial cells of the endometrium secrete leukemia inhibitory factor (LIF) during the preimplantation and implantation period. Therefore, in some embodiments, the addition of LIF to the culture medium is encompassed to enhancing the in vitro development of the hSCNT-derived embryos. The use of LIF for embryonic or stem-like cell cultures has been described in U.S. Pat. No. 5,712,156, which is herein incorporated by reference.

Another maintenance medium is described in U.S. Pat. No. 5,096,822 to Rosenkrans, Jr. et al., which is incorporated herein by reference. This embryo medium, named CR1, contains the nutritional substances necessary to support an embryo. CR1 contains hemicalcium L-lactate in amounts ranging from 1.0 mM to 10 mM, preferably 1.0 mM to 5.0 mM. Hemicalcium L-lactate is L-lactate with a hemicalcium salt incorporated thereon. Also, suitable culture medium for maintaining human embryonic stem cells in culture as discussed in Thomson et al., Science, 282:1145-1147 (1998) and Proc. Natl. Acad. Sci., USA, 92:7844-7848 (1995).

In some embodiments, the feeder cells will comprise mouse embryonic fibroblasts. Means for preparation of a suitable fibroblast feeder layer are described in the example which follows and is well within the skill of the ordinary artisan.

Methods of deriving human ES cells (e.g., human NT-ESCs or hNT-ESCs) from blastocyst-stage human SCNT embryos (or the equivalent thereof) are well known in the art. Such techniques can be used to derive human ES cells (e.g., hNT-ESCs) from human SCNT embryos, where the hSCNT embryos used to generate hNT-ESCs have a reduced level of H3K9me3 in the nuclear genetic material donated from the human somatic donor cell, as compared to hSCNTs which were not treated with a member of the KDM4 demethylase family and/or an inhibitor of the histone methyltransferase SUV39h1/SUV39h2. Additionally or alternatively, hNT-ESCs can be derived from cloned human SCNT embryos during earlier stages of development.

In certain embodiments, blastomeres generated from human SCNT embryos generated using the methods, compositions and kits as disclosed herein can be dissociated using a glass pipette to obtain totipotent cells. In some embodiments, dissociation may occur in the presence of 0.25% trypsin (Collas and Robl, 43 BIOL. REPROD. 877-84, 1992; Stice and Robl, 39 BIOL. REPROD. 657-664, 1988; Kanka et al., 43 MOL. REPROD. DEV. 135-44, 1996).

In certain embodiments, the resultant blastocysts, or blastocyst-like clusters from the hSCNT embryos can be used to obtain embryonic stem cell lines, eg., nuclear transfer ESC (ntESC) cell lines. Such lines can be obtained, for example, according to the culturing methods reported by Thomson et al., Science, 282:1145-1147 (1998) and Thomson et al., Proc. Natl. Acad. Sci., USA, 92:7544-7848 (1995), incorporated by reference in their entirety herein.

Pluripotent embryonic stem cells can also be generated from a single blastomere removed from a hSCNT embryo without interfering with the embryo's normal development to birth. See U.S. application Nos. 60/624,827, filed Nov. 4, 2004; 60/662,489, filed Mar. 14, 2005; 60/687,158, filed Jun. 3, 2005; 60/723,066, filed Oct. 3, 2005; 60/726,775, filed Oct. 14, 2005; Ser. No. 11/267,555 filed Nov. 4, 2005; PCT application no. PCT/US05/39776, filed Nov. 4, 2005, the disclosures of which are incorporated by reference in their entirety; see also Chung et al., Nature, Oct. 16, 2005 (electronically published ahead of print) and Chung et al., Nature V. 439, pp. 216-219 (2006), the entire disclosure of each of which is incorporated by reference in its entirety). In such a case, an hSCNT embryo is not destroyed for the generation of pluripotent stem cells.

In one aspect of the invention, the method comprises the utilization of cells derived from the hSCNT embryo in research and in therapy. Such human pluripotent stem cells (PSCs) or totipotent stem cells (TSC) can be differentiated into any of the cells in the body including, without limitation, skin, cartilage, bone, skeletal muscle, cardiac muscle, renal, hepatic, blood and blood forming, vascular precursor and vascular endothelial, pancreatic beta, neurons, glia, retinal, inner ear follicle, intestinal, lung, cells.

In another embodiment of the invention, the hSCNT embryo, or blastocyst, or pluripotent or totipotent cells obtained from a hSCNT embryo (e.g., NT-ESCs), can be exposed to one or more inducers of differentiation to yield other therapeutically-useful cells such as retinal pigment epithelium, hematopoietic precursors and hemangioblastic progenitors as well as many other useful cell types of the ectoderm, mesoderm, and endoderm. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3, 6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropoietin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocytemacrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thromopoietin, transforming growth factor-(alpha, beta 1,2,3,4,5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin, extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan. Other inducers include cells or components derived from cells from defined tissues used to provide inductive signals to the differentiating cells derived from the reprogrammed cells of the present invention. Such inducer cells may derive from human, non-human mammal, or avian, such as specific pathogen-free (SPF) embryonic or adult cells.

Blastomere Culturing.

In one embodiment, the hSCNT embryos can be used to generate blastomeres and utilize in vitro techniques related to those currently used in pre-implantation genetic diagnosis (PGD) to isolate single blastomeres from a hSCNT embryo, generated by the methods as disclosed herein, without destroying the hSCNT embryos or otherwise significantly altering their viability. As demonstrated herein, pluripotent human embryonic stem (hES) cells and cell lines can be generated from a single blastomere removed from a hSCNT embryo as disclosed herein without interfering with the embryo's normal development to birth.

Therapeutic Cloning

The discoveries of Wilmut et al. (Wilmut, et al, Nature 385, 810 (1997) in sheep cloning of “Dolly”, together with those of Thomson et al. (Thomson et al., Science 282, 1145 (1998)) in deriving hESCs, have generated considerable enthusiasm for regenerative cell transplantation based on the establishment of patient-specific hESCs derived from hSCNT-embryos or hSCNT-engineered cell masses generated from a patient's own nuclei. This strategy, aimed at avoiding immune rejection through autologous transplantation, is perhaps the strongest clinical rationale for hSCNT. By the same token, derivations of complex disease-specific SCNT-hESCs may accelerate discoveries of disease mechanisms. For cell transplantations, innovative treatments of murine SCID and PD models with the individual mouse's own SCNT-derived mESCs are encouraging (Rideout et al, Cell 109, 17 (2002); Barberi, Nat. Biotechnol. 21, 1200 (2003)). Ultimately, the ability to create banks of SCNT-derived stem cells with broad tissue compatibility would reduce the need for an ongoing supply of new oocytes.

In certain embodiments of the invention, pluripotent or totipotent cells obtained from a hSCNT embryo (e.g., hNT-ESCs) can be optionally differentiated, and introduced into the tissues in which they normally reside in order to exhibit therapeutic utility. For example, pluripotent or totipotent cells obtained from a hSCNT embryo can be introduced into the tissues. In certain other embodiments, pluripotent or totipotent cells obtained from a hSCNT embryo can be introduced systemically or at a distance from a cite at which therapeutic utility is desired. In such embodiments, the pluripotent or totipotent cells obtained from a hSCNT embryo can act at a distance or may hone to the desired cite.

In certain embodiments of the invention, cloned cells, pluripotent or totipotent cells obtained from a hSCNT embryo can be utilized in inducing the differentiation of other pluripotent stem cells. The generation of single cell-derived populations of cells capable of being propagated in vitro while maintaining an embryonic pattern of gene expression is useful in inducing the differentiation of other pluripotent stem cells. Cell-cell induction is a common means of directing differentiation in the early embryo. Many potentially medically-useful cell types are influenced by inductive signals during normal embryonic development including spinal cord neurons, cardiac cells, pancreatic beta cells, and definitive hematopoietic cells. Single cell-derived populations of cells capable of being propagated in vitro while maintaining an embryonic pattern of gene expression can be cultured in a variety of in vitro, in ovo, or in vivo culture conditions to induce the differentiation of other pluripotent stem cells to become desired cell or tissue types.

The pluripotent or totipotent cells obtained from a hSCNT embryo (e.g., ntESCs) can be used to obtain any desired differentiated cell type. Therapeutic usages of such differentiated human cells are unparalleled. For example, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Hematopoietic stem cells can be obtained, e.g., by fusing an donor adult terminally differentiated somatic cells obtained from a human cancer or AIDS patient, e.g., epithelial cells or lymphocytes with a recipient enucleated human oocyte, thereby obtaining a hSCNT embryo according to the methods as disclosed herein, which can then subsequently be used to obtain patient-specific pluripotent or totipotent cells or stem-like cells as described above, and culturing such cells under conditions which favor differentiation, until hematopoietic stem cells are obtained. Such hematopoietic cells may be used in the treatment of diseases including cancer and AIDS. As discussed herein, the human adult donor cell, or the recipient human oocyte, the hybrid oocyte or hSCNT embryo can be treated with a KDM4 histone dimethylase activator and/or H3K9 methyltransferase inhibitor according to the methods as disclosed herein.

Alternatively, the donor human cells can be adult somatic cells from a human patient with a neurological disorder, and the generated hSCNT embryos can be used to produce patient-specific, or disease-specific pluripotent or totipotent cells which can be cultured under differentiation conditions to produce neural cell lines. Such NT-ESCs can be used in therapeutic cloning to treat neurological disorders, or in disease modeling of neurological and neurodegenerative disorders. Such hNT-ESCs can be directionally differentiated along neuronal lineages by methods commonly known by persons of ordinary skill in the art. Specific diseases treatable by cell-based therapy and transplantation of such human neural cells include, by way of example, Parkinson's disease, Alzheimer's disease, ALS, MS and cerebral palsy, among others. In the specific case of Parkinson's disease (PD), it has been demonstrated that transplanted fetal brain neural cells make the proper connections with surrounding cells and produce dopamine. This can result in long-term reversal of Parkinson's disease symptoms. Accordingly, in some embodiments, patient-specific NT-ESCs differentiated along a neuronal lineage can be used in a method to treat a PD patient, where the NT-ESC are obtained from a hSCNT embryo, and where the hSCNT embryo was created from the fusion of the nuclear genetic material from a somatic cell obtained from the subject with PD with a human enucleated oocyte, which had been treated with a KDM4 agonist or mRNA and/or inhibitor of SUV39h1 and/or SUV39h2.

In some embodiments, the pluripotent or totipotent cells obtained from the hSCNT embryo (e.g., NT-ESCs) can be differentiated into cells with a dermatological prenatal pattern of gene expression that is highly elastogenic or capable of regeneration without causing scar formation. Dermal fibroblasts of mammalian fetal skin, especially corresponding to areas where the integument benefits from a high level of elasticity, such as in regions surrounding the joints, are responsible for synthesizing de novo the intricate architecture of elastic fibrils that function for many years without turnover. In addition, early embryonic skin is capable of regenerating without scar formation. Cells from this point in embryonic development from pluripotent or totipotent cells obtained from the SCNT embryo are useful in promoting scarless regeneration of the skin including forming normal elastin architecture. This is particularly useful in treating the symptoms of the course of normal human aging, or in actinic skin damage, where there can be a profound elastolysis of the skin resulting in an aged appearance including sagging and wrinkling of the skin.

To allow for specific selection of differentiated cells of the NT-ESCs after they have differentiated along different lineages, in some embodiments, donor human somatic cells may be transfected with selectable markers expressed via inducible promoters, thereby permitting selection or enrichment of particular cell lineages when differentiation is induced. For example, CD34-neo may be used for selection of hematopoietic cells, Pwl-neo for muscle cells, Mash-1-neo for sympathetic neurons, Mal-neo for human CNS neurons of the grey matter of the cerebral cortex, etc.

The great advantage of the present invention is that by increasing the efficiency of hSCNT, it provides an essentially limitless supply of isogenic or syngeneic human ES cells, particularly pluripotent that are not induced pluripotent stem cells (e.g., not iPSCs). Such NT-ESCs have advantages over iPSCs and are suitable for transplantation, as they do not partially pluripotent, and do not have viral transgenes or forced expression of reprogramming factors to direct their reprogramming.

In some embodiments, the hNT-ESCs generated from the hSCNTs are patient-specific pluripotent obtained from hSCNT embryos, where the donor human cell was obtained from a subject to be treated with the pluripotent stem cells or differentiated progeny thereof. Therefore, it will obviate the significant problem associated with current transplantation methods, i.e., rejection of the transplanted tissue which may occur because of host-vs-graft or graft-vs-host rejection. Conventionally, rejection is prevented or reduced by the administration of anti-rejection drugs such as cyclosporin. However, such drugs have significant adverse side-effects, e.g., immunosuppression, carcinogenic properties, as well as being very expensive. The present invention should eliminate, or at least greatly reduce, the need for anti-rejection drugs, such as cyclosporine, imulan, FK-506, glucocorticoids, and rapamycin, and derivatives thereof.

Other diseases and conditions treatable by isogenic cell therapy include, by way of example include, but are not limited to, spinal cord injuries, multiple sclerosis (MS), muscular dystrophy, diabetes, liver diseases, i.e., hypercholesterolemia, heart diseases, cartilage replacement, diabetes, burns, foot ulcers, gastrointestinal diseases, vascular diseases, kidney disease, urinary tract disease, and aging related diseases, including Age-related macular degeneration (AMD) and similar conditions.

### Uses for Human NT-ESCs e.g., Human Pluripotent Stem Cells (PSC) and Human Totipotent Stem Cells (TSCs)

The methods and composition as described herein for increasing the efficiency of hSCNT have numerous important uses that will advance the field of stem cell research and developmental biology. For example, the hSCNT embryos can be used to generate hES cells, hES cell lines, human totipotent stem (TS) cells and cell lines, and cells differentiated therefrom can be used to study basic developmental biology as well as specific diseases, and can be used therapeutically in the treatment of numerous diseases and conditions. Additionally, these hNT-ESCs can be used in screening assays to identify factors and conditions that can be used to modulate the growth, differentiation, survival, or migration of these cells. Identified agents can be used to regulate cell behavior in vitro and in vivo, and may form the basis of cellular or cell-free therapies.

The isolation of pluripotent human embryonic stem cells and breakthroughs in SCNT in mammals have raised the possibility of performing human SCNT to generate potentially unlimited sources of undifferentiated cells for use in research, with potential applications in tissue repair and transplantation medicine.

This concept, sometimes called “therapeutic cloning,” refers to the transfer of the nucleus of a somatic cell into an enucleated donor oocyte (Lanza, et al., Nature Med. 5,975 (1999)). In theory, the oocyte's cytoplasm would reprogram the transferred nucleus by silencing all of the somatic cell genes and activating the embryonic ones. ES cells (i.e., ntESCs) are isolated from the inner cell mass (ICM) of the cloned pre-implantation stage embryos. When applied in a therapeutic setting, these cells would carry the nuclear genome of the patient; therefore, it is proposed that after directed cell differentiation, the cells could be transplanted without immune rejection to treat degenerative disorders such as diabetes, osteoarthritis, and Parkinson's disease (among others). Previous reports have described the generation of bovine ES-like cells (Cibelli et al., Nature Biotechnol. 16, 642 (1998)), and mouse ES cells from the ICMs of cloned blastocysts (Munsie et al., Curro Biol 10, 989 (2000); Kawase, et al., Genesis 28, 156 (2000); Wakayama et al., Science 292, 740 (2001)) and the development of cloned human embryos to the 8- to 10-cell stage and blastocysts (Cibelli et al., Regen. Med. 26, 25 (2001); Shu, et al., Fertil. Steril. 78, S286 (2002)). Here, the present invention can be used to generate human, patient-specific ES cells from SCNT-engineered cell masses generated by the methods as disclosed herein. Such ES cells generated from SCNTs are referred to herein as “ntESCs” and can include patient-specific isogenic embryonic stem cell lines.

The present technique for producing human lines of hESCs utilizes excess IVF clinic embryos, and does not yield patient-specific ES cells. Patient-specific, immune-matched hESCs are anticipated to be of great biomedical importance for studies of disease and development and to advance methods of therapeutic stem cell transplantation. Accordingly, the present invention can be used to establish hESC lines from hSCNT generated from human donor skin cells, human donor cumulus cells, or other human donor somatic cells from informed donors whose nucleus is inserted into a donated, enucleated oocytes. These lines of hSCNT-derived hESCs will be grown on animal protein-free culture media.

The major histocompatibility complex identity of each SCNT-derived hESCs (i.e., hNT-ESCs) can be compared to the patient's own to show immunological compatibility, which is important for eventual transplantation. With the generation of these SCNT-derived hESCs (i.e., hNT-ESCs), evaluations of genetic and epigenetic stability will be made.

Many human injuries and diseases result from defects in a single cell type. If defective cells could be replaced with appropriate stem cells, progenitor cells, or cells differentiated in vitro, and if immune rejection of transplanted cells could be avoided, it might be possible to treat disease and injury at the cellular level in the clinic (Thomson et al., Science 282, 1145 (1998)). By generating hESCs from human SCNT embryos or SCNT-engineered cell masses, in which the somatic cell nucleus comes from the individual patient—a situation where the nuclear (though not mitochondrial DNA (mtDNA) genome is identical to that of the donor—the possibility of immune rejection might be eliminated if these cells were to be used for human treatment (Jaenisch, N. Engl. Med. 351, 2787 (2004); Drukker, Benvenisty, Trends Biotechnol. 22, 136 (2004)). Recently, mouse models of severe combined immunodeficiency (SCID) and Parkinson's disease (PD) (Barberi et al., Nat. Biotechnol. 21, 1200 (2003) have been successfully treated through the transplantation of autologous differentiated mouse embryonic stem cells (mESCs) derived from NT blastocysts, a process also referred to as therapeutic cloning.

Generating hESCs from human SCNT embryos or SCNT-engineered cell masses generated using the methods as disclosed herein can be assessed for the expression of hESC pluripotency markers, including alkaline phosphatase (AP), stage-specific embryonic antigen 4 (SSEA-4), SSEA-3, tumor rejection antigen 1-81 (Tra-I-81), Tra-I-60, and octamer-4 (Oct-4). DNA fingerprinting with human short tandem-repeat probes can also be used to show with high certainty that every NT-hESC line derived originated from the respective donor of the somatic human cell and that these lines were not the result of enucleation failures and subsequent parthenogenetic activation. Stem cells are defined by their ability to self-renew as well as differentiate into somatic cells from all three embryonic germ layers: ectoderm, mesoderm, and endoderm. Differentiation will be analyzed in terms of teratoma formation and embryoid body (EB) formation as demonstrated by IM injection into appropriate animal models.

In summary, the present method to increase the efficiency of hSCNT provides an alternative to the current methods for deriving ES cells. However, unlike current approaches, hSCNT can be used to generate ES cell lines histocompatible with donor tissue. As such, hSCNT embryos produced by the methods as disclosed herein may provide the opportunity in the future to develop cellular therapies histocompatible with particular patients in need of treatment.

In some embodiments, the methods, systems, kits and devices as disclosed herein can be performed by a service provider, for example, where an investigator can request a service provider to provide a hSCNT embryo, or pluripotent stem cells, or totipotent stem cells derived from a hSCNT embryo which has been generated using the methods as disclosed herein in a laboratory operated by the service provider. In such an embodiment, after obtaining a donor human somatic cell, the service provider can performs the method as disclosed herein to generate the hSCNT embryo, or blastocysts derived from such a hSCNT-embryo, or generate the hNT-ESCs from such a hSCNT embryo, and then the service provider can provide the investigator with the hSCNT embryo, or blastocysts derived from such a SCNT-embryo or hNT-ESCs from such a hSCNT embryo. In some embodiments, the investigator can send the donor human somatic cell samples to the service provider via any means, e.g., via mail, express mail, etc., or alternatively, the service provider can provide a service to collect the donor human somatic cell samples from the investigator and transport them to the laboratories of the service provider. In some embodiments, the investigator can deposit the donor human somatic cell samples to be used in the hSCNT method at the location of the service provider laboratories. In alternative embodiments, the service provider provides a stop-by service, where the service provider send personnel to the laboratories of the investigator and also provides the kits, apparatus, and reagents for performing the hSCNT methods and systems of the invention as disclosed herein of the investigators desired/preferred donor human somatic cell (e.g., a patient-specific somatic cell) in the investigators laboratories. Such a service is useful for therapeutic cloning, e.g., for obtaining hNT-ESCs and/or pluripotent stem cells from blastocyst from the hSCNT-embryos, e.g., for patient-specific pluripotent stem cells for transplantation into a subject in need of regenerative cell- or tissue therapy.

Also provided herein are therapeutic compositions comprised of transplantable cells which have been derived (produced) from NT-ESCs in a formulation suitable for administration to a human. In one embodiment, the recipient for transplantation is the donor human that is the source of the donor somatic cell. In some embodiment, the therapeutic compositions include multipotent cells, lineage-specific stem cells, as well as partly or fully differentiated cells derived from the hNT-ESCs provided herein.

The preparations of hNT-ESCs cells derived from the hSCNTs allows for methods for providing cells to an individual in need thereof by administering an effective amount of one or more preparations of transplantable cells to the individual in need thereof. The cells will be matched at one or more loci of the major histocompatibility complex (MHC). In one embodiment, there is a complete match at every MHC loci. In one embodiment the hNT-ESCs cells derived from the hSCNTs is made by the transfer of a nucleus from a somatic cell of the individual of interest into an enucleated host cell (e.g., oocyte) from a second individual. The hNT-ESCs cells derived from the hSCNTs can then be cultured as described above to produce pluripotent stem cells and multipotent stem cells (MPSCs). A therapeutically effective amount of the multipotent cells can then be utilized in the subject of interest. In one embodiment, cells matched at one or more MHC loci to the treated individual are generated and cultured using the teachings provided herein, such as by SCNT. In a preferred embodiment, the cells are cultured in media free of serum. In another preferred embodiment, the cells have not been cultured with xenogeneic cells (e.g., non-human fibroblasts such as mouse embryonic fibroblasts).

Methods for treating disease are provided that comprise transplanting hNT-ESCs cells derived from the hSCNTs in a human afflicted with a disease characterized by damaged or degenerative somatic cells. Such cells can be multipotent cells or any other type of transplantable cells.

hNT-ESCs derived from the hSCNTs described herein are useful for the generation of cells of desired cell types. In some embodiments, the hNT-ESCs derived from the hSCNTs are used to derive mesenchymal, neural, and/or hematopoietic stem cells. In other embodiments, the hNT-ESCs derived from the hSCNTs are used to generate cells, including but not limited to, pancreatic, liver, bone, epithelial, endothelial, tendons, cartilage, and muscle cells, and their progenitor cells. Thus, transplantable hNT-ESCs cells derived from the hSCNTs can be administered to an individual in need of one or more cell types to treat a disease, disorder, or condition. Examples of diseases, disorders, or conditions that may be treated or prevented include neurological, endocrine, structural, skeletal, vascular, urinary, digestive, integumentary, blood, immune, auto-immune, inflammatory, kidney, bladder, cardiovascular, cancer, circulatory, hematopoietic, metabolic, reproductive and muscular diseases, disorders and conditions. In some embodiments, a hematopoietic stem cell derived from hNT-ESCs derived from the hSCNTs is used to treat cancer. In some embodiments, these cells are used for reconstructive applications, such as for repairing or replacing tissues or organs.

The hNT-ESCs derived from the hSCNTs described herein can be used to generate multipotent stem cells or transplantable cells. In one example, the transplantable cells are mesenchymal stem cells. Mesenchymal stem cells give rise to a very large number of distinct tissues (Caplan, J. Orth. Res 641-650, 1991). Mesenchymal stem cells capable of differentiating into bone, muscles, tendons, adipose tissue, stromal cells and cartilage have also been isolated from marrow (Caplan, J. Orth. Res. 641-650, 1991). U.S. Pat. No. 5,226,914 describes an exemplary method for isolating mesenchymal stem cells from bone marrow. In other examples, epithelial progenitor cells or keratinocytes can be generated for use in treating conditions of the skin and the lining of the gut (Rheinwald, Meth. Cell Bio. 21A:229, 1980). The cells can also be used to produce liver precursor cells (see PCT Publication No. WO 94/08598) or kidney precursor cells (see Karp et al., Dev. Biol. 91:5286-5290, 1994). The cells can also be used to produce inner ear precursor cells (see Li et al., TRENDS Mol. Med. 10: 309, 2004).

The transplantable cells derived from hNT-ESCs derived from the hSCNTs can also be neuronal cells. The volume of a cell suspension, such as a neuronal cell suspension, administered to a subject will vary depending on the site of implantation, treatment goal and amount of cells in solution. Typically the amount of cells administered to a subject will be a therapeutically effective amount. For example, where the treatment is for Parkinson's disease, transplantation of a therapeutically effective amount of cells will typically produce a reduction in the amount and/or severity of the symptoms associated with that disorder, e.g., rigidity, akinesia and gait disorder. In one example, a severe Parkinson's patient needs at least about 100,000 surviving dopamine cells per grafted site to have a substantial beneficial effect from the transplantation. As cell survival is low in brain tissue transplantation in general (5-10%) at least 1 million cells are administered, such as from about 1 million to about 4 million dopaminergic neurons are transplanted. In one embodiment, the cells are administered to the subject's brain. The cells can be implanted within the parenchyma of the brain, in the space containing cerebrospinal fluids, such as the sub-arachnoid space or ventricles, or extaneurally. Thus, in one example, the cells are transplanted to regions of the subject which are not within the central nervous system or peripheral nervous system, such as the celiac ganglion or sciatic nerve. In another embodiment, the cells are transplanted into the central nervous system, which includes all structures within the dura mater. Injections of neuronal cells can generally be made with a sterilized syringe having an 18-21 gauge needle. Although the exact size needle will depend on the species being treated, the needle should not be bigger than 1 mm diameter in any species. Those of skill in the art are familiar with techniques for administering cells to the brain of a subject.

Generally a therapeutically effective amount of hNT-ESCs derived from the hSCNTs is administered to an individual. The cells can be administered in a pharmaceutical carrier. The pharmaceutically acceptable carriers of use are conventional. For example, Remington's Pharmaceutical Sciences, by E. W. Martin, Mack Publishing Co., Easton, Pa., 15th Edition (1975), describes compositions and formulations suitable for pharmaceutical delivery of the cells herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The individual can be any subject of interest. Suitable subjects include those subjects that would benefit from proliferation of cells derived from stem cells or precursor cells. In one embodiment, the individual is in need of proliferation of neuronal precursor cells and/or glial precursor cells. For example, the individual can have a neurodegenerative disorder or have had an ischemic event, such as a stroke. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis. Suitable individual also include those subjects that are aged, such as individuals who are at least about 65, at least about 70, at least about 75, at least about 80 or at least about 85 years of age. In additional examples, the individual can have a spinal cord injury, Batten's disease or spina bifida. In further examples, the individual can have hearing loss, such as a subject who is deaf, or can be in need of the proliferation of stem cells from the inner ear to prevent hearing loss.

In some embodiments, hNT-ESCs derived from the hSCNTs produced using the methods disclosed herein are capable of contributing to the germ line. Thus, somatic cells from a subject of interest can be used to produce ES cells which subsequently can be differentiated into oocytes or sperm. These oocytes or sperm can then be used for fertilization, allowing an infertile subject to produce children that are genetically related to the subject. Such a method is useful for female subjects that have a mitochondrial disease, where the female with such a disease is the source for the human donor somatic cell for the method, thereby enabling the production of NT-ESCs from the hSCNT, which can be differentiated into an oocyte, which can be used in producing children by the female without the defects in the mtDNA. In addition, ES cell-derived eggs are of use in research. For example, these eggs can in turn be used to make human SCNT-derived ES cells. This availability of these oocytes can reduce the use of donated human eggs for research.

hNT-ESCs derived from the hSCNTs can also be used to generate extra embryonic cells, such as trophectoderm, that are of use in cell culture. In one embodiment, the use of autologous cells (e.g., trophectoderm) as feeder cells can be helpful to generate stem cells that in turn have the capacity to differentiate into differentiated organ-specific cells. In other embodiments, the use of allogeneic feeder cells, obtained by using culturing totipotent stem cells in such a manner to allow the generation of such feeder layer component, is useful to avoid xeno-contamination and thus, allow for easier FDA approval of the differentiated cells cultured thereupon for therapeutic purposes.

Cells produced by the methods disclosed herein, such as hNT-ESCs derived from the hSCNTs are also of use for testing agents of interest, such as to determine if an agent affects differentiation or cell proliferation. For example, hNT-ESCs derived from the hSCNTs are contacted with the agent, and the ability of the cells to differentiate or proliferate is assessed in the presence and the absence of the agent. Thus, hNT-ESCs derived from the hSCNTs produced by the methods disclosed herein can also be used in to screen pharmaceutical agents to select for agents that affect specific human cell types, such as agents that affect neuronal cells. hNT-ESCs derived from the hSCNTs produced by the methods disclosed herein can also be used to screen agent to select those that affect differentiation. The test compound can be any compound of interest, including chemical compounds, small molecules, polypeptides or other biological agents (for example antibodies or cytokines). In several examples, a panel of potential agents are screened, such as a panel of cytokines or growth factors is screened.

Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991), a peptide library (U.S. Pat. No. 5,264,563); a peptidomimetic library (Blondelle et al., Trends Anal Chem. 14:83-92, 1995); a nucleic acid library (O'Connell et al., Proc. Natl Acad. Sci., USA 93:5883-5887, 1996; Tuerk and Gold, Science 249:505-510, 1990; Gold et al., Ann. Rev. Biochem. 64:763-797, 1995); an oligosaccharide library (York et al., Carb. Res. 285:99-128, 1996; Liang et al., Science 274:1520-1522, 1996; Ding et al., Adv. Expt. Med. Biol. 376:261-269, 1995); a lipoprotein library (de Kruif et al., FEBS Lett. 3 99:23 2-23 6, 1996); a glycoprotein or glycolipid library (Karaoglu et al., J. Cell Biol. 130.567-577, 1995); or a chemical library containing, for example, drugs or other pharmaceutical agents (Gordon et al., J. Med. Chem. 37.1385-1401, 1994; Ecker and Crooke, BioTechnology 13:351-360, 1995). Polynucleotides can be particularly useful as agents that can alter a function pluripotent or totipotent cells because nucleic acid molecules having binding specificity for cellular targets, including cellular polypeptides, exist naturally, and because synthetic molecules having such specificity can be readily prepared and identified (see, for example, U.S. Pat. No. 5,750,342).

In one embodiment, for a high throughput format, hNT-ESCs derived from the hSCNTs or MPSCs produced by the methods disclosed herein can be introduced into wells of a multiwell plate or of a glass slide or microchip, and can be contacted with the test agent. Generally, the cells are organized in an array, particularly an addressable array, such that robotics conveniently can be used for manipulating the cells and solutions and for monitoring the cells, particularly with respect to the function being examined. An advantage of using a high throughput format is that a number of test agents can be examined in parallel, and, if desired, control reactions also can be run under identical conditions as the test conditions. As such, the methods disclosed herein provide a means to screen one, a few, or a large number of test agents in order to identify an agent that can alter a function of the hNT-ESCs derived from the hSCNTs, for example, an agent that induces the hNT-ESCs to differentiate into a desired cell type, or that prevents spontaneous differentiation, for example, by maintaining a high level of expression of regulatory molecules.

The hNT-ESCs are contacted with test compounds sufficient for the compound to interact with the cell. When the compound binds a discrete receptor, the cells are contacted for a sufficient time for the agent to bind its receptor. In some embodiments, the cells are incubated with the test compound for an amount of time sufficient to affect phosphorylation of a substrate. In some embodiments, hNT-ESCs are treated in vitro with test compounds at 37° C. in a 5% CO₂ humidified atmosphere. Following treatment with test compounds, cells are washed with Ca²+ and Mg²+ free PBS and total protein is extracted as described (Haldar et al., Cell Death Diff 1:109-115, 1994; Haldar et al., Nature 342:195-198, 1989; Haldar et al., Cancer Res. 54:2095-2097, 1994). In additional embodiments, serial dilutions of test compound are used.

### Compositions and Kits.

Another aspect of the present invention relates to a population of hNT-ESCs obtained from a SCNT produced by the methods as disclosed herein. In some embodiments, the hNT-ESCs are human ntESCs, for example patient-specific hNT-ESCs, and/or patient-specific isogenic hNT-ESCs. In some embodiments, the hNT-ESCs are present in culture medium, such as a culture medium which maintains the hNT-ESCs in a totipotent or pluripotent state. In some embodiments, the culture medium is a medium suitable for cryopreservation. In some embodiments, the population of hNT-ESCs are cryopreserved. Cryogenic preservation is useful, for example, to store the hNT-ESCs for future use, e.g., for therapeutic use, or for other uses, e.g., research use. The hNT-ESCs may be amplified and a portion of the amplified hNT-ESCs may be used and another portion may be cryogenically preserved. The ability to amplify and preserve hNT-ESCs allows considerable flexibility, for example, production of multiple patient-specific human hNT-ESCs as well as the choice of donor somatic cells for use in the SCNT procedure. For example, cells from a histocompatible donor, may be amplified and used in more than one recipient. Cryogenic preservation of hNT-ESCs can be provided by a tissue bank. hNT-ESCs may be cryopreserved along with histocompatibility data. hNT-ESCs produced using the methods as disclosed herein can be cryopreserved according to routine procedures. For example, cryopreservation can be carried out on from about one to ten million cells in “freeze” medium which can include a suitable proliferation medium, 10% BSA and 7.5% dimethylsulfoxide. hNT-ESCs are centrifuged. Growth medium is aspirated and replaced with freeze culture medium. hNT-ESCs are resuspended as spheres. Cells are slowly frozen, by, e.g., placing in a container at −80° C. Frozen hNT-ESCss are thawed by swirling in a 37° C. bath, resuspended in fresh stem cell medium, and grown as described above.

In some embodiments, the hNT-ESCs are generated from a SCNT embryo that was generated from injection of nuclear genetic material from a donor somatic cell into the cytoplasm of a recipient oocyte, where the recipient oocyte comprises mtDNA from a third donor subject.

The present invention also relates to a hSCNT embryo produced by the methods as disclosed herein. In some embodiments, the hSCNT embryo is a human embryo. In some embodiments, the human SCNT embryo is genetically modified, e.g., at least one transgene was modified (e.g., introduced or deleted or changed) in the genetic material of the donor nucleus prior to the SCNT procedure (i.e., prior to collecting the donor nucleus and fusing with the cytoplasm of the recipient oocyte). In some embodiments, the hSCNT embryo comprises nuclear DNA from the human donor somatic cell, cytoplasm from the human recipient oocyte, and mtDNA from a third human donor subject.

Another aspect of the present invention relates to a composition comprising at least one of at one of; a human SCNT embryo or a blastocyst thereof, or a recipient human oocyte (nucleated or enucleated) and at least one of; (i) an agent which increases the expression or activity of the KDM4 family of histone demethylases; or (ii) an agent which inhibits an H3K9 methyltransferase.

In another embodiment, this invention provides kits for the practice of the methods of this invention. Another aspect of the present invention relates to a kit, including one or more containers comprising (i) an agent which increases the expression or activity of the KDM4 family of histone demethylases and/or an agent which inhibits an H3K9 methyltransferase, and (ii) a human oocyte. The kit may optionally comprise culture medium for the recipient oocyte, and/or the SCNT embryo, as well as one or more reagents for activation (e.g., fusion) of the donor nuclear genetic material with the cytoplasm of the recipient oocyte. In some embodiments, the human oocyte is an enucleated oocyte. In some embodiments, the human oocyte is not enucleated. In some embodiments, the human oocyte is frozen and/or present in a cryopreservation freezing medium. In some embodiments, the human oocyte is obtained from a donor female subject that has a mitochondrial disease or has a mutation or abnormality in a mtDNA. In some embodiments, the oocyte is obtained from a donor female subject that does not has a mitochondrial disease, or does not have a mutation in mtDNA. In some embodiments, the oocyte comprises mtDNA from a third subject.

The kit may also optionally include appropriate systems (e.g. opaque containers) or stabilizers (e.g. antioxidants) to prevent degradation of the agent which increases the expression or activity of the KDM4 family of histone demethylases and/or the agent which inhibits an H3K9 methyltransferase by light or other adverse conditions.

The kit may optionally include instructional materials containing directions (i.e., protocols) for performing hSCNT procedure (e.g., for enucleating an oocyte, and/or injecting the nuclear genetic material of the donor somatic cell into the recipient oocyte and/or fusion/activation, and/or culturing the hSCNT embryo), as well as instructions of contacting at least one of a donor somatic cell and/or recipient oocyte, and/or hSCNT embryo with at least one of an agent which increases the expression or activity of the KDM4 family of histone demethylases and/or an agent which inhibits an H3K9 methyltransferase.

In order that the invention herein described may be fully understood, the following detailed description is set forth.

The present invention can further be defined in any of the following numbered paragraphs:
1. A method for increasing the efficiency of human somatic nuclear transfer (hSCNT) comprising contacting a hybrid oocyte with an agent which increases expression of a member of the KDM4 family of histone demethylases, wherein the hybrid oocyte is an enucleated human oocyte comprising the genetic material of a human somatic cell.
2. The method of paragraph 1, wherein the contacting occurs after activation or fusion of the hybrid oocyte, but before human zygotic genome activation (ZGA) begins.
3. A method for increasing the efficiency of human somatic cell nuclear transfer (SCNT) comprising at least one of:
   (i) contacting a donor human somatic cell or a recipient human oocyte with at least one agent which decreases H3K9me3 methylation in the donor human somatic cell or the recipient human oocyte, wherein the recipient human oocyte is a nucleated or enucleated oocyte; enucleating the recipient human oocyte if the human oocyte is nucleated; transferring the nuclei from the donor human somatic cell to the enucleated oocyte to form a hybrid oocyte; and activating the hybrid oocyte to form a human SCNT embryo; or
   (ii) contacting a hybrid oocyte with at least one agent which decreases H3K9me3 methylation in the hybrid oocyte, where the hybrid oocyte is an enucleated human oocyte comprising the genetic material of a human somatic cell, and activating the hybrid oocyte to form a human SCNT embryo; or
   (iii) contacting a human SCNT embryo after activation with at least one agent which decreases H3K9me3 methylation in the human SCNT embryo, wherein the SCNT embryo is generated from the fusion of an enucleated human oocyte with the genetic material of a human somatic cell;
   wherein the decrease of H3K9me3 methylation in any one of the donor human somatic cell, recipient human oocyte, hybrid oocyte or the human SCNT embryo increases the efficiency of the SCNT.
4. A method for producing a human nuclear transfer embryonic stem cell (hNT-ESC), comprising;
   a. at least one of: (i) contacting a donor human somatic cell or a recipient human oocyte with at least one agent which decreases H3K9me3 methylation in the donor human somatic cell or the recipient human oocyte; wherein the recipient human oocyte is a nucleated or enucleated oocyte; enucleating the recipient human oocyte if the human oocyte is nucleated; transferring the nuclei from the donor human somatic cell to the enucleated oocyte to form a hybrid oocyte; and activating the hybrid oocyte to form a human SCNT embryo; or
   (ii) contacting a hybrid oocyte with at least one agent which decreases H3K9me3 methylation in the hybrid oocyte, where the hybrid oocyte is an enucleated human oocyte comprising the genetic material of a human somatic cell, and activating the hybrid oocyte to form a human SCNT embryo; or
   (iii) contacting a human SCNT embryo after activation with at least one agent which decreases H3K9me3 methylation in the SCNT embryo, wherein the SCNT embryo is generated from the fusion of an enucleated human oocyte with the genetic material of a human somatic cell; b. incubating the SCNT embryo for a sufficient amount of time to form a blastocyst; and collecting at least one blastomere from the blastocyst and culturing the at least one blastomere to form at least one human NT-ESC.
5. A method for producing a human somatic cell nuclear transfer (SCNT) embryo, comprising:
   contacting at least one of; a donor human somatic cell, a recipient human oocyte or a human somatic cell nuclear transfer (SCNT) embryo with at least one agent which decreases H3K9me3 methylation in the donor human somatic cell, the recipient human oocyte or the human SCNT embryo, wherein the recipient human oocyte is a nucleated or enucleated oocyte;
   enucleating the recipient human oocyte if the human oocyte is nucleated;
   transferring the nuclei from the donor human somatic cell to the enucleated oocyte to form a hybrid oocyte; activating the hybrid oocyte and
   incubating the hybrid oocyte for a sufficient amount of time to form the human SCNT embryo.
6. The method of any of paragraphs 2 to 5, wherein in agent which decreases H3K9me3 methylation is an agent increases expression of a member of the human KDM4 family of histone demethylases.
7. The method of paragraph 6, wherein the agent increases the expression or activity of the human KDM4 (JMJD2) family of histone demethylases.
8. The method of any of paragraphs 1 to 7, wherein the agent increases the expression or activity of at least one of: KDM4A (JMJD2A), KDM4B (JMJD2B), KDM4C (JMJD2C), KDM4D (JMJD4D) or KDM4E (JMJD2E).
9. The method of any of paragraphs 1 to 8, wherein the agent increases the expression or activity of KDM4A (JMJD2A)
10. The method of any of paragraphs 1 to 9, wherein the agent comprises a nucleic acid sequence corresponding to SEQ ID NO: 1-4 or SEQ ID NO: 45, or a biologically active fragment thereof which increases the efficiency of SCNT to a similar or greater extent as compared to the corresponding sequence of SEQ ID NO: 1-4 or SEQ ID NO: 45.
11. The method of paragraph 6, wherein the agent comprises a nucleic acid sequence corresponding to SEQ ID NO: 1, or a biologically active fragment thereof which increases the efficiency of SCNT to a similar or greater extent as compared to the nucleic acid sequence of SEQ ID NO: 1.
12. The method of any of paragraphs 1 to 11, wherein the agent is an inhibitor of a H3K9 methyltransferase.
13. The method of paragraph 12, wherein the H3K9 methyltransferase is SUV39h1 or SUV39h2.
14. The method of paragraph 12, wherein the H3K9 methyltransferase is SETDB1.
15. The method of paragraph 12, wherein two or more of SUV39h1, SUV39h2 and SETDB1 are inhibited.
16. The method of paragraph 12, wherein the agent which inhibits H3K9 methyltransferase is selected from the group consisting of; an RNAi agent, CRISPR/Cas9, CRISPR/Cpfl oligonucleotide, neutralizing antibody or antibody fragment, aptamer, small molecule, peptide inhibitor, protein inhibitor, avidimir, and functional fragments or derivatives thereof.
17. The method of paragraph 16, wherein the RNAi agent is a siRNA or shRNA molecule.
18. The method of any of paragraphs 1 to 17, wherein the agent comprises a nucleic acid inhibitor to inhibit the expression of any of SEQ ID NOS: 14-16, 47, 49, 51, 52 or 53.
19. The method of paragraph 17, wherein the RNAi agent hybridizes to at least a portion of SEQ ID NOS: 14-16, 47, 49, 51, 52 or 53.
20. The method of paragraph 17, wherein the RNAi agent comprises any one of, or a combination of nucleic acids of SEQ ID NO: 7, 8 or SEQ ID NO: 18 or 19 or a fragment of at least 10 consecutive nucleic acid thereof, or a homologue having a sequence that is at least 80% identical to SEQ ID NO: 7, 8 or SEQ ID NO: 18 or 19.
21. The method of any of paragraphs 1 to 20, wherein the recipient human oocyte is an enucleated human oocyte.
22. The method of any of paragraphs 1 to 20, wherein the human SCNT embryo is selected from any of; a 1-cell stage SCNT embryo, a SCNT embryo 5 hours post activation (5 hpa), a SCNT embryo between 10-12 hours post activation (10-12 hpa), a SCNT embryo 20-28 hours post activation (20-28 hpa), a 2-cell stage SCNT embryo.
23. The method of any of paragraphs 1 to 22, wherein the agent contacts a recipient human oocyte or enucleated human oocyte prior to nuclear transfer.
24. The method of any of paragraphs 1 to 22, wherein the agent contacts the human SCNT embryo prior to, or at about 5 hours post activation, or when the SCNT embryo is at the 1-cell stage.
25. The method of any of paragraphs 1 to 22, wherein the agent contacts the human SCNT embryo after 5 hours post activation (5 hpa), or 12 hours post activation (hpa), or 20 hours post activation (20 hpa), or when the SCNT embryo is at the 2-cell stage, or any time between 5 hpa and 28 hpa.
26. The method of any of paragraphs 1 to 22, wherein the contacting the recipient human oocyte or hybrid oocyte, or human SCNT embryo with the agent comprises injecting the agent into the nuclei or cytoplasm of the recipient human oocyte or hybrid oocyte, or human SCNT embryo.
27. The method of any of paragraphs 1 to 26, wherein the agent increases the expression or activity of the KDM4 family of histone demethylases.
28. The method of any of paragraphs 1 to 22, wherein the agent contacts the cytoplasm or nuclei of the donor human somatic cell prior to removal of the nuclei for injection into an enucleated human oocyte.
29. The method of any of paragraph 28, wherein the donor human somatic cell is contacted at least 24 hours prior to, or for at least 1 day prior to, injection of the nuclei of the donor human somatic cell into an enucleated human oocyte.
30. The method of any of paragraph 28, wherein the agent contacts the donor human somatic cell for at least 24 hours, or at least 48 hours, or at least 3 days, prior to injection of the nuclei of the donor human somatic cell into an enucleated human oocyte.
31. The method of any of paragraphs 28 to 30, wherein the agent inhibits H3K9 methyltransferase.
32. The method of any of paragraphs 28 to 30, wherein the H3K9 methyltransferase is SUV39h1 or SUV39h2, or SUV39h1 and SUV39h2 (SUV39h1/2).
33. The method of any of paragraphs 1 to 32, wherein the donor human somatic cell is a terminally differentiated somatic cell.
34. The method of any of paragraphs 1 to 33, wherein the donor human somatic cell is not an embryonic stem cell, or an induced pluripotent stem (iPS) cell, or a fetal cell, or an embryonic cell.
35. The method of any of paragraphs 1 to 34, wherein the donor human somatic cell is selected from the group consisting of cumulus cell, epithelial cell, fibroblast, neural cell, keratinocyte, hematopoietic cell, melanocyte, chondrocyte, erythrocyte, macrophage, monocyte, muscle cell, B lymphocyte, T lymphocyte, embryonic stem cell, embryonic germ cell, fetal cell, placenta cell, and adult cell.
36. The method of any of paragraphs 1 to 35, wherein the donor human somatic cell is a fibroblast or a cumulus cell.
37. The method of any of paragraphs 1 or 36, wherein the agent contacts the nuclei of the donor human somatic cell to removal of the nuclei from the donor human somatic cell for injection into an enucleated recipient human oocyte.
38. The method of any of paragraphs 1 to 37, wherein the method results in an at least a 10% increase in efficiency of hSCNT to blastocyst stage as compared to hSCNT performed in the absence of an agent which decreases H3K9me3 methylation.
39. The method of any of paragraphs 1 to 38, wherein the method results in a 10-20% increase in efficiency of hSCNT as compared to hSCNT performed in the absence of an agent which decreases H3K9me3 methylation.
40. The method of any of paragraphs 1 to 39, wherein the method results in a greater than 20% increase in efficiency of hSCNT as compared to hSCNT performed in the absence of an agent which decreases H3K9me3 methylation.
41. The method of any of paragraphs 38 to 40, wherein the increase in SCNT efficiency is an increase in the development of the human SCNT embryo to blastocyst stage.
42. The method of any of paragraphs 38 to 40, wherein the increase in SCNT efficiency is an increase in the derivation of human SCNT embryo-derived embryonic stem cells (hNT-ESCs).
43. The method of any of paragraphs 1 to 42, wherein the donor human somatic cell is a genetically modified donor human cell.
44. The method of paragraph 5, further comprising in vitro culturing the human SCNT embryo to form a human blastocyst.
45. The method of paragraph 44, wherein the human SCNT embryo is at least a 4-celled human SCNT embryo.
46. The method of paragraph 44, wherein the human SCNT embryo is at least a 4-celled SCNT embryo.
47. The method of paragraph 44, further comprising isolating a cell from an inner cell mass from the human blastocyst; and culturing the cell from the inner cell mass in an undifferentiated state to form a human embryonic stem (ES) cell.
48. The method of any of paragraphs 1 to 48, wherein any one or more of the donor human somatic cell, recipient human oocyte or human SCNT embryo have been frozen and thawed.
49. A population of human SCNT embryo derived embryonic stem cells (hNT-ESCs) produced from the methods of any of paragraphs 1 to 48.
50. The population of hNT-ESCs of paragraph 49, wherein the hNT-ESCs are genetically modified hNT-ESCs.
51. The population of hNT-ESCs of paragraph 49, wherein the hNT-ESCs are pluripotent stem cells or totipotent stem cells.
52. The population of hNT-ESCs of paragraph 49, wherein the hNT-ESCs are present in a culture medium.
53. The population of hNT-ESCs of paragraph 52, wherein the culture medium maintains the hNT-ESCs in a pluripotent or totipotent state.
54. The population of hNT-ESCs of paragraph 52, wherein the culture medium is a medium suitable for freezing or cryopreservation of the hNT-ESCs.
55. The population of hNT-ESCs of paragraph 54, wherein the population of hNT-ESC are frozen or cryopreserved.
56. A human SCNT embryo produced by the methods of paragraph 1 to 55.
57. The human SCNT embryo of paragraph 56, wherein the human SCNT embryo is genetically modified.
58. The human SCNT embryo of paragraph 56, wherein the human SCNT embryo comprises mitochondrial DNA (mtDNA) that is not from the recipient human oocyte.
59. The human SCNT embryo of paragraph 56, wherein the human SCNT embryo is present in a culture medium.
60. The human SCNT embryo of paragraph 59, wherein the culture medium is a medium suitable for freezing or cryopreservation of the human SCNT.
61. The human SCNT embryo of paragraph 60, wherein the human embryo is frozen or cryopreserved.
62. A composition comprising at least one of; a human SCNT embryo, recipient human oocyte, a human hybrid oocyte or a blastocyst and at least one of;
   a. an agent which increases the expression or activity of the KDM4 family of histone demethylases; or
   b. an agent which inhibits an H3K9 methyltransferase.
63. The composition of paragraph 62, wherein the agent that increases the expression or activity of the KDM4 (JMJD2) family of histone demethylases increases the expression or activity of at least one of: KDM4A (JMJD2A), KDM4B (JMJD2B), KDM4C (JMJD2C), KDM4D (JMJD2D) or KDM4E (JMJD2E).
64. The composition of paragraph 63, wherein the agent increases the expression or activity of KDM4D (JMJD2D) or KDM4A (JMJD2A).
65. The composition of paragraph 64, wherein the agent comprises a nucleic acid corresponding to SEQ ID NO: 1-4 or SEQ ID NO: 45, or a biologically active fragment thereof which increases the efficiency of human SCNT to a similar or greater extent as compared to the corresponding sequence of SEQ ID NO: 1-4 or SEQ ID NO: 45.
66. The composition of paragraph 64, wherein the agent comprises a nucleic acid corresponding to SEQ ID NO: 1, or a biologically active fragment thereof which increases the efficiency of SCNT to a similar or greater extent as compared to the nucleic acid sequence of SEQ ID NO: 1.
67. The composition of paragraph 62, wherein the inhibitor of the H3K9 methyltransferase inhibits at least one or any combination of SUV39h1, SUV39h2, or SETDB1.
68. The composition of paragraph 62, wherein the human SCNT embryo is at 1-cell, 2-cell stage or 4-cell stage human SCNT embryo.
69. The composition of paragraph 62, wherein the recipient human oocyte is an enucleated recipient human oocyte.
70. The composition of paragraph 62, wherein the human SCNT embryo is produced from the injection of the nuclei of a terminally differentiated human somatic cell, or wherein the blastocyst is developed from a human SCNT embryo produced from the injection of the nuclei of a terminally differentiated human somatic cell into an enucleated human oocyte.
71. A kit comprising (i) an agent which increases the expression or activity of the human KDM4 family of histone demethylases and/or an agent which inhibits an H3K9 methyltransferase, and (ii) a human oocyte.
72. The kit of paragraph 92, wherein the human oocyte is an enucleated oocyte.
73. The kit of paragraph 92, wherein the human oocyte is a non-human oocyte.

Unless defined otherwise, all technical and scientific terms used herein have the same meaning as those commonly understood by one of ordinary skill in the art to which this invention belongs. Although methods and materials similar or equivalent to those described herein can be used in the invention or testing of the present invention, suitable methods and materials are described below. The materials, methods and examples are illustrative only, and are not intended to be limiting.

All publications, patents, patent publications and applications and other documents mentioned herein are incorporated by reference in their entirety.

As summarized above, the present invention provides methods for deriving ES cells, ES cell lines, and differentiated cell types from single blastomeres of an early stage embryo without necessarily destroying the embryo. Various features of the method a described in detail below. All of the combinations of the various aspects and embodiments of the invention detailed above and below are contemplated.

### EXAMPLES

The examples presented herein relate to methods and compositions to increase the efficiency of human SCNT by decreasing or removing H3K9me3 by either (i) increasing the expression or activity a member of the human KDM4 family of histone demethylases, e.g., KDM4A and/or (ii) inhibiting any one of the human methyl transferases hSUV39h1 or hSUV39h2 in the human SCNT embryo and/or in the human donor nuclei of a human somatic cell. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### EXPERIMENTAL PROCEDURES

Human SCNT Procedure and KDM4A mRNA Injection

All MII stage human oocytes with distinctive 1st polar bodies were enucleated under an inverted microscope equipped with a Poloscope (Oosight®, Cambridge Research & Instrumentation). The enucleation and nuclear donor cell fusion were carried out in the presence of caffeine (1.25 mM). For enucleation, oocytes were pre-incubated in Global HTF medium with Hepes (Life Global) containing 0.5 μg/ml cytochalasin B and caffeine (1.25 mM) for 5 minutes. Then, the spindle complex was removed using a PIEZO actuator (Primetech, Japan). Dermal fibroblast cells resuspended in a drop containing HVJ-E extract (Cosmo Bio, USA) were inserted into the perivitelline space of the enucleated oocytes. The reconstructed oocytes were kept in the manipulation medium containing caffeine (1.25 mM) until the cell fusion was confirmed, and then the reconstructed oocytes were transferred into Global medium 10% SPS, and incubated for 1-1.5 hours before activation. Activation was carried out by applying electropulses (2×50μs DC pulses, 2.7 kV/cm) in 0.25M d-sorbitol buffer and 6-DMAP (2 mM, 4 hrs) as previously described (Tachibana et al., 2013). The activated embryos were transferred to Global 10% SPS medium supplemented with Trichostatin A (TSA, 10 nM, Sigma) for 12 hrs, then the embryos were transferred to Global 10% FBS without TSA and cultured for up to 7 days in an incubator with atmosphere of 6% CO2/5% O2/89% N2 at 37° C. The culture medium was changed on day 3.

For mRNA injection, the activated SCNT embryos were washed and cultured in Global 10% SPS for 1 hr before the KDM4A mRNA injection. Approximately 10 μl of KDM4A mRNA were injected into the SCNT embryos at 5 hours after activation in Hepes-HTF 10% SPS medium using a PIEZO actuator as described previously (Matoba et al., 2014). More details on donor cell preparation, mRNA preparation, RNA-seq and other procedures can be found in the Supplemental Experimental Procedures.

Identification of Human Reprogramming Resistant Regions

A sliding window (size 20 kb, step size 10 kb) was used to assess the genome-wide expression level of 4-cell and 8-cell human embryos. For each window, the expression level was quantified with normalized RPM (reads per millions of uniquely mapped reads). The significantly activated regions in 8-cell relative to 4-cell IVF embryos were identified with stringent criteria (FC>5, RPM>5 in 8-cell IVF embryos), and the overlapping regions were merged. These activated regions were classed into three groups based on their expression differences in human SCNT and IVF 8-cell embryos.

Mice

B6D2F1/J (BDF1) mice were produced by crossing C57BL/6J females with DBA/2J males, and were used for the collection of both oocyte and somatic nuclear donor for SCNT. All animal experiments were approved by the Institutional Animal Care and Use Committee of Harvard Medical School.

In Vitro Transcription of Human KDM4A mRNA

In vitro transcription was performed as described previously (Matoba et al., 2014). Briefly, full length human KDM4A/JHDM3A cDNA was cloned into a pcDNA3.1 plasmid containing poly(A)83 at the 3′ end of cloning site. The catalytic defective mutant form of KDM4A (H188A) was generated using Prime STAR mutagenesis kit (TAKARA # R045A). mRNA was synthesized using the mMESSAGE mMACHINE T7 Ultra Kit (Life technologies # AM1345). The synthesized mRNA was dissolved in nuclease-free water. The concentration of mRNA was measured by NanoDrop ND-1000 spectrophotometer (NanoDrop Technologies). Aliquots of mRNA were stored at −80° C. until use.

Mouse SCNT and KDM4A mRNA Injection

Mouse somatic cell nuclear transfer was carried out as described previously (Matoba et al., 2014). Briefly, both recipient MII oocytes and donor cumulus cells were collected from adult BDF 1 female mice through superovulation by injecting 7.5 IU of pregnant mare serum gonadotropin (PMSG; Millipore #367222) and 7.5 IU of human chorionic gonadotropin (hCG; Millipore #230734). Fifteen to seventeen hours after the hCG injection, cumulus-oocyte complexes (COCs) were collected from the oviducts and treated briefly with Hepes-buffered potassium simplex-optimized medium (KSOM) containing 300 U/ml bovine testicular hyaluronidase (Calbiochem #385931) to obtain dissociated MII oocytes and cumulus cells. Isolated MII oocytes were enucleated in Hepes-buffered KSOM medium containing 7.5 jig/ml of cytochalasin B (Calbiochem #250233) by using Piezo-driven micromanipulator (Primetech # PMM-150FU). The nuclei of donor cumulus cells were injected into the enucleated oocytes. After 1 h incubation in KSOM, reconstructed SCNT oocytes were activated by incubating in Ca-free KSOM containing 2.5 mM SrCl2 and 5 jig/ml cytochalasin B for 1 h, and further cultured in KSOM with cytochalasin B for 4 h. Activated SCNT embryos were washed 5 hrs after the onset of SrCl2 treatment (hours post activation, hpa) and cultured in KSOM in a humidified atmosphere with 5% CO2 at 37.8° C. The SCNT embryos were injected with ˜10 μl of water (control), 1500 ng/μl wild-type or mutant (H188A) human KDM4A mRNA at 5-6 hpa by using a Piezo-driven micromanipulator. Preimplantation developmental rates were analyzed by Student's T-test.

Preparation of Human Oocytes

The protocol for human oocyte experiments (CHA001) was approved by both the CHA Regenerative Medicine Institute (CHARMI) Stem Cell Research Oversight (SCRO) Committee and the Pearl Institutional Review Board (PIRB). Initial oocyte donor recruitment was performed via web-based advertisement as described previously (Chung et al., 2014). All donors were voluntary participants that were screened on the basis of their reproductive, medical, and psychological health according to the guidelines of the American Society for Reproductive Medicine (ASRM). Oocyte donors were financially reimbursed for their time, effort, loss of wages, travel related expenses, discomfort, and other related expenses associated with the donation processes pursuant to the guidelines established by ASRM.

Ovarian stimulation was carried out as described previously (Chung et al., 2014). Briefly, a combination of human recombinant follicle-stimulating hormone (rFSH, 225-3001U, Merck) and human menopausal gonadotropin (Menopur 751U, Ferring) were used to stimulate ovary for 9-11 days with GnRH antagonist (Ganirelix acetate, Merck) suppression. Lupron 4 mg was used to mimic the LH surge when 1 or 2 follicles reached 18 mm in diameter. All medications were administered through subcutaneous injections. Transvaginal oocyte retrieval was performed approximately 36 hours after the Lupron injection. The collected COCs were denuded with 5080 IU/ml hyaluronidase (Sigma-Aldrich) within 1-2 hours after retrieval. Then, they were kept in Global medium supplemented with 10% serum protein supplement (SPS; Cooper Surgical) (IVF Online) until use.

Donated Human IVF Embryos

The IVF embryos used for this study were obtained from the patients who had the desired number of children after standard IVF procedures, and the remaining embryos were cryopreserved in storage for several years (2-6 years). All donors voluntarily donated their embryos (multicell cleavage stage) for researches by signing an informed consent form. The embryo donation program for the research was approved by CHA Gangnam Medical Center's IRB.

Human Donor Somatic Cell Preparation and Characterization

To prepare human nuclear donor somatic cells, small pieces of abdominal skin (0.5 cm×0.3 cm) were biopsied under local anesthesia and washed 3 times in PBS supplemented with an antibiotic/antimycotic solution (anti-anti 1×, Invitrogen) to remove any possible contaminants. All the somatic cell donors used in this study were AMD patients (AMD subtype: Central Areolar Choroidal Dystrophy). DFB-6 was derived from a 52-year old female. DFB-7 was derived from a 42-year old female. DFB-8 was derived from a 59-year old male.

The procedures for somatic nuclear donor cell preparation are essentially the same as previously described (Chung et al., 2014). Briefly, the skin explant was mechanically minced and treated with collagenase (type I, 200 unit/ml, Worthington-biochem) in DMEM supplemented with 10 μg/ml penicillin-streptomycin solution to dissociate the skin tissue. After incubation overnight, the dissociated cells were collected, washed twice and seeded onto 60-mm culture dishes containing DMEM (Invitrogen, with 10% FBS, 1% non-essential amino acids and 10 μg/mL penicillin-streptomycin) solution at 37° C. and 5% CO2. Once the cells reached 80% confluency, 1/2 of initial outgrowths were cryopreserved, and the remaining cells were kept passaged several times, with cells from each passage being cryopreserved. Frozen cells were subsequently thawed prior to SCNT and cultured in a 4-well dish (Nunc) until they reached confluency. They were then cultured in serum-starved DMEM (0.5% FBS) for 2-3 days to synchronize the cell cycle before use.

Derivation of Human NTK-ESCs from KDM4A-Assisted SCNT Blastocysts

All expanded blastocysts were treated with acid Tyrode solution to remove the zona pellucida, then the entire blastocysts (without removing trophectoderm) were plated onto mitotically-inactivated mouse fibroblasts (MEFs, Global Stem Inc.) in knockout-DMEM supplemented with Knockout Serum Replacement (10% SR, Invitrogen), FBS (10% Hyclone), bFGF (30 ng/ml), human LIF (2000 units/ml, Sigma-Aldrich), and ROCK inhibitor (1 uM, Sigma-Aldrich). The derivation medium was not changed for the next 3 days, then 1/2 medium was replaced with fresh medium without the ROCK inhibitor daily as previously described (Chung et al., 2008). After 3 passages, the amount of FBS was reduced to 2%, replacing it with SR. After 5 passages, the ES cells were cultured in DMEM/F12 supplemented with FGF (8 ng/ml, Invitrogen), SR (18%, Invitrogen), and FBS (2% Hyclone). After the 10 passages, the ES cells were maintained in DMEM/F12 supplemented with FGF (8 ng/ml) and 20% SR.

Preparation of 8-Cell Human Embryos for ZGA Analysis

The SCNT embryos used for ZGA analysis were generated using oocytes donated by a single healthy female (#64) and dermal fibroblast cells from an AMD patient (DFB-8). SCNT and IVF embryos were cultured up to late 8-cell stage, when the compaction of blastomeres is initiated, then they were treated briefly with acid Tyrode solution to remove zona pellucida. To prepare for the 8-cell SCNT embryo, oocytes from a single oocyte donor, and skin fibroblast cells from a single somatic nuclear donor were used. All the procedures are the same as described in the “Human SCNT procedure and KMD4A mRNA injection” section. Only embryos that reached the late 8-cell stage synchronically 74 hours post activation were collected and used for this experiment.

For preparation of the control IVF embryos, several donated early 8-cell stage IVF embryos were thawed and cultured for 5-7 hours to allow them to reach late 8-cell stage before being processed. After removal of the zona pellucida, the denuded embryos were washed 3 times in PBS, loaded into RNAse and DNAse free PCR tubes, spin downed, and snap frozen in liquid nitrogen. Then, they were kept at −80° C. until use. As controls, dermal fibroblast cells of somatic nuclear donors were also prepared. Those fibroblast cells were cultured in a 25 cm² flask in DMEM 10% FBS, and approximately 10,000 cells/donor were collected, snap frozen, and stored at −80° C. until use.

Immunostaining

Mouse 1-cell SCNT embryos, undifferentiated human ESC colonies or differentiated embryoid bodied (EBs) were fixed by 4% paraformaldehyde (PFA) for 20 min at room temperature. After three washes with PBS containing 10 mg/ml BSA (PBS/BSA), the fixed samples were permeabilized for 15 min by incubation with 0.5% Triton-X 100. After blocking in PBS/BSA for 1 h at room temperature, these were incubated in a mixture of primary antibodies at 4° C. overnight. The primary antibodies used are as follows: anti-H3K9me3 (Abcam, ab71604, 1:500), anti-NANOG (Abcam, ab109250, 1:200), anti-OCT-4 (Santa Cruz, sc-8628, 1:100), anti-TRA 160 (Millipore, MAB4360, 1:100), anti-SOX2 (R&D, AF2018, 1:200), anti-SSEA4 (Millipore, MAB4304, 1:100), anti-AFP (Alpha-1-Fetoprotein; Dako A0008, 1:100), anti-BRACHYURY (Abcam ab20680, 1:100), and TUJ1 (B-Tubulin; Covance PRB-435P, rabbit, 1:100). Following three washes, the samples were incubated with secondary antibodies that include donkey anti-goat TRITC (Jackson ImmunoResearch, 705-026-147), donkey anti-mouse 488 (Jackson ImmunoResearch, 715-486-151), donkey anti-goat 649 (Jackson ImmunoResearch, 705-496147), donkey anti-rabbit TRITC (Jackson ImmunoResearch, 711026-152) for 1 h at room temperature. The nuclei were co-stained with DAPI (Vector Laboratories).

In Vitro Differentiation and Teratomas Assays of ESCs

For in vitro differentiation assay, ESCs were culture in low-attachment dishes in ESC medium without bFGF for 1 week until they formed embryoid bodies (EBs). Thereafter, EBs were transferred to four-well dishes (Nunc) coated with matrigel (BD Biosciences) and cultured for an additional week. After washing, blocking and permiabilization in PBS containing 1% BSA and 0.1% Triton-X, EBs were incubated with the primary antibodies overnight. After three washes with PBS containing 1% BSA, EBs were stained with secondary antibody and DAPI for 1h and observed under fluorescent microscopy. For teratoma assay, approximately 1×10⁵ of undifferentiated NTK-ESCs were injected into the testicle of a NOD/SCID mouse. For each NTK-ESC line, at least 3 animals were used. After 12 weeks, teratomas were excised, fixed in PFA, embedded in paraffin, sectioned and then analyzed histologically after staining as described previously (Chung et al., 2014).

Chromosome Analysis

Chromosome analyses for both NTK-ESC lines were performed by a standard protocol as previously described (Chung et al., 2014). Metaphase spreads were stained by GTG (G-bands by trypsin using Giemsa)-banding technique and 20 metaphases were analyzed and karyotyped by two cytogenetics experts. The ideogram was produced by the Ikaros karyotyping system (MetaSystems, Germany).

RNA-Sequencing Analysis

Five 8-cell embryos for each group were directly lysed and used for cDNA synthesis using SMART-Seq v4 Ultra Low Input RNA Kit (Clontech). For MEF donor, 10 ng total RNA was used for cDNA synthesis using SMART-Seq v4 Ultra Low Input RNA Kit. After amplification, the cDNA samples were fragmented using Covaris sonicator M220 to an average size of 150 bp (Covaris). Sequencing libraries were made with the fragmented DNA using NEBNext Ultra DNA Library Prep Kit for Illumina according to manufacturer's instruction (New England Biolabs) with different barcodes. For each RNA-seq analysis of hESCs, 1 μg total RNA was used for mRNA purification. Barcoded RNA-seq libraries were generated using NEBNext Ultra Directional RNA Library Prep Kit for Illumina (New England Biolabs). Single end 50 bp sequencing was performed on a HiSeq 2500 sequencer (Illumina). Sequencing reads were mapped to the human genome (hg 19) with Tophat2. All programs were performed with default settings (unless otherwise specified). At least 22 million uniquely mapped reads were obtained for each sequencing library, and subsequently assembled into transcripts guided by the reference annotation (Refseq gene models) with Cufflinks v2.0.2. Expression level of each gene was quantified with normalized FPKM (fragments per kilobase of exon per million mapped fragments). Statistical analyses were performed with R (available at: “www.r-project.org/”). Independent 2 group Wilcoxon rank sum tests were used to compare distributions using the wilcox.test function in R. Pearson's r coefficient was calculated using the cor function with default parameters. The hierarchical clustering analysis of the global gene expression pattern in different samples was carried out using heatmap.2 function (gplots package) in R.

Analyses of published ChIP-seq and DNA methylation data sets

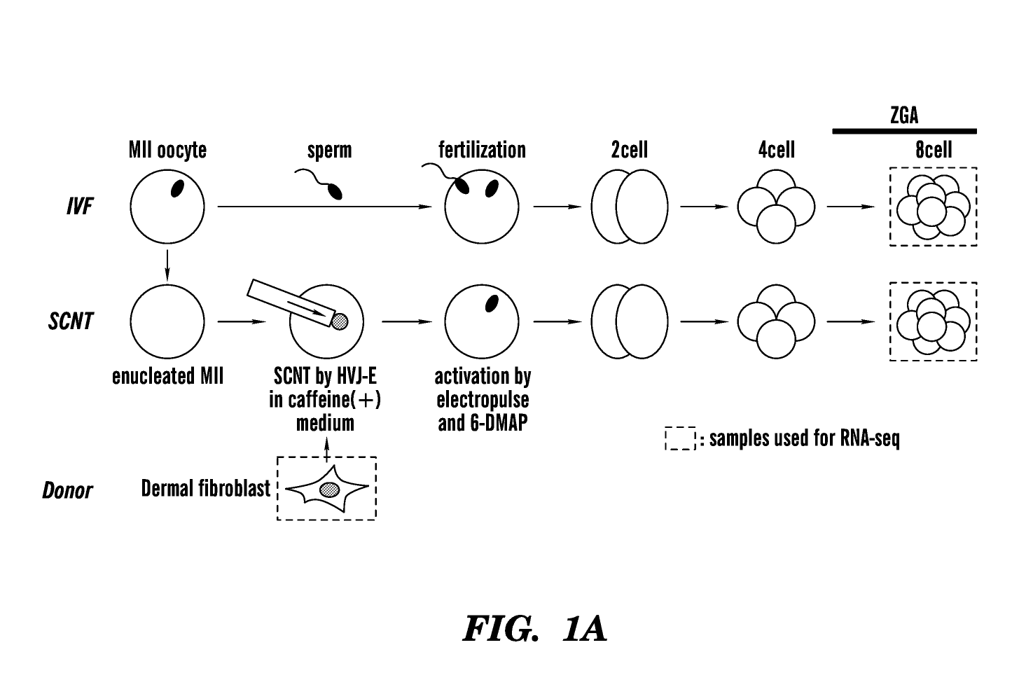

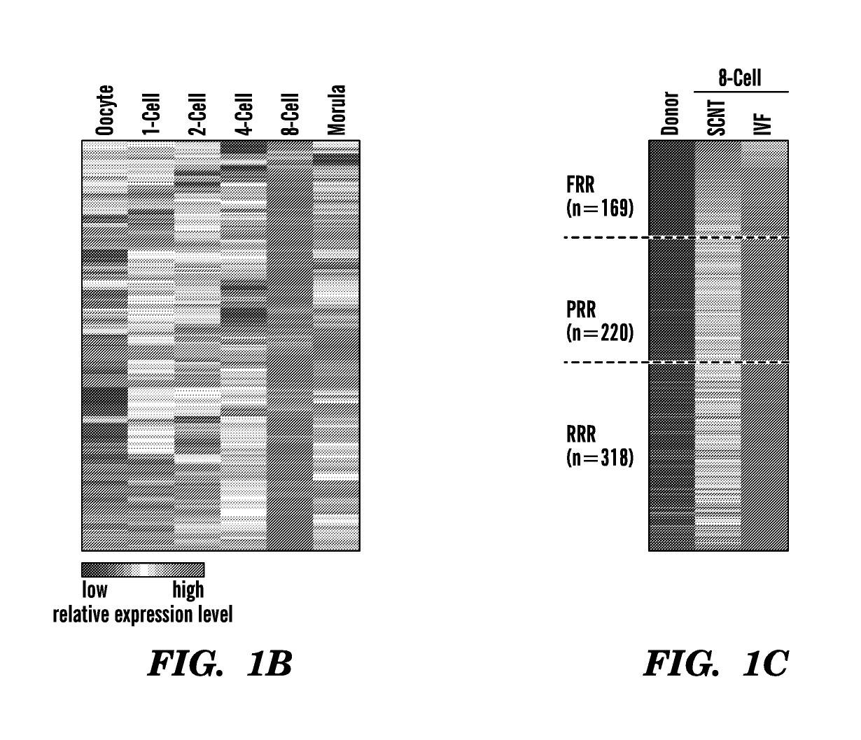

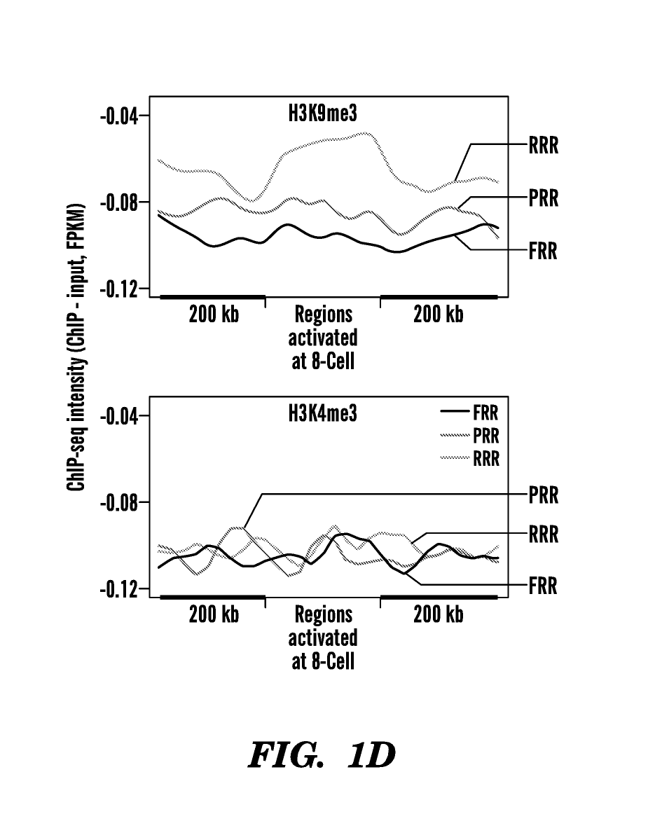

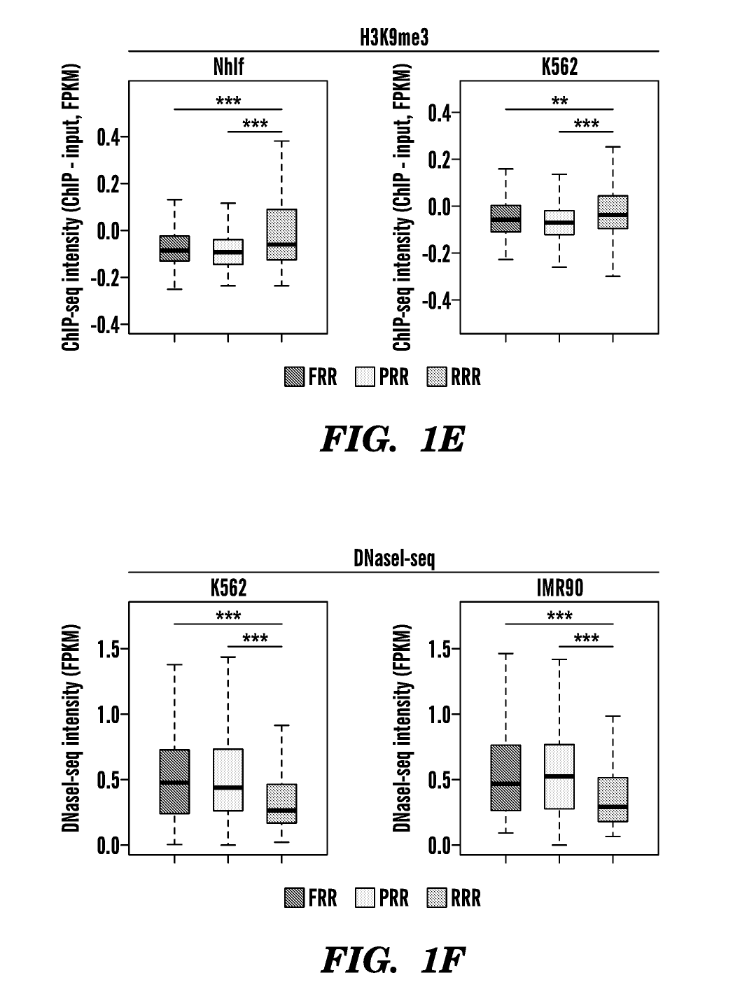

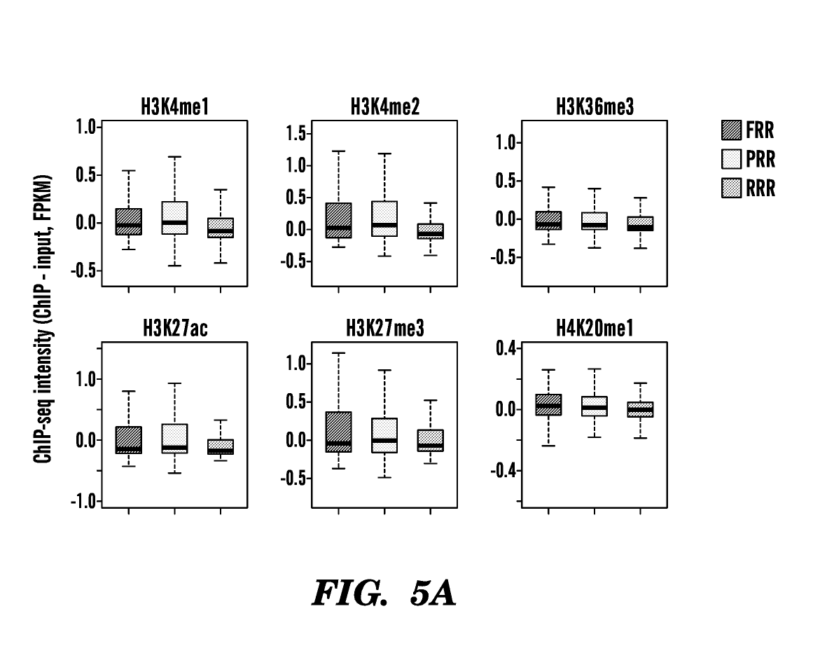

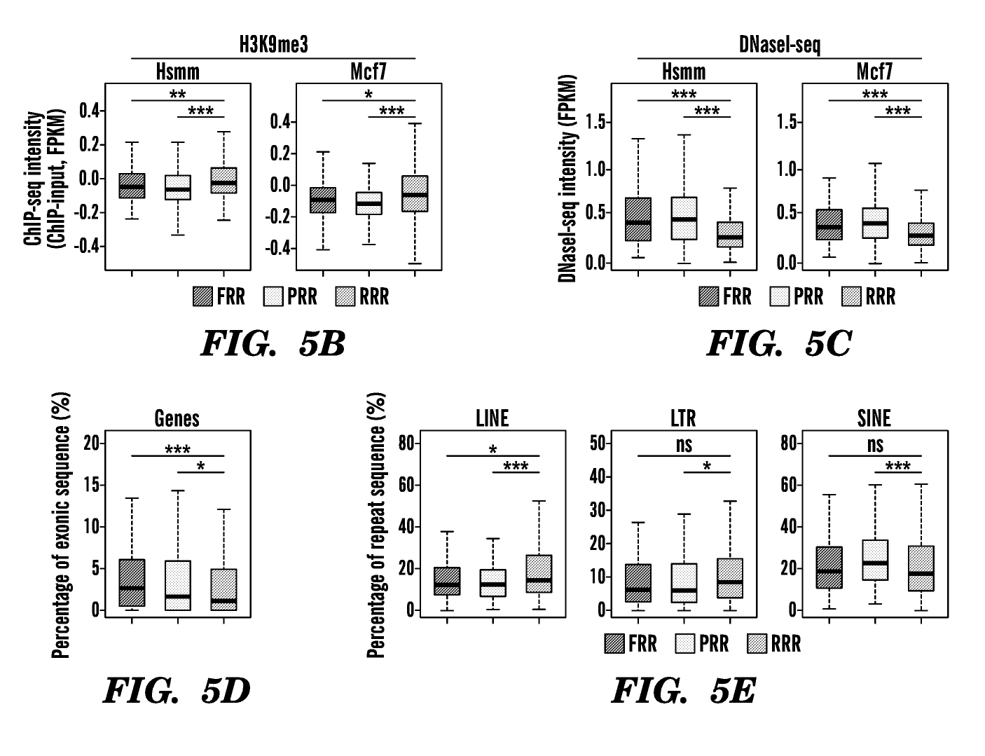

To perform the histone modification enrichment analyses in FIGS. 1, and 5, the inventors used the following published ChIP-seq and DNaseI-seq data sets: H3K9me3, H3K4me1, H3K4me2, H3K4me3, H3K27me3, H3K36me3, H3K27ac and H4K20me1 ChIP-seq in Nhlf fibroblast cells (ENCODE/Broad Histone project), H3K9me3 ChIP-seq in Hsmm and K562 cells (ENCODE/Broad Histone project), H3K9me3 ChIP-seq in Mcf7 cells (ENCODE/Sydh Histone project), DnaseI-seq in IMR90, Hsmm, K562 and Mcf7 cells (ENCODE/OpenChromDnase project). The invenotrs also used whole genome bisulfite sequence data sets of IMR90 cells from Roadmap Epigenomics project for DNA methylation analysis (Roadmap Epigenomics et al., 2015). The processed DNA methylation data in IMR90 was downloaded from world-wide web at “egg2.wustl.edu/roadmap/web_portal/”. ChIP-seq intensity was quantified with normalized FPKM. Position wise coverage of the genome by sequencing reads was determined and visualized as custom tracks in the UCSC genome browser. Independent 2-group Wilcoxon rank sum tests were used to compare the ChIP-seq distributions between each group using the wilcox.test function in R.

### Example 1

Identification of Reprogramming Resistant Regions in 8-Cell Human SCNT Embryos.

Unlike mouse zygotic genome activation (ZGA), which takes place at 2-cell stage, human zygotic genome activation (ZGA) takes place during the late 4-cell to the late 8-cell stages (Niakan et al., 2012) (FIG. 1A). To identify the genomic regions activated during ZGA of normal human IVF embryos, the inventors analyzed published human preimplantation embryo RNA-sequencing (RNA-seq) datasets (Xue et al., 2013) and identified 707 genomic regions ranging 20-160 kb in sizes (Table 5) that were activated at least 5-fold at the 8-cell stage compared to the 4-cell stage (FIG. 1B).

To determine whether ZGA takes place properly in human SCNT, the inventors collected late 8-cell stage embryos (5/group), derived either from SCNT or IVF, and performed RNA-seq (FIG. 1A). In parallel, the inventors also performed RNA-seq of the donor dermal fibroblast cells (DFB-8, see method). Analysis of the 707 genomic regions defined above (FIG. 1B, and Table 5) indicates that the majority of the ZGA regions are activated in the SCNT embryos compared to those in donor fibroblasts (FIG. 1C). However, the level of activation is not comparable to that in IVF embryos (FIG. 1C). Of the 707 genomic regions, 169 were activated at a level comparable to those in IVF embryos (FC<=2, IVF vs SCNT), and were thus termed fully-reprogrammed regions (FRRs) following our previous definition (Matoba et al., 2014). Similarly, 220 regions were partially activated (2<FC<=5) in SCNT compared to IVF embryos and were termed “partially reprogrammed regions” (PRRs). However, the remaining 318 regions (Table 6), termed “reprogramming resistant regions” (RRRs), failed to be activated in SCNT embryos (FC>5). Thus, comparative transcriptome analysis allowed us to identify 318 RRRs that were refractory to transcriptional reprogramming in human 8-cell SCNT embryos.

TABLE 5
Expression levels of transcripts from human ZGA-activated regions (related to Figure 1).
Table 5

| | | | | | Expression level | | Fold Change | |
|---|---|---|---|---|---|---|---|---|
| | | | | | (FPKM)^{#} | | (log2) | |
| Chromosome | start | end | length | name | 4-cell | 8-cell | [8-cell/4-cell] | Category |
| chr19 | 48290001 | 48320000 | 30000 | chr19_48290001_48320000 | 0.15 | 564.65 | 11.14 | RRR |
| chr19 | 48350001 | 48390000 | 40000 | chr19_48350001_48390000 | 0.2 | 585.17 | 10.93 | RRR |
| chr3 | 1.21E+08 | 1.21E+08 | 50000 | chr3_121270001_121320000 | 0.18 | 269.03 | 9.91 | RRR |
| chr1 | 1.61E+08 | 1.61E+08 | 50000 | chr1_160940001_160990000 | 0.14 | 223.72 | 9.87 | RRR |
| chr16 | 49300001 | 49330000 | 30000 | chr16_49300001_49330000 | 0.06 | 146.95 | 9.84 | PRR |
| chr19 | 54120001 | 54160000 | 40000 | chr19_54120001_54160000 | 0.1 | 154.74 | 9.6 | RRR |
| chr3 | 1.09E+08 | 1.09E+08 | 60000 | chr3_109000001_109060000 | 0.05 | 115.21 | 9.59 | RRR |
| chr3 | 42280001 | 42320000 | 40000 | chr3_42280001_42320000 | 0.02 | 85.86 | 9.48 | FRR |
| chr17 | 48340001 | 48370000 | 30000 | chr17_48340001_48370000 | 0.04 | 92.97 | 9.38 | RRR |
| chr19 | 51490001 | 51540000 | 50000 | chr19_51490001_51540000 | 0.11 | 135.93 | 9.34 | RRR |
| chr3 | 1.41E+08 | 1.41E+08 | 30000 | chr3_141230001_141260000 | 0 | 62.02 | 9.28 | RRR |
| chrX | 30220001 | 30280000 | 60000 | chrX_30220001_30280000 | 0 | 57.8 | 9.18 | RRR |
| chr17 | 66260001 | 66280000 | 20000 | chr17_66260001_66280000 | 0.03 | 72.54 | 9.13 | RRR |
| chr7 | 63820001 | 63860000 | 40000 | chr7_63820001_63860000 | 0.01 | 50.06 | 8.83 | RRR |
| chr18 | 19750001 | 19800000 | 50000 | chr18_19750001_19800000 | 0.25 | 150.02 | 8.74 | RRR |
| chr19 | 23430001 | 23470000 | 40000 | chr19_23430001_23470000 | 0.02 | 50.42 | 8.72 | RRR |
| chr7 | 57500001 | 57560000 | 60000 | chr7_57500001_57560000 | 0.08 | 74.89 | 8.7 | RRR |
| chrX | 1.51E+08 | 1.51E+08 | 30000 | chrX_151080001_151110000 | 0.02 | 46.74 | 8.61 | RRR |
| chr3 | 1.27E+08 | 1.27E+08 | 50000 | chr3_126970001_127020000 | 3.12 | 1250.92 | 8.6 | RRR |
| chr13 | 52140001 | 52180000 | 40000 | chr13_52140001_52180000 | 0.01 | 40.9 | 8.54 | RRR |
| chr4 | 63290001 | 63320000 | 30000 | chr4_63290001_63320000 | 0.03 | 47.69 | 8.52 | RRR |
| chr2 | 96270001 | 96310000 | 40000 | chr2_96270001_96310000 | 0.25 | 120.7 | 8.43 | RRR |
| chr9 | 6780001 | 6830000 | 50000 | chr9_6780001_6830000 | 0.04 | 48.12 | 8.43 | RRR |
| chrX | 1.47E+08 | 1.47E+08 | 70000 | chrX_147050001_147120000 | 0.03 | 44.45 | 8.42 | RRR |
| chr19 | 6520001 | 6550000 | 30000 | chr19_6520001_6550000 | 0.01 | 37.55 | 8.42 | RRR |
| chr7 | 63650001 | 63740000 | 90000 | chr7_63650001_63740000 | 0.79 | 302.56 | 8.41 | RRR |
| chr19 | 23840001 | 23880000 | 40000 | chr19_23840001_23880000 | 0.04 | 45.44 | 8.35 | RRR |
| chr6 | 1.07E+08 | 1.07E+08 | 30000 | chr6_107320001_107350000 | 0.03 | 39.91 | 8.27 | RRR |
| chr4 | 180001 | 240000 | 60000 | chr4_180001_240000 | 0 | 30.67 | 8.27 | RRR |
| chr14 | 47110001 | 47140000 | 30000 | chr14_47110001_47140000 | 0.06 | 48.22 | 8.24 | RRR |
| chr1 | 1.52E+08 | 1.52E+08 | 40000 | chr1_152060001_152100000 | 0 | 28.8 | 8.17 | PRR |
| chr2 | 16070001 | 16100000 | 30000 | chr2_16070001_16100000 | 0.01 | 31.3 | 8.16 | PRR |
| chr10 | 61480001 | 61530000 | 50000 | chr10_61480001_61530000 | 0 | 26.96 | 8.08 | RRR |
| chr12 | 65550001 | 65580000 | 30000 | chr12_65550001_65580000 | 0.01 | 29.21 | 8.06 | PRR |
| chr2 | 96100001 | 96140000 | 40000 | chr2_96100001_96140000 | 0.37 | 118.53 | 7.98 | RRR |
| chr2 | 1.79E+08 | 1.79E+08 | 30000 | chr2_178690001_178720000 | 0.04 | 35.14 | 7.98 | RRR |
| chr13 | 52620001 | 52650000 | 30000 | chr13_52620001_52650000 | 0.42 | 130.18 | 7.97 | RRR |
| chr17 | 8080001 | 8100000 | 20000 | chr17_8080001_8100000 | 0 | 24.89 | 7.97 | PRR |
| chr4 | 99870001 | 99910000 | 40000 | chr4_99870001_99910000 | 0.03 | 31.66 | 7.93 | RRR |
| chr19 | 30230001 | 30270000 | 40000 | chr19_30230001_30270000 | 0 | 23.77 | 7.9 | PRR |
| chr6 | 74270001 | 74300000 | 30000 | chr6_74270001_74300000 | 0.12 | 50.88 | 7.86 | RRR |
| chr3 | 1.36E+08 | 1.36E+08 | 30000 | chr3_136440001_136470000 | 0.02 | 27.38 | 7.84 | RRR |
| chr4 | 1.4E+08 | 1.4E+08 | 30000 | chr4_140040001_140070000 | 0 | 22.12 | 7.8 | RRR |
| chr4 | 48280001 | 48310000 | 30000 | chr4_48280001_48310000 | 0 | 21.42 | 7.75 | RRR |
| chr8 | 67840001 | 67880000 | 40000 | chr8_67840001_67880000 | 0.06 | 34.03 | 7.74 | PRR |
| chrX | 1.18E+08 | 1.18E+08 | 40000 | chrX_118190001_118230000 | 0.02 | 25.62 | 7.74 | FRR |
| chr12 | 15040001 | 15070000 | 30000 | chr12_15040001_15070000 | 0 | 21.11 | 7.73 | PRR |
| chr19 | 18110001 | 18140000 | 30000 | chr19_18110001_18140000 | 0.92 | 211.76 | 7.7 | RRR |
| chrX | 37880001 | 37920000 | 40000 | chrX_37880001_37920000 | 0.06 | 32.35 | 7.66 | RRR |
| chr6 | 86360001 | 86380000 | 20000 | chr6_86360001_86380000 | 0.02 | 23.95 | 7.65 | RRR |
| chrX | 47260001 | 47290000 | 30000 | chrX_47260001_47290000 | 0.06 | 31.29 | 7.62 | RRR |
| chr15 | 85910001 | 85970000 | 60000 | chr15_85910001_85970000 | 0.01 | 20.81 | 7.57 | PRR |
| chrX | 40680001 | 40710000 | 30000 | chrX_40680001_40710000 | 0.01 | 20.21 | 7.53 | RRR |
| chr16 | 29470001 | 29490000 | 20000 | chr16_29470001_29490000 | 0 | 18.15 | 7.51 | RRR |
| chr12 | 49140001 | 49160000 | 20000 | chr12_49140001_49160000 | 0 | 17.88 | 7.49 | RRR |
| chr8 | 1.26E+08 | 1.26E+08 | 40000 | chr8_126430001_126470000 | 0.03 | 23.06 | 7.48 | RRR |
| chr10 | 61400001 | 61430000 | 30000 | chr10_61400001_61430000 | 0.01 | 19.38 | 7.47 | RRR |
| chr17 | 1120001 | 1150000 | 30000 | chr17_1120001_1150000 | 0.01 | 19.22 | 7.46 | RRR |
| chr2 | 98240001 | 98260000 | 20000 | chr2_98240001_98260000 | 0.25 | 60.61 | 7.44 | RRR |
| chr1 | 13460001 | 13540000 | 80000 | chr1_13460001_13540000 | 2.23 | 390.55 | 7.39 | RRR |
| chr12 | 14420001 | 14450000 | 30000 | chr12_14420001_14450000 | 0.09 | 31.46 | 7.38 | RRR |
| chr7 | 1.52E+08 | 1.52E+08 | 30000 | chr7_151710001_151740000 | 0 | 16.27 | 7.35 | PRR |
| chr9 | 78920001 | 78980000 | 60000 | chr9_78920001_78980000 | 0.12 | 35.71 | 7.35 | RRR |
| chr1 | 13680001 | 13760000 | 80000 | chr1_13680001_13760000 | 2.23 | 379.86 | 7.35 | RRR |
| chr15 | 86020001 | 86050000 | 30000 | chr15_86020001_86050000 | 0.02 | 19.36 | 7.34 | PRR |
| chr16 | 88650001 | 88670000 | 20000 | chr16_88650001_88670000 | 0.11 | 34.03 | 7.34 | FRR |
| chr7 | 64710001 | 64740000 | 30000 | chr7_64710001_64740000 | 0.01 | 17.38 | 7.31 | PRR |
| chr5 | 1.23E+08 | 1.23E+08 | 30000 | chr5_123080001_123110000 | 0 | 15.73 | 7.31 | FRR |
| chr3 | 1.12E+08 | 1.12E+08 | 40000 | chr3_112170001_112210000 | 0.01 | 17.17 | 7.29 | RRR |
| chr14 | 1.04E+08 | 1.04E+08 | 20000 | chr14_103810001_103830000 | 0.04 | 21.76 | 7.29 | RRR |
| chrX | 52770001 | 52810000 | 40000 | chrX_52770001_52810000 | 0.02 | 18.53 | 7.28 | RRR |
| chr17 | 20850001 | 20900000 | 50000 | chr17_20850001_20900000 | 0.01 | 16.41 | 7.23 | RRR |
| chr16 | 30210001 | 30230000 | 20000 | chr16_30210001_30230000 | 0.02 | 17.8 | 7.22 | RRR |
| chr16 | 3050001 | 3070000 | 20000 | chr16_3050001_3070000 | 0.01 | 16.1 | 7.2 | RRR |
| chr7 | 64820001 | 64860000 | 40000 | chr7_64820001_64860000 | 0.01 | 16.07 | 7.2 | RRR |
| chr1 | 6590001 | 6640000 | 50000 | chr1_6590001_6640000 | 0.73 | 121.11 | 7.19 | RRR |
| chrX | 8740001 | 8780000 | 40000 | chrX_8740001_8780000 | 0.02 | 17.25 | 7.18 | RRR |
| chr1 | 1.83E+08 | 1.83E+08 | 40000 | chr1_182690001_182730000 | 0.01 | 15.79 | 7.17 | RRR |
| chr1 | 1.93E+08 | 1.93E+08 | 40000 | chr1_192810001_192850000 | 0.38 | 68.39 | 7.16 | RRR |
| chr12 | 38550001 | 38580000 | 30000 | chr12_38550001_38580000 | 0 | 14.11 | 7.15 | RRR |
| chr17 | 47270001 | 47340000 | 70000 | chr17_47270001_47340000 | 0.93 | 146.32 | 7.15 | PRR |
| chr5 | 1.16E+08 | 1.16E+08 | 30000 | chr5_115890001_115920000 | 0.01 | 15.53 | 7.15 | PRR |
| chr11 | 1.08E+08 | 1.08E+08 | 20000 | chr11_107780001_107800000 | 0.4 | 71.07 | 7.15 | PRR |
| chrX | 52710001 | 52740000 | 30000 | chrX_52710001_52740000 | 0.03 | 18.19 | 7.14 | RRR |
| chr3 | 1.28E+08 | 1.28E+08 | 30000 | chr3_128460001_128490000 | 0.01 | 15.41 | 7.14 | PRR |
| chr5 | 62880001 | 62930000 | 50000 | chr5_62880001_62930000 | 0.01 | 15.34 | 7.13 | RRR |
| chr17 | 37540001 | 37560000 | 20000 | chr17_37540001_37560000 | 0.03 | 18.05 | 7.13 | PRR |
| chr13 | 34490001 | 34520000 | 30000 | chr13_34490001_34520000 | 0.01 | 14.95 | 7.1 | RRR |
| chr7 | 1.01E+08 | 1.01E+08 | 20000 | chr7_100890001_100910000 | 0.12 | 30.17 | 7.1 | RRR |
| chr6 | 26240001 | 26270000 | 30000 | chr6_26240001_26270000 | 0.01 | 15 | 7.1 | FRR |
| chr6 | 76130001 | 76160000 | 30000 | chr6_76130001_76160000 | 0.01 | 14.82 | 7.08 | RRR |
| chr19 | 58600001 | 58630000 | 30000 | chr19_58600001_58630000 | 0.04 | 18.89 | 7.08 | RRR |
| chr19 | 22140001 | 22210000 | 70000 | chr19_22140001_22210000 | 0.13 | 30.7 | 7.07 | RRR |
| chr16 | 30490001 | 30530000 | 40000 | chr16_30490001_30530000 | 0.01 | 14.68 | 7.07 | FRR |
| chrX | 48120001 | 48140000 | 20000 | chrX_48120001_48140000 | 0.02 | 15.86 | 7.06 | RRR |
| chr17 | 37480001 | 37530000 | 50000 | chr17_37480001_37530000 | 0.01 | 14.28 | 7.03 | RRR |
| chr14 | 21780001 | 21810000 | 30000 | chr14_21780001_21810000 | 0 | 12.97 | 7.03 | PRR |
| chr7 | 63910001 | 63960000 | 50000 | chr7_63910001_63960000 | 0 | 12.88 | 7.02 | RRR |
| chr10 | 70320001 | 70350000 | 30000 | chr10_70320001_70350000 | 0 | 12.68 | 7 | PRR |
| chr1 | 1.12E+08 | 1.12E+08 | 30000 | chr1_112010001_112040000 | 0.01 | 13.98 | 7 | PRR |
| chr1 | 65600001 | 65630000 | 30000 | chr1_65600001_65630000 | 0.32 | 53.29 | 6.99 | RRR |
| chr9 | 15290001 | 15320000 | 30000 | chr9_15290001_15320000 | 0.03 | 16.44 | 6.99 | PRR |
| chr7 | 57170001 | 57210000 | 40000 | chr7_57170001_57210000 | 0.09 | 23.78 | 6.97 | RRR |
| chr9 | 3950001 | 3980000 | 30000 | chr9_3950001_3980000 | 0 | 12.39 | 6.96 | RRR |
| chr19 | 12300001 | 12350000 | 50000 | chr19_12300001_12350000 | 0.11 | 25.94 | 6.95 | PRR |
| chr17 | 78690001 | 78720000 | 30000 | chr17_78690001_78720000 | 0.04 | 17.23 | 6.95 | PRR |
| chr5 | 16800001 | 16830000 | 30000 | chr5_16800001_16830000 | 0.09 | 23.19 | 6.94 | FRR |
| chr14 | 83600001 | 83630000 | 30000 | chr14_83600001_83630000 | 0.03 | 15.82 | 6.94 | RRR |
| chr1 | 26910001 | 26940000 | 30000 | chr1_26910001_26940000 | 0.01 | 13.31 | 6.93 | PRR |
| chr2 | 65640001 | 65670000 | 30000 | chr2_65640001_65670000 | 0.01 | 13.25 | 6.92 | PRR |
| chr13 | 34530001 | 34560000 | 30000 | chr13_34530001_34560000 | 0.06 | 19.06 | 6.9 | RRR |
| chr3 | 1.56E+08 | 1.56E+08 | 20000 | chr3_155770001_155790000 | 0 | 11.84 | 6.9 | RRR |
| chr11 | 57120001 | 57160000 | 40000 | chr11_57120001_57160000 | 0.06 | 19.04 | 6.9 | FRR |
| chr13 | 21260001 | 21290000 | 30000 | chr13_21260001_21290000 | 0.55 | 76.84 | 6.89 | PRR |
| chr2 | 1.76E+08 | 1.76E+08 | 30000 | chr2_176130001_176160000 | 0 | 11.75 | 6.89 | FRR |
| chr12 | 53280001 | 53310000 | 30000 | chr12_53280001_53310000 | 0.04 | 16.43 | 6.88 | FRR |
| chrX | 48240001 | 48280000 | 40000 | chrX_48240001_48280000 | 0.01 | 12.82 | 6.88 | RRR |
| chr9 | 19440001 | 19470000 | 30000 | chr9_19440001_19470000 | 0.01 | 12.8 | 6.87 | PRR |
| chr14 | 83550001 | 83580000 | 30000 | chr14_83550001_83580000 | 0.01 | 12.65 | 6.86 | RRR |
| chr18 | 70900001 | 70930000 | 30000 | chr18_70900001_70930000 | 0.04 | 16.09 | 6.85 | FRR |
| chr10 | 81680001 | 81710000 | 30000 | chr10_81680001_81710000 | 0 | 11.33 | 6.84 | RRR |
| chr13 | 56030001 | 56060000 | 30000 | chr13_56030001_56060000 | 0 | 11.25 | 6.83 | RRR |
| chr12 | 42610001 | 42640000 | 30000 | chr12_42610001_42640000 | 0.01 | 12.22 | 6.81 | PRR |
| chr16 | 9060001 | 9090000 | 30000 | chr16_9060001_9090000 | 0.01 | 12.14 | 6.8 | RRR |
| chr11 | 64650001 | 64680000 | 30000 | chr11_64650001_64680000 | 0.3 | 44.37 | 6.8 | RRR |
| chrX | 70920001 | 70940000 | 20000 | chrX_70920001_70940000 | 0.19 | 32.28 | 6.8 | RRR |
| chr10 | 15230001 | 15250000 | 20000 | chr10_15230001_15250000 | 0.03 | 14.25 | 6.79 | RRR |
| chr1 | 1510001 | 1530000 | 20000 | chr1_1510001_1530000 | 0.01 | 12.03 | 6.78 | FRR |
| chr1 | 26500001 | 26530000 | 30000 | chr1_26500001_26530000 | 0.08 | 19.51 | 6.77 | PRR |
| chr18 | 8750001 | 8780000 | 30000 | chr18_8750001_8780000 | 0.13 | 24.96 | 6.77 | RRR |
| chr16 | 2840001 | 2870000 | 30000 | chr16_2840001_2870000 | 0 | 10.81 | 6.77 | PRR |
| chr16 | 61220001 | 61250000 | 30000 | chr16_61220001_61250000 | 0.01 | 11.79 | 6.76 | RRR |
| chr6 | 1.17E+08 | 1.17E+08 | 30000 | chr6_117060001_117090000 | 0 | 10.69 | 6.75 | RRR |
| chr15 | 60950001 | 60970000 | 20000 | chr15_60950001_60970000 | 0 | 10.52 | 6.73 | PRR |
| chr8 | 37710001 | 37730000 | 20000 | chr8_37710001_37730000 | 0.01 | 11.58 | 6.73 | PRR |
| chr12 | 40470001 | 40500000 | 30000 | chr12_40470001_40500000 | 0 | 10.45 | 6.72 | RRR |
| chr12 | 53500001 | 53530000 | 30000 | chr12_53500001_53530000 | 0.12 | 23.03 | 6.72 | RRR |
| chr3 | 1.39E+08 | 1.39E+08 | 70000 | chr3_138710001_138780000 | 0.72 | 83.86 | 6.68 | RRR |
| chr8 | 83390001 | 83450000 | 60000 | chr8_83390001_83450000 | 0.13 | 23.48 | 6.68 | RRR |
| chr7 | 26300001 | 26330000 | 30000 | chr7_26300001_26330000 | 0.11 | 21.32 | 6.67 | RRR |
| chr7 | 1.4E+08 | 1.4E+08 | 30000 | chr7_140200001_140230000 | 0.01 | 10.99 | 6.66 | FRR |
| chr14 | 19970001 | 20000000 | 30000 | chr14_19970001_20000000 | 0.01 | 10.91 | 6.65 | RRR |
| chrY | 6790001 | 6820000 | 30000 | chrY_6790001_6820000 | 0.01 | 10.98 | 6.65 | PRR |
| chr1 | 1.14E+08 | 1.14E+08 | 30000 | chr1_113930001_113960000 | 0 | 9.84 | 6.64 | FRR |
| chr1 | 46470001 | 46490000 | 20000 | chr1_46470001_46490000 | 0 | 9.79 | 6.63 | RRR |
| chr17 | 66000001 | 66030000 | 30000 | chr17_66000001_66030000 | 0.01 | 10.73 | 6.62 | RRR |
| chr12 | 22780001 | 22810000 | 30000 | chr12_22780001_22810000 | 0.06 | 15.59 | 6.62 | PRR |
| chr6 | 30470001 | 30500000 | 30000 | chr6_30470001_30500000 | 0.01 | 10.64 | 6.61 | RRR |
| chr11 | 77560001 | 77580000 | 20000 | chr11_77560001_77580000 | 0 | 9.64 | 6.61 | RRR |
| chr7 | 63460001 | 63490000 | 30000 | chr7_63460001_63490000 | 0.2 | 28.85 | 6.59 | RRR |
| chr12 | 32100001 | 32130000 | 30000 | chr12_32100001_32130000 | 0.34 | 42.2 | 6.59 | PRR |
| chr5 | 79600001 | 79620000 | 20000 | chr5_79600001_79620000 | 0 | 9.56 | 6.59 | PRR |
| chr2 | 36830001 | 36850000 | 20000 | chr2_36830001_36850000 | 0.05 | 14.29 | 6.58 | RRR |
| chr18 | 6770001 | 6800000 | 30000 | chr18_6770001_6800000 | 0.04 | 13.22 | 6.57 | RRR |
| chr17 | 37150001 | 37180000 | 30000 | chr17_37150001_37180000 | 0 | 9.31 | 6.56 | RRR |
| chr10 | 32410001 | 32440000 | 30000 | chr10_32410001_32440000 | 0.04 | 12.97 | 6.54 | RRR |
| chr10 | 12050001 | 12070000 | 20000 | chr10_12050001_12070000 | 0 | 9.08 | 6.52 | RRR |
| chr17 | 29270001 | 29300000 | 30000 | chr17_29270001_29300000 | 0 | 9.09 | 6.52 | FRR |
| chr9 | 75470001 | 75500000 | 30000 | chr9_75470001_75500000 | 0.01 | 9.93 | 6.51 | RRR |
| chr3 | 1.37E+08 | 1.37E+08 | 20000 | chr3_136580001_136600000 | 0 | 9.01 | 6.51 | RRR |
| chr5 | 1.4E+08 | 1.4E+08 | 20000 | chr5_140000001_140020000 | 0.04 | 12.7 | 6.51 | PRR |
| chr11 | 31730001 | 31760000 | 30000 | chr11_31730001_31760000 | 0 | 8.83 | 6.48 | RRR |
| chr2 | 27510001 | 27530000 | 20000 | chr2_27510001_27530000 | 0.03 | 11.52 | 6.48 | PRR |
| chr4 | 25660001 | 25700000 | 40000 | chr4_25660001_25700000 | 8.86 | 797.06 | 6.48 | PRR |
| chr10 | 43160001 | 43190000 | 30000 | chr10_43160001_43190000 | 0.01 | 9.73 | 6.48 | PRR |
| chr9 | 79620001 | 79650000 | 30000 | chr9_79620001_79650000 | 0.08 | 15.82 | 6.47 | FRR |
| chr3 | 48760001 | 48780000 | 20000 | chr3_48760001_48780000 | 0 | 8.6 | 6.44 | PRR |
| chr16 | 54310001 | 54330000 | 20000 | chr16_54310001_54330000 | 0.01 | 9.42 | 6.44 | PRR |
| chr7 | 65780001 | 65820000 | 40000 | chr7_65780001_65820000 | 0.03 | 11.09 | 6.43 | FRR |
| chr16 | 46740001 | 46760000 | 20000 | chr16_46740001_46760000 | 0.01 | 9.34 | 6.42 | RRR |
| chr7 | 1.4E+08 | 1.4E+08 | 30000 | chr7_139860001_139890000 | 0.06 | 13.6 | 6.42 | FRR |
| chr2 | 23610001 | 23640000 | 30000 | chr2_23610001_23640000 | 0 | 8.38 | 6.41 | RRR |
| chr8 | 80860001 | 80890000 | 30000 | chr8_80860001_80890000 | 0.01 | 9.23 | 6.41 | PRR |
| chr6 | 1.33E+08 | 1.33E+08 | 30000 | chr6_133140001_133170000 | 0.01 | 9.24 | 6.41 | FRR |
| chr17 | 44310001 | 44350000 | 40000 | chr17_44310001_44350000 | 0.14 | 20.22 | 6.4 | RRR |
| chr6 | 15180001 | 15210000 | 30000 | chr6_15180001_15210000 | 0.04 | 11.74 | 6.4 | RRR |
| chr12 | 86090001 | 86120000 | 30000 | chr12_86090001_86120000 | 0.01 | 9.06 | 6.38 | RRR |
| chr19 | 34380001 | 34410000 | 30000 | chr19_34380001_34410000 | 0.44 | 44.99 | 6.38 | PRR |
| chr6 | 1.27E+08 | 1.28E+08 | 20000 | chr6_127490001_127510000 | 0 | 8.22 | 6.38 | RRR |
| chr14 | 39880001 | 39900000 | 20000 | chr14_39880001_39900000 | 0 | 8.17 | 6.37 | FRR |
| chr12 | 1870001 | 1890000 | 20000 | chr12_1870001_1890000 | 0.07 | 13.89 | 6.36 | RRR |
| chr12 | 7830001 | 7860000 | 30000 | chr12_7830001_7860000 | 0.01 | 8.92 | 6.36 | PRR |
| chr17 | 42020001 | 42050000 | 30000 | chr17_42020001_42050000 | 0.04 | 11.22 | 6.34 | PRR |
| chr10 | 12180001 | 12200000 | 20000 | chr10_12180001_12200000 | 0 | 7.93 | 6.33 | RRR |
| chr6 | 28940001 | 28970000 | 30000 | chr6_28940001_28970000 | 0.04 | 11.17 | 6.33 | PRR |
| chr1 | 1.11E+08 | 1.11E+08 | 30000 | chr1_111430001_111460000 | 0.04 | 11.07 | 6.32 | FRR |
| chr7 | 75000001 | 75030000 | 30000 | chr7_75000001_75030000 | 0.02 | 9.41 | 6.31 | RRR |
| chr19 | 22560001 | 22580000 | 20000 | chr19_22560001_22580000 | 0.01 | 8.6 | 6.31 | RRR |
| chr8 | 59570001 | 59590000 | 20000 | chr8_59570001_59590000 | 0.03 | 10.23 | 6.31 | RRR |
| chr3 | 48050001 | 48070000 | 20000 | chr3_48050001_48070000 | 0.02 | 9.44 | 6.31 | PRR |
| chr1 | 1.15E+08 | 1.15E+08 | 20000 | chr1_115330001_115350000 | 0.03 | 10.12 | 6.3 | RRR |
| chr11 | 1.08E+08 | 1.08E+08 | 20000 | chr11_107680001_107700000 | 0 | 7.7 | 6.29 | RRR |
| chr9 | 1.2E+08 | 1.2E+08 | 30000 | chr9_119590001_119620000 | 0.03 | 10.05 | 6.29 | PRR |
| chr16 | 29590001 | 29620000 | 30000 | chr16_29590001_29620000 | 0.01 | 8.49 | 6.29 | FRR |
| chr12 | 69620001 | 69640000 | 20000 | chr12_69620001_69640000 | 0 | 7.65 | 6.28 | RRR |
| chr16 | 51780001 | 51800000 | 20000 | chr16_51780001_51800000 | 0.01 | 8.41 | 6.27 | RRR |
| chrX | 99530001 | 99560000 | 30000 | chrX_99530001_99560000 | 0.06 | 12.18 | 6.26 | FRR |
| chr6 | 7260001 | 7280000 | 20000 | chr6_7260001_7280000 | 0 | 7.59 | 6.26 | FRR |
| chr14 | 1.03E+08 | 1.03E+08 | 30000 | chr14_103210001_103240000 | 0.02 | 9.06 | 6.25 | RRR |
| chrX | 70970001 | 71000000 | 30000 | chrX_70970001_71000000 | 0.32 | 31.84 | 6.25 | RRR |
| chr17 | 4670001 | 4690000 | 20000 | chr17_4670001_4690000 | 0.01 | 8.26 | 6.25 | RRR |
| chr16 | 67410001 | 67440000 | 30000 | chr16_67410001_67440000 | 0.02 | 9.04 | 6.25 | FRR |
| chr4 | 46720001 | 46750000 | 30000 | chr4_46720001_46750000 | 0.17 | 20.26 | 6.24 | RRR |
| chr19 | 42810001 | 42830000 | 20000 | chr19_42810001_42830000 | 0.17 | 20.07 | 6.22 | PRR |
| chr12 | 31790001 | 31820000 | 30000 | chr12_31790001_31820000 | 0 | 7.35 | 6.22 | PRR |
| chr2 | 69820001 | 69850000 | 30000 | chr2_69820001_69850000 | 0.22 | 23.82 | 6.22 | FRR |
| chr17 | 75760001 | 75790000 | 30000 | chr17_75760001_75790000 | 0.14 | 17.85 | 6.22 | RRR |
| chr2 | 25940001 | 25960000 | 20000 | chr2_25940001_25960000 | 0 | 7.31 | 6.21 | PRR |
| chr5 | 60620001 | 60650000 | 30000 | chr5_60620001_60650000 | 0 | 7.28 | 6.21 | PRR |
| chr19 | 20400001 | 20430000 | 30000 | chr19_20400001_20430000 | 0.52 | 45.58 | 6.2 | RRR |
| chr1 | 28610001 | 28650000 | 40000 | chr1_28610001_28650000 | 0.14 | 17.57 | 6.2 | RRR |
| chr6 | 34680001 | 34710000 | 30000 | chr6_34680001_34710000 | 0 | 7.21 | 6.19 | PRR |
| chr13 | 43600001 | 43630000 | 30000 | chr13_43600001_43630000 | 0.04 | 10.07 | 6.18 | RRR |
| chr9 | 35010001 | 35040000 | 30000 | chr9_35010001_35040000 | 0.01 | 7.85 | 6.18 | FRR |
| chr1 | 1.56E+08 | 1.56E+08 | 30000 | chr1_155520001_155550000 | 0.24 | 24.33 | 6.17 | PRR |
| chr16 | 8150001 | 8180000 | 30000 | chr16_8150001_8180000 | 0 | 7.08 | 6.17 | RRR |
| chr3 | 1.22E+08 | 1.23E+08 | 30000 | chr3_122480001_122510000 | 0.01 | 7.82 | 6.17 | FRR |
| chr12 | 46360001 | 46380000 | 20000 | chr12_46360001_46380000 | 0.3 | 28.59 | 6.16 | RRR |
| chr1 | 1.96E+08 | 1.96E+08 | 30000 | chr1_195680001_195710000 | 0.01 | 7.74 | 6.16 | RRR |
| chr2 | 1.14E+08 | 1.14E+08 | 30000 | chr2_113840001_113870000 | 0 | 7.02 | 6.15 | PRR |
| chr1 | 1.47E+08 | 1.47E+08 | 30000 | chr1_146870001_146900000 | 0.02 | 8.36 | 6.14 | RRR |
| chr1 | 11530001 | 11560000 | 30000 | chr1_11530001_11560000 | 0.01 | 7.62 | 6.13 | PRR |
| chr14 | 1.06E+08 | 1.06E+08 | 30000 | chr14_106310001_106340000 | 0.01 | 7.62 | 6.13 | RRR |
| chr2 | 60950001 | 60980000 | 30000 | chr2_60950001_60980000 | 0.35 | 31.15 | 6.12 | PRR |
| chr19 | 54250001 | 54270000 | 20000 | chr19_54250001_54270000 | 0 | 6.75 | 6.1 | RRR |
| chr19 | 40210001 | 40240000 | 30000 | chr19_40210001_40240000 | 0.82 | 62.56 | 6.09 | RRR |
| chr2 | 1.57E+08 | 1.57E+08 | 30000 | chr2_156810001_156840000 | 0.01 | 7.37 | 6.09 | RRR |
| chr15 | 23500001 | 23530000 | 30000 | chr15_23500001_23530000 | 0 | 6.7 | 6.09 | RRR |
| chr14 | 64200001 | 64230000 | 30000 | chr14_64200001_64230000 | 0.01 | 7.39 | 6.09 | PRR |
| chr9 | 38050001 | 38070000 | 20000 | chr9_38050001_38070000 | 0.03 | 8.59 | 6.06 | PRR |
| chr12 | 49770001 | 49800000 | 30000 | chr12_49770001_49800000 | 0 | 6.56 | 6.06 | PRR |
| chr2 | 8110001 | 8130000 | 20000 | chr2_8110001_8130000 | 0.01 | 7.17 | 6.05 | RRR |
| chr19 | 11840001 | 11860000 | 20000 | chr19_11840001_11860000 | 0 | 6.51 | 6.05 | RRR |
| chr17 | 29380001 | 29420000 | 40000 | chr17_29380001_29420000 | 0.12 | 14.46 | 6.05 | RRR |
| chr5 | 1.51E+08 | 1.51E+08 | 90000 | chr5_150670001_150760000 | 1.28 | 91.08 | 6.05 | RRR |
| chr19 | 1890001 | 1910000 | 20000 | chr19_1890001_1910000 | 0.11 | 13.61 | 6.03 | RRR |
| chr5 | 1.51E+08 | 1.51E+08 | 30000 | chr5_150780001_150810000 | 0.02 | 7.73 | 6.03 | PRR |
| chrX | 70880001 | 70900000 | 20000 | chrX_70880001_70900000 | 0 | 6.41 | 6.02 | PRR |
| chr6 | 28470001 | 28500000 | 30000 | chr6_28470001_28500000 | 0.03 | 8.27 | 6.01 | PRR |
| chr15 | 65580001 | 65600000 | 20000 | chr15_65580001_65600000 | 0.01 | 6.95 | 6 | PRR |
| chr10 | 43850001 | 43880000 | 30000 | chr10_43850001_43880000 | 0.17 | 17.1 | 5.99 | PRR |
| chr5 | 82370001 | 82390000 | 20000 | chr5_82370001_82390000 | 0.03 | 8.08 | 5.98 | RRR |
| chr2 | 1.31E+08 | 1.31E+08 | 20000 | chr2_130880001_130900000 | 0.01 | 6.84 | 5.98 | FRR |
| chrX | 1.52E+08 | 1.52E+08 | 160000 | chrX_151790001_151950000 | 4.27 | 273.22 | 5.97 | RRR |
| chr7 | 72680001 | 72710000 | 30000 | chr7_72680001_72710000 | 0.04 | 8.69 | 5.97 | RRR |
| chr15 | 75440001 | 75470000 | 30000 | chr15_75440001_75470000 | 0.09 | 11.7 | 5.96 | RRR |
| chr1 | 31970001 | 32000000 | 30000 | chr1_31970001_32000000 | 0.03 | 8.02 | 5.96 | PRR |
| chr1 | 1.1E+08 | 1.1E+08 | 20000 | chr1_109990001_110010000 | 0 | 6.07 | 5.95 | PRR |
| chr12 | 88940001 | 88970000 | 30000 | chr12_88940001_88970000 | 0 | 6.09 | 5.95 | FRR |
| chrX | 8990001 | 9010000 | 20000 | chrX_8990001_9010000 | 0 | 6.02 | 5.94 | RRR |
| chr6 | 32490001 | 32520000 | 30000 | chr6_32490001_32520000 | 0 | 6.06 | 5.94 | RRR |
| chr17 | 47070001 | 47090000 | 20000 | chr17_47070001_47090000 | 0 | 6.03 | 5.94 | RRR |
| chr8 | 53610001 | 53640000 | 30000 | chr8_53610001_53640000 | 0 | 5.97 | 5.92 | RRR |
| chr19 | 58060001 | 58080000 | 20000 | chr19_58060001_58080000 | 0 | 5.96 | 5.92 | RRR |
| chr7 | 64020001 | 64090000 | 70000 | chr7_64020001_64090000 | 0.61 | 42.89 | 5.92 | RRR |
| chrX | 99650001 | 99670000 | 20000 | chrX_99650001_99670000 | 0.01 | 6.5 | 5.91 | RRR |
| chr19 | 36970001 | 37000000 | 30000 | chr19_36970001_37000000 | 0.17 | 16.14 | 5.91 | RRR |
| chr3 | 1.3E+08 | 1.3E+08 | 20000 | chr3_130170001_130190000 | 0.03 | 7.66 | 5.9 | FRR |
| chr4 | 1.36E+08 | 1.36E+08 | 30000 | chr4_135920001_135950000 | 0.01 | 6.49 | 5.9 | RRR |
| chr1 | 1.62E+08 | 1.62E+08 | 30000 | chr1_162390001_162420000 | 0.04 | 8.26 | 5.9 | RRR |
| chr5 | 74710001 | 74730000 | 20000 | chr5_74710001_74730000 | 0.01 | 6.48 | 5.9 | PRR |
| chr5 | 19020001 | 19050000 | 30000 | chr5_19020001_19050000 | 0 | 5.86 | 5.9 | RRR |
| chr14 | 19600001 | 19620000 | 20000 | chr14_19600001_19620000 | 0 | 5.84 | 5.89 | RRR |
| chr13 | 41610001 | 41630000 | 20000 | chr13_41610001_41630000 | 0.07 | 9.82 | 5.87 | RRR |
| chr7 | 72920001 | 72950000 | 30000 | chr7_72920001_72950000 | 0.02 | 6.93 | 5.87 | FRR |
| chr19 | 9580001 | 9600000 | 20000 | chr19_9580001_9600000 | 0.26 | 20.88 | 5.86 | PRR |
| chr1 | 46130001 | 46150000 | 20000 | chr1_46130001_46150000 | 0 | 5.71 | 5.86 | PRR |
| chr2 | 9750001 | 9770000 | 20000 | chr2_9750001_9770000 | 0.01 | 6.29 | 5.86 | FRR |
| chr4 | 54880001 | 54910000 | 30000 | chr4_54880001_54910000 | 0.09 | 10.83 | 5.85 | PRR |
| chr9 | 34030001 | 34060000 | 30000 | chr9_34030001_34060000 | 0 | 5.68 | 5.85 | PRR |
| chr9 | 37150001 | 37180000 | 30000 | chr9_37150001_37180000 | 0 | 5.67 | 5.85 | PRR |
| chr6 | 99710001 | 99740000 | 30000 | chr6_99710001_99740000 | 0 | 5.67 | 5.85 | FRR |
| chr6 | 26500001 | 26520000 | 20000 | chr6_26500001_26520000 | 0.02 | 6.67 | 5.82 | RRR |
| chr7 | 1.29E+08 | 1.29E+08 | 30000 | chr7_129410001_129440000 | 0.09 | 10.67 | 5.82 | FRR |
| chr19 | 55840001 | 55870000 | 30000 | chr19_55840001_55870000 | 0.1 | 11.11 | 5.81 | FRR |
| chr6 | 56750001 | 56770000 | 20000 | chr6_56750001_56770000 | 0 | 5.5 | 5.81 | RRR |
| chr5 | 81430001 | 81450000 | 20000 | chr5_81430001_81450000 | 0 | 5.51 | 5.81 | RRR |
| chr10 | 1.17E+08 | 1.17E+08 | 20000 | chr10_116540001_116560000 | 0 | 5.47 | 5.8 | RRR |
| chr16 | 87380001 | 87430000 | 50000 | chr16_87380001_87430000 | 0.46 | 31.16 | 5.8 | RRR |
| chr7 | 16750001 | 16770000 | 20000 | chr7_16750001_16770000 | 0 | 5.44 | 5.79 | RRR |
| chr13 | 19750001 | 19770000 | 20000 | chr13_19750001_19770000 | 0 | 5.45 | 5.79 | RRR |
| chr19 | 37250001 | 37300000 | 50000 | chr19_37250001_37300000 | 0.99 | 60.04 | 5.79 | FRR |
| chr7 | 26190001 | 26220000 | 30000 | chr7_26190001_26220000 | 0.25 | 19.24 | 5.79 | PRR |
| chr5 | 95170001 | 95200000 | 30000 | chr5_95170001_95200000 | 0.07 | 9.27 | 5.78 | FRR |
| chr16 | 18920001 | 18950000 | 30000 | chr16_18920001_18950000 | 0.39 | 26.71 | 5.77 | RRR |
| chr16 | 70250001 | 70270000 | 20000 | chr16_70250001_70270000 | 0.38 | 25.89 | 5.76 | RRR |
| chrX | 47970001 | 47990000 | 20000 | chrX_47970001_47990000 | 0.28 | 20.53 | 5.76 | RRR |
| chr5 | 32190001 | 32220000 | 30000 | chr5_32190001_32220000 | 0.01 | 5.82 | 5.75 | RRR |
| chr7 | 1.43E+08 | 1.43E+08 | 30000 | chr7_142750001_142780000 | 0.19 | 15.44 | 5.74 | RRR |
| chr4 | 37010001 | 37040000 | 30000 | chr4_37010001_37040000 | 0 | 5.21 | 5.73 | RRR |
| chr1 | 1.61E+08 | 1.61E+08 | 50000 | chr1_161360001_161410000 | 0.43 | 27.94 | 5.73 | PRR |
| chr1 | 1.1E+08 | 1.1E+08 | 20000 | chr1_109610001_109630000 | 0.08 | 9.23 | 5.7 | PRR |
| chr7 | 1.4E+08 | 1.4E+08 | 30000 | chr7_140160001_140190000 | 0.01 | 5.6 | 5.7 | PRR |
| chr17 | 34310001 | 34340000 | 30000 | chr17_34310001_34340000 | 0.01 | 5.58 | 5.69 | RRR |
| chr15 | 80520001 | 80550000 | 30000 | chr15_80520001_80550000 | 0.03 | 6.62 | 5.69 | PRR |
| chr19 | 20650001 | 20670000 | 20000 | chr19_20650001_20670000 | 0 | 5.08 | 5.69 | RRR |
| chr17 | 61520001 | 61550000 | 30000 | chr17_61520001_61550000 | 0.11 | 10.72 | 5.69 | FRR |
| chr3 | 42130001 | 42160000 | 30000 | chr3_42130001_42160000 | 0.01 | 5.59 | 5.69 | PRR |
| chr14 | 77090001 | 77150000 | 60000 | chr14_77090001_77150000 | 0.35 | 23.04 | 5.68 | PRR |
| chr9 | 1.23E+08 | 1.23E+08 | 20000 | chr9_123240001_123260000 | 0.02 | 6.05 | 5.68 | RRR |
| chr18 | 29570001 | 29670000 | ####### | chr18_29570001_29670000 | 2.63 | 138.73 | 5.67 | PRR |
| chr12 | 1.08E+08 | 1.08E+08 | 30000 | chr12_108260001_108290000 | 16.33 | 807.48 | 5.62 | PRR |
| chr16 | 19000001 | 19020000 | 20000 | chr16_19000001_19020000 | 0.01 | 5.32 | 5.62 | PRR |
| chr11 | 82830001 | 82860000 | 30000 | chr11_82830001_82860000 | 0.06 | 7.78 | 5.62 | PRR |
| chr18 | 57850001 | 57880000 | 30000 | chr18_57850001_57880000 | 0.09 | 9.16 | 5.61 | RRR |
| chr14 | 21650001 | 21670000 | 20000 | chr14_21650001_21670000 | 0.01 | 5.18 | 5.58 | PRR |
| chr12 | 3260001 | 3280000 | 20000 | chr12_3260001_3280000 | 0.01 | 5.12 | 5.57 | RRR |
| chr1 | 22790001 | 22820000 | 30000 | chr1_22790001_22820000 | 0.08 | 8.48 | 5.57 | FRR |
| chr4 | 71750001 | 71780000 | 30000 | chr4_71750001_71780000 | 0.13 | 10.81 | 5.57 | PRR |
| chr7 | 26050001 | 26080000 | 30000 | chr7_26050001_26080000 | 0.41 | 23.94 | 5.56 | PRR |
| chr11 | 94750001 | 94770000 | 20000 | chr11_94750001_94770000 | 1.34 | 67.17 | 5.55 | FRR |
| chr13 | 1.07E+08 | 1.07E+08 | 20000 | chr13_107170001_107190000 | 0.05 | 6.91 | 5.55 | PRR |
| chr11 | 43710001 | 43740000 | 30000 | chr11_43710001_43740000 | 0.09 | 8.65 | 5.53 | FRR |
| chr19 | 50230001 | 50260000 | 30000 | chr19_50230001_50260000 | 0.17 | 12.4 | 5.53 | PRR |
| chr14 | 20070001 | 20100000 | 30000 | chr14_20070001_20100000 | 0.07 | 7.72 | 5.52 | RRR |
| chr6 | 95560001 | 95590000 | 30000 | chr6_95560001_95590000 | 0.04 | 6.29 | 5.51 | PRR |
| chr15 | 89470001 | 89500000 | 30000 | chr15_89470001_89500000 | 0.03 | 5.79 | 5.5 | RRR |
| chr6 | 1.08E+08 | 1.08E+08 | 30000 | chr6_108020001_108050000 | 0.12 | 9.87 | 5.5 | FRR |
| chr7 | 57570001 | 57610000 | 40000 | chr7_57570001_57610000 | 0.18 | 12.46 | 5.49 | RRR |
| chr13 | 51910001 | 51930000 | 20000 | chr13_51910001_51930000 | 0.03 | 5.76 | 5.49 | PRR |
| chr1 | 35390001 | 35410000 | 20000 | chr1_35390001_35410000 | 0.03 | 5.7 | 5.48 | RRR |
| chr12 | 14660001 | 14700000 | 40000 | chr12_14660001_14700000 | 0.38 | 21.07 | 5.46 | RRR |
| chr2 | 1.12E+08 | 1.12E+08 | 70000 | chr2_111870001_111940000 | 1.26 | 59.02 | 5.44 | FRR |
| chr16 | 87510001 | 87540000 | 30000 | chr16_87510001_87540000 | 0.12 | 9.28 | 5.41 | RRR |
| chrX | 70570001 | 70590000 | 20000 | chrX_70570001_70590000 | 0.03 | 5.4 | 5.4 | PRR |
| chr1 | 1.13E+08 | 1.13E+08 | 30000 | chr1_113420001_113450000 | 0.45 | 22.88 | 5.38 | RRR |
| chr5 | 1400001 | 1460000 | 60000 | chr5_1400001_1460000 | 0.64 | 30.62 | 5.38 | FRR |
| chr6 | 1.13E+08 | 1.13E+08 | 20000 | chr6_112820001_112840000 | 0.04 | 5.67 | 5.37 | RRR |
| chr2 | 48540001 | 48560000 | 20000 | chr2_48540001_48560000 | 0.08 | 7.33 | 5.37 | PRR |
| chr17 | 7540001 | 7570000 | 30000 | chr17_7540001_7570000 | 0.35 | 18.52 | 5.37 | FRR |
| chr18 | 8920001 | 8950000 | 30000 | chr18_8920001_8950000 | 0.03 | 5.23 | 5.36 | FRR |
| chr6 | 390001 | 420000 | 30000 | chr6_390001_420000 | 0.3 | 16.17 | 5.35 | FRR |
| chr1 | 40990001 | 41010000 | 20000 | chr1_40990001_41010000 | 0.09 | 7.58 | 5.34 | RRR |
| chr19 | 57040001 | 57060000 | 20000 | chr19_57040001_57060000 | 0.05 | 5.9 | 5.32 | RRR |
| chr18 | 11900001 | 11920000 | 20000 | chr18_11900001_11920000 | 0.06 | 6.31 | 5.32 | PRR |
| chr12 | 68810001 | 68840000 | 30000 | chr12_68810001_68840000 | 0.36 | 18.06 | 5.3 | RRR |
| chr6 | 27080001 | 27100000 | 20000 | chr6_27080001_27100000 | 0.71 | 31.87 | 5.3 | FRR |
| chr10 | 43690001 | 43710000 | 20000 | chr10_43690001_43710000 | 0.21 | 12 | 5.29 | PRR |
| chr17 | 65220001 | 65240000 | 20000 | chr17_65220001_65240000 | 0.04 | 5.3 | 5.27 | PRR |
| chr12 | 56450001 | 56490000 | 40000 | chr12_56450001_56490000 | 0.71 | 30.63 | 5.25 | PRR |
| chr19 | 15050001 | 15080000 | 30000 | chr19_15050001_15080000 | 0.4 | 18.82 | 5.24 | FRR |
| chr1 | 1.44E+08 | 1.44E+08 | 30000 | chr1_144000001_144030000 | 1.41 | 56.94 | 5.24 | PRR |
| chr7 | 64360001 | 64380000 | 20000 | chr7_64360001_64380000 | 0.06 | 5.89 | 5.23 | RRR |
| chr17 | 20790001 | 20840000 | 50000 | chr17_20790001_20840000 | 0.42 | 19.17 | 5.21 | PRR |
| chr17 | 19520001 | 19550000 | 30000 | chr17_19520001_19550000 | 0.04 | 5.07 | 5.21 | RRR |
| chr19 | 47350001 | 47380000 | 30000 | chr19_47350001_47380000 | 0.37 | 17.13 | 5.2 | PRR |
| chr1 | 1.5E+08 | 1.5E+08 | 30000 | chr1_150150001_150180000 | 1.93 | 74.77 | 5.2 | RRR |
| chr19 | 6920001 | 6960000 | 40000 | chr19_6920001_6960000 | 0.43 | 19.23 | 5.19 | PRR |
| chr1 | 1.47E+08 | 1.47E+08 | 60000 | chr1_146940001_147000000 | 0.97 | 38.34 | 5.17 | RRR |
| chr6 | 34750001 | 34770000 | 20000 | chr6_34750001_34770000 | 0.12 | 7.83 | 5.17 | RRR |
| chr7 | 1.05E+08 | 1.05E+08 | 50000 | chr7_104520001_104570000 | 0.55 | 23.28 | 5.17 | PRR |
| chr1 | 21700001 | 21730000 | 30000 | chr1_21700001_21730000 | 0.29 | 13.85 | 5.16 | RRR |
| chr7 | 64390001 | 64410000 | 20000 | chr7_64390001_64410000 | 0.08 | 6.32 | 5.16 | RRR |
| chr4 | 8420001 | 8450000 | 30000 | chr4_8420001_8450000 | 0.3 | 14.04 | 5.14 | PRR |
| chr4 | 87890001 | 87930000 | 40000 | chr4_87890001_87930000 | 0.48 | 20.25 | 5.13 | FRR |
| chr5 | 53680001 | 53730000 | 50000 | chr5_53680001_53730000 | 0.63 | 25.1 | 5.11 | RRR |
| chr9 | 90700001 | 90740000 | 40000 | chr9_90700001_90740000 | 0.53 | 21.7 | 5.11 | RRR |
| chr15 | 20830001 | 20890000 | 60000 | chr15_20830001_20890000 | 2.43 | 87.45 | 5.11 | RRR |
| chr19 | 57630001 | 57690000 | 60000 | chr19_57630001_57690000 | 72.44 | 2490.01 | 5.1 | RRR |
| chr17 | 28910001 | 28940000 | 30000 | chr17_28910001_28940000 | 0.22 | 10.7 | 5.08 | PRR |
| chr10 | 66860001 | 66920000 | 60000 | chr10_66860001_66920000 | 1.11 | 39.95 | 5.05 | RRR |
| chr19 | 23270001 | 23300000 | 30000 | chr19_23270001_23300000 | 0.17 | 8.8 | 5.04 | PRR |
| chr6 | 1.15E+08 | 1.15E+08 | 40000 | chr6_115300001_115340000 | 4.07 | 136.3 | 5.03 | RRR |
| chr19 | 22830001 | 22900000 | 70000 | chr19_22830001_22900000 | 0.58 | 22.18 | 5.03 | PRR |
| chr15 | 41330001 | 41350000 | 20000 | chr15_41330001_41350000 | 0.09 | 6.06 | 5.02 | RRR |
| chr14 | 45350001 | 45370000 | 20000 | chr14_45350001_45370000 | 0.21 | 9.75 | 4.99 | RRR |
| chr4 | 85490001 | 85510000 | 20000 | chr4_85490001_85510000 | 0.09 | 5.92 | 4.99 | PRR |
| chrX | 55100001 | 55120000 | 20000 | chrX_55100001_55120000 | 0.08 | 5.61 | 4.99 | RRR |
| chrX | 54340001 | 54380000 | 40000 | chrX_54340001_54380000 | 0.63 | 22.95 | 4.98 | RRR |
| chr2 | 39800001 | 39820000 | 20000 | chr2_39800001_39820000 | 0.3 | 12.55 | 4.98 | FRR |
| chr2 | 53200001 | 53230000 | 30000 | chr2_53200001_53230000 | 0.2 | 9.15 | 4.95 | RRR |
| chr11 | 89800001 | 89840000 | 40000 | chr11_89800001_89840000 | 1.07 | 35.7 | 4.94 | RRR |
| chr9 | 97230001 | 97250000 | 20000 | chr9_97230001_97250000 | 0.07 | 5.05 | 4.92 | PRR |
| chr13 | 32570001 | 32590000 | 20000 | chr13_32570001_32590000 | 0.17 | 8.04 | 4.91 | RRR |
| chr19 | 340001 | 400000 | 60000 | chr19_340001_400000 | 1.42 | 45.67 | 4.91 | PRR |
| chr10 | 98630001 | 98660000 | 30000 | chr10_98630001_98660000 | 0.22 | 9.5 | 4.91 | FRR |
| chr10 | 63610001 | 63640000 | 30000 | chr10_63610001_63640000 | 0.41 | 15.11 | 4.9 | RRR |
| chr14 | 1.02E+08 | 1.02E+08 | 30000 | chr14_102130001_102160000 | 0.19 | 8.53 | 4.9 | RRR |
| chr11 | 82650001 | 82680000 | 30000 | chr11_82650001_82680000 | 0.25 | 10.33 | 4.9 | RRR |
| chr13 | 84640001 | 84670000 | 30000 | chr13_84640001_84670000 | 0.38 | 14.18 | 4.89 | RRR |
| chr11 | 60920001 | 60940000 | 20000 | chr11_60920001_60940000 | 0.08 | 5.22 | 4.89 | PRR |
| chr5 | 17800001 | 17820000 | 20000 | chr5_17800001_17820000 | 0.17 | 7.84 | 4.88 | PRR |
| chr10 | 51550001 | 51570000 | 20000 | chr10_51550001_51570000 | 0.11 | 5.86 | 4.83 | PRR |
| chr1 | 13310001 | 13380000 | 70000 | chr1_13310001_13380000 | 19.07 | 540.6 | 4.82 | RRR |
| chr7 | 1.29E+08 | 1.29E+08 | 30000 | chr7_129260001_129290000 | 0.43 | 14.84 | 4.82 | RRR |
| chr12 | 1.13E+08 | 1.13E+08 | 30000 | chr12_112690001_112720000 | 0.13 | 6.34 | 4.81 | FRR |
| chr19 | 23550001 | 23600000 | 50000 | chr19_23550001_23600000 | 2.05 | 60.05 | 4.81 | RRR |
| chr17 | 66080001 | 66110000 | 30000 | chr17_66080001_66110000 | 0.46 | 15.66 | 4.81 | PRR |
| chr6 | 1.06E+08 | 1.06E+08 | 20000 | chr6_105520001_105540000 | 0.38 | 13.26 | 4.8 | RRR |
| chr14 | 71370001 | 71390000 | 20000 | chr14_71370001_71390000 | 0.13 | 6.19 | 4.77 | RRR |
| chr18 | 19160001 | 19190000 | 30000 | chr18_19160001_19190000 | 0.14 | 6.46 | 4.77 | FRR |
| chr5 | 76090001 | 76150000 | 60000 | chr5_76090001_76150000 | 1.31 | 37.73 | 4.75 | PRR |
| chrX | 1.36E+08 | 1.36E+08 | 30000 | chrX_136390001_136420000 | 0.93 | 27.56 | 4.75 | FRR |
| chr10 | 99080001 | 99110000 | 30000 | chr10_99080001_99110000 | 12.99 | 348.7 | 4.74 | RRR |
| chr13 | 36990001 | 37030000 | 40000 | chr13_36990001_37030000 | 20.32 | 543.57 | 4.73 | PRR |
| chr15 | 99020001 | 99050000 | 30000 | chr15_99020001_99050000 | 0.66 | 19.79 | 4.71 | FRR |
| chr1 | 13790001 | 13820000 | 30000 | chr1_13790001_13820000 | 0.96 | 27.23 | 4.69 | FRR |
| chr11 | 82130001 | 82160000 | 30000 | chr11_82130001_82160000 | 1.23 | 33.91 | 4.68 | RRR |
| chr12 | 85780001 | 85840000 | 60000 | chr12_85780001_85840000 | 0.9 | 25.46 | 4.68 | RRR |
| chr3 | 5140001 | 5180000 | 40000 | chr3_5140001_5180000 | 1.61 | 43.16 | 4.66 | RRR |
| chr19 | 23130001 | 23180000 | 50000 | chr19_23130001_23180000 | 2.37 | 61.7 | 4.65 | RRR |
| chr7 | 62740001 | 62770000 | 30000 | chr7_62740001_62770000 | 0.42 | 13 | 4.65 | RRR |
| chr2 | 34900001 | 34920000 | 20000 | chr2_34900001_34920000 | 0.11 | 5.12 | 4.64 | FRR |
| chr19 | 12100001 | 12120000 | 20000 | chr19_12100001_12120000 | 0.17 | 6.65 | 4.64 | RRR |
| chr19 | 2180001 | 2210000 | 30000 | chr19_2180001_2210000 | 1.05 | 28.37 | 4.63 | FRR |
| chr19 | 41660001 | 41710000 | 50000 | chr19_41660001_41710000 | 1 | 27.2 | 4.63 | PRR |
| chr1 | 1.57E+08 | 1.57E+08 | 20000 | chr1_157100001_157120000 | 1.28 | 33.69 | 4.61 | PRR |
| chr1 | 45870001 | 45890000 | 20000 | chr1_45870001_45890000 | 0.12 | 5.27 | 4.61 | PRR |
| chr2 | 1.73E+08 | 1.73E+08 | 20000 | chr2_172760001_172780000 | 0.12 | 5.21 | 4.59 | RRR |
| chr15 | 21890001 | 21920000 | 30000 | chr15_21890001_21920000 | 0.65 | 17.75 | 4.57 | PRR |
| chr18 | 11860001 | 11880000 | 20000 | chr18_11860001_11880000 | 0.14 | 5.53 | 4.55 | RRR |
| chr12 | 1.2E+08 | 1.2E+08 | 50000 | chr12_120420001_120470000 | 0.74 | 19.61 | 4.55 | FRR |
| chr14 | 54400001 | 54450000 | 50000 | chr14_54400001_54450000 | 1.51 | 37.48 | 4.54 | FRR |
| chr4 | 1.29E+08 | 1.29E+08 | 30000 | chr4_128870001_128900000 | 0.13 | 5.16 | 4.52 | RRR |
| chr14 | 29290001 | 29320000 | 30000 | chr14_29290001_29320000 | 0.49 | 13.42 | 4.52 | RRR |
| chr3 | 1.38E+08 | 1.38E+08 | 20000 | chr3_137860001_137880000 | 0.29 | 8.77 | 4.51 | PRR |
| chr10 | 88840001 | 88860000 | 20000 | chr10_88840001_88860000 | 0.16 | 5.75 | 4.49 | RRR |
| chr2 | 1.5E+08 | 1.5E+08 | 30000 | chr2_149620001_149650000 | 4.44 | 99.92 | 4.46 | FRR |
| chr2 | 1.4E+08 | 1.4E+08 | 50000 | chr2_140190001_140240000 | 0.92 | 22.28 | 4.46 | RRR |
| chr2 | 10590001 | 10610000 | 20000 | chr2_10590001_10610000 | 0.55 | 14.14 | 4.45 | RRR |
| chr6 | 78290001 | 78310000 | 20000 | chr6_78290001_78310000 | 0.14 | 5.11 | 4.44 | RRR |
| chr9 | 99690001 | 99730000 | 40000 | chr9_99690001_99730000 | 1.06 | 24.85 | 4.43 | PRR |
| chr1 | 44570001 | 44620000 | 50000 | chr1_44570001_44620000 | 33.17 | 719.27 | 4.43 | RRR |
| chr6 | 40330001 | 40360000 | 30000 | chr6_40330001_40360000 | 0.26 | 7.66 | 4.43 | FRR |
| chr5 | 42880001 | 42940000 | 60000 | chr5_42880001_42940000 | 90.18 | 1940.68 | 4.43 | PRR |
| chr19 | 37940001 | 37970000 | 30000 | chr19_37940001_37970000 | 0.43 | 11.31 | 4.43 | FRR |
| chr10 | 8080001 | 8110000 | 30000 | chr10_8080001_8110000 | 0.75 | 18.11 | 4.42 | FRR |
| chrX | 1.53E+08 | 1.53E+08 | 20000 | chrX_152940001_152960000 | 0.9 | 21.3 | 4.42 | RRR |
| chr3 | 75540001 | 75560000 | 20000 | chr3_75540001_75560000 | 0.25 | 7.37 | 4.42 | RRR |
| chr1 | 90440001 | 90470000 | 30000 | chr1_90440001_90470000 | 0.49 | 12.44 | 4.41 | RRR |
| chr1 | 1.55E+08 | 1.55E+08 | 20000 | chr1_155040001_155060000 | 0.86 | 20.31 | 4.41 | PRR |
| chr12 | 10090001 | 10120000 | 30000 | chr12_10090001_10120000 | 0.22 | 6.59 | 4.39 | RRR |
| chr1 | 63770001 | 63800000 | 30000 | chr1_63770001_63800000 | 0.3 | 8.25 | 4.38 | FRR |
| chr4 | 1.4E+08 | 1.4E+08 | 30000 | chr4_140360001_140390000 | 0.38 | 9.85 | 4.37 | FRR |
| chr6 | 27440001 | 27470000 | 30000 | chr6_27440001_27470000 | 0.56 | 13.47 | 4.36 | PRR |
| chr1 | 27430001 | 27500000 | 70000 | chr1_27430001_27500000 | 4.57 | 95.03 | 4.35 | FRR |
| chr1 | 28960001 | 28980000 | 20000 | chr1_28960001_28980000 | 0.19 | 5.77 | 4.34 | PRR |
| chr6 | 5120001 | 5150000 | 30000 | chr6_5120001_5150000 | 0.32 | 8.34 | 4.33 | RRR |
| chr7 | 76250001 | 76270000 | 20000 | chr7_76250001_76270000 | 0.35 | 8.81 | 4.31 | PRR |
| chr19 | 55650001 | 55680000 | 30000 | chr19_55650001_55680000 | 4.15 | 83.24 | 4.29 | PRR |
| chr19 | 750001 | 800000 | 50000 | chr19_750001_800000 | 1.19 | 25.1 | 4.29 | PRR |
| chr6 | 27840001 | 27860000 | 20000 | chr6_27840001_27860000 | 0.22 | 6.07 | 4.27 | FRR |
| chr11 | 64990001 | 65020000 | 30000 | chr11_64990001_65020000 | 0.35 | 8.56 | 4.27 | FRR |
| chr3 | 46530001 | 46550000 | 20000 | chr3_46530001_46550000 | 0.34 | 8.22 | 4.24 | PRR |
| chr3 | 1.29E+08 | 1.29E+08 | 30000 | chr3_128540001_128570000 | 1 | 20.64 | 4.24 | PRR |
| chr1 | 13620001 | 13660000 | 40000 | chr1_13620001_13660000 | 18.81 | 355.08 | 4.23 | RRR |
| chr1 | 1.13E+08 | 1.13E+08 | 30000 | chr1_113330001_113360000 | 0.25 | 6.48 | 4.23 | FRR |
| chr18 | 77420001 | 77480000 | 60000 | chr18_77420001_77480000 | 0.93 | 19.2 | 4.23 | FRR |
| chr19 | 41130001 | 41160000 | 30000 | chr19_41130001_41160000 | 0.41 | 9.35 | 4.21 | RRR |
| chr2 | 26550001 | 26580000 | 30000 | chr2_26550001_26580000 | 0.53 | 11.53 | 4.21 | PRR |
| chr5 | 53600001 | 53630000 | 30000 | chr5_53600001_53630000 | 0.3 | 7.19 | 4.19 | PRR |
| chr11 | 3650001 | 3690000 | 40000 | chr11_3650001_3690000 | 0.86 | 17.19 | 4.17 | FRR |
| chr1 | 95540001 | 95570000 | 30000 | chr1_95540001_95570000 | 0.19 | 5.13 | 4.17 | FRR |
| chrX | 37290001 | 37310000 | 20000 | chrX_37290001_37310000 | 0.56 | 11.75 | 4.17 | RRR |
| chr1 | 13400001 | 13440000 | 40000 | chr1_13400001_13440000 | 19.85 | 356.6 | 4.16 | RRR |
| chr14 | 27390001 | 27420000 | 30000 | chr14_27390001_27420000 | 0.29 | 6.83 | 4.15 | PRR |
| chr19 | 58730001 | 58750000 | 20000 | chr19_58730001_58750000 | 0.22 | 5.58 | 4.15 | RRR |
| chr8 | 6340001 | 6410000 | 70000 | chr8_6340001_6410000 | 1 | 19.46 | 4.15 | PRR |
| chr1 | 97210001 | 97240000 | 30000 | chr1_97210001_97240000 | 0.54 | 11.23 | 4.15 | FRR |
| chr16 | 22300001 | 22320000 | 20000 | chr16_22300001_22320000 | 0.62 | 12.7 | 4.15 | RRR |
| chr14 | 1.03E+08 | 1.03E+08 | 30000 | chr14_103050001_103080000 | 1.3 | 24.6 | 4.14 | PRR |
| chr11 | 62110001 | 62140000 | 30000 | chr11_62110001_62140000 | 0.22 | 5.55 | 4.14 | RRR |
| chr12 | 19580001 | 19630000 | 50000 | chr12_19580001_19630000 | 0.84 | 16.28 | 4.12 | PRR |
| chr3 | 48290001 | 48310000 | 20000 | chr3_48290001_48310000 | 0.57 | 11.51 | 4.12 | FRR |
| chr14 | 68060001 | 68080000 | 20000 | chr14_68060001_68080000 | 0.59 | 11.82 | 4.11 | PRR |
| chr16 | 5230001 | 5260000 | 30000 | chr16_5230001_5260000 | 0.25 | 5.93 | 4.11 | PRR |
| chr19 | 22460001 | 22520000 | 60000 | chr19_22460001_22520000 | 1.37 | 25.1 | 4.1 | RRR |
| chr19 | 48770001 | 48810000 | 40000 | chr19_48770001_48810000 | 0.96 | 18.12 | 4.1 | RRR |
| chr6 | 64250001 | 64280000 | 30000 | chr6_64250001_64280000 | 0.61 | 11.87 | 4.08 | PRR |
| chr10 | 1.04E+08 | 1.04E+08 | 30000 | chr10_104260001_104290000 | 1.93 | 34.14 | 4.08 | FRR |
| chr16 | 72550001 | 72580000 | 30000 | chr16_72550001_72580000 | 0.5 | 9.97 | 4.07 | PRR |
| chr5 | 1.09E+08 | 1.09E+08 | 30000 | chr5_109250001_109280000 | 0.4 | 8.24 | 4.06 | RRR |
| chr1 | 13090001 | 13160000 | 70000 | chr1_13090001_13160000 | 19.72 | 327.64 | 4.05 | RRR |
| chrX | 50020001 | 50050000 | 30000 | chrX_50020001_50050000 | 1 | 18.16 | 4.05 | FRR |
| chr19 | 22350001 | 22390000 | 40000 | chr19_22350001_22390000 | 2.29 | 39.3 | 4.04 | RRR |
| chr7 | 1.01E+08 | 1.01E+08 | 30000 | chr7_100980001_101010000 | 0.97 | 17.53 | 4.04 | FRR |
| chr7 | 1.42E+08 | 1.43E+08 | 50000 | chr7_142450001_142500000 | 0.84 | 15.32 | 4.04 | FRR |
| chr12 | 49680001 | 49710000 | 30000 | chr12_49680001_49710000 | 1.19 | 21.09 | 4.04 | RRR |
| chr1 | 1.13E+08 | 1.13E+08 | 30000 | chr1_113380001_113410000 | 0.66 | 12.22 | 4.02 | FRR |
| chr19 | 30290001 | 30330000 | 40000 | chr19_30290001_30330000 | 39.47 | 630.9 | 4 | PRR |
| chr2 | 1.32E+08 | 1.32E+08 | 20000 | chr2_131810001_131830000 | 1.18 | 20.09 | 3.98 | PRR |
| chr3 | 23370001 | 23400000 | 30000 | chr3_23370001_23400000 | 0.27 | 5.72 | 3.98 | PRR |
| chr5 | 64780001 | 64800000 | 20000 | chr5_64780001_64800000 | 0.37 | 7.22 | 3.96 | RRR |
| chr18 | 12730001 | 12780000 | 50000 | chr18_12730001_12780000 | 4.14 | 65.05 | 3.94 | PRR |
| chr7 | 1.4E+08 | 1.4E+08 | 30000 | chr7_139930001_139960000 | 1.41 | 22.93 | 3.93 | PRR |
| chr1 | 1.49E+08 | 1.49E+08 | 30000 | chr1_148840001_148870000 | 0.44 | 8.12 | 3.93 | PRR |
| chr2 | 1.29E+08 | 1.29E+08 | 20000 | chr2_128630001_128650000 | 0.74 | 12.66 | 3.93 | PRR |
| chr3 | 10170001 | 10190000 | 20000 | chr3_10170001_10190000 | 0.33 | 6.44 | 3.93 | FRR |
| chr10 | 28960001 | 28990000 | 30000 | chr10_28960001_28990000 | 6.35 | 97.98 | 3.93 | RRR |
| chr2 | 1.33E+08 | 1.33E+08 | 30000 | chr2_132710001_132740000 | 0.48 | 8.58 | 3.9 | RRR |
| chr7 | 7700001 | 7730000 | 30000 | chr7_7700001_7730000 | 0.58 | 10.07 | 3.9 | RRR |
| chr5 | 12480001 | 12510000 | 30000 | chr5_12480001_12510000 | 0.32 | 6.19 | 3.9 | PRR |
| chr1 | 45810001 | 45830000 | 20000 | chr1_45810001_45830000 | 2.37 | 36.72 | 3.9 | PRR |
| chr7 | 98230001 | 98270000 | 40000 | chr7_98230001_98270000 | 1.67 | 25.73 | 3.87 | PRR |
| chr19 | 23980001 | 24010000 | 30000 | chr19_23980001_24010000 | 0.87 | 14 | 3.86 | PRR |
| chr19 | 46070001 | 46100000 | 30000 | chr19_46070001_46100000 | 0.75 | 12.23 | 3.86 | FRR |
| chr6 | 71180001 | 71210000 | 30000 | chr6_71180001_71210000 | 0.63 | 10.49 | 3.86 | FRR |
| chr6 | 31860001 | 31880000 | 20000 | chr6_31860001_31880000 | 0.84 | 13.51 | 3.86 | PRR |
| chr19 | 5940001 | 5960000 | 20000 | chr19_5940001_5960000 | 0.61 | 10.12 | 3.85 | PRR |
| chr12 | 31590001 | 31650000 | 60000 | chr12_31590001_31650000 | 1.59 | 24.2 | 3.85 | FRR |
| chr11 | 59540001 | 59560000 | 20000 | chr11_59540001_59560000 | 1.35 | 20.72 | 3.84 | PRR |
| chr19 | 40260001 | 40290000 | 30000 | chr19_40260001_40290000 | 70.6 | 1003.72 | 3.83 | FRR |
| chr13 | 1.13E+08 | 1.13E+08 | 30000 | chr13_113330001_113360000 | 0.57 | 9.43 | 3.83 | FRR |
| chr1 | 1.61E+08 | 1.61E+08 | 70000 | chr1_160860001_160930000 | 6.27 | 89.3 | 3.81 | FRR |
| chr17 | 37430001 | 37460000 | 30000 | chr17_37430001_37460000 | 0.67 | 10.59 | 3.8 | RRR |
| chrX | 80050001 | 80080000 | 30000 | chrX_80050001_80080000 | 0.3 | 5.49 | 3.8 | RRR |
| chr3 | 1.33E+08 | 1.33E+08 | 20000 | chr3_133280001_133300000 | 0.97 | 14.84 | 3.8 | PRR |
| chr8 | 1.05E+08 | 1.05E+08 | 20000 | chr8_105290001_105310000 | 0.27 | 5.07 | 3.8 | FRR |
| chr16 | 9170001 | 9190000 | 20000 | chr16_9170001_9190000 | 1.46 | 21.63 | 3.8 | FRR |
| chr6 | 1.35E+08 | 1.35E+08 | 40000 | chr6_134600001_134640000 | 1.1 | 16.14 | 3.76 | PRR |
| chr10 | 89190001 | 89220000 | 30000 | chr10_89190001_89220000 | 0.87 | 12.89 | 3.74 | RRR |
| chr6 | 32320001 | 32360000 | 40000 | chr6_32320001_32360000 | 1.88 | 26.13 | 3.73 | RRR |
| chr10 | 21830001 | 21860000 | 30000 | chr10_21830001_21860000 | 0.7 | 10.45 | 3.72 | FRR |
| chr5 | 5420001 | 5450000 | 30000 | chr5_5420001_5450000 | 0.55 | 8.42 | 3.71 | FRR |
| chr3 | 25880001 | 25910000 | 30000 | chr3_25880001_25910000 | 0.45 | 7.05 | 3.7 | RRR |
| chr19 | 3840001 | 3870000 | 30000 | chr19_3840001_3870000 | 1.52 | 20.97 | 3.7 | FRR |
| chrX | 1.35E+08 | 1.35E+08 | 20000 | chrX_134510001_134530000 | 1.05 | 14.62 | 3.68 | RRR |
| chr10 | 1.26E+08 | 1.26E+08 | 30000 | chr10_125780001_125810000 | 0.51 | 7.74 | 3.68 | FRR |
| chr2 | 54470001 | 54500000 | 30000 | chr2_54470001_54500000 | 0.8 | 11.33 | 3.67 | RRR |
| chr12 | 22890001 | 22920000 | 30000 | chr12_22890001_22920000 | 0.91 | 12.76 | 3.67 | FRR |
| chr1 | 53880001 | 53900000 | 20000 | chr1_53880001_53900000 | 0.38 | 5.99 | 3.67 | PRR |
| chr15 | 63390001 | 63410000 | 20000 | chr15_63390001_63410000 | 0.69 | 9.86 | 3.66 | RRR |
| chr19 | 21370001 | 21390000 | 20000 | chr19_21370001_21390000 | 0.45 | 6.72 | 3.63 | RRR |
| chr18 | 76940001 | 76970000 | 30000 | chr18_76940001_76970000 | 0.45 | 6.71 | 3.63 | FRR |
| chr8 | 42550001 | 42580000 | 30000 | chr8_42550001_42580000 | 2.73 | 34.63 | 3.62 | PRR |
| chr7 | 1.52E+08 | 1.52E+08 | 60000 | chr7_152410001_152470000 | 1.31 | 17.25 | 3.62 | FRR |
| chr14 | 96810001 | 96830000 | 20000 | chr14_96810001_96830000 | 0.73 | 10.08 | 3.62 | FRR |
| chr7 | 12520001 | 12550000 | 30000 | chr7_12520001_12550000 | 0.34 | 5.3 | 3.62 | FRR |
| chr14 | 20790001 | 20810000 | 20000 | chr14_20790001_20810000 | 1.91 | 24.22 | 3.6 | RRR |
| chr5 | 17640001 | 17670000 | 30000 | chr5_17640001_17670000 | 41.04 | 497.68 | 3.6 | PRR |
| chr14 | 77160001 | 77190000 | 30000 | chr14_77160001_77190000 | 1.02 | 13.36 | 3.59 | PRR |
| chr2 | 1.32E+08 | 1.32E+08 | 20000 | chr2_132220001_132240000 | 0.8 | 10.71 | 3.59 | RRR |
| chr6 | 1.44E+08 | 1.44E+08 | 30000 | chr6_143920001_143950000 | 0.77 | 10.27 | 3.58 | PRR |
| chr17 | 41390001 | 41420000 | 30000 | chr17_41390001_41420000 | 2.2 | 27.38 | 3.58 | PRR |
| chr7 | 1.03E+08 | 1.03E+08 | 30000 | chr7_102900001_102930000 | 0.81 | 10.79 | 3.58 | FRR |
| chr1 | 12980001 | 13070000 | 90000 | chr1_12980001_13070000 | 42.37 | 504.52 | 3.57 | RRR |
| chr1 | 9600001 | 9640000 | 40000 | chr1_9600001_9640000 | 2.09 | 25.94 | 3.57 | FRR |
| chrX | 24010001 | 24040000 | 30000 | chrX_24010001_24040000 | 1.05 | 13.54 | 3.57 | PRR |
| chr17 | 19910001 | 19940000 | 30000 | chr17_19910001_19940000 | 0.82 | 10.81 | 3.57 | FRR |
| chr7 | 91770001 | 91790000 | 20000 | chr7_91770001_91790000 | 0.35 | 5.19 | 3.56 | RRR |
| chr12 | 31450001 | 31470000 | 20000 | chr12_31450001_31470000 | 0.44 | 6.23 | 3.55 | PRR |
| chr14 | 76350001 | 76370000 | 20000 | chr14_76350001_76370000 | 0.39 | 5.61 | 3.54 | PRR |
| chr3 | 1.14E+08 | 1.14E+08 | 30000 | chr3_113940001_113970000 | 0.63 | 8.4 | 3.54 | FRR |
| chr12 | 1.17E+08 | 1.17E+08 | 70000 | chr12_117080001_117150000 | 2.06 | 25 | 3.54 | PRR |
| chr14 | 76030001 | 76060000 | 30000 | chr14_76030001_76060000 | 0.75 | 9.73 | 3.53 | PRR |
| chr15 | 41130001 | 41170000 | 40000 | chr15_41130001_41170000 | 10.02 | 115.15 | 3.51 | FRR |
| chr19 | 20740001 | 20770000 | 30000 | chr19_20740001_20770000 | 0.53 | 7 | 3.49 | PRR |
| chrX | 4450001 | 4470000 | 20000 | chrX_4450001_4470000 | 0.39 | 5.42 | 3.49 | RRR |
| chr3 | 53380001 | 53400000 | 20000 | chr3_53380001_53400000 | 0.41 | 5.56 | 3.47 | PRR |
| chr4 | 15200001 | 15230000 | 30000 | chr4_15200001_15230000 | 0.46 | 6.05 | 3.46 | RRR |
| chr19 | 21610001 | 21660000 | 50000 | chr19_21610001_21660000 | 9.33 | 103.39 | 3.46 | RRR |
| chr14 | 31700001 | 31730000 | 30000 | chr14_31700001_31730000 | 0.66 | 8.18 | 3.45 | PRR |
| chr2 | 85790001 | 85810000 | 20000 | chr2_85790001_85810000 | 0.37 | 5.01 | 3.44 | FRR |
| chr19 | 49820001 | 49840000 | 20000 | chr19_49820001_49840000 | 0.45 | 5.81 | 3.43 | FRR |
| chr17 | 29190001 | 29210000 | 20000 | chr17_29190001_29210000 | 0.45 | 5.79 | 3.42 | PRR |
| chr19 | 56690001 | 56720000 | 30000 | chr19_56690001_56720000 | 75.87 | 810.62 | 3.42 | RRR |
| chr7 | 1.43E+08 | 1.43E+08 | 20000 | chr7_143090001_143110000 | 1.97 | 21.87 | 3.41 | PRR |
| chrX | 1.54E+08 | 1.54E+08 | 30000 | chrX_153540001_153570000 | 4.27 | 45.69 | 3.39 | PRR |
| chr2 | 1.79E+08 | 1.79E+08 | 30000 | chr2_179070001_179100000 | 1.52 | 16.83 | 3.39 | PRR |
| chr17 | 78510001 | 78530000 | 20000 | chr17_78510001_78530000 | 0.5 | 6.21 | 3.39 | PRR |
| chr8 | 81440001 | 81460000 | 20000 | chr8_81440001_81460000 | 0.79 | 9.17 | 3.38 | RRR |
| chr8 | 9030001 | 9070000 | 40000 | chr8_9030001_9070000 | 5.56 | 58.76 | 3.38 | FRR |
| chr11 | 10420001 | 10450000 | 30000 | chr11_10420001_10450000 | 1.41 | 15.51 | 3.37 | RRR |
| chr14 | 1.01E+08 | 1.01E+08 | 30000 | chr14_100710001_100740000 | 1.03 | 11.61 | 3.37 | RRR |
| chr1 | 16150001 | 16170000 | 20000 | chr1_16150001_16170000 | 0.65 | 7.58 | 3.36 | PRR |
| chrX | 69890001 | 69920000 | 30000 | chrX_69890001_69920000 | 0.64 | 7.44 | 3.35 | RRR |
| chr1 | 92440001 | 92540000 | ####### | chr1_92440001_92540000 | 47.67 | 486.02 | 3.35 | RRR |
| chr9 | 97300001 | 97320000 | 20000 | chr9_97300001_97320000 | 0.44 | 5.39 | 3.35 | PRR |
| chr11 | 50250001 | 50270000 | 20000 | chr11_50250001_50270000 | 0.58 | 6.81 | 3.35 | PRR |
| chrX | 1.09E+08 | 1.09E+08 | 30000 | chrX_109090001_109120000 | 0.97 | 10.76 | 3.34 | RRR |
| chr4 | 1.53E+08 | 1.53E+08 | 30000 | chr4_153320001_153350000 | 6.4 | 65.81 | 3.34 | PRR |
| chr7 | 77150001 | 77170000 | 20000 | chr7_77150001_77170000 | 0.61 | 7.11 | 3.34 | RRR |
| chr11 | 82320001 | 82360000 | 40000 | chr11_82320001_82360000 | 5.96 | 60.99 | 3.33 | RRR |
| chr6 | 10820001 | 10850000 | 30000 | chr6_10820001_10850000 | 0.68 | 7.72 | 3.33 | FRR |
| chr18 | 76860001 | 76890000 | 30000 | chr18_76860001_76890000 | 0.96 | 10.48 | 3.32 | RRR |
| chr17 | 2150001 | 2200000 | 50000 | chr17_2150001_2200000 | 2.86 | 29.53 | 3.32 | PRR |
| chr2 | 1.79E+08 | 1.79E+08 | 30000 | chr2_178520001_178550000 | 0.89 | 9.71 | 3.31 | RRR |
| chr12 | 8100001 | 8130000 | 30000 | chr12_8100001_8130000 | 0.69 | 7.68 | 3.3 | PRR |
| chr4 | 1.47E+08 | 1.47E+08 | 20000 | chr4_146610001_146630000 | 0.66 | 7.34 | 3.29 | FRR |
| chr6 | 10400001 | 10420000 | 20000 | chr6_10400001_10420000 | 0.61 | 6.73 | 3.27 | FRR |
| chr8 | 83340001 | 83370000 | 30000 | chr8_83340001_83370000 | 0.64 | 7 | 3.26 | RRR |
| chr15 | 64470001 | 64490000 | 20000 | chr15_64470001_64490000 | 1.66 | 16.76 | 3.26 | PRR |
| chr11 | 89460001 | 89500000 | 40000 | chr11_89460001_89500000 | 2.83 | 27.79 | 3.25 | FRR |
| chr1 | 1.72E+08 | 1.72E+08 | 40000 | chr1_171800001_171840000 | 2 | 19.89 | 3.25 | PRR |
| chr7 | 7750001 | 7780000 | 30000 | chr7_7750001_7780000 | 1.24 | 12.65 | 3.25 | RRR |
| chrX | 35630001 | 35660000 | 30000 | chrX_35630001_35660000 | 1.04 | 10.72 | 3.25 | FRR |
| chr11 | 2890001 | 2920000 | 30000 | chr11_2890001_2920000 | 0.89 | 9.32 | 3.25 | FRR |
| chr16 | 48420001 | 48440000 | 20000 | chr16_48420001_48440000 | 1.54 | 15.43 | 3.24 | RRR |
| chr5 | 76240001 | 76280000 | 40000 | chr5_76240001_76280000 | 1.21 | 11.96 | 3.2 | FRR |
| chr5 | 1.31E+08 | 1.31E+08 | 40000 | chr5_131260001_131300000 | 3.3 | 30.95 | 3.19 | PRR |
| chr11 | 1.19E+08 | 1.2E+08 | 30000 | chr11_119470001_119500000 | 17.82 | 161.97 | 3.18 | FRR |
| chr12 | 1.04E+08 | 1.04E+08 | 30000 | chr12_104220001_104250000 | 1.25 | 12.01 | 3.17 | FRR |
| chr19 | 47510001 | 47560000 | 50000 | chr19_47510001_47560000 | 3.04 | 28.1 | 3.17 | PRR |
| chr5 | 65800001 | 65830000 | 30000 | chr5_65800001_65830000 | 1.69 | 15.78 | 3.15 | PRR |
| chr8 | 92930001 | 92960000 | 30000 | chr8_92930001_92960000 | 1.23 | 11.73 | 3.15 | PRR |
| chr10 | 93520001 | 93560000 | 40000 | chr10_93520001_93560000 | 2.12 | 19.44 | 3.14 | RRR |
| chr19 | 22000001 | 22040000 | 40000 | chr19_22000001_22040000 | 1.82 | 16.83 | 3.14 | RRR |
| chr13 | 22600001 | 22630000 | 30000 | chr13_22600001_22630000 | 0.73 | 7.18 | 3.13 | PRR |
| chr10 | 1.05E+08 | 1.05E+08 | 20000 | chr10_105120001_105140000 | 2.72 | 24.57 | 3.13 | FRR |
| chr15 | 75780001 | 75800000 | 20000 | chr15_75780001_75800000 | 0.72 | 6.96 | 3.11 | RRR |
| chr6 | 10760001 | 10780000 | 20000 | chr6_10760001_10780000 | 1.39 | 12.59 | 3.09 | FRR |
| chr16 | 1430001 | 1460000 | 30000 | chr16_1430001_1460000 | 1.61 | 14.47 | 3.09 | PRR |
| chrX | 73010001 | 73040000 | 30000 | chrX_73010001_73040000 | 2.27 | 19.9 | 3.08 | FRR |
| chr14 | 1.07E+08 | 1.07E+08 | 40000 | chr14_106830001_106870000 | 1.64 | 14.54 | 3.07 | PRR |
| chr16 | 28560001 | 28580000 | 20000 | chr16_28560001_28580000 | 0.67 | 6.31 | 3.06 | PRR |
| chr16 | 75700001 | 75760000 | 60000 | chr16_75700001_75760000 | 91.1 | 751.4 | 3.04 | PRR |
| chr10 | 93050001 | 93080000 | 30000 | chr10_93050001_93080000 | 2.24 | 19.13 | 3.04 | FRR |
| chr11 | 49060001 | 49120000 | 60000 | chr11_49060001_49120000 | 4.89 | 40.75 | 3.03 | FRR |
| chr16 | 29750001 | 29780000 | 30000 | chr16_29750001_29780000 | 2.2 | 18.74 | 3.03 | PRR |
| chr11 | 20580001 | 20610000 | 30000 | chr11_20580001_20610000 | 5.36 | 44.25 | 3.02 | RRR |
| chr1 | 28050001 | 28080000 | 30000 | chr1_28050001_28080000 | 2.83 | 23.51 | 3.01 | FRR |
| chr19 | 39280001 | 39300000 | 20000 | chr19_39280001_39300000 | 3.09 | 25.68 | 3.01 | PRR |
| chr4 | 1.53E+08 | 1.53E+08 | 30000 | chr4_153440001_153470000 | 1.36 | 11.39 | 2.98 | FRR |
| chr6 | 1.19E+08 | 1.19E+08 | 50000 | chr6_118810001_118860000 | 4.36 | 34.83 | 2.97 | RRR |
| chr5 | 40790001 | 40810000 | 20000 | chr5_40790001_40810000 | 0.56 | 5.06 | 2.97 | RRR |
| chr1 | 13200001 | 13230000 | 30000 | chr1_13200001_13230000 | 10.41 | 82.42 | 2.97 | FRR |
| chrX | 12950001 | 12980000 | 30000 | chrX_12950001_12980000 | 1.43 | 11.83 | 2.96 | FRR |
| chr6 | 1.08E+08 | 1.09E+08 | 20000 | chr6_108480001_108500000 | 0.85 | 7.2 | 2.94 | FRR |
| chr17 | 57560001 | 57590000 | 30000 | chr17_57560001_57590000 | 0.92 | 7.72 | 2.94 | RRR |
| chr12 | 25100001 | 25130000 | 30000 | chr12_25100001_25130000 | 0.79 | 6.69 | 2.93 | PRR |
| chr12 | 19640001 | 19660000 | 20000 | chr12_19640001_19660000 | 0.63 | 5.48 | 2.93 | PRR |
| chr4 | 89380001 | 89440000 | 60000 | chr4_89380001_89440000 | 5.02 | 38.99 | 2.93 | PRR |
| chr6 | 27540001 | 27560000 | 20000 | chr6_27540001_27560000 | 0.68 | 5.79 | 2.92 | FRR |
| chr7 | 1.4E+08 | 1.4E+08 | 20000 | chr7_139650001_139670000 | 0.91 | 7.43 | 2.9 | RRR |
| chr10 | 96250001 | 96280000 | 30000 | chr10_96250001_96280000 | 1 | 8.03 | 2.89 | RRR |
| chr16 | 50650001 | 50680000 | 30000 | chr16_50650001_50680000 | 2.02 | 15.66 | 2.89 | FRR |
| chr12 | 94650001 | 94680000 | 30000 | chr12_94650001_94680000 | 1.94 | 14.87 | 2.88 | FRR |
| chr19 | 42980001 | 43010000 | 30000 | chr19_42980001_43010000 | 0.79 | 6.4 | 2.87 | FRR |
| chr17 | 42640001 | 42660000 | 20000 | chr17_42640001_42660000 | 0.78 | 6.34 | 2.87 | FRR |
| chr17 | 19710001 | 19740000 | 30000 | chr17_19710001_19740000 | 0.81 | 6.52 | 2.86 | RRR |
| chr6 | 11090001 | 11110000 | 20000 | chr6_11090001_11110000 | 0.86 | 6.85 | 2.86 | FRR |
| chr7 | 25170001 | 25190000 | 20000 | chr7_25170001_25190000 | 1.01 | 7.87 | 2.84 | PRR |
| chr10 | 94060001 | 94090000 | 30000 | chr10_94060001_94090000 | 1.05 | 8.13 | 2.84 | RRR |
| chr2 | 38910001 | 38930000 | 20000 | chr2_38910001_38930000 | 0.8 | 6.34 | 2.84 | PRR |
| chr10 | 70260001 | 70280000 | 20000 | chr10_70260001_70280000 | 1.14 | 8.7 | 2.83 | PRR |
| chr6 | 26280001 | 26300000 | 20000 | chr6_26280001_26300000 | 8.03 | 57.38 | 2.82 | FRR |
| chr2 | 88060001 | 88080000 | 20000 | chr2_88060001_88080000 | 0.72 | 5.66 | 2.81 | PRR |
| chr14 | 1.07E+08 | 1.07E+08 | 40000 | chr14_106920001_106960000 | 9.64 | 66.58 | 2.78 | FRR |
| chr9 | 99800001 | 99820000 | 20000 | chr9_99800001_99820000 | 1.95 | 13.85 | 2.77 | RRR |
| chr16 | 18880001 | 18910000 | 30000 | chr16_18880001_18910000 | 1.98 | 14.12 | 2.77 | FRR |
| chr5 | 1630001 | 1650000 | 20000 | chr5_1630001_1650000 | 0.73 | 5.57 | 2.77 | FRR |
| chr16 | 24530001 | 24560000 | 30000 | chr16_24530001_24560000 | 5.2 | 35.89 | 2.76 | FRR |
| chr11 | 18130001 | 18160000 | 30000 | chr11_18130001_18160000 | 2.66 | 18.54 | 2.76 | RRR |
| chr1 | 1.13E+08 | 1.13E+08 | 30000 | chr1_113250001_113280000 | 1.96 | 13.9 | 2.76 | PRR |
| chr17 | 19680001 | 19700000 | 20000 | chr17_19680001_19700000 | 4.79 | 32.95 | 2.76 | RRR |
| chr1 | 45940001 | 45970000 | 30000 | chr1_45940001_45970000 | 3.14 | 21.88 | 2.76 | FRR |
| chr18 | 56480001 | 56510000 | 30000 | chr18_56480001_56510000 | 1.36 | 9.76 | 2.76 | FRR |
| chrX | 1.49E+08 | 1.49E+08 | 20000 | chrX_148690001_148710000 | 1.94 | 13.56 | 2.74 | RRR |
| chr7 | 1.01E+08 | 1.01E+08 | 20000 | chr7_100930001_100950000 | 2.95 | 20.34 | 2.74 | FRR |
| chr2 | 75120001 | 75140000 | 20000 | chr2_75120001_75140000 | 2.43 | 16.53 | 2.72 | FRR |
| chr7 | 43690001 | 43710000 | 20000 | chr7_43690001_43710000 | 0.89 | 6.37 | 2.71 | FRR |
| chr10 | 43810001 | 43840000 | 30000 | chr10_43810001_43840000 | 3.36 | 22.44 | 2.7 | PRR |
| chr6 | 36390001 | 36420000 | 30000 | chr6_36390001_36420000 | 0.83 | 5.93 | 2.7 | FRR |
| chr2 | 87210001 | 87240000 | 30000 | chr2_87210001_87240000 | 1.09 | 7.58 | 2.69 | PRR |
| chr16 | 75100001 | 75130000 | 30000 | chr16_75100001_75130000 | 1.52 | 10.35 | 2.69 | PRR |
| chr18 | 3820001 | 3850000 | 30000 | chr18_3820001_3850000 | 1.19 | 8.19 | 2.68 | PRR |
| chr6 | 88400001 | 88420000 | 20000 | chr6_88400001_88420000 | 6.15 | 40.01 | 2.68 | FRR |
| chr14 | 35010001 | 35030000 | 20000 | chr14_35010001_35030000 | 1.23 | 8.36 | 2.67 | PRR |
| chr3 | 32920001 | 32950000 | 30000 | chr3_32920001_32950000 | 6.22 | 39.86 | 2.66 | FRR |
| chr10 | 81510001 | 81540000 | 30000 | chr10_81510001_81540000 | 1.08 | 7.33 | 2.65 | RRR |
| chr6 | 1.19E+08 | 1.19E+08 | 30000 | chr6_118870001_118900000 | 4.51 | 28.68 | 2.64 | RRR |
| chr19 | 11750001 | 11800000 | 50000 | chr19_11750001_11800000 | 7.57 | 47.87 | 2.64 | FRR |
| chr7 | 72730001 | 72750000 | 20000 | chr7_72730001_72750000 | 1.49 | 9.61 | 2.61 | FRR |
| chr6 | 90490001 | 90520000 | 30000 | chr6_90490001_90520000 | 1.09 | 7.1 | 2.6 | FRR |
| chr13 | 96200001 | 96230000 | 30000 | chr13_96200001_96230000 | 2.13 | 13.42 | 2.6 | FRR |
| chr10 | 27530001 | 27560000 | 30000 | chr10_27530001_27560000 | 2.37 | 14.82 | 2.59 | PRR |
| chr19 | 23010001 | 23070000 | 60000 | chr19_23010001_23070000 | 17.39 | 105.36 | 2.59 | RRR |
| chr10 | 57720001 | 57750000 | 30000 | chr10_57720001_57750000 | 2.5 | 15.33 | 2.57 | PRR |
| chr8 | 7200001 | 7230000 | 30000 | chr8_7200001_7230000 | 1.28 | 8.12 | 2.57 | FRR |
| chr9 | 89640001 | 89670000 | 30000 | chr9_89640001_89670000 | 1.54 | 9.63 | 2.57 | FRR |
| chr15 | 98270001 | 98300000 | 30000 | chr15_98270001_98300000 | 2.79 | 17.03 | 2.57 | FRR |
| chr16 | 2380001 | 2420000 | 40000 | chr16_2380001_2420000 | 1.78 | 11.05 | 2.57 | PRR |
| chr10 | 1.27E+08 | 1.27E+08 | 20000 | chr10_126650001_126670000 | 2.37 | 14.43 | 2.56 | FRR |
| chr1 | 37980001 | 38000000 | 20000 | chr1_37980001_38000000 | 1.15 | 7.14 | 2.53 | FRR |
| chr18 | 43820001 | 43840000 | 20000 | chr18_43820001_43840000 | 2.25 | 13.35 | 2.52 | PRR |
| chr13 | 51270001 | 51300000 | 30000 | chr13_51270001_51300000 | 4.82 | 27.94 | 2.51 | RRR |
| chr3 | 1270001 | 1290000 | 20000 | chr3_1270001_1290000 | 1.1 | 6.74 | 2.51 | FRR |
| chr7 | 5970001 | 5990000 | 20000 | chr7_5970001_5990000 | 1.78 | 10.6 | 2.51 | RRR |
| chr12 | 1.11E+08 | 1.11E+08 | 30000 | chr12_111380001_111410000 | 3.86 | 22.33 | 2.5 | PRR |
| chr6 | 33080001 | 33110000 | 30000 | chr6_33080001_33110000 | 1.52 | 8.99 | 2.49 | FRR |
| chr19 | 56820001 | 56840000 | 20000 | chr19_56820001_56840000 | 0.87 | 5.33 | 2.48 | FRR |
| chrX | 1.19E+08 | 1.19E+08 | 30000 | chrX_119200001_119230000 | 10.17 | 57.39 | 2.48 | RRR |
| chr12 | 91200001 | 91230000 | 30000 | chr12_91200001_91230000 | 0.89 | 5.37 | 2.47 | RRR |
| chr8 | 170001 | 190000 | 20000 | chr8_170001_190000 | 1.17 | 6.95 | 2.47 | FRR |
| chr14 | 1.04E+08 | 1.04E+08 | 20000 | chr14_104230001_104250000 | 1.05 | 6.24 | 2.46 | PRR |
| chr11 | 1.15E+08 | 1.15E+08 | 40000 | chr11_115080001_115120000 | 4.82 | 26.9 | 2.46 | FRR |
| chr2 | 30350001 | 30370000 | 20000 | chr2_30350001_30370000 | 2.52 | 14.35 | 2.46 | FRR |
| chr10 | 1.22E+08 | 1.22E+08 | 40000 | chr10_121530001_121570000 | 6.62 | 36.66 | 2.45 | FRR |
| chr19 | 45050001 | 45080000 | 30000 | chr19_45050001_45080000 | 1.73 | 9.87 | 2.45 | FRR |
| chr14 | 1.07E+08 | 1.07E+08 | 30000 | chr14_106730001_106760000 | 1.68 | 9.58 | 2.44 | RRR |
| chr5 | 79460001 | 79490000 | 30000 | chr5_79460001_79490000 | 3.94 | 21.82 | 2.44 | FRR |
| chr14 | 74020001 | 74040000 | 20000 | chr14_74020001_74040000 | 1.31 | 7.49 | 2.43 | PRR |
| chr4 | 270001 | 300000 | 30000 | chr4_270001_300000 | 1.76 | 9.93 | 2.43 | FRR |
| chr12 | 50360001 | 50380000 | 20000 | chr12_50360001_50380000 | 1.58 | 8.88 | 2.42 | RRR |
| chr1 | 22300001 | 22330000 | 30000 | chr1_22300001_22330000 | 1.73 | 9.65 | 2.41 | RRR |
| chr5 | 51560001 | 51580000 | 20000 | chr5_51560001_51580000 | 5.85 | 31.4 | 2.4 | RRR |
| chr5 | 1.16E+08 | 1.16E+08 | 20000 | chr5_116080001_116100000 | 2.86 | 15.57 | 2.4 | FRR |
| chr19 | 5830001 | 5860000 | 30000 | chr19_5830001_5860000 | 3.34 | 18 | 2.4 | PRR |
| chr14 | 19640001 | 19660000 | 20000 | chr14_19640001_19660000 | 0.89 | 5.11 | 2.4 | PRR |
| chr7 | 64120001 | 64140000 | 20000 | chr7_64120001_64140000 | 1.2 | 6.75 | 2.4 | FRR |
| chr17 | 80320001 | 80340000 | 20000 | chr17_80320001_80340000 | 7.21 | 38.56 | 2.4 | PRR |
| chr2 | 64170001 | 64190000 | 20000 | chr2_64170001_64190000 | 1.02 | 5.76 | 2.39 | RRR |
| chr10 | 15190001 | 15210000 | 20000 | chr10_15190001_15210000 | 1.67 | 9.15 | 2.39 | PRR |
| chr3 | 78620001 | 78640000 | 20000 | chr3_78620001_78640000 | 1.56 | 8.45 | 2.36 | PRR |
| chr13 | 49790001 | 49810000 | 20000 | chr13_49790001_49810000 | 1.82 | 9.79 | 2.36 | FRR |
| chr18 | 44410001 | 44430000 | 20000 | chr18_44410001_44430000 | 3.19 | 16.5 | 2.34 | FRR |
| chr10 | 35680001 | 35700000 | 20000 | chr10_35680001_35700000 | 0.94 | 5.12 | 2.33 | RRR |

^{#}RNA-seq datasets were obtained from Xue et al., 2013.
^{#}= RNA-seq datasets were obtained from Xue et al., 2013.

TABLE 6
Table 6: Expression levels of transcripts from human Reprogramming Resistant Regions (RRRs), (Related to FIG. 1)

| | | | | | | | | Fold Change (log2) |
|---|---|---|---|---|---|---|---|---|
| | | | | | Expression level (FPKM) | | | [IVF 8-cell/ |
| chromosome | start | end | length | name | donor | IVF 8-cell | SCNT 8-cell | SCNT 8-cell] |
| chr5 | 62880001 | 62930000 | 50000 | chr5_62880001_62930000 | 0 | 64.75 | 0 | 9.34 |
| chr14 | 83550001 | 83580000 | 30000 | chr14_83550001_83580000 | 0 | 115.27 | 0.08 | 9.32 |
| chr4 | 99870001 | 99910000 | 40000 | chr4_99870001_99910000 | 0 | 47.35 | 0 | 8.89 |
| chr4 | 135920001 | 135950000 | 30000 | chr4_135920001_135950000 | 0 | 41.27 | 0 | 8.69 |
| chr14 | 83600001 | 83630000 | 30000 | chr14_83600001_83630000 | 0 | 54.44 | 0.04 | 8.61 |
| chr14 | 47110001 | 47140000 | 30000 | chr14_47110001_47140000 | 0.04 | 89.8 | 0.15 | 8.49 |
| chrX | 147050001 | 147120000 | 70000 | chrX_147050001_147120000 | 0 | 73.22 | 0.12 | 8.38 |
| chr13 | 51270001 | 51300000 | 30000 | chr13_51270001_51300000 | 0.92 | 77.18 | 0.15 | 8.27 |
| chr4 | 46720001 | 46750000 | 30000 | chr4_46720001_46750000 | 0.21 | 50.64 | 0.08 | 8.14 |
| chr10 | 61480001 | 61530000 | 50000 | chr10_61480001_61530000 | 0.13 | 123.03 | 0.35 | 8.1 |
| chr1 | 192810001 | 192850000 | 40000 | chr1_192810001_192850000 | 0.04 | 54.36 | 0.15 | 7.77 |
| chr5 | 51560001 | 51580000 | 20000 | chr5_51560001_51580000 | 0 | 26.06 | 0.04 | 7.55 |
| chr6 | 127490001 | 127510000 | 20000 | chr6_127490001_127510000 | 0 | 17.32 | 0 | 7.44 |
| chr16 | 3050001 | 3070000 | 20000 | chr16_3050001_3070000 | 0.21 | 331.71 | 2.08 | 7.25 |
| chrX | 30220001 | 30280000 | 60000 | chrX_30220001_30280000 | 0 | 102.22 | 0.62 | 7.15 |
| chr6 | 112820001 | 112840000 | 20000 | chr6_112820001_112840000 | 0 | 13.95 | 0 | 7.13 |
| chr16 | 51780001 | 51800000 | 20000 | chr16_51780001_51800000 | 0 | 24.69 | 0.08 | 7.11 |
| chr13 | 32570001 | 32590000 | 20000 | chr13_32570001_32590000 | 0 | 11.56 | 0 | 6.87 |
| chr6 | 117060001 | 117090000 | 30000 | chr6_117060001_117090000 | 0.17 | 43.23 | 0.27 | 6.87 |
| chrX | 151790001 | 151950000 | 160000 | chrX_151790001_151950000 | 0.33 | 393.05 | 3.35 | 6.83 |
| chr12 | 91200001 | 91230000 | 30000 | chr12_91200001_91230000 | 0 | 11.09 | 0 | 6.81 |
| chr3 | 121270001 | 121320000 | 50000 | chr3_121270001_121320000 | 0.67 | 534.82 | 5.36 | 6.61 |
| chr12 | 86090001 | 86120000 | 30000 | chr12_86090001_86120000 | 0 | 9.56 | 0 | 6.59 |
| chrX | 37880001 | 37920000 | 40000 | chrX_37880001_37920000 | 0.08 | 16.85 | 0.08 | 6.56 |
| chr6 | 76130001 | 76160000 | 30000 | chr6_76130001_76160000 | 0.04 | 13.13 | 0.04 | 6.56 |
| chr6 | 115300001 | 115340000 | 40000 | chr6_115300001_115340000 | 0.04 | 198.41 | 2.12 | 6.48 |
| chr8 | 83340001 | 83370000 | 30000 | chr8_83340001_83370000 | 0 | 12.23 | 0.04 | 6.46 |
| chr9 | 75470001 | 75500000 | 30000 | chr9_75470001_75500000 | 0.04 | 11.88 | 0.04 | 6.42 |
| chr11 | 10420001 | 10450000 | 30000 | chr11_10420001_10450000 | 0.04 | 168.7 | 1.93 | 6.38 |
| chr18 | 6770001 | 6800000 | 30000 | chr18_6770001_6800000 | 0 | 14.85 | 0.08 | 6.38 |
| chrX | 55100001 | 55120000 | 20000 | chrX_55100001_55120000 | 0 | 49.97 | 0.5 | 6.38 |
| chr17 | 48340001 | 48370000 | 30000 | chr17_48340001_48370000 | 1.25 | 128.29 | 1.46 | 6.36 |
| chr6 | 78290001 | 78310000 | 20000 | chr6_78290001_78310000 | 0 | 8.07 | 0 | 6.35 |
| chrX | 151080001 | 151110000 | 30000 | chrX_151080001_151110000 | 0 | 93.21 | 1.04 | 6.35 |
| chr10 | 61400001 | 61430000 | 30000 | chr10_61400001_61430000 | 0.04 | 7.6 | 0 | 6.27 |
| chr6 | 26500001 | 26520000 | 20000 | chr6_26500001_26520000 | 0 | 7.45 | 0 | 6.24 |
| chr11 | 82130001 | 82160000 | 30000 | chr11_82130001_82160000 | 0 | 12.46 | 0.08 | 6.12 |
| chrX | 8740001 | 8780000 | 40000 | chrX_8740001_8780000 | 0 | 40.37 | 0.5 | 6.08 |
| chr5 | 53680001 | 53730000 | 50000 | chr5_53680001_53730000 | 0.04 | 21.91 | 0.23 | 6.06 |
| chr13 | 34490001 | 34520000 | 30000 | chr13_34490001_34520000 | 0.08 | 6.43 | 0 | 6.03 |
| chr1 | 195680001 | 195710000 | 30000 | chr1_195680001_195710000 | 0 | 14.58 | 0.15 | 5.88 |
| chr19 | 54120001 | 54160000 | 40000 | chr19_54120001_54160000 | 0.08 | 190.45 | 3.24 | 5.83 |
| chr13 | 34530001 | 34560000 | 30000 | chr13_34530001_34560000 | 1.29 | 22.89 | 0.31 | 5.81 |
| chr13 | 43600001 | 43630000 | 30000 | chr13_43600001_43630000 | 0.04 | 34.41 | 0.54 | 5.75 |
| chr4 | 37010001 | 37040000 | 30000 | chr4_37010001_37040000 | 0 | 5.02 | 0 | 5.68 |
| chr17 | 34310001 | 34340000 | 30000 | chr17_34310001_34340000 | 0.21 | 28.65 | 0.5 | 5.58 |
| chr12 | 49140001 | 49160000 | 20000 | chr12_49140001_49160000 | 0 | 23.48 | 0.5 | 5.3 |
| chr2 | 53200001 | 53230000 | 30000 | chr2_53200001_53230000 | 0 | 13.95 | 0.27 | 5.25 |
| chr13 | 56030001 | 56060000 | 30000 | chr13_56030001_56060000 | 0.04 | 16.93 | 0.35 | 5.24 |
| chr5 | 150670001 | 150760000 | 90000 | chr5_150670001_150760000 | 0.5 | 197.7 | 5.17 | 5.23 |
| chr13 | 84640001 | 84670000 | 30000 | chr13_84640001_84670000 | 0 | 19.09 | 0.42 | 5.21 |
| chr7 | 63460001 | 63490000 | 30000 | chr7_63460001_63490000 | 0.13 | 61.26 | 1.62 | 5.16 |
| chr17 | 4670001 | 4690000 | 20000 | chr17_4670001_4690000 | 0.08 | 41.39 | 1.08 | 5.14 |
| chr3 | 25880001 | 25910000 | 30000 | chr3_25880001_25910000 | 0 | 41.94 | 1.12 | 5.11 |
| chr10 | 89190001 | 89220000 | 30000 | chr10_89190001_89220000 | 0.92 | 16.66 | 0.39 | 5.1 |
| chr12 | 14660001 | 14700000 | 40000 | chr12_14660001_14700000 | 0.33 | 8.27 | 0.15 | 5.07 |
| chr16 | 8150001 | 8180000 | 30000 | chr16_8150001_8180000 | 0 | 12.31 | 0.27 | 5.07 |
| chrX | 47970001 | 47990000 | 20000 | chrX_47970001_47990000 | 0 | 16.62 | 0.42 | 5.01 |
| chr2 | 132710001 | 132740000 | 30000 | chr2_132710001_132740000 | 0 | 14.19 | 0.35 | 4.99 |
| chr19 | 23840001 | 23880000 | 40000 | chr19_23840001_23880000 | 1.38 | 57.58 | 1.73 | 4.98 |
| chr12 | 53500001 | 53530000 | 30000 | chr12_53500001_53530000 | 1.13 | 71.33 | 2.31 | 4.89 |
| chr7 | 100890001 | 100910000 | 20000 | chr7_100890001_100910000 | 1.75 | 25.32 | 0.77 | 4.87 |
| chr17 | 37150001 | 37180000 | 30000 | chr17_37150001_37180000 | 0 | 37.39 | 1.19 | 4.86 |
| chr1 | 160940001 | 160990000 | 50000 | chr1_160940001_160990000 | 0.71 | 469.63 | 16.19 | 4.85 |
| chr7 | 16750001 | 16770000 | 20000 | chr7_16750001_16770000 | 0.04 | 7.02 | 0.15 | 4.83 |
| chr14 | 45350001 | 45370000 | 20000 | chr14_45350001_45370000 | 0.42 | 6.94 | 0.15 | 4.82 |
| chr14 | 102130001 | 102160000 | 30000 | chr14_102130001_102160000 | 1.96 | 14.03 | 0.42 | 4.76 |
| chr17 | 66260001 | 66280000 | 20000 | chr17_66260001_66280000 | 0.33 | 113.39 | 4.16 | 4.74 |
| chr5 | 19020001 | 19050000 | 30000 | chr5_19020001_19050000 | 0 | 19.99 | 0.69 | 4.67 |
| chr17 | 37430001 | 37460000 | 30000 | chr17_37430001_37460000 | 0.67 | 38.18 | 1.43 | 4.64 |
| chr19 | 22460001 | 22520000 | 60000 | chr19_22460001_22520000 | 0.04 | 111.59 | 4.39 | 4.64 |
| chrX | 119200001 | 119230000 | 30000 | chrX_119200001_119230000 | 0.08 | 204.64 | 8.1 | 4.64 |
| chr6 | 5120001 | 5150000 | 30000 | chr6_5120001_5150000 | 0.04 | 11.76 | 0.39 | 4.6 |
| chrX | 69890001 | 69920000 | 30000 | chrX_69890001_69920000 | 0.04 | 14.07 | 0.5 | 4.56 |
| chr9 | 3950001 | 3980000 | 30000 | chr9_3950001_3980000 | 1.04 | 15.64 | 0.58 | 4.53 |
| chr11 | 77560001 | 77580000 | 20000 | chr11_77560001_77580000 | 0.42 | 5.49 | 0.15 | 4.48 |
| chr6 | 30470001 | 30500000 | 30000 | chr6_30470001_30500000 | 0.46 | 24.18 | 1 | 4.46 |
| chr9 | 90700001 | 90740000 | 40000 | chr9_90700001_90740000 | 0 | 47.78 | 2.08 | 4.46 |
| chr16 | 30210001 | 30230000 | 20000 | chr16_30210001_30230000 | 1.25 | 31.43 | 1.35 | 4.44 |
| chr17 | 44310001 | 44350000 | 40000 | chr17_44310001_44350000 | 0 | 24.22 | 1.04 | 4.42 |
| chr7 | 7750001 | 7780000 | 30000 | chr7_7750001_7780000 | 0.88 | 15.91 | 0.69 | 4.34 |
| chr19 | 20400001 | 20430000 | 30000 | chr19_20400001_20430000 | 0.04 | 30.81 | 1.43 | 4.34 |
| chr16 | 29470001 | 29490000 | 20000 | chr16_29470001_29490000 | 1.29 | 30.61 | 1.46 | 4.3 |
| chr16 | 61220001 | 61250000 | 30000 | chr16_61220001_61250000 | 0 | 5.6 | 0.19 | 4.3 |
| chr19 | 51490001 | 51540000 | 50000 | chr19_51490001_51540000 | 0.67 | 160.7 | 8.1 | 4.29 |
| chr5 | 109250001 | 109280000 | 30000 | chr5_109250001_109280000 | 0 | 7.88 | 0.31 | 4.28 |
| chr16 | 70250001 | 70270000 | 20000 | chr16_70250001_70270000 | 1 | 107.08 | 5.44 | 4.27 |
| chr2 | 64170001 | 64190000 | 20000 | chr2_64170001_64190000 | 1.46 | 51.78 | 2.62 | 4.25 |
| chr13 | 19750001 | 19770000 | 20000 | chr13_19750001_19770000 | 0 | 10.31 | 0.46 | 4.22 |
| chr10 | 81680001 | 81710000 | 30000 | chr10_81680001_81710000 | 0.17 | 9.41 | 0.42 | 4.19 |
| chr6 | 56750001 | 56770000 | 20000 | chr6_56750001_56770000 | 0.38 | 8.82 | 0.39 | 4.19 |
| chr10 | 32410001 | 32440000 | 30000 | chr10_32410001_32440000 | 0.08 | 42.76 | 2.27 | 4.18 |
| chr10 | 63610001 | 63640000 | 30000 | chr10_63610001_63640000 | 0 | 9.29 | 0.42 | 4.17 |
| chr4 | 48280001 | 48310000 | 30000 | chr4_48280001_48310000 | 0 | 14.78 | 0.73 | 4.16 |
| chrX | 70970001 | 71000000 | 30000 | chrX_70970001_71000000 | 0 | 35.35 | 1.89 | 4.15 |
| chr11 | 18130001 | 18160000 | 30000 | chr11_18130001_18160000 | 0.33 | 55.03 | 3.01 | 4.15 |
| chr3 | 136580001 | 136600000 | 20000 | chr3_136580001_136600000 | 1.17 | 25.4 | 1.35 | 4.14 |
| chr1 | 150150001 | 150180000 | 30000 | chr1_150150001_150180000 | 0.33 | 163.64 | 9.21 | 4.14 |
| chr10 | 12180001 | 12200000 | 20000 | chr10_12180001_12200000 | 1.92 | 21.48 | 1.16 | 4.1 |
| chr10 | 66860001 | 66920000 | 60000 | chr10_66860001_66920000 | 0.04 | 86.46 | 4.97 | 4.09 |
| chr3 | 112170001 | 112210000 | 40000 | chr3_112170001_112210000 | 0 | 32.1 | 1.81 | 4.08 |
| chr7 | 57500001 | 57560000 | 60000 | chr7_57500001_57560000 | 0.25 | 243.95 | 14.3 | 4.08 |
| chr18 | 57850001 | 57880000 | 30000 | chr18_57850001_57880000 | 0 | 5.45 | 0.23 | 4.07 |
| chr3 | 126970001 | 127020000 | 50000 | chr3_126970001_127020000 | 0.08 | 1099.22 | 65.99 | 4.06 |
| chr11 | 31730001 | 31760000 | 30000 | chr11_31730001_31760000 | 1 | 8.03 | 0.39 | 4.05 |
| chr6 | 107320001 | 107350000 | 30000 | chr6_107320001_107350000 | 0.33 | 44.96 | 2.62 | 4.05 |
| chr17 | 29380001 | 29420000 | 40000 | chr17_29380001_29420000 | 0.33 | 15.64 | 0.85 | 4.05 |
| chr8 | 83390001 | 83450000 | 60000 | chr8_83390001_83450000 | 0.75 | 19.36 | 1.08 | 4.04 |
| chr1 | 35390001 | 35410000 | 20000 | chr1_35390001_35410000 | 0 | 19.68 | 1.12 | 4.02 |
| chr19 | 22350001 | 22390000 | 40000 | chr19_22350001_22390000 | 0.17 | 150.74 | 9.29 | 4.01 |
| chr18 | 19750001 | 19800000 | 50000 | chr18_19750001_19800000 | 0.42 | 78.08 | 4.74 | 4.01 |
| chr2 | 23610001 | 23640000 | 30000 | chr2_23610001_23640000 | 0.08 | 9.45 | 0.5 | 3.99 |
| chr2 | 10590001 | 10610000 | 20000 | chr2_10590001_10610000 | 0.13 | 77.68 | 4.78 | 3.99 |
| chr1 | 22300001 | 22330000 | 30000 | chr1_22300001_22330000 | 0 | 32.14 | 1.93 | 3.99 |
| chr19 | 1890001 | 1910000 | 20000 | chr19_1890001_1910000 | 0.33 | 39.94 | 2.43 | 3.98 |
| chr19 | 23430001 | 23470000 | 40000 | chr19_23430001_23470000 | 1.84 | 72.28 | 4.55 | 3.96 |
| chr7 | 63650001 | 63740000 | 90000 | chr7_63650001_63740000 | 0.04 | 1059.4 | 68.19 | 3.96 |
| chr19 | 36970001 | 37000000 | 30000 | chr19_36970001_37000000 | 0.96 | 60.63 | 3.85 | 3.94 |
| chr15 | 89470001 | 89500000 | 30000 | chr15_89470001_89500000 | 0 | 10.9 | 0.62 | 3.93 |
| chr15 | 63390001 | 63410000 | 20000 | chr15_63390001_63410000 | 0.04 | 7.37 | 0.39 | 3.93 |
| chr11 | 64650001 | 64680000 | 30000 | chr11_64650001_64680000 | 1.54 | 10.74 | 0.62 | 3.91 |
| chrX | 47260001 | 47290000 | 30000 | chrX_47260001_47290000 | 0 | 19.87 | 1.27 | 3.87 |
| chr8 | 81440001 | 81460000 | 20000 | chr8_81440001_81460000 | 0.21 | 10.7 | 0.66 | 3.83 |
| chr19 | 23130001 | 23180000 | 50000 | chr19_23130001_23180000 | 0.04 | 246.42 | 17.23 | 3.83 |
| chr19 | 11840001 | 11860000 | 20000 | chr19_11840001_11860000 | 0.17 | 19.48 | 1.31 | 3.8 |
| chr19 | 54250001 | 54270000 | 20000 | chr19_54250001_54270000 | 0 | 6.15 | 0.35 | 3.8 |
| chrX | 37290001 | 37310000 | 20000 | chrX_37290001_37310000 | 0.67 | 6.55 | 0.39 | 3.76 |
| chr7 | 75000001 | 75030000 | 30000 | chr7_75000001_75030000 | 1.54 | 13.8 | 0.93 | 3.75 |
| chr9 | 78920001 | 78980000 | 60000 | chr9_78920001_78980000 | 0.13 | 28.65 | 2.04 | 3.75 |
| chr10 | 116540001 | 116560000 | 20000 | chr10_116540001_116560000 | 0.04 | 7.41 | 0.46 | 3.75 |
| chr15 | 75440001 | 75470000 | 30000 | chr15_75440001_75470000 | 0.04 | 13.09 | 0.89 | 3.74 |
| chr14 | 19600001 | 19620000 | 20000 | chr14_19600001_19620000 | 0 | 58.64 | 4.36 | 3.72 |
| chr12 | 49680001 | 49710000 | 30000 | chr12_49680001_49710000 | 0.08 | 64.28 | 4.82 | 3.71 |
| chr19 | 21610001 | 21660000 | 50000 | chr19_21610001_21660000 | 0.04 | 137.34 | 10.45 | 3.7 |
| chr1 | 162390001 | 162420000 | 30000 | chr1_162390001_162420000 | 0 | 22.77 | 1.66 | 3.7 |
| chr1 | 146940001 | 147000000 | 60000 | chr1_146940001_147000000 | 0 | 140.24 | 10.95 | 3.67 |
| chr12 | 68810001 | 68840000 | 30000 | chr12_68810001_68840000 | 0.08 | 9.6 | 0.66 | 3.67 |
| chr15 | 23500001 | 23530000 | 30000 | chr15_23500001_23530000 | 0.04 | 8.58 | 0.58 | 3.67 |
| chr12 | 69620001 | 69640000 | 20000 | chr12_69620001_69640000 | 1.13 | 24.18 | 1.81 | 3.67 |
| chr2 | 8110001 | 8130000 | 20000 | chr2_8110001_8130000 | 0 | 8 | 0.54 | 3.66 |
| chr14 | 19970001 | 20000000 | 30000 | chr14_19970001_20000000 | 1.84 | 100.54 | 7.86 | 3.66 |
| chr19 | 22560001 | 22580000 | 20000 | chr19_22560001_22580000 | 0.33 | 21.95 | 1.66 | 3.65 |
| chr16 | 9060001 | 9090000 | 30000 | chr16_9060001_9090000 | 0.08 | 43.35 | 3.35 | 3.65 |
| chr19 | 58730001 | 58750000 | 20000 | chr19_58730001_58750000 | 1.79 | 20.46 | 1.58 | 3.61 |
| chr19 | 18110001 | 18140000 | 30000 | chr19_18110001_18140000 | 0.92 | 24.65 | 1.93 | 3.61 |
| chr8 | 59570001 | 59590000 | 20000 | chr8_59570001_59590000 | 0.17 | 10.27 | 0.77 | 3.58 |
| chr1 | 40990001 | 41010000 | 20000 | chr1_40990001_41010000 | 1.96 | 32.57 | 2.66 | 3.57 |
| chr7 | 63820001 | 63860000 | 40000 | chr7_63820001_63860000 | 0.46 | 28.34 | 2.31 | 3.56 |
| chr19 | 23010001 | 23070000 | 60000 | chr19_23010001_23070000 | 0 | 208.24 | 17.54 | 3.56 |
| chr10 | 28960001 | 28990000 | 30000 | chr10_28960001_28990000 | 0.38 | 30.38 | 2.51 | 3.55 |
| chr1 | 13310001 | 13380000 | 70000 | chr1_13310001_13380000 | 0 | 262.76 | 22.59 | 3.53 |
| chr2 | 178520001 | 178550000 | 30000 | chr2_178520001_178550000 | 0 | 9.52 | 0.73 | 3.53 |
| chr2 | 36830001 | 36850000 | 20000 | chr2_36830001_36850000 | 0 | 13.91 | 1.12 | 3.52 |
| chr13 | 52620001 | 52650000 | 30000 | chr13_52620001_52650000 | 0.46 | 52.48 | 4.47 | 3.52 |
| chr3 | 136440001 | 136470000 | 30000 | chr3_136440001_136470000 | 0.29 | 24.89 | 2.08 | 3.52 |
| chr17 | 66000001 | 66030000 | 30000 | chr17_66000001_66030000 | 0 | 17.29 | 1.43 | 3.51 |
| chrX | 40680001 | 40710000 | 30000 | chrX_40680001_40710000 | 0.08 | 8.58 | 0.66 | 3.51 |
| chr2 | 172760001 | 172780000 | 20000 | chr2_172760001_172780000 | 0.71 | 44.92 | 3.85 | 3.51 |
| chr19 | 57040001 | 57060000 | 20000 | chr19_57040001_57060000 | 1.54 | 12.31 | 1 | 3.5 |
| chr1 | 113420001 | 113450000 | 30000 | chr1_113420001_113450000 | 0.04 | 55.5 | 4.82 | 3.5 |
| chr7 | 5970001 | 5990000 | 20000 | chr7_5970001_5990000 | 0.04 | 32.1 | 2.74 | 3.5 |
| chr10 | 12050001 | 12070000 | 20000 | chr10_12050001_12070000 | 1.17 | 7.02 | 0.54 | 3.48 |
| chr1 | 13460001 | 13540000 | 80000 | chr1_13460001_13540000 | 0 | 286.71 | 25.67 | 3.48 |
| chr17 | 1120001 | 1150000 | 30000 | chr17_1120001_1150000 | 0 | 36.22 | 3.16 | 3.48 |
| chr19 | 22140001 | 22210000 | 70000 | chr19_22140001_22210000 | 0.46 | 129.27 | 11.56 | 3.47 |
| chr19 | 58600001 | 58630000 | 30000 | chr19_58600001_58630000 | 0.46 | 23.63 | 2.04 | 3.47 |
| chrX | 152940001 | 152960000 | 20000 | chrX_152940001_152960000 | 1.25 | 6.98 | 0.54 | 3.47 |
| chr14 | 29290001 | 29320000 | 30000 | chr14_29290001_29320000 | 0 | 17.64 | 1.5 | 3.47 |
| chr6 | 118870001 | 118900000 | 30000 | chr6_118870001_118900000 | 1.96 | 40.88 | 3.62 | 3.46 |
| chr12 | 40470001 | 40500000 | 30000 | chr12_40470001_40500000 | 0.13 | 5.25 | 0.39 | 3.45 |
| chr13 | 41610001 | 41630000 | 20000 | chr13_41610001_41630000 | 0.42 | 7.33 | 0.58 | 3.45 |
| chr19 | 21370001 | 21390000 | 20000 | chr19_21370001_21390000 | 0.71 | 6.39 | 0.5 | 3.44 |
| chr3 | 141230001 | 141260000 | 30000 | chr3_141230001_141260000 | 0.21 | 81.8 | 7.44 | 3.44 |
| chr12 | 38550001 | 38580000 | 30000 | chr12_38550001_38580000 | 0 | 6.78 | 0.54 | 3.43 |
| chr18 | 8750001 | 8780000 | 30000 | chr18_8750001_8780000 | 0.71 | 12.15 | 1.04 | 3.43 |
| chr1 | 146870001 | 146900000 | 30000 | chr1_146870001_146900000 | 0 | 8.31 | 0.69 | 3.41 |
| chr17 | 19710001 | 19740000 | 30000 | chr17_19710001_19740000 | 0.17 | 31.94 | 2.93 | 3.4 |
| chrX | 109090001 | 109120000 | 30000 | chrX_109090001_109120000 | 0 | 35.59 | 3.28 | 3.4 |
| chrX | 148690001 | 148710000 | 20000 | chrX_148690001_148710000 | 0.46 | 10.78 | 0.96 | 3.36 |
| chr2 | 54470001 | 54500000 | 30000 | chr2_54470001_54500000 | 0 | 7.21 | 0.62 | 3.34 |
| chr12 | 14420001 | 14450000 | 30000 | chr12_14420001_14450000 | 0 | 30.3 | 2.93 | 3.33 |
| chr19 | 48350001 | 48390000 | 40000 | chr19_48350001_48390000 | 0.13 | 224.47 | 22.2 | 3.33 |
| chrX | 70920001 | 70940000 | 20000 | chrX_70920001_70940000 | 0.08 | 28.85 | 2.78 | 3.33 |
| chr1 | 13680001 | 13760000 | 80000 | chr1_13680001_13760000 | 0.08 | 278.28 | 27.99 | 3.31 |
| chr2 | 178690001 | 178720000 | 30000 | chr2_178690001_178720000 | 0 | 29.71 | 2.93 | 3.3 |
| chr12 | 85780001 | 85840000 | 60000 | chr12_85780001_85840000 | 0.17 | 50.99 | 5.13 | 3.29 |
| chr7 | 129260001 | 129290000 | 30000 | chr7_129260001_129290000 | 0.75 | 42.02 | 4.2 | 3.29 |
| chr19 | 23550001 | 23600000 | 50000 | chr19_23550001_23600000 | 0.96 | 70.32 | 7.21 | 3.27 |
| chr5 | 81430001 | 81450000 | 20000 | chr5_81430001_81450000 | 0.13 | 6.04 | 0.54 | 3.26 |
| chr1 | 65600001 | 65630000 | 30000 | chr1_65600001_65630000 | 0.96 | 32.73 | 3.35 | 3.25 |
| chrX | 8990001 | 9010000 | 20000 | chrX_8990001_9010000 | 0 | 6 | 0.54 | 3.25 |
| chr18 | 76860001 | 76890000 | 30000 | chr18_76860001_76890000 | 0.88 | 19.36 | 1.97 | 3.23 |
| chr11 | 20580001 | 20610000 | 30000 | chr11_20580001_20610000 | 0.04 | 142.12 | 15.07 | 3.23 |
| chr3 | 155770001 | 155790000 | 20000 | chr3_155770001_155790000 | 0 | 22.42 | 2.31 | 3.22 |
| chr6 | 15180001 | 15210000 | 30000 | chr6_15180001_15210000 | 0 | 5.88 | 0.54 | 3.22 |
| chr1 | 90440001 | 90470000 | 30000 | chr1_90440001_90470000 | 1.21 | 32.1 | 3.39 | 3.21 |
| chr6 | 74270001 | 74300000 | 30000 | chr6_74270001_74300000 | 0.21 | 36.02 | 3.85 | 3.19 |
| chr6 | 32320001 | 32360000 | 40000 | chr6_32320001_32360000 | 0.17 | 20.42 | 2.16 | 3.18 |
| chr1 | 182690001 | 182730000 | 40000 | chr1_182690001_182730000 | 0.04 | 13.88 | 1.5 | 3.13 |
| chr7 | 62740001 | 62770000 | 30000 | chr7_62740001_62770000 | 0 | 58.91 | 6.63 | 3.13 |
| chr7 | 64390001 | 64410000 | 20000 | chr7_64390001_64410000 | 1.17 | 21.32 | 2.35 | 3.13 |
| chr17 | 20850001 | 20900000 | 50000 | chr17_20850001_20900000 | 0 | 7.49 | 0.77 | 3.13 |
| chr11 | 107680001 | 107700000 | 20000 | chr11_107680001_107700000 | 1.88 | 14.5 | 1.58 | 3.12 |
| chr1 | 92440001 | 92540000 | 1.00E+05 | chr1_92440001_92540000 | 0.08 | 527.68 | 60.87 | 3.11 |
| chr2 | 96100001 | 96140000 | 40000 | chr2_96100001_96140000 | 1.34 | 194.25 | 22.74 | 3.09 |
| chr12 | 10090001 | 10120000 | 30000 | chr12_10090001_10120000 | 1.38 | 7.29 | 0.77 | 3.09 |
| chr1 | 46470001 | 46490000 | 20000 | chr1_46470001_46490000 | 1.84 | 7.33 | 0.77 | 3.09 |
| chr1 | 28610001 | 28650000 | 40000 | chr1_28610001_28650000 | 0 | 57.46 | 6.71 | 3.08 |
| chr19 | 40210001 | 40240000 | 30000 | chr19_40210001_40240000 | 0 | 17.72 | 2.04 | 3.06 |
| chr2 | 96270001 | 96310000 | 40000 | chr2_96270001_96310000 | 1.67 | 205.89 | 24.59 | 3.06 |
| chr16 | 48420001 | 48440000 | 20000 | chr16_48420001_48440000 | 0 | 45.11 | 5.32 | 3.06 |
| chr9 | 99800001 | 99820000 | 20000 | chr9_99800001_99820000 | 0.04 | 72.39 | 8.6 | 3.06 |
| chr19 | 6520001 | 6550000 | 30000 | chr19_6520001_6550000 | 0.21 | 11.68 | 1.31 | 3.06 |
| chr6 | 118810001 | 118860000 | 50000 | chr6_118810001_118860000 | 1.25 | 37.43 | 4.43 | 3.05 |
| chr2 | 140190001 | 140240000 | 50000 | chr2_140190001_140240000 | 0 | 5.88 | 0.62 | 3.05 |
| chr7 | 139650001 | 139670000 | 20000 | chr7_139650001_139670000 | 0 | 7.96 | 0.89 | 3.03 |
| chr14 | 20070001 | 20100000 | 30000 | chr14_20070001_20100000 | 0 | 8.47 | 0.96 | 3.02 |
| chr19 | 12100001 | 12120000 | 20000 | chr19_12100001_12120000 | 1.92 | 18.07 | 2.16 | 3.01 |
| chrX | 52770001 | 52810000 | 40000 | chrX_52770001_52810000 | 0 | 25.71 | 3.12 | 3 |
| chr1 | 21700001 | 21730000 | 30000 | chr1_21700001_21730000 | 0 | 9.92 | 1.16 | 2.99 |
| chr10 | 15230001 | 15250000 | 20000 | chr10_15230001_15250000 | 0 | 7.45 | 0.85 | 2.99 |
| chrX | 48240001 | 48280000 | 40000 | chrX_48240001_48280000 | 0 | 37.74 | 4.7 | 2.98 |
| chr19 | 22000001 | 22040000 | 40000 | chr19_22000001_22040000 | 1.84 | 36.06 | 4.51 | 2.97 |
| chr16 | 18920001 | 18950000 | 30000 | chr16_18920001_18950000 | 0.21 | 12.93 | 1.58 | 2.96 |
| chr7 | 64360001 | 64380000 | 20000 | chr7_64360001_64380000 | 0.96 | 20.22 | 2.54 | 2.94 |
| chr10 | 35680001 | 35700000 | 20000 | chr10_35680001_35700000 | 0.17 | 8.66 | 1.04 | 2.94 |
| chr12 | 50360001 | 50380000 | 20000 | chr12_50360001_50380000 | 0.33 | 8.9 | 1.08 | 2.93 |
| chrX | 52710001 | 52740000 | 30000 | chrX_52710001_52740000 | 0 | 23.75 | 3.08 | 2.91 |
| chr14 | 103810001 | 103830000 | 20000 | chr14_103810001_103830000 | 0.96 | 9.09 | 1.12 | 2.91 |
| chr1 | 12980001 | 13070000 | 90000 | chr1_12980001_13070000 | 0.17 | 191.19 | 25.52 | 2.9 |
| chr14 | 100710001 | 100740000 | 30000 | chr14_100710001_100740000 | 1.5 | 8.98 | 1.12 | 2.9 |
| chr7 | 142750001 | 142780000 | 30000 | chr7_142750001_142780000 | 0 | 9.29 | 1.16 | 2.9 |
| chr10 | 81510001 | 81540000 | 30000 | chr10_81510001_81540000 | 0.71 | 9.56 | 1.19 | 2.9 |
| chrX | 134510001 | 134530000 | 20000 | chrX_134510001_134530000 | 0.04 | 13.44 | 1.73 | 2.89 |
| chr9 | 6780001 | 6830000 | 50000 | chr9_6780001_6830000 | 1.88 | 51.66 | 6.86 | 2.89 |
| chr10 | 94060001 | 94090000 | 30000 | chr10_94060001_94090000 | 1.21 | 7.45 | 0.93 | 2.87 |
| chr7 | 64820001 | 64860000 | 40000 | chr7_64820001_64860000 | 1.29 | 54.48 | 7.36 | 2.87 |
| chr14 | 106730001 | 106760000 | 30000 | chr14_106730001_106760000 | 0 | 5.64 | 0.69 | 2.86 |
| chr14 | 103210001 | 103240000 | 30000 | chr14_103210001_103240000 | 0.13 | 5.64 | 0.69 | 2.86 |
| chr17 | 37480001 | 37530000 | 50000 | chr17_37480001_37530000 | 0.17 | 33.08 | 4.47 | 2.86 |
| chr17 | 75760001 | 75790000 | 30000 | chr17_75760001_75790000 | 0 | 8.47 | 1.08 | 2.86 |
| chr5 | 82370001 | 82390000 | 20000 | chr5_82370001_82390000 | 0.38 | 12.31 | 1.62 | 2.85 |
| chr16 | 87510001 | 87540000 | 30000 | chr16_87510001_87540000 | 0.67 | 9.09 | 1.19 | 2.83 |
| chrX | 99650001 | 99670000 | 20000 | chrX_99650001_99670000 | 0.13 | 5.25 | 0.66 | 2.82 |
| chr7 | 63910001 | 63960000 | 50000 | chr7_63910001_63960000 | 0.17 | 21.6 | 2.97 | 2.82 |
| chr4 | 180001 | 240000 | 60000 | chr4_180001_240000 | 1.17 | 39.16 | 5.47 | 2.82 |
| chr1 | 13620001 | 13660000 | 40000 | chr1_13620001_13660000 | 0.04 | 154.62 | 21.74 | 2.82 |
| chr13 | 52140001 | 52180000 | 40000 | chr13_52140001_52180000 | 1.71 | 32.45 | 4.55 | 2.81 |
| chr18 | 11860001 | 11880000 | 20000 | chr18_11860001_11880000 | 0.29 | 6.86 | 0.89 | 2.81 |
| chr3 | 109000001 | 109060000 | 60000 | chr3_109000001_109060000 | 0.58 | 142.87 | 20.51 | 2.79 |
| chr19 | 48770001 | 48810000 | 40000 | chr19_48770001_48810000 | 0.58 | 14.46 | 2 | 2.79 |
| chr16 | 87380001 | 87430000 | 50000 | chr16_87380001_87430000 | 1.54 | 35.08 | 5.09 | 2.76 |
| chr19 | 48290001 | 48320000 | 30000 | chr19_48290001_48320000 | 0.08 | 231.68 | 34.27 | 2.75 |
| chr6 | 32490001 | 32520000 | 30000 | chr6_32490001_32520000 | 0 | 22.22 | 3.24 | 2.74 |
| chr11 | 62110001 | 62140000 | 30000 | chr11_62110001_62140000 | 0.21 | 12.62 | 1.81 | 2.74 |
| chr11 | 82320001 | 82360000 | 40000 | chr11_82320001_82360000 | 0 | 14.27 | 2.08 | 2.72 |
| chr12 | 1870001 | 1890000 | 20000 | chr12_1870001_1890000 | 1.84 | 16.31 | 2.39 | 2.72 |
| chr1 | 13090001 | 13160000 | 70000 | chr1_13090001_13160000 | 0 | 167.01 | 25.48 | 2.71 |
| chr1 | 44570001 | 44620000 | 50000 | chr1_44570001_44620000 | 1.59 | 279.07 | 42.94 | 2.7 |
| chr7 | 91770001 | 91790000 | 20000 | chr7_91770001_91790000 | 0.04 | 28.97 | 4.36 | 2.7 |
| chr8 | 53610001 | 53640000 | 30000 | chr8_53610001_53640000 | 0.5 | 28.1 | 4.28 | 2.69 |
| chr10 | 99080001 | 99110000 | 30000 | chr10_99080001_99110000 | 0.79 | 63.57 | 9.75 | 2.69 |
| chr5 | 32190001 | 32220000 | 30000 | chr5_32190001_32220000 | 0 | 6.66 | 0.96 | 2.67 |
| chr7 | 64020001 | 64090000 | 70000 | chr7_64020001_64090000 | 0.71 | 43.23 | 6.71 | 2.67 |
| chr15 | 75780001 | 75800000 | 20000 | chr15_75780001_75800000 | 1.42 | 6.9 | 1 | 2.67 |
| chr1 | 13400001 | 13440000 | 40000 | chr1_13400001_13440000 | 0 | 150.63 | 23.67 | 2.66 |
| chr7 | 72680001 | 72710000 | 30000 | chr7_72680001_72710000 | 1.34 | 14.19 | 2.16 | 2.66 |
| chr7 | 77150001 | 77170000 | 20000 | chr7_77150001_77170000 | 0.54 | 11.44 | 1.73 | 2.66 |
| chr19 | 41130001 | 41160000 | 30000 | chr19_41130001_41160000 | 0.92 | 8.27 | 1.23 | 2.65 |
| chrX | 54340001 | 54380000 | 40000 | chrX_54340001_54380000 | 0.25 | 50.72 | 8.02 | 2.65 |
| chr15 | 20830001 | 20890000 | 60000 | chr15_20830001_20890000 | 0.08 | 280.09 | 44.6 | 2.65 |
| chr12 | 3260001 | 3280000 | 20000 | chr12_3260001_3280000 | 0 | 5.72 | 0.85 | 2.62 |
| chr4 | 15200001 | 15230000 | 30000 | chr4_15200001_15230000 | 0 | 17.25 | 2.74 | 2.61 |
| chr10 | 88840001 | 88860000 | 20000 | chr10_88840001_88860000 | 1.34 | 6.35 | 0.96 | 2.61 |
| chr19 | 57630001 | 57690000 | 60000 | chr19_57630001_57690000 | 0.54 | 2323.72 | 382.66 | 2.6 |
| chr10 | 93520001 | 93560000 | 40000 | chr10_93520001_93560000 | 0.42 | 41 | 6.75 | 2.58 |
| chr14 | 71370001 | 71390000 | 20000 | chr14_71370001_71390000 | 0.42 | 15.87 | 2.58 | 2.58 |
| chrX | 4450001 | 4470000 | 20000 | chrX_4450001_4470000 | 0.04 | 9.01 | 1.43 | 2.57 |
| chr14 | 20790001 | 20810000 | 20000 | chr14_20790001_20810000 | 1.38 | 23.4 | 3.85 | 2.57 |
| chr1 | 6590001 | 6640000 | 50000 | chr1_6590001_6640000 | 1.92 | 49.19 | 8.25 | 2.56 |
| chr17 | 19680001 | 19700000 | 20000 | chr17_19680001_19700000 | 1.34 | 29.47 | 4.9 | 2.56 |
| chr9 | 123240001 | 123260000 | 20000 | chr9_123240001_123260000 | 1.5 | 6.35 | 1 | 2.55 |
| chr14 | 106310001 | 106340000 | 30000 | chr14_106310001_106340000 | 0 | 7.45 | 1.19 | 2.55 |
| chr7 | 7700001 | 7730000 | 30000 | chr7_7700001_7730000 | 1.42 | 13.05 | 2.16 | 2.54 |
| chr3 | 75540001 | 75560000 | 20000 | chr3_75540001_75560000 | 0 | 15.4 | 2.58 | 2.53 |
| chr17 | 57560001 | 57590000 | 30000 | chr17_57560001_57590000 | 0.04 | 7.37 | 1.19 | 2.53 |
| chr4 | 63290001 | 63320000 | 30000 | chr4_63290001_63320000 | 0 | 5.96 | 0.96 | 2.52 |
| chr19 | 56690001 | 56720000 | 30000 | chr19_56690001_56720000 | 0.38 | 868.21 | 150.99 | 2.52 |
| chr17 | 47070001 | 47090000 | 20000 | chr17_47070001_47090000 | 0.79 | 8.86 | 1.46 | 2.52 |
| chr16 | 22300001 | 22320000 | 20000 | chr16_22300001_22320000 | 0.42 | 7.72 | 1.27 | 2.51 |
| chr15 | 41330001 | 41350000 | 20000 | chr15_41330001_41350000 | 1.54 | 5.29 | 0.85 | 2.5 |
| chr6 | 105520001 | 105540000 | 20000 | chr6_105520001_105540000 | 0.04 | 18.58 | 3.2 | 2.5 |
| chr2 | 156810001 | 156840000 | 30000 | chr2_156810001_156840000 | 0 | 5.02 | 0.81 | 2.49 |
| chr2 | 98240001 | 98260000 | 20000 | chr2_98240001_98260000 | 0 | 92.07 | 16.27 | 2.49 |
| chrX | 80050001 | 80080000 | 30000 | chrX_80050001_80080000 | 0.46 | 11.01 | 1.89 | 2.48 |
| chr5 | 40790001 | 40810000 | 20000 | chr5_40790001_40810000 | 0.5 | 10.35 | 1.77 | 2.48 |
| chr11 | 89800001 | 89840000 | 40000 | chr11_89800001_89840000 | 0.04 | 303.37 | 55.16 | 2.46 |
| chr8 | 126430001 | 126470000 | 40000 | chr8_126430001_126470000 | 0.96 | 9.17 | 1.58 | 2.46 |
| chrX | 48120001 | 48140000 | 20000 | chrX_48120001_48140000 | 0 | 12.93 | 2.27 | 2.46 |
| chr11 | 82650001 | 82680000 | 30000 | chr11_82650001_82680000 | 0.21 | 8.66 | 1.5 | 2.45 |
| chr7 | 57170001 | 57210000 | 40000 | chr7_57170001_57210000 | 0 | 47.27 | 8.6 | 2.44 |
| chr6 | 86360001 | 86380000 | 20000 | chr6_86360001_86380000 | 0.71 | 22.18 | 4.01 | 2.44 |
| chr6 | 34750001 | 34770000 | 20000 | chr6_34750001_34770000 | 0.08 | 5.84 | 1 | 2.43 |
| chr4 | 140040001 | 140070000 | 30000 | chr4_140040001_140070000 | 0.96 | 28.46 | 5.24 | 2.42 |
| chr16 | 46740001 | 46760000 | 20000 | chr16_46740001_46760000 | 0.29 | 21.52 | 4.01 | 2.4 |
| chr7 | 57570001 | 57610000 | 40000 | chr7_57570001_57610000 | 0.04 | 20.62 | 3.85 | 2.39 |
| chr12 | 46360001 | 46380000 | 20000 | chr12_46360001_46380000 | 1.29 | 26.06 | 4.93 | 2.38 |
| chr5 | 64780001 | 64800000 | 20000 | chr5_64780001_64800000 | 0.71 | 13.68 | 2.54 | 2.38 |
| chr17 | 19520001 | 19550000 | 30000 | chr17_19520001_19550000 | 0 | 6.27 | 1.12 | 2.38 |
| chr4 | 128870001 | 128900000 | 30000 | chr4_128870001_128900000 | 1.59 | 9.21 | 1.7 | 2.37 |
| chr1 | 115330001 | 115350000 | 20000 | chr1_115330001_115350000 | 0 | 11.99 | 2.24 | 2.37 |
| chr7 | 26300001 | 26330000 | 30000 | chr7_26300001_26330000 | 0 | 35.31 | 6.75 | 2.37 |
| chr2 | 132220001 | 132240000 | 20000 | chr2_132220001_132240000 | 0.96 | 13.56 | 2.54 | 2.37 |
| chr3 | 5140001 | 5180000 | 40000 | chr3_5140001_5180000 | 1.96 | 50.17 | 9.68 | 2.36 |
| chr19 | 58060001 | 58080000 | 20000 | chr19_58060001_58080000 | 0.67 | 12.62 | 2.39 | 2.35 |
| chr19 | 20650001 | 20670000 | 20000 | chr19_20650001_20670000 | 0 | 10.31 | 1.97 | 2.33 |
| chr10 | 96250001 | 96280000 | 30000 | chr10_96250001_96280000 | 0.42 | 9.09 | 1.73 | 2.33 |
| chr3 | 138710001 | 138780000 | 70000 | chr3_138710001_138780000 | 0.17 | 9.92 | 1.89 | 2.33 |

The Heterochromatin Features of RRRs are Conserved in Human Somatic Cells

The inventors next assessed whether the human RRRs possess the heterochromatin features like that of the mouse RRRs. Analysis of the publically-available ChIP-seq datasets of eight major histone modifications from human fibroblast cells (Bernstein et al., 2012; The Encode Consortium Project, 2011) revealed specific enrichment of H3K9me3 in human RRRs (FIGS. 1D and 5A). The enrichment of H3K9me3 is unique to RRRs, as a similar enrichment was not observed in FRRs or PRRs (FIGS. 1D and 5A). Similar analysis also revealed the enrichment of H3K9me3 at RRRs in K562 erythroleukemic cells, Hsmm skeletal muscle myoblasts, and Mcf7 breast adenocarcinoma cells (FIGS. 1E and 5B), indicating H3K9me3 enrichment in RRRs is a common feature of somatic cells.

Next, the inventors analyzed the DNaseI hypersensitivity of four different somatic cell types using the datasets generated by the ENCODE project. The analysis revealed that RRRs are significantly less sensitive to DNaseI compared to FRR and PRR in all human somatic cell-types analyzed (FIGS. 1F and 5C). Consistent with their heterochromatin feature, human RRRs are relatively gene-poor compared to FRRs or PRRs (FIG. 5D), and are enriched with specific repeat sequences such as LINE and LTR, but not SINE (FIG. 5E). Collectively, these results indicate that the heterochromatin features of RRRs, enrichment of H3K9me3 and decreased accessibility to DNaseI, are conserved in both mouse and human somatic cells.

### Example 2

Human KDM4A mRNA injection improves development of mouse SCNT embryos

Having established that human RRRs are enriched for H3K9me3, the inventors next assessed whether removal of H3K9me3 could help overcome the reprogramming barrier in human SCNT embryos. The inventors previously demonstrated using mouse SCNT model that the H3K9me3 barrier could be removed by injecting mRNAs encoding the mouse H3K9me3 demethylase, KDM4d (Matoba et al., 2014). Before moving into human SCNT model, given that multiple members of the KDM4 family with H3K9me3 demethylase activity exist in mouse and human (Klose et al., 2006; Krishnan and Trievel, 2013; Whetstine et al., 2006), the inventors, instead of using KDM4D in facilitating SCNT reprogramming, assessed if other members of the KDM4 family, such as KDM4A could be used. In addition, the inventors also assessed if KDM4 family members could function across species.

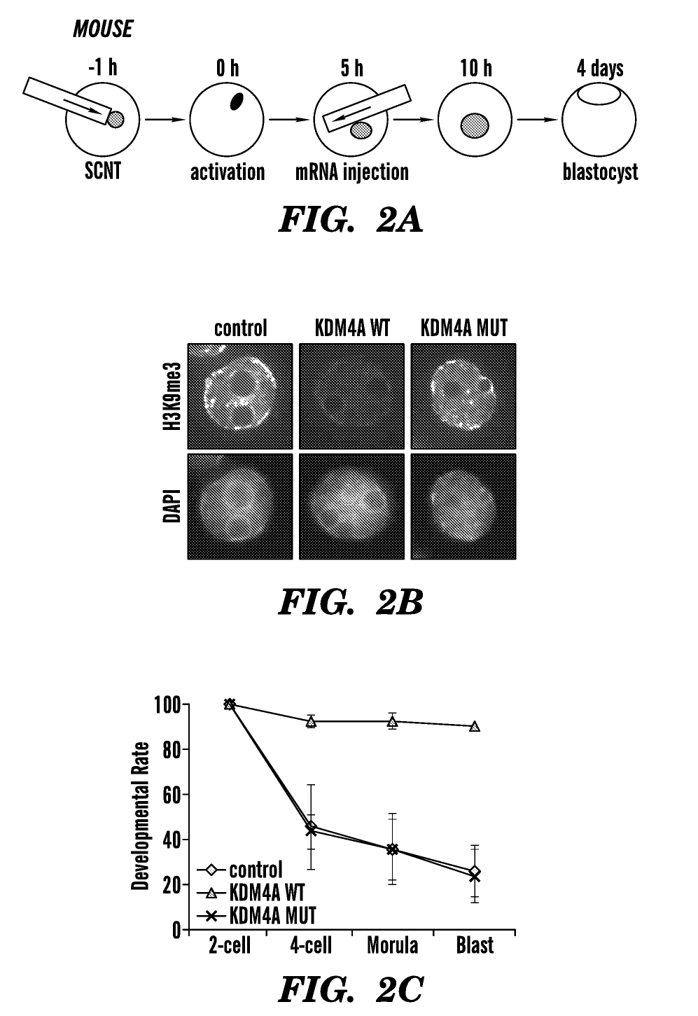

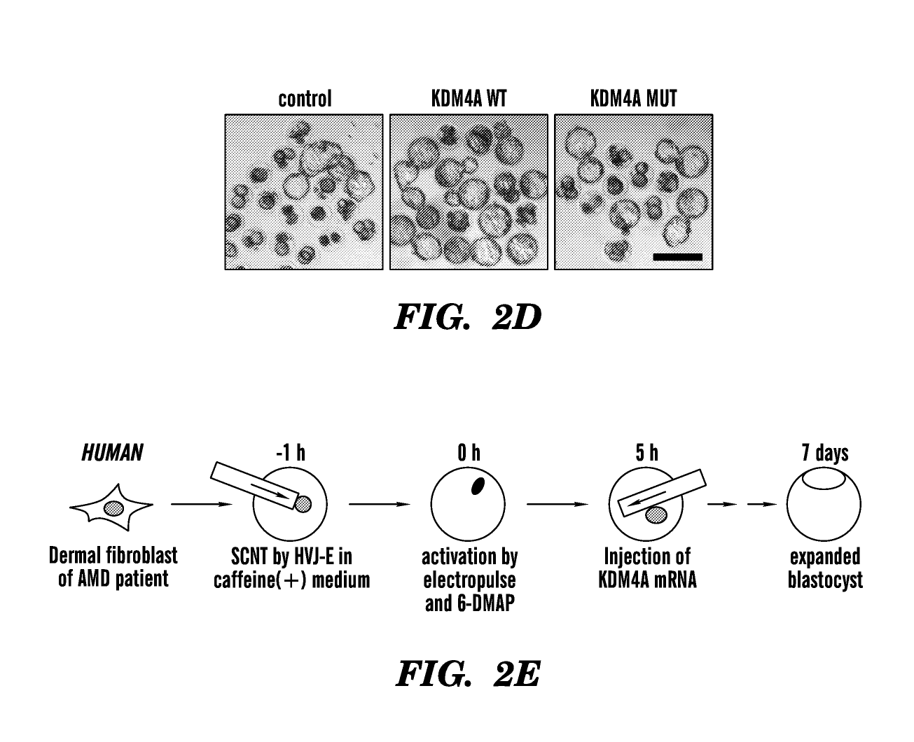

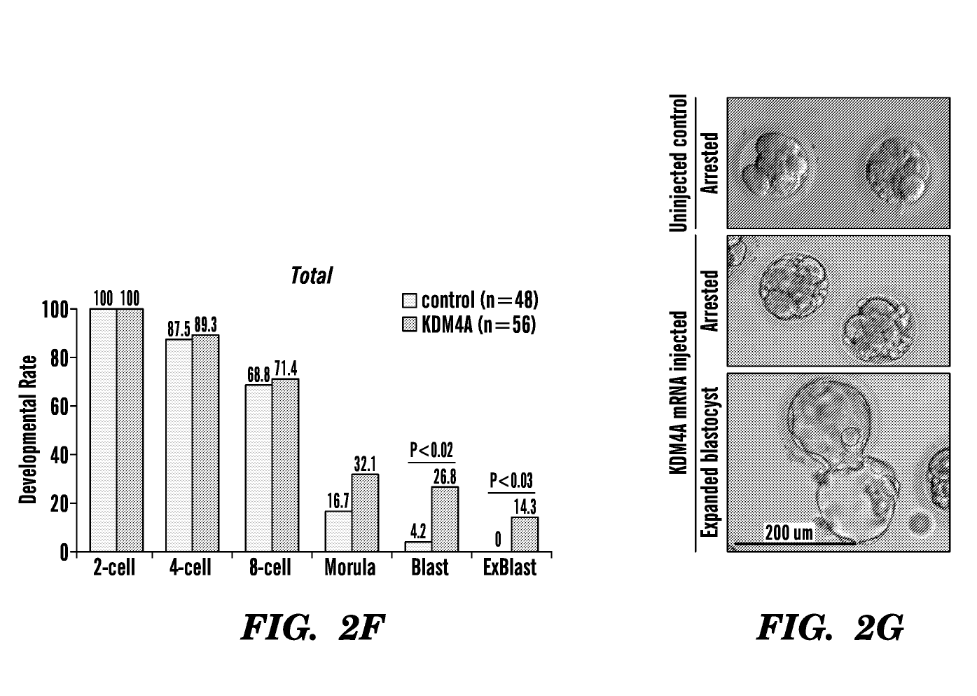

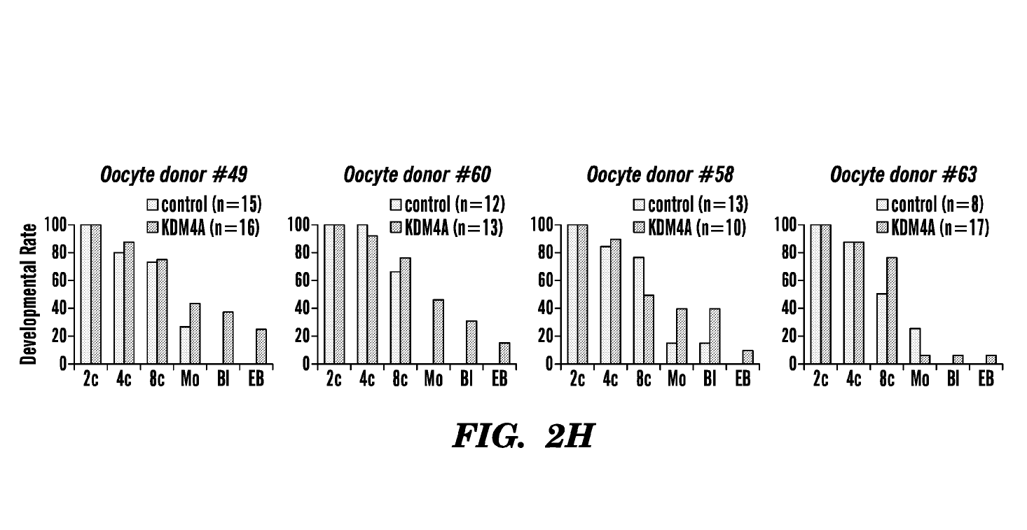

To this end, the inventors performed SCNT using cumulus cells of adult female mice as nuclear donors and injected human KDM4A mRNA at 5 hours post-activation (hpa) following the same procedure used in the inventors previous study (FIG. 2A) (Matoba et al., 2014). Immunostaining revealed that injection of wild-type, but not a catalytic mutant, human KDM4A mRNA greatly reduced H3K9me3 levels in the nucleus of mouse SCNT embryos (FIG. 1B). Importantly, injection of KDM4A mRNA greatly increased the developmental potential of SCNT embryos with 90.3% of them develop to the blastocyst stage, which is in contrast to the 26% blastocyst formation rate in control (FIGS. 2C and 2D, Table 3). The extremely high efficiency of blastocyst formation is similar to the 88.6% observed in KDM4d-injected mouse SCNT embryos (Matoba et al., 2014). These results surprisingly demonstrate that the reprogramming barrier, H3K9me3 in the somatic cell genome, can be removed by any member of the KDM4 family demethylases as long as it contains H3K9me3 demethylase activity.

TABLE 3
Preimplantation development of KDM4A-assisted mouse SCNT embryos, Related to FIG. 2

| | | Concentratio | | No. of | | | | |
|---|---|---|---|---|---|---|---|---|
| Donor | mRNA | of mRNA | No. of | reconstructed | % cleaved per | % 4-cell per | % morula per | % blast per |
| cell-type | injected | (ng/μl) | replicates | 1-cell embryos | 1-cell ± SD | 2-cell ± SD | 2-cell ± SD | 2-cell ± SD |
| Cumulus | Water | | 5 | 91 | 94.8 ± 2.9 | 45.6 ± 18.9 | 35.8 ± 5.6 | 26.0 ± 11.3 |
| | KDM4A WT | 1680 | 3 | 75 | 97.0 ± 5.2 | 96.8 ± 2.7* | 92.5 ± 3.6* | 90.3 ± 0.3* |
| | KDM4A | 1930 | 3 | 74 | 93.7 ± 2.7 | 43.3 ± 7.6 | 35.5 ± 13.5 | 23.7 ± 11.6 |

*P < 0.01 as compared with water injected control.

KDM4A mRNA Injection Significantly Increases the Blastocyst Formation Rate of Human SCNT Embryos

The inventors next assessed if KDM4A mRNA injection could also help overcome the reprogramming barrier in human SCNT using the optimized SCNT conditions including the use of histone deacetylase inhibitor, Trichostatin A (TSA) (Tachibana et al., 2013). With the future clinical application of KDM4A-assisted SCNT in mind, the inventors used dermal fibroblasts of Age-related Macular Degeneration (AMD) patients (Bressler et al., 1988) as nuclear donors.

To reaffirm the beneficial effect of the KDM4A on human SCNT, the inventors choose oocyte donors whose oocytes failed to develop to the expanded blastocyst in prior past attempts using the regular IVF procedures (Chung et al., 2014). Following enucleation, a total of 114 MII oocytes collected from four oocyte donors were fused to donor fibroblast cells by HVJ-E. Upon activation, 63 of the reconstructed SCNT oocytes were injected with human KDM4A mRNA and the rest (51) served as non-injected controls (FIG. 2E, Table 4). The inventors monitored the developmental process of these SCNT embryos and found that the two groups featured similar cleavage efficiencies to form 2-cell embryos (control: 48/51=94.1%, KDM4A: 56/63=88.9%) (Table 4). As expected, KDM4A mRNA injection did not show any beneficial effect on the developmental rate of SCNT embryos before ZGA finishes at the end of 8-cell stage (68.8% vs 71.4%) (FIG. 2F and Table 4). However, the beneficial effect became clear at the morula stage (16.7% vs 32.1%) (FIG. 2F and Table 4). Surprisingly, at day 6, 26.8% (15/56) of the KDM4A-injected embryos had successfully reached the blastocyst stage, as compared to only 4.2% (2/48) of control embryos. On day 7, 14.3% of KDM4A-injected embryos developed to the expanded blastocyst stage, while none of the control embryos developed into this stage (FIGS. 2F and 2G). Importantly, the beneficial effect of KDM4A was observed in all four donors examined (FIG. 2H). Thus, the inventors clearly demonstrate that KDM4A mRNA injection can improve the developmental potential of human SCNT embryos especially beyond ZGA.

TABLE 4
Preimplantation development of KDM4A-assisted human SCNT embryos, Related to FIG. 2.

| Oocyte | | | | | No. of | No. of | No. of | No. of | No. of | No. of | No. of | No. of |
|---|---|---|---|---|---|---|---|---|---|---|---|---|
| donor | Somatic cell donor | | | | donated | reconst | cleaved | 4-cell | cell | morula | blast | ex- |
| [symbol] Age | | | Age | mRNA | MII | 1-cell | (% per | (% per | (% per | (% per | (% per | (% per |
| (years) | ID | Sex | (years) | injected* | oocyte | embry | 1-cell) | 2-cell) | 2-cell) | 2-cell) | 2-cell) | 2-cell) |
| [symbol] 30 | DFB-8 | XY | 59 | — | 15 | 15 | 15 (100) | 12 (80) | 11 (73) | 4 (27) | 0 (0) | 0 (0) |
| | | | | KDM4A | 17 | 17 | 16 (94) | 14 (88) | 12 (75) | 7 (44) | 6 (38) | 4 (25) |
| [symbol] 23 | DFB-7 | XX | 42 | — | 13 | 13 | 13 (100) | 11 (85) | 10 (77) | 2 (15) | 2 (15) | 0 (0) |
| | | | | KDM4A | 11 | 10 | 10 (100) | 9 (90) | 5 (50) | 4 (40) | 4 (40) | 1 (10) |
| [symbol] 27 | DFB-6 | XX | 52 | — | 12 | 12 | 12 (100) | 12 (100) | 8 (67) | 0 (0) | 0 (0) | 0 (0) |
| | | | | KDM4A | 14 | 14 | 13 (93) | 12 (92) | 10 (77) | 6 (46) | 4 (31) | 2 (15) |
| [symbol] 23 | DFB-6 | XX | 52 | — | 12 | 11 | 8 (73) | 7 (88) | 4 (50) | 2 (25) | 0 (0) | 0 (0) |
| | | | | KDM4A | 22 | 22 | 17 (77) | 15 (88) | 13 (76) | 1 (6) | 1 (6) | 1 (6) |

*Concentration of injected human KDM4A mRNAs is 1500 ng/μl. Control embryos are non-injected, blast: blastocyst. ex-blast: expanded blastocyst. [symbol]
indicates data missing or illegible when filed

### Example 3

Establishment and Characterization of Human ESCs Derived from KDM4A-Injected SCNT Blastocysts

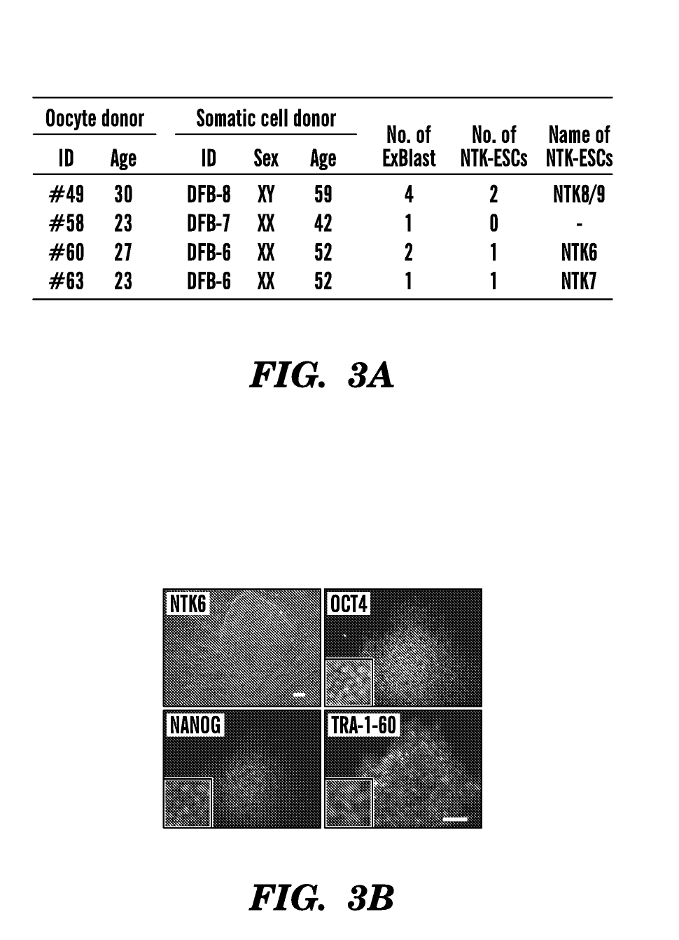

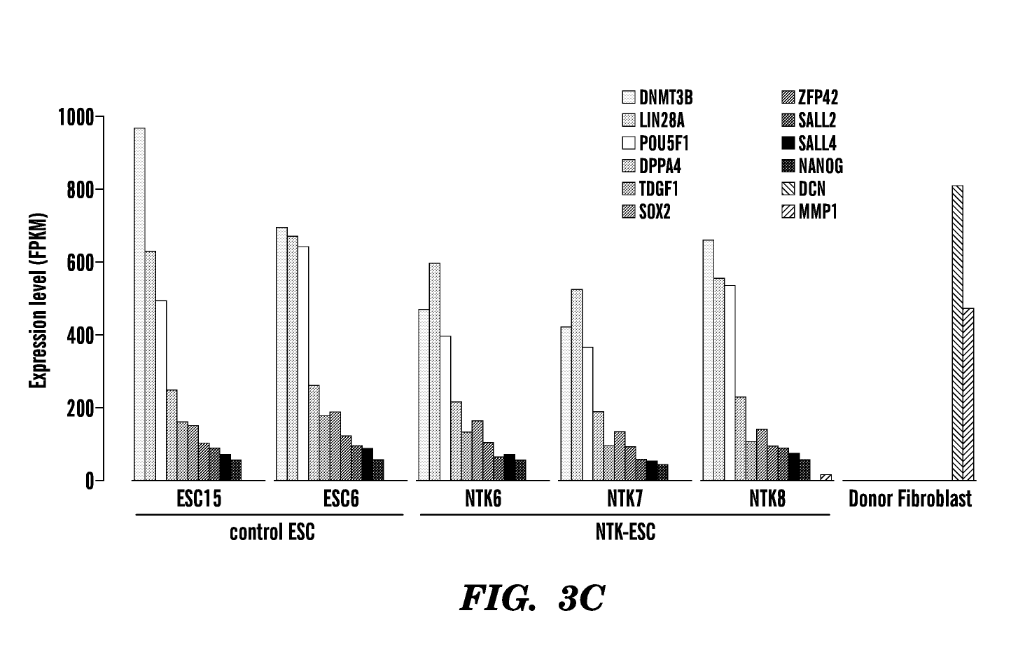

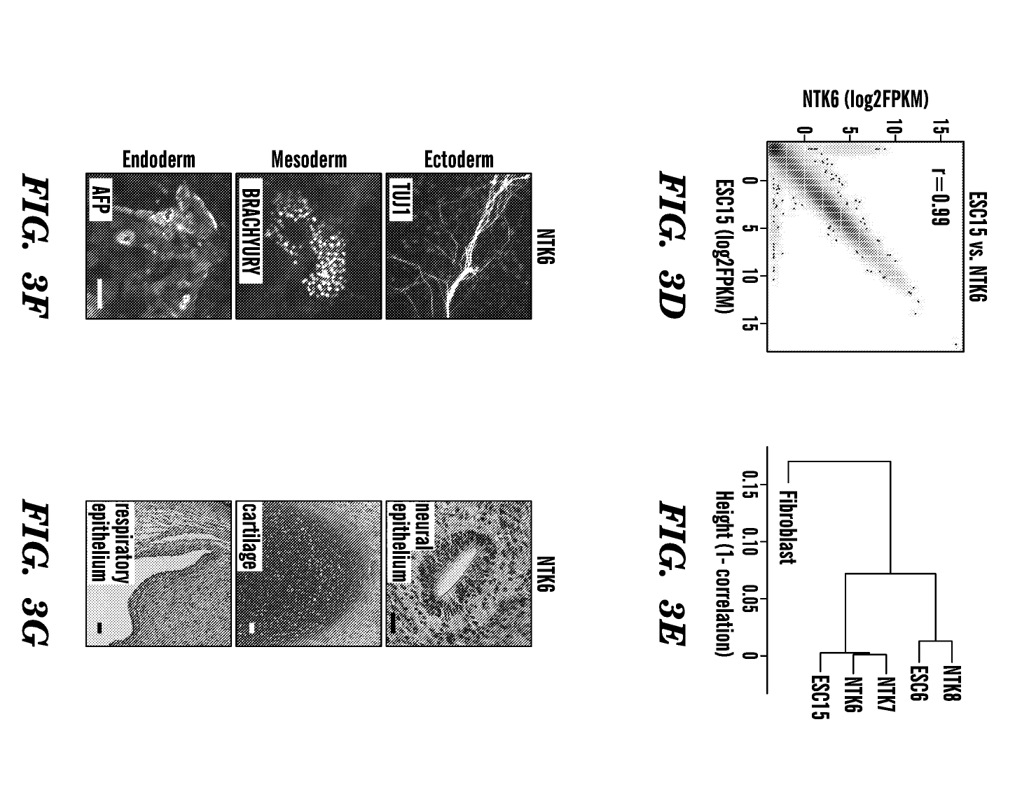

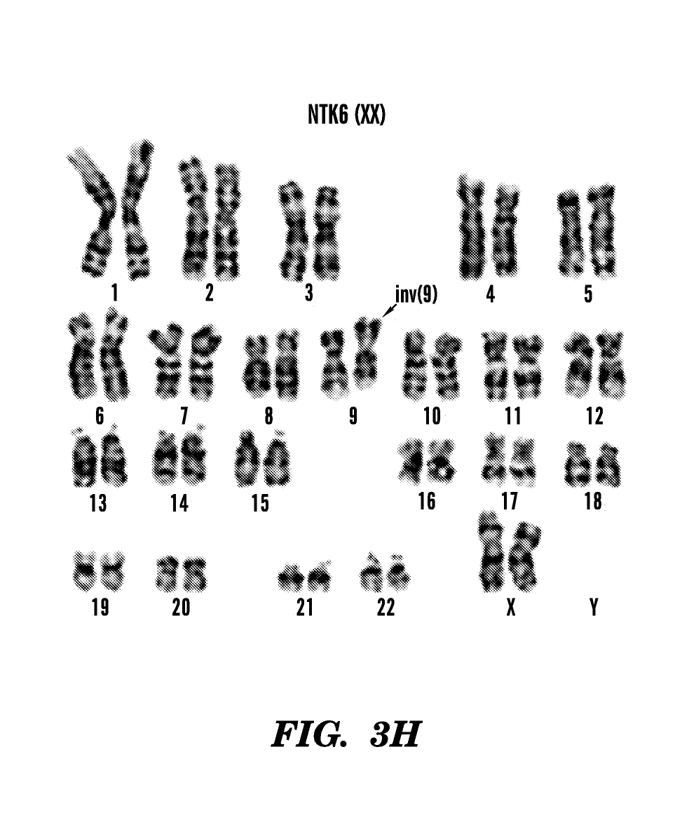

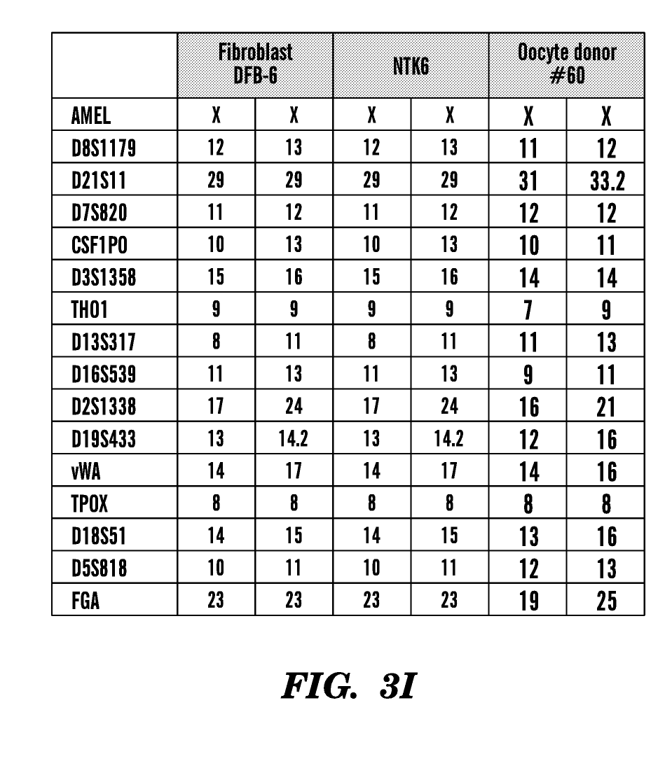

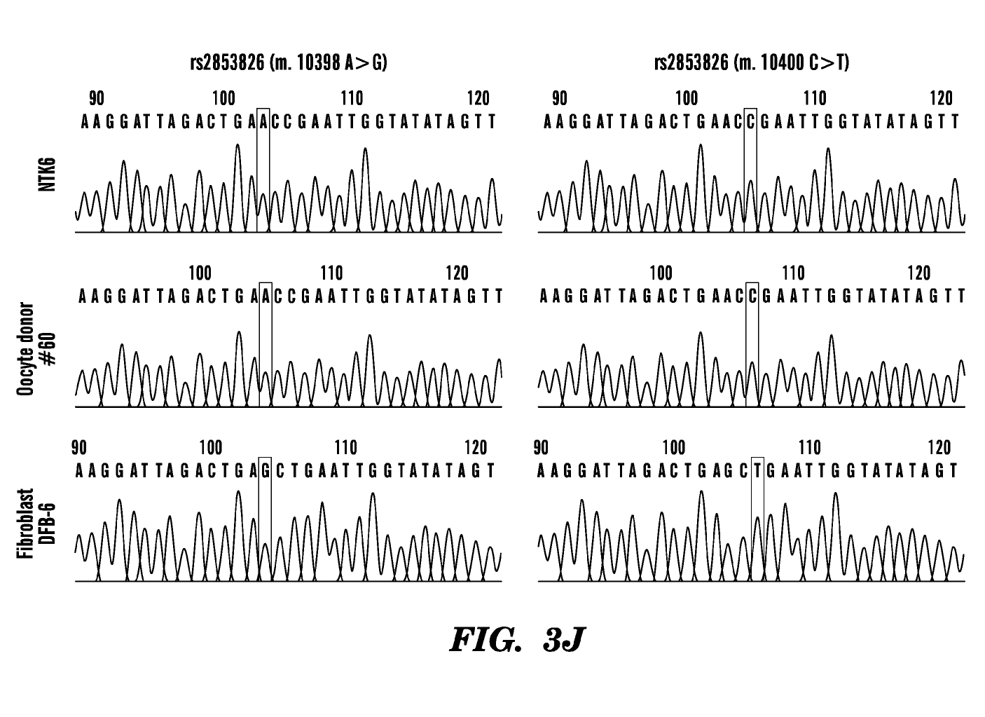

The inventors next to derived nuclear transfer ESCs (NT-ESCs) from KDM4A-injected SCNT blastocysts. The inventors obtained a total of eight expanded blastocysts from KDM4A-injected SCNT embryos (FIG. 3A and Table 4). After removal of the zona pellucida, the expanded blastocysts were cultured on irradiated mouse embryonic fibroblasts (MEF) in a conventional ESC derivation medium. Seven out of the eight blastocysts attached to the MEF feeder cells and initiated outgrowth. After five passages, the inventors successfully derived four stable NT-ESC lines, which were designated as NTK (NT assisted by KDM4A)-ESC #6-9, respectively (FIG. 3A, also named CHA-NT #6-9).

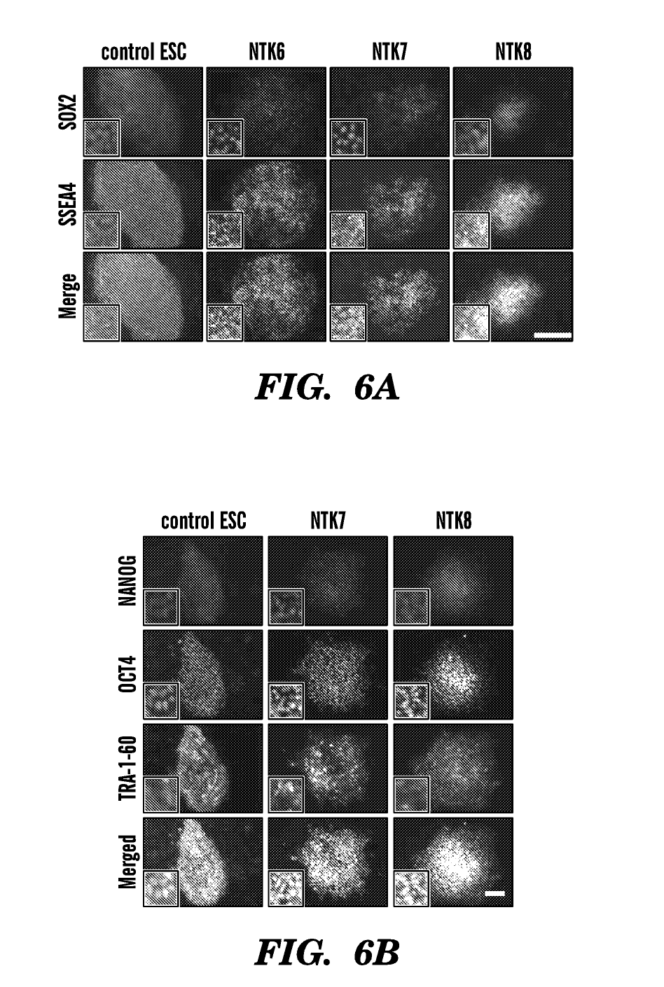

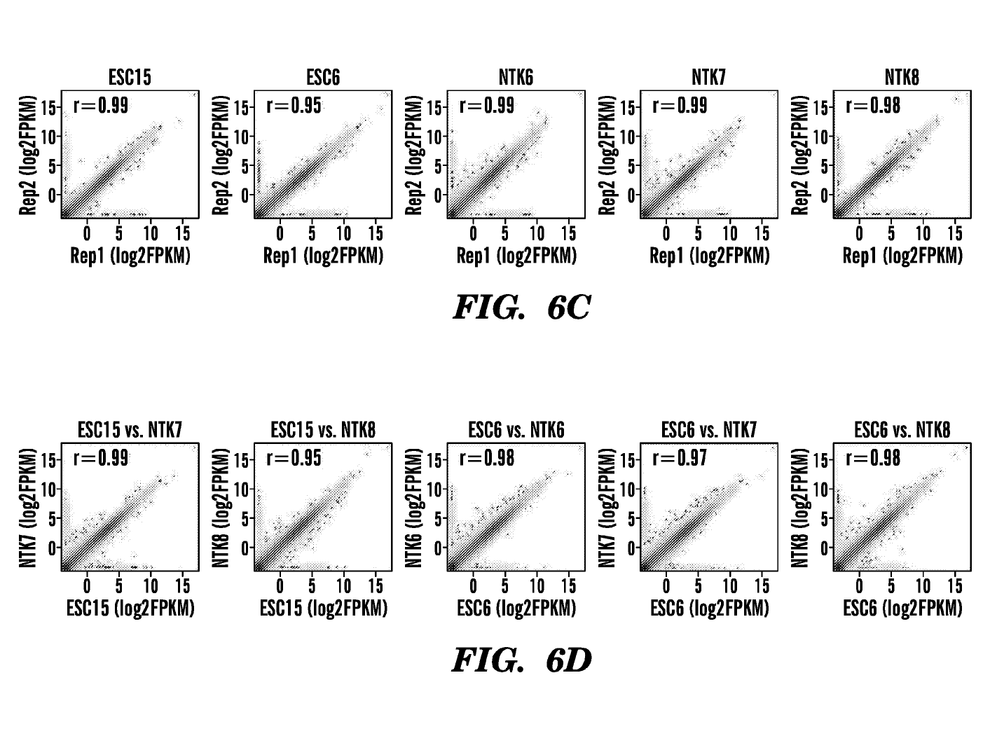

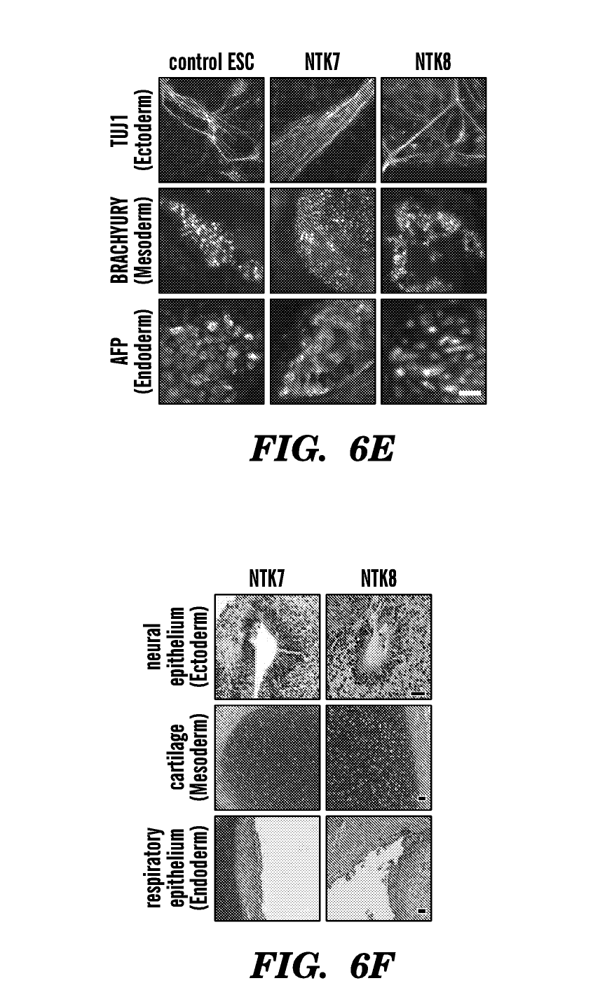

Immunostaining revealed that OCT4, NANOG, SOX2, SSEA-4 and TRA1-60 were all expressed with similar patterns to those of a control human ESC line derived by IVF (FIGS. 3B, FIGS. 6A and 6B). RNA-seq (FIG. 6C) revealed that the NTK-ESCs express pluripotency marker genes at similar levels as control ESCs (FIG. 3C). Pairwise comparison of global transcriptomes revealed a high correlation between NTK-ESCs and control ESCs (FIGS. 3D and 6D). Hierarchical clustering analyses of transcriptomes revealed that NTK-ESCs are clustered together with control ESCs (FIG. 3E). These results suggest that NTK-ESCs are indistinguishable from control ESCs at the molecular level.

The inventors examined the differentiation capacity of the NTK-ESCs by in vitro differentiation and in vivo teratoma assays. Immunostaining of embryoid bodies (EBs) after 2 weeks of in vitro culture revealed that the NTK-ESCs could efficiently give rise to all three germ layer cells (FIGS. 3F and 6E). Moreover, the NTK-ESCs formed teratomas containing all the three germ layer cells within 12 weeks of transplantation (FIGS. 3G and 6F). These results indicate that the NTK-ESCs are pluripotent.

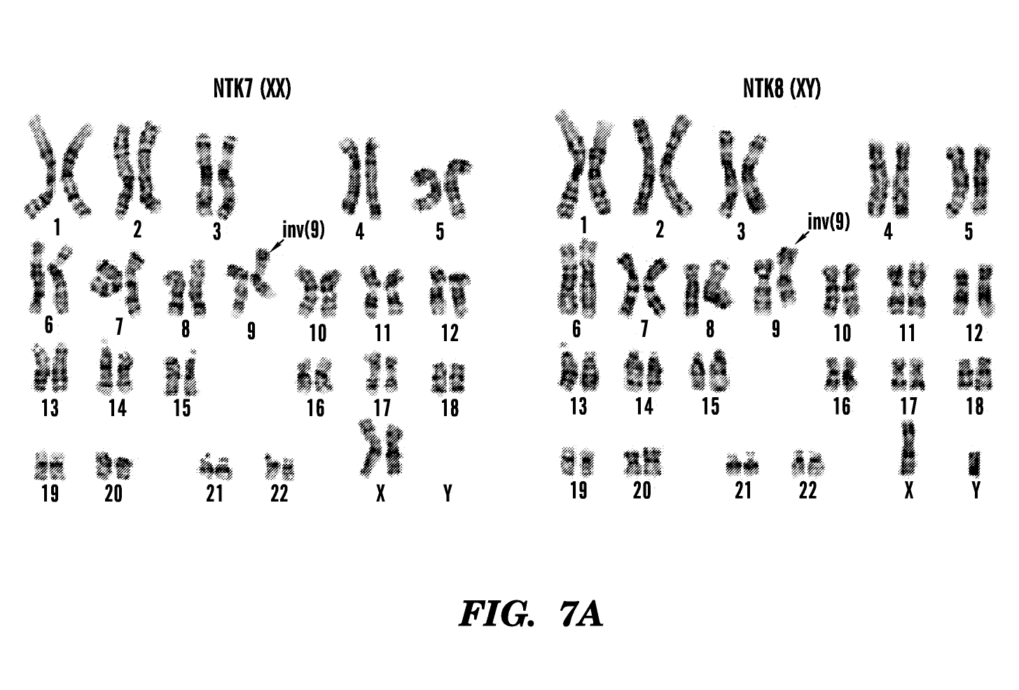

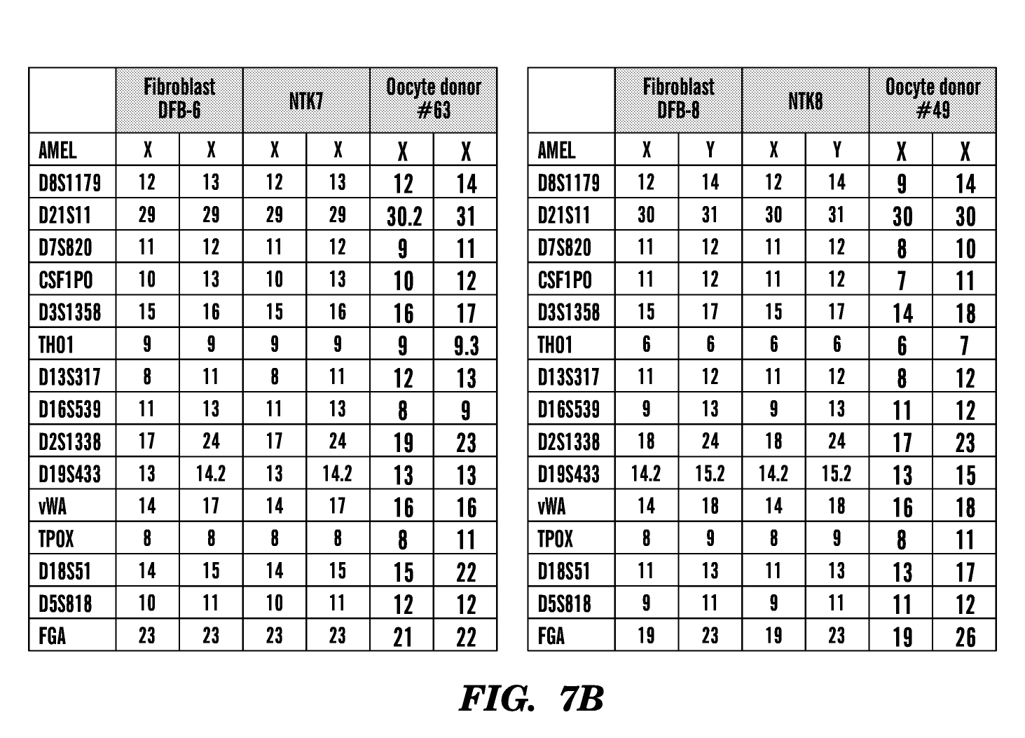

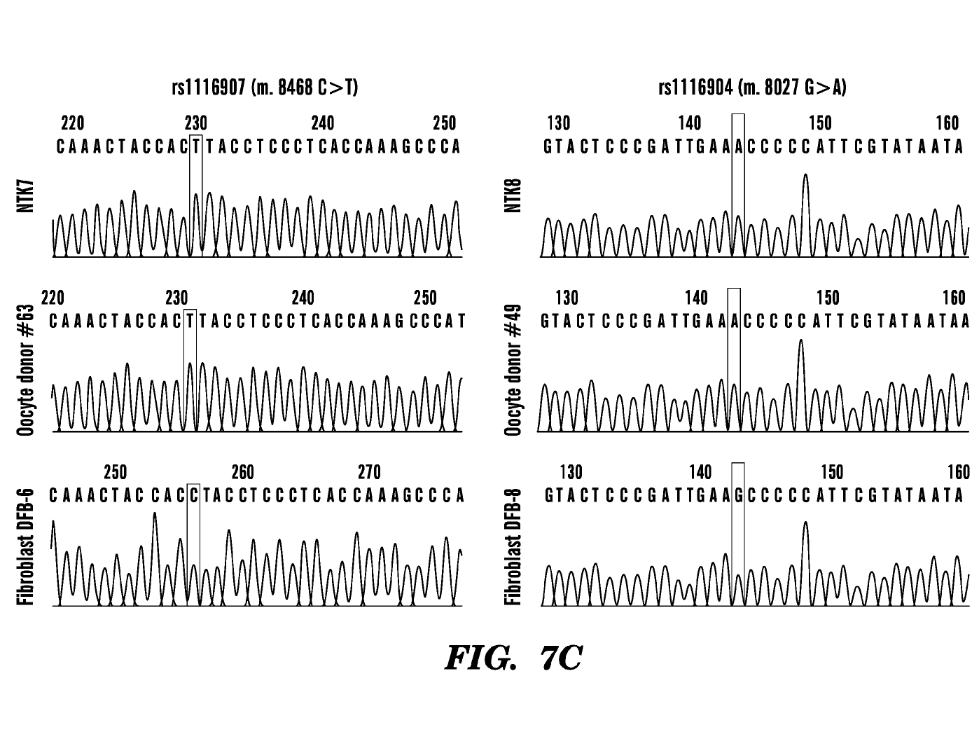

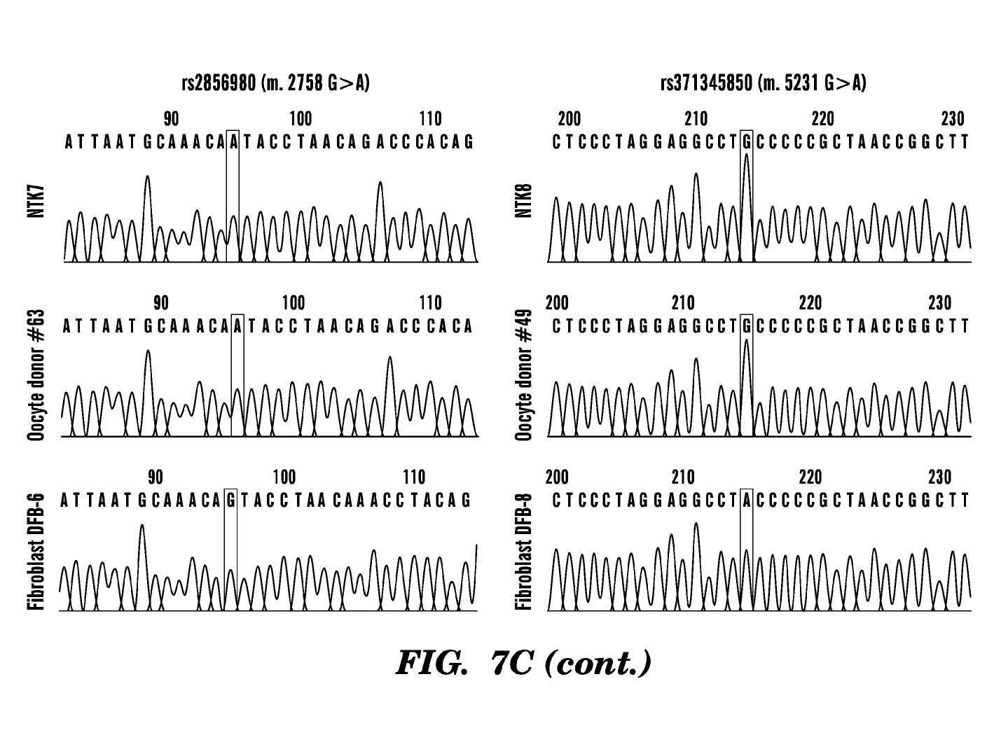

Karyotyping demonstrated that these NTK-ESCs maintain normal number of chromosomes and have the same expected pair of sex chromosomes as those of the nuclear donor somatic cells (46, XX for NTK6/7; 46, XY for NTK8; FIGS. 3H and S3A). Short Tandem Repeat (STR) analysis demonstrated that all the sixteen repeat markers located across the genome showed perfect match between donor somatic cells and their derivative NTK-ESCs (FIGS. 3I and 7B). Mitochondrial DNA sequence analysis revealed that both SNPs of NTK-ESCs matched exactly those of oocyte-donors, but not those of nuclear donors (FIGS. 3J and 7C). Collectively, these results establish the reliability of our SCNT method, and demonstrate that KDM4A mRNA injection improves SCNT-mediated ESC derivation without compromising pluripotency or genomic stability of the established NTK-ESCs.

KDM4A Facilitates ZGA of RRRs in 8-Cell SCNT Embryos

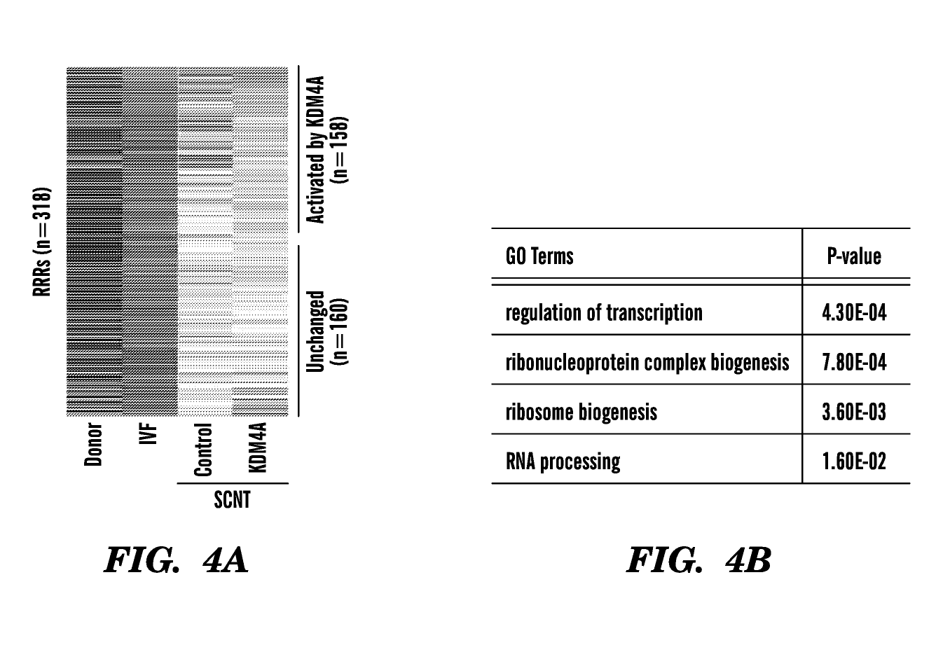

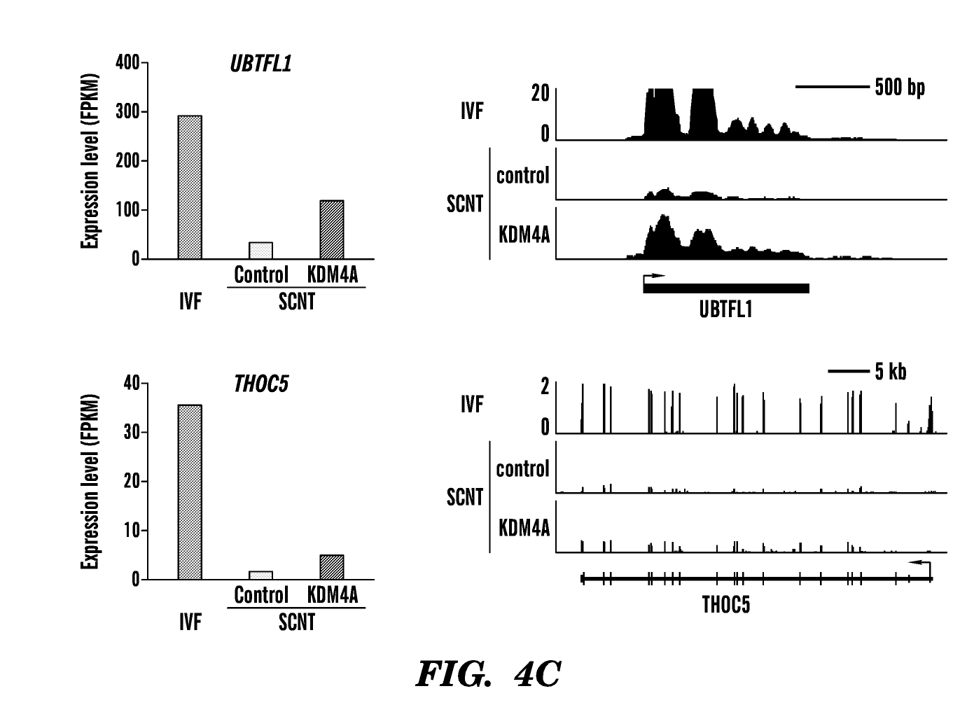

The fact that KDM4A mRNA injection significantly improves hSCNT embryo development post ZGA demonstrates that H3K9me3 in donor somatic cell genome indeed functions as a barrier for ZGA in human SCNT embryos. To determine to what extent the injection of KDM4A mRNAs could overcome ZGA defects in the SCNT embryos, the inventors performed RNA-seq of 8-cell SCNT embryos with or without KDM4A injection. Comparative transcriptome analyses indicated that as much as 50% (158) of the 318 RRRs were markedly up-regulated by KDM4A mRNA injection (FIG. 4A, FC>2), indicating that erasure of H3K9me3 can at least partly facilitate ZGA in SCNT embryos.

To identify candidate gene(s) that might help explain the improved development of KDM4A injected SCNT embryos, the inventors focused analysis of the identified genes. 206 genes (Table 7) whose expression was significantly up-regulated by KDM4A injection (FPKM>5, FC>2). Gene ontology analysis revealed that these genes were enriched for transcriptional regulation, ribosomal biogenesis and RNA processing (FIG. 4B), suggesting that dysregulation of these developmentally important machineries might be a cause of developmental arrest of SCNT embryos. Although the function of the majority of the 206 genes in preimplantation development is currently unknown, two of them, UBTFL1 and THOC5 (FIG. 4C), are known to be required for normal preimplantation development in mice (Wang et al., 2013; Yamada et al., 2009). Therefore, defective activation of these genes is at least partly responsible for the poor development of human SCNT embryos.

Table 7: Expression levels of KDM4A-responsive ZGA genes (Related to FIG. 4).

TABLE 7

| | Expression level (FPKM) | | | | Fold Change (log2) | Fold Change (log2) |
|---|---|---|---|---|---|---|
| | | | Control | KDM4A | [IVF/ | [KDM4A SCNT/ |
| gene | donor | IVF | SCNT | SCNT | Control SCNT] | Control SCNT] |
| ATP5J2- | 0.55 | 18.93 | 0 | 4.51 | 7.57 | 5.53 |
| PTCD1 | | | | | | |
| SPINK7 | 0 | 14.61 | 0.2 | 12.41 | 5.62 | 5.38 |
| RNU11 | 0 | 10.02 | 0 | 3.75 | 6.66 | 5.27 |
| DLEU7 | 0.15 | 33.73 | 0 | 2.91 | 8.4 | 4.91 |
| M1 | 0 | 411.03 | 0 | 2.52 | 12.01 | 4.71 |
| KPTN | 3.47 | 71.09 | 0.24 | 6.84 | 7.71 | 4.35 |
| FAM9A | 0 | 25.61 | 0 | 1.32 | 8.01 | 3.83 |
| CLC | 0 | 31.23 | 0.98 | 13.82 | 4.86 | 3.69 |
| CSAG1 | 0.11 | 144.65 | 0.14 | 2.22 | 9.24 | 3.27 |
| MAGEB2 | 0 | 62.18 | 0 | 0.79 | 9.28 | 3.15 |
| COX7B2 | 0.3 | 149.63 | 0 | 0.79 | 10.55 | 3.15 |
| CCL2 | 80.96 | 5.94 | 0.65 | 6.1 | 3.01 | 3.05 |
| CCL15 | 0 | 26.92 | 0.04 | 0.89 | 7.59 | 2.82 |
| SGCG | 4.03 | 5.12 | 0.07 | 1.03 | 4.94 | 2.73 |
| ZNF826P | 2.09 | 16.57 | 0.24 | 2.07 | 5.62 | 2.67 |
| FAM19A3 | 0.41 | 16.11 | 0.2 | 1.7 | 5.76 | 2.58 |
| PTPN22 | 1.63 | 9.07 | 0.11 | 1.15 | 5.45 | 2.57 |
| ZNF100 | 2.32 | 50.76 | 0.57 | 3.76 | 6.25 | 2.53 |
| SFTPD | 0 | 5.18 | 0.03 | 0.61 | 5.34 | 2.45 |
| PRAMEF3 | 0 | 18.13 | 0.2 | 1.48 | 5.93 | 2.4 |
| MTERF | 11.7 | 36.41 | 0.52 | 3.15 | 5.88 | 2.39 |
| KITLG | 28.76 | 35.88 | 0.1 | 0.93 | 7.49 | 2.36 |
| LOC100289211 | 0 | 20.14 | 1.54 | 8.18 | 3.63 | 2.34 |
| ZNF625- | 1.16 | 6.2 | 0.31 | 1.95 | 3.94 | 2.32 |
| ZNF20 | | | | | | |
| VPS54 | 3.9 | 26.91 | 0.7 | 3.79 | 5.08 | 2.28 |
| LOC284408 | 1.46 | 46.82 | 0.21 | 1.36 | 7.24 | 2.24 |
| VAMP1 | 5.82 | 23.55 | 2.7 | 13.05 | 3.08 | 2.23 |
| CXorf61 | 0 | 12.63 | 0.11 | 0.85 | 5.92 | 2.18 |
| PPP2CB | 24.75 | 14.8 | 0.16 | 1.07 | 5.84 | 2.17 |
| C20orf7 | 10.11 | 11.37 | 0.44 | 2.28 | 4.41 | 2.14 |
| ZNF679 | 0 | 311.32 | 19.78 | 86.94 | 3.97 | 2.13 |
| LOC653513 | 0.45 | 11.37 | 0.16 | 1.03 | 5.46 | 2.12 |
| 12-Sep | 0 | 11.35 | 0 | 0.3 | 6.84 | 2 |
| LIM2 | 0 | 14.74 | 0.43 | 2.01 | 4.81 | 1.99 |
| FAM151A | 1.19 | 150.04 | 0.18 | 1.01 | 9.07 | 1.99 |
| BTAF1 | 2.79 | 11.71 | 0.31 | 1.46 | 4.85 | 1.93 |
| KHDC1 | 3.65 | 244.62 | 22.95 | 85.25 | 3.41 | 1.89 |
| ALG5 | 75.12 | 92.57 | 2.55 | 9.71 | 5.13 | 1.89 |
| ZNF675 | 6.14 | 137.41 | 4.05 | 15.04 | 5.05 | 1.87 |
| ZNF625 | 1.87 | 15.14 | 0.2 | 1 | 5.67 | 1.87 |
| SNAR-C3 | 0 | 2211.75 | 27.24 | 98.14 | 6.34 | 1.85 |
| IL13RA2 | 58.59 | 26.81 | 0 | 0.26 | 8.07 | 1.85 |
| H3F3A | 16.86 | 137.56 | 3.98 | 14.47 | 5.08 | 1.84 |
| RP2 | 17.73 | 6.63 | 0.3 | 1.31 | 4.07 | 1.82 |
| NUDT9 | 21.53 | 9.06 | 0.31 | 1.35 | 4.48 | 1.82 |
| RPS6KB2 | 37.1 | 13.09 | 0.02 | 0.32 | 6.78 | 1.81 |
| MAGEA12 | 0 | 36.15 | 0 | 0.25 | 8.5 | 1.81 |
| UBTFL1 | 0.05 | 291.04 | 34.32 | 120.09 | 3.08 | 1.8 |
| PDE4DIP | 9.38 | 18.06 | 1.1 | 4.04 | 3.92 | 1.79 |
| NANOGNB | 0.14 | 224.47 | 8.87 | 30.92 | 4.65 | 1.79 |
| C12orf60 | 3.01 | 94.51 | 13.42 | 46.22 | 2.81 | 1.78 |
| VCX2 | 0 | 9.84 | 0 | 0.24 | 6.64 | 1.77 |
| CNPY4 | 28.16 | 9.11 | 1.45 | 5.19 | 2.57 | 1.77 |
| KLK11 | 0 | 146.34 | 2.02 | 7.07 | 6.11 | 1.76 |
| ZNF729 | 0.01 | 15.68 | 0.21 | 0.94 | 5.67 | 1.75 |
| LOC401397 | 200.17 | 80.87 | 3.25 | 11.19 | 4.6 | 1.75 |
| ZNF486 | 1.78 | 14.55 | 0.76 | 2.77 | 4.09 | 1.74 |
| TTC28-AS1 | 4.77 | 47.69 | 2.27 | 7.79 | 4.33 | 1.74 |
| FBXL12 | 8.39 | 8.63 | 0.47 | 1.8 | 3.94 | 1.74 |
| SHFM1 | 642.03 | 338.42 | 6.31 | 21.11 | 5.72 | 1.73 |
| FAM162A | 74.24 | 75.16 | 5.77 | 19.35 | 3.68 | 1.73 |
| VCX | 0 | 7.47 | 0 | 0.23 | 6.24 | 1.72 |
| TTC27 | 18.22 | 22.84 | 0.1 | 0.56 | 6.84 | 1.72 |
| SNAR-E | 132.48 | 27727.6 | 747.8 | 2468.68 | 5.21 | 1.72 |
| SNAPC1 | 23.13 | 53.15 | 5.33 | 17.7 | 3.29 | 1.71 |
| SIAH1 | 4.57 | 569.25 | 27.31 | 88.93 | 4.38 | 1.7 |
| PLBD1 | 0.06 | 5.02 | 0.16 | 0.74 | 4.3 | 1.69 |
| SUZ12P | 0.58 | 6.08 | 0.34 | 1.31 | 3.81 | 1.68 |
| PPM1N | 0.03 | 9.05 | 0.21 | 0.89 | 4.88 | 1.68 |
| KLK4 | 0 | 5.38 | 0.37 | 1.41 | 3.54 | 1.68 |
| C1D | 106.39 | 610.21 | 30.49 | 97 | 4.32 | 1.67 |
| TEN1 | 28.72 | 10.56 | 1.02 | 3.43 | 3.25 | 1.66 |
| CLDN6 | 0.13 | 276.58 | 1.06 | 3.54 | 7.9 | 1.65 |
| PLVAP | 0 | 12 | 0.46 | 1.63 | 4.43 | 1.63 |
| LOC100506668 | 8.5 | 27.33 | 2.77 | 8.79 | 3.26 | 1.63 |
| CELA3B | 0 | 12.68 | 0.09 | 0.49 | 6.07 | 1.63 |
| DRAM1 | 12.86 | 11.34 | 0.14 | 0.63 | 5.57 | 1.6 |
| MRPL28 | 137.28 | 22.89 | 0.45 | 1.56 | 5.39 | 1.59 |
| GIP | 0.69 | 192 | 4.23 | 12.94 | 5.47 | 1.59 |
| ZNHIT6 | 7.27 | 10.63 | 1.23 | 3.88 | 3.01 | 1.58 |
| PIK3R4 | 9.7 | 16.52 | 0.37 | 1.31 | 5.14 | 1.58 |
| LOC100505854 | 3.44 | 26.67 | 1.53 | 4.77 | 4.04 | 1.58 |
| GOLGB1 | 13.56 | 7.74 | 0.39 | 1.36 | 4 | 1.58 |
| ZNF208 | 0.06 | 11.9 | 0.52 | 1.74 | 4.27 | 1.57 |
| POTEM | 0 | 9.89 | 0.6 | 1.96 | 3.84 | 1.56 |
| BLVRB | 34.36 | 20.53 | 0.65 | 2.11 | 4.78 | 1.56 |
| TRIML2 | 0 | 41.08 | 0.55 | 1.8 | 5.99 | 1.55 |
| GALM | 12.76 | 23.12 | 1.19 | 3.67 | 4.17 | 1.55 |
| MPV17L2 | 4.85 | 9.17 | 1.47 | 4.48 | 2.56 | 1.54 |
| KHDC1L | 1.75 | 16683.7 | 230.38 | 668.53 | 6.18 | 1.54 |
| ZNF684 | 4.91 | 54.59 | 2.44 | 7.21 | 4.43 | 1.53 |
| ZNF700 | 2.96 | 7.21 | 0.6 | 1.86 | 3.38 | 1.49 |
| ZFYVE19 | 18.4 | 15.45 | 1.38 | 4.05 | 3.39 | 1.49 |
| FMR1NB | 0 | 80.4 | 0 | 0.18 | 9.65 | 1.49 |
| CNKSR3 | 0.73 | 6.07 | 0.04 | 0.29 | 5.46 | 1.48 |
| ZNF345 | 1.3 | 11.48 | 0.45 | 1.41 | 4.4 | 1.46 |
| THOC5 | 29.48 | 35.42 | 1.76 | 5.02 | 4.26 | 1.46 |
| RPF2 | 120.62 | 546.39 | 37.07 | 102.42 | 3.88 | 1.46 |
| PHOSPHO1 | 0 | 7.05 | 0.02 | 0.23 | 5.9 | 1.46 |
| PRAMEF17 | 0 | 6.91 | 0.18 | 0.66 | 4.65 | 1.44 |
| MED31 | 28.81 | 268.28 | 9.99 | 27.22 | 4.73 | 1.44 |
| LOC643955 | 0 | 20.31 | 1.11 | 3.18 | 4.08 | 1.44 |
| COX17 | 282.86 | 364.43 | 9.72 | 26.62 | 5.21 | 1.44 |
| C18orf56 | 3.07 | 8.65 | 0.06 | 0.33 | 5.77 | 1.43 |
| TMEM92 | 0.43 | 48.78 | 0.3 | 0.97 | 6.93 | 1.42 |
| SNHG12 | 3.48 | 34.45 | 2.56 | 7.02 | 3.7 | 1.42 |
| TIMM10 | 146.61 | 606.02 | 9.42 | 25.23 | 5.99 | 1.41 |
| SGPP1 | 5.74 | 7.64 | 0.46 | 1.39 | 3.79 | 1.41 |
| TCN2 | 2.19 | 5.92 | 0.2 | 0.69 | 4.33 | 1.4 |
| PRPF39 | 9.9 | 31.33 | 1.26 | 3.48 | 4.53 | 1.4 |
| NOP58 | 95.17 | 464.08 | 20.99 | 55.54 | 4.46 | 1.4 |
| MFSD11 | 11.68 | 24.72 | 2.94 | 7.94 | 3.03 | 1.4 |
| RAB9A | 31.41 | 186.19 | 0.46 | 1.37 | 8.38 | 1.39 |
| LINC00263 | 0.64 | 12.46 | 1.39 | 3.81 | 3.08 | 1.39 |
| ZNF791 | 16.38 | 25.28 | 1.39 | 3.79 | 4.09 | 1.38 |
| TAC1 | 0.17 | 13.69 | 2.14 | 5.72 | 2.62 | 1.38 |
| ZNF326 | 9.16 | 23.42 | 1.02 | 2.8 | 4.39 | 1.37 |
| ZNF254 | 4.02 | 107.91 | 0.45 | 1.32 | 7.62 | 1.37 |
| SEPX1 | 33.35 | 5.15 | 0.85 | 2.34 | 2.47 | 1.36 |
| RASA2 | 1.46 | 7.97 | 0.33 | 1 | 4.23 | 1.36 |
| LOC347411 | 0 | 8.55 | 0.57 | 1.62 | 3.69 | 1.36 |
| GJA1 | 66.62 | 23.99 | 0.58 | 1.64 | 5.15 | 1.36 |
| UTP23 | 8.59 | 27.75 | 1.23 | 3.29 | 4.39 | 1.35 |
| STAG3L2 | 2.31 | 34.62 | 1.01 | 2.72 | 4.97 | 1.35 |
| LARP6 | 17.8 | 9.07 | 0.1 | 0.41 | 5.52 | 1.35 |
| ZNF280A | 0.18 | 875.26 | 33.4 | 84.94 | 4.71 | 1.34 |
| POLR3K | 57.96 | 569.5 | 43.15 | 109.26 | 3.72 | 1.34 |
| C11orf67 | 98.51 | 68.59 | 11.09 | 28.21 | 2.62 | 1.34 |
| FAM83D | 7.53 | 16.01 | 0.95 | 2.53 | 3.94 | 1.32 |
| ACBD5 | 17.46 | 20.05 | 0.59 | 1.62 | 4.87 | 1.32 |
| PRAMEF11 | 0 | 55.08 | 7.28 | 18.18 | 2.9 | 1.31 |
| SNHG9 | 246.15 | 1263.55 | 25.71 | 63.59 | 5.61 | 1.3 |
| ZNF676 | 0.04 | 40.96 | 1.82 | 4.61 | 4.42 | 1.29 |
| RFK | 4.26 | 75.03 | 1.11 | 2.86 | 5.96 | 1.29 |
| FOXN2 | 9.46 | 38.72 | 2.2 | 5.53 | 4.08 | 1.29 |
| CUL2 | 34.51 | 546.12 | 5.8 | 14.32 | 6.53 | 1.29 |
| CSTF3 | 71.39 | 228.17 | 11.82 | 28.96 | 4.26 | 1.29 |
| ZNF789 | 3.86 | 12.25 | 2.08 | 5.2 | 2.5 | 1.28 |
| UTS2 | 0 | 6.73 | 0.41 | 1.14 | 3.74 | 1.28 |
| TSEN34 | 6.76 | 5.45 | 0.66 | 1.75 | 2.87 | 1.28 |
| NMNAT1 | 6.92 | 20.03 | 1.47 | 3.71 | 3.68 | 1.28 |
| LUC7L | 21.44 | 47.3 | 1.14 | 2.91 | 5.26 | 1.28 |
| C2orf74 | 35.69 | 6.14 | 0.11 | 0.41 | 4.89 | 1.28 |
| STAP2 | 1.66 | 5.6 | 0.29 | 0.83 | 3.87 | 1.25 |
| ALPPL2 | 0 | 29.71 | 1.48 | 3.65 | 4.24 | 1.25 |
| ZNF735 | 0.05 | 352.37 | 13.97 | 33.19 | 4.65 | 1.24 |
| ZNF174 | 1.53 | 16.97 | 0.42 | 1.13 | 5.04 | 1.24 |
| PAGE5 | 0 | 26.69 | 0.73 | 1.86 | 5.01 | 1.24 |
| C16orf91 | 22.55 | 89.35 | 4.15 | 9.96 | 4.4 | 1.24 |
| SRA1 | 127.15 | 42.34 | 2.29 | 5.51 | 4.15 | 1.23 |
| CAB39L | 5.35 | 33.49 | 1.67 | 4.06 | 4.25 | 1.23 |
| ZCCHC10 | 19.07 | 93.41 | 6.67 | 15.69 | 3.79 | 1.22 |
| CLK4 | 5.85 | 95.8 | 8.56 | 19.9 | 3.47 | 1.21 |
| ZNF487P | 1.53 | 9.18 | 1.26 | 3.02 | 2.77 | 1.2 |
| PLD2 | 6.69 | 6 | 0.38 | 1 | 3.67 | 1.2 |
| LOC100506305 | 0.37 | 6.63 | 0.34 | 0.91 | 3.94 | 1.2 |
| KLF17 | 0.21 | 78.88 | 3.62 | 8.4 | 4.41 | 1.19 |
| BUD31 | 158.08 | 520.53 | 33.31 | 76.07 | 3.96 | 1.19 |
| AASDH | 4.18 | 6.74 | 1.19 | 2.84 | 2.41 | 1.19 |
| ZNF680 | 5.56 | 41.6 | 2.41 | 5.58 | 4.05 | 1.18 |
| WDR77 | 54 | 11.44 | 1.47 | 3.45 | 2.88 | 1.18 |
| EIF1AD | 9.39 | 115.55 | 4.12 | 9.43 | 4.78 | 1.18 |
| TMEM159 | 12.8 | 35.26 | 3.75 | 8.54 | 3.2 | 1.17 |
| STAG3L4 | 9.22 | 20.16 | 0.99 | 2.35 | 4.22 | 1.17 |
| FAM200A | 5.95 | 9.4 | 0.49 | 1.23 | 4.01 | 1.17 |
| NDUFAF2 | 135.33 | 38.57 | 2.8 | 6.37 | 3.74 | 1.16 |
| SC01 | 13.51 | 18.35 | 0.84 | 1.99 | 4.29 | 1.15 |
| NOC4L | 8.34 | 8.7 | 0.22 | 0.61 | 4.78 | 1.15 |
| LOC723809 | 0.06 | 17.08 | 1.67 | 3.84 | 3.28 | 1.15 |
| CCAR1 | 42.64 | 48.69 | 2.03 | 4.62 | 4.52 | 1.15 |
| TMEM41B | 28.8 | 128.12 | 8.64 | 19.19 | 3.87 | 1.14 |
| SAMD8 | 7.22 | 43.46 | 1.91 | 4.34 | 4.44 | 1.14 |
| DDX26B | 1.11 | 5.7 | 0.75 | 1.77 | 2.77 | 1.14 |
| TCEANC2 | 4.59 | 7.91 | 1.27 | 2.9 | 2.55 | 1.13 |
| SERTAD1 | 44.92 | 271.45 | 3.17 | 7.03 | 6.38 | 1.12 |
| GUSBP4 | 0.8 | 6.82 | 1.06 | 2.42 | 2.58 | 1.12 |
| ZNF273 | 1.16 | 22.99 | 2 | 4.43 | 3.46 | 1.11 |
| PDCD11 | 13.33 | 13.1 | 0.82 | 1.88 | 3.84 | 1.11 |
| MATR3 | 89.04 | 188.2 | 4.31 | 9.43 | 5.42 | 1.11 |
| LEMD3 | 3.09 | 5.62 | 0.22 | 0.59 | 4.16 | 1.11 |
| GUSBP1 | 9.68 | 107.53 | 3.64 | 7.96 | 4.85 | 1.11 |
| DNASE2 | 30.05 | 18.63 | 0.23 | 0.61 | 5.83 | 1.11 |
| SSX3 | 0 | 20.42 | 2.61 | 5.71 | 2.92 | 1.1 |
| FAM133B | 4.76 | 21.24 | 2.78 | 6.06 | 2.89 | 1.1 |
| CENPC1 | 9.51 | 38.75 | 3.4 | 7.41 | 3.47 | 1.1 |
| CCDC86 | 46.08 | 8.05 | 1.29 | 2.88 | 2.55 | 1.1 |
| TRIM39- | 1 | 5.9 | 0.54 | 1.26 | 3.23 | 1.09 |
| RPP21 | | | | | | |
| ECE2 | 15.32 | 33.28 | 0.5 | 1.18 | 5.8 | 1.09 |
| C17orf89 | 14.37 | 5.47 | 0.3 | 0.75 | 3.8 | 1.09 |
| BTK | 0.02 | 43.99 | 0.73 | 1.67 | 5.73 | 1.09 |
| ZNF669 | 3.46 | 131.98 | 3.79 | 8.14 | 5.09 | 1.08 |
| UTP3 | 12.64 | 39.85 | 1.36 | 2.98 | 4.77 | 1.08 |
| PRAMEF6 | 0.03 | 37.49 | 6.7 | 14.29 | 2.47 | 1.08 |
| XAGE5 | 0 | 14.36 | 0 | 0.11 | 7.18 | 1.07 |
| DEFB122 | 0 | 13.98 | 0.24 | 0.61 | 5.37 | 1.06 |
| PRAMEF10 | 0 | 140.56 | 23.74 | 49.42 | 2.56 | 1.05 |
| IFI30 | 22.64 | 151.39 | 12.34 | 25.62 | 3.61 | 1.05 |
| FASTKD5 | 9.26 | 78.82 | 2.08 | 4.41 | 5.18 | 1.05 |
| BEX2 | 0.16 | 10.46 | 1.24 | 2.68 | 2.98 | 1.05 |
| ZNF724P | 2.07 | 78.22 | 6.36 | 13.16 | 3.6 | 1.04 |
| ZNF92 | 6.55 | 165.41 | 10.67 | 21.84 | 3.94 | 1.03 |
| LOC100129515 | 0 | 25.79 | 4.52 | 9.35 | 2.49 | 1.03 |
| APOC1P1 | 0.12 | 5.44 | 0.16 | 0.43 | 4.41 | 1.03 |
| PRAMEF4 | 0 | 110.38 | 8.98 | 18.29 | 3.6 | 1.02 |
| GUCA1B | 0.37 | 11.28 | 0.36 | 0.83 | 4.63 | 1.02 |
| ELL2 | 30.17 | 7.07 | 0.64 | 1.39 | 3.28 | 1.01 |

### Example 5

After decades of efforts, human NT-ESCs were finally derived recently (Chung et al., 2014; Tachibana et al., 2013; Yamada et al., 2014). These advances were mainly due to optimization of SCNT derivation conditions. However, the intrinsic defects in epigenetic reprogramming that cause the developmental arrest of human SCNT embryos have not been identified. Herein, the inventors demonstrate that H3K9me3 in somatic cell genome presents a barrier for human SCNT reprogramming. Removal of this barrier by overexpressing the H3K9me3 demethylase, KDM4A, facilitates transcriptional reprogramming at ZGA, thereby allowing human SCNT embryos to develop more efficiently to generate blastocysts, from which the inventors successfully established multiple AMD patient-specific NT-ESC lines without compromising genomic stability or pluripotency. Thus, the inventors demonstrate that H3K9me3 as a general reprogramming barrier in reprogramming human somatic cells by SCNT, but also establishes a practical approach for improving cloning efficiency.

It has been well known that the ability of human oocytes to support SCNT embryo development varies greatly among oocyte donors. Indeed, human NT-ESCs can be derived only when high-quality oocytes donated by a small group of females were used as recipients (Chung et al., 2014; Tachibana et al., 2013; Yamada et al., 2014), although the reason for the dependence on oocyte quality remains elusive. Consistently, oocytes from only one (ID #58) out of the four donors supported SCNT blastocyst formation without KDM4A mRNA injection even under the presence of TSA, which has been reported to enhance blastocyst formation (Tachibana et al., 2013) (FIG. 2H and Table 4). In contrast, oocytes of all four donors tested supported blastocyst formation when KDM4A mRNAs were injected, indicating that KDM4A can overcome the donor variation problem. Whether KDM4A can improve IVF embryo development remains to be determined.

Although the developmental potential of human SCNT embryos reaching the blastocyst stage was significantly and consistently improved by KDM4A mRNA injection, the magnitude of improvement was not as drastic as that of mice (90% in mice vs. 27% in human). It is possible that species differences and/or the quality of human oocytes varies greatly even within the same batch of oocytes derived from a single ovulation, and only a fraction of them have the capacity to support development to blastocyst stage even by IVF, which has a varying success rate of 15-60% (Shapiro et al., 2002; Stone et al., 2014). This is in clear contrast to mouse IVF where more than 90% of embryos can develop to the blastocyst stage. Therefore, it is surprising that hSCNT efficiency was improved with KDM4A injection given the lower quality of the human oocytes as compared to the mouse oocytes. It is also possible that some of the human oocytes used in the experiments could not support blastocyst formation even by IVF.

In addition to demonstrating the efficacy of KDM4A in improving human SCNT efficiency and NT-ESC derivation, another important discovery is that KDM4A can facilitate human SCNT reprogramming. Considering that human KDM4A can function in mouse SCNT embryos to achieve a similar effect as KDM4d does, the inventors have demonstrated that all members of the KDM4 family can be used to facilitate hSCNT as long as they possess H3K9me3 demethylase activity.

In summary, the inventors herein have demonstrated an improved KDM4-assisted human SCNT method. Using this method, the inventors have derived human blastocysts from adult AMD patient cells and subsequently established multiple NT-ESCs (NTK-ESCs) with genomes identical to those of donor patients. This provides unique and important cell sources for understanding AMD as well as for therapeutic drug screening for AMD treatments. Given that the same strategy can be applied to the studies of other human diseases, the inventors have demonstrated a new method for generating patient-specific NT-ESCs which will have a general impact on human therapeutics. Additionally, since hSCNT allows replacement of somatic cell mitochondria with that of recipient oocyte, as demonstrated herein (FIGS. 3H-J and 7), the methods, compositions and kits as disclosed herein provides an opportunity to treat mitochondrial DNA-related diseases. Indeed, a recent study demonstrated that a metabolic syndrome phenotype caused by mtDNA mutation can be corrected by replacing mtDNA through SCNT (Ma et al., 2015). Thus, the KDM4-assisted SCNT method as disclosed herein is also useful for mtDNA-replacement therapies.

### REFERENCES

The references disclosed herein are incorporated in their entirety by reference.
Chung, Y., Klimanskaya, I., Becker, S., Li, T., Maserati, M., Lu, S. J., Zdravkovic, T., Ilic, D., Genbacev, O., Fisher, S., et al. (2008). Human Embryonic Stem Cell Lines Generated without Embryo Destruction. Cell Stem Cell 2, 113-117.
Matoba, S., Liu, Y., Lu, F., Iwabuchi, K. A., Shen, L., Inoue, A., and Zhang, Y. (2014). Embryonic Development following Somatic Cell Nuclear Transfer Impeded by Persisting Histone Methylation. Cell 159, 884-895.
Bernstein, B. E., Birney, E., Dunham, I., Green, E. D., Gunter, C., and Snyder, M.
(2012). An integrated encyclopedia of DNA elements in the human genome. Nature 489, 57-74. Chung, Y. G., Eum, J. H., Lee, J. E., Shim, S. H., Sepilian, V., Hong, S. W., Lee, Y., Treff, N. R., Choi, Y. H., Kimbrel, E. a, et al. (2014). Human Somatic Cell Nuclear Transfer Using Adult Cells. Cell Stem Cell 14, 777-780.
French, A. J., Adams, C. a, Anderson, L. S., Kitchen, J. R., Hughes, M. R., and Wood, S. H. (2008). Development of human cloned blastocysts following somatic cell nuclear transfer with adult fibroblasts. Stem Cells 26, 485-493.
Gurdon, J. B. (1962). The developmental capacity of nuclei taken from intestinal epithelium cells of feeding tadpoles. J. Embryol. Exp. Morphol. 10, 622-640.
Hochedlinger, K., and Jaenisch, R. (2003). Nuclear transplantation, embryonic stem cells, and the potential for cell therapy. N. Engl. J. Med. 349, 275-286.
Klose, R. J., Yamane, K., Bae, Y., Zhang, D., Erdjument-Bromage, H., Tempst, P., Wong, J., and Zhang, Y. (2006). The transcriptional repressor JHDM3A demethylates trimethyl histone H3 lysine 9 and lysine 36. Nature 442, 312-316.
Krishnan, S., and Trievel, R. C. (2013). Structural and functional analysis of JMJD2D reveals molecular basis for site-specific demethylation among JMJD2 demethylases. Structure 21, 98-108.
Ma, H., Folmes, C. D. L., Wu, J., Morey, R., Mora-Castilla, S., Ocampo, A., Ma, L., Poulton, J., Wang, X., Ahmed, R., et al. (2015). Metabolic rescue in pluripotent cells from patients with mtDNA disease. Nature.
Matoba, S., Liu, Y., Lu, F., Iwabuchi, K. A., Shen, L., Inoue, A., and Zhang, Y. (2014). Embryonic Development following Somatic Cell Nuclear Transfer Impeded by Persisting Histone Methylation. Cell 159, 884-895.
Niakan, K. K., Han, J., Pedersen, R. A., Simon, C., and Pera, R. A. R. (2012). Human pre-implantation embryo development. Development 841, 829-841.
Noggle, S., Fung, H.-L., Gore, A., Martinez, H., Satriani, K. C., Prosser, R., Oum, K., Paull, D., Druckenmiller, S., Freeby, M., et al. (2011). Human oocytes reprogram somatic cells to a pluripotent state. Nature 478, 70-75.
Rodriguez-Osorio, N., Urrego, R., Cibelli, J. B., Eilertsen, K., and Memili, E. (2012). Reprogramming mammalian somatic cells. Theriogenology 78, 1869-1886.
Shapiro, B. S., Richter, K. S., Harris, D. C., and Daneshmand, S. T. (2002). Implantation and pregnancy rates are higher for oocyte donor cycles after blastocyst-stage embryo transfer. Fertil. Steril. 77, 1296-1297.
Simerly, C., Dominko, T., Navara, C., Payne, C., Capuano, S., Gosman, G., Chong, K., Takahashi, D., Chace, C., Compton, D., et al. (2003). Molecular correlates of primate nuclear transfer failures. Science (80-.). 300, 297.
Stone, B. a., March, C. M., Ringler, G. E., Baek, K. J., and Marrs, R. P. (2014). Casting for determinants of blastocyst yield and of rates ofimplantation and of pregnancy after blastocyst transfers. Fertil. Steril. 102, 1055-1064.
Tachibana, M., Amato, P., Sparman, M., Gutierrez, N. M., Tippner-Hedges, R., Ma, H., Kang, E., Fulati, A., Lee, H.-S., Sritanaudomchai, H., et al. (2013). Human embryonic stem cells derived by somatic cell nuclear transfer. Cell 153, 1228-1238.
The Encode Consortium Project (2011). A user's guide to the encyclopedia of DNA elements (ENCODE). PLoS Biol. 9, e1001046.
Wakayama, T., Tabar, V., Rodriguez, I., Perry, a C., Studer, L., and Mombaerts, P. (2001). Differentiation of embryonic stem cell lines generated from adult somatic cells by nuclear transfer. Science 292, 740-743.
Wang, L., Miao, Y. L., Zheng, X., Lackford, B., Zhou, B., Han, L., Yao, C., Ward, J. M., Burkholder, A., Lipchina, I., et al. (2013). The THO complex regulates pluripotency gene mRNA export and controls embryonic stem cell self-renewal and somatic cell reprogramming. Cell Stem Cell 13, 676-690.
Whetstine, J R., Nottke, A., Lan, F., Huarte, M., Smolikov, S., Chen, Z., Spooner, E., Li, E., Zhang, G., Colaiacovo, M., et al. (2006). Reversal of histone lysine trimethylation by the JMJD2 family of histone demethylases. Cell 125, 467-481.
Xue, Z., Huang, K., Cai, C., Cai, L., Jiang, C., Feng, Y., Liu, Z., Zeng, Q., Cheng, L., Sun, Y. E., et al. (2013). Genetic programs in human and mouse early embryos revealed by single-cell RNA sequencing. Nature 500, 593-597.
Yamada, M., Hamatani, T., Akutsu, H., Chikazawa, N., Kuji, N., Yoshimura, Y., and Umezawa, A. (2009). Involvement of a novel preimplantation-specific gene encoding the high mobility group box protein Hmgpi in early embryonic development. Hum. Mol. Genet. 19, 480-493.
Yamada, M., Johannesson, B., Sagi, I., Burnett, L. C., Kort, D. H., Prosser, R. W., Paull, D., Nestor, M. W., Freeby, M., Greenberg, E., et al. (2014). Human oocytes reprogram adult somatic nuclei of a type 1 diabetic to diploid pluripotent stem cells. Nature 510, 533-536.
Yang, X., Smith, S. L., Tian, X. C., Lewin, H. A., Renard, J., and Wakayama, T. (2007). Nuclear reprogramming of cloned embryos and its implications for therapeutic cloning. Nat. Genet. 39, 295-302.

1. A method for increasing the efficiency of human somatic nuclear transfer (hSCNT) comprising contacting a hybrid oocyte with an agent which increases expression of a member of the KDM4 family of histone demethylases, wherein the hybrid oocyte is an enucleated human oocyte comprising the genetic material of a human somatic cell.

2. The method of claim 1, wherein the contacting occurs after activation or fusion of the hybrid oocyte, but before human zygotic genome activation (ZGA) begins.

3. A method for increasing the efficiency of human somatic cell nuclear transfer (SCNT)
comprising at least one of:
(iv) contacting a donor human somatic cell or a recipient human oocyte with at least one agent which decreases H3K9me3 methylation in the donor human somatic cell or the recipient human oocyte, wherein the recipient human oocyte is a nucleated or enucleated oocyte; enucleating the recipient human oocyte if the human oocyte is nucleated; transferring the nuclei from the donor human somatic cell to the enucleated occyte to form a hybrid oocyte; and activating the hybrid oocyte to form a human SCNT embryo; or
(v) contacting a hybrid oocyte with at least one agent which decreases H3K9me3 methylation in the hybrid oocyte, where the hybrid oocyte is an enucleated human oocyte comprising the genetic material of a human somatic cell, and activating the hybrid oocyte to form a human SCNT embryo; or
(vi) contacting a human SCNT embryo after activation with at least one agent which decreases H3K9me3 methylation in the human SCNT embryo, wherein the SCNT embryo is generated from the fusion of an enucleated human oocyte with the genetic material of a human somatic cell;
wherein the decrease of H3K9me3 methylation in any one of the donor human somatic cell, recipient human oocyte, hybrid oocyte or the human SCNT embryo increases the efficiency of the SCNT.

4. A method for producing a human nuclear transfer embryonic stem cell (hNT-ESC), comprising;
a. at least one of: (i) contacting a donor human somatic cell or a recipient human oocyte with at least one agent which decreases H3K9me3 methylation in the donor human somatic cell or the recipient human oocyte; wherein the recipient human oocyte is a nucleated or enucleated oocyte; enucleating the recipient human oocyte if the human oocyte is nucleated; transferring the nuclei from the donor human somatic cell to the enucleated oocyte to form a hybrid oocyte; and
activating the hybrid oocyte to form a human SCNT embryo; or
(ii) contacting a hybrid oocyte with at least one agent which decreases
H3K9me3 methylation in the hybrid oocyte, where the hybrid oocyte is an enucleated human oocyte comprising the genetic material of a human somatic cell, and activating the hybrid oocyte to form a human SCNT embryo; or
(iii) contacting a human SCNT embryo after activation with at least one agent
which decreases H3K9me3 methylation in the SCNT embryo, wherein the SCNT embryo is generated from the fusion of an enucleated human oocyte with the genetic material of a human somatic cell;
b. incubating the SCNT embryo for a sufficient amount of time to form a blastocyst; and collecting at least one blastomere from the blastocyst and culturing the at least one blastomere to form at least one human NT-ESC.

5. (canceled)

6. The method of claim 2, wherein in agent which decreases H3K9me3 methylation is an agent increases expression of a member of the human KDM4 family of histone demethylases.

7. The method of claim 6, wherein the agent increases the expression or activity of the human KDM4 (JMJD2) family of histone demethylases.

8. The method of claim 1, wherein the agent increases the expression or activity of at least one of: KDM4A (JMJD2A), KDM4B (JMJD2B), KDM4C (JMJD2C), KDM4D (JMJD4D) or KDM4E (JMJD2E).

9. The method of claim 1, wherein the agent increases the expression or activity of KDM4A (JMJD2A)

10. The method of claim 1, wherein the agent comprises a nucleic acid sequence corresponding to SEQ ID NO: 1-4 or SEQ ID NO: 45, or a biologically active fragment thereof which increases the efficiency of SCNT to a similar or greater extent as compared to the corresponding sequence of SEQ ID NO: 1-4 or SEQ ID NO: 45.

11. The method of claim 6, wherein the agent comprises a nucleic acid sequence corresponding to
SEQ ID NO: 1, or a biologically active fragment thereof which increases the efficiency of
SCNT to a similar or greater extent as compared to the nucleic acid sequence of SEQ ID NO: 1.

12. The method of claim 1, wherein the agent is an inhibitor of a H3K9 methyltransferase.

13. The method of claim 12, wherein the H3K9 methyltransferase is SUV39h1 or SUV39h2.

14. The method of claim 12, wherein the H3K9 methyltransferase is SETDB1.

15. The method of claim 12, wherein two or more of SUV39h1, SUV39h2 and SETDB1 are inhibited.

16. The method of claim 12, wherein the agent which inhibits H3K9 methyltransferase is selected
from the group consisting of; an RNAi agent, CRISPR/Cas9, CRISPR/Cpfl oligonucleotide,
neutralizing antibody or antibody fragment, aptamer, small molecule, peptide inhibitor,
protein inhibitor, avidimir, and functional fragments or derivatives thereof.

17. The method of claim 16, wherein the RNAi agent is a siRNA or shRNA molecule.

18. The method of claim 1, wherein the agent comprises a nucleic acid inhibitor to inhibit the expression of any of SEQ ID NOS: 14-16, 47, 49, 51, 52 or 53.

19. The method of claim 17, wherein the RNAi agent hybridizes to at least a portion of SEQ ID NOS: 14-16, 47, 49, 51, 52 or 53.

20. The method of claim 17, wherein the RNAi agent comprises any one of, or a combination of
nucleic acids of SEQ ID NO: 7, 8 or SEQ ID NO: 18 or 19 or a fragment of at least consecutive nucleic acid thereof, or a homologue having a sequence that is at least 80%
identical to SEQ ID NO: 7, 8 or SEQ ID NO: 18 or 19.

21. The method of claim 1, wherein the recipient human oocyte is an enucleated human oocyte.

22-73. (canceled)
